(19) United States
(12) Patent Application Publication
(10) Pub. No.: US 2025/0163032 A1
(43) Pub. Date: May 22, 2025

(54) NOVEL MOLECULES AS INHIBITORS OF DNA DAMAGE REPAIR PATHWAY

(71) Applicant: VRise Therapeutics, Inc., Cambridge, MA (US)

(72) Inventors: Prashant Kashinath BHAVAR, Hyderabad (IN); Anuj Ramesh Kshirsagar, Hyderabad (IN); Partha Pritam Sarma, Hyderabad (IN); Appaji Baburao Mandhare, Hyderabad (IN); Uday Kumar Surampudi, Hyderabad (IN)

(21) Appl. No.: 18/943,004

(22) Filed: Nov. 11, 2024

Related U.S. Application Data
(60) Provisional application No. 63/690,894, filed on Sep. 5, 2024.

(30) Foreign Application Priority Data
Nov. 10, 2023 (IN) 202341076811
Jan. 2, 2024 (IN) 202441000286
Jan. 9, 2024 (IN) 202441001631
May 11, 2024 (IN) 202441037320

(51) Int. Cl. C07D 403/14; A61K 31/4439; A61K 31/506; A61K 31/517; A61K 31/519; A61K 31/52; A61K 31/53; A61K 31/5377; A61P 35/00; C07D 401/12; C07D 401/14; C07D 403/12; C07D 473/34; C07D 487/04

(52) U.S. Cl. CPC C07D 403/14; A61K 31/4439; A61K 31/506; A61K 31/517; A61K 31/519; A61K 31/52; A61K 31/53; A61K 31/5377; A61P 35/00; C07D 401/12; C07D 401/14; C07D 403/12; C07D 473/34; C07D 487/04

(57) ABSTRACT

The present application relates to novel compounds described herein, the method of preparing the same, its pharmaceutical composition and method for use thereof. In particular, the invention relates to compounds of formula (I) or their pharmaceutically acceptable salts thereof as inhibitors of ubiquitin-specific protease 1 (USP1) protein and useful in treatment, prevention and/or amelioration of diseases or disorders associated with ubiquitin-specific protease 1 (USP1) including Cancer.

This application claims the benefit of priority to Indian provisional patent applications number 202341076811 filed dated 10 Nov. 2023; 202441000286 filed dated 2 Jan. 2024; 202441001631 filed dated 9 Jan. 2024; 202441037320 filed dated 11 May 2024 and U.S. provisional patent application No. 63/690,894 filed dated 5 Sep. 2024, the disclosures of each of which are incorporated herein by reference in their entirety.

### FIELD OF THE INVENTION

The present application relates to novel compounds described herein, the method of preparing the same, its pharmaceutical composition and method for use thereof. In particular, the invention relates to compounds of formula (I) or their pharmaceutically acceptable salts thereof as inhibitors of ubiquitin-specific protease 1 (USP1) protein and useful in treatment, prevention and/or amelioration of diseases or disorders associated with ubiquitin-specific protease 1 (USP1) including Cancer.

### BACKGROUND OF THE INVENTION

Ubiquitin is a small, highly conserved protein of 76 amino acids that is post-translationally conjugated to substrate proteins, including itself, via a three-step enzymatic reaction. The initial covalent attachment primarily occurs between the C-terminal glycine of ubiquitin and the F-amino group of lysine residue(s) of the target protein. Additional ubiquitin molecules can be ligated to one of the seven internal lysines of ubiquitin, resulting in diverse ubiquitin chain topologies. The biological outcome of ubiquitination is determined by both the length and linkage topology. For example, Lys48-linked polyubiquitin chains are almost exclusively associated with allocating proteins for proteasome-dependent degradation, while monoubiquitination or chains linked through different lysines have been shown to serve multiple nonproteolytic functions. Like other types of post-translational modifications, ubiquitination is a reversible process counter-regulated by enzymes known as deubiquitinases (DUBs), which catalyze the removal of ubiquitin from modified proteins. More importantly, dysfunction in ubiquitin-dependent signalling pathways has been linked to various human diseases, suggesting inhibition of ubiquitin pathway components as novel therapeutic targets for drug discovery.

Pharmacological intervention within the ubiquitin-proteasome system has been well established by the successful use of proteasome inhibitors for anticancer treatment. Nevertheless, the ubiquitin-proteasome system offers additional opportunities for therapeutic intervention that could afford increased specificity and the possibility for improved clinical efficacy, which proteasome inhibitors are currently lacking. The most apparent targets include enzymes involved in ubiquitin conjugation and deconjugation (i.e., ubiquitin ligases and DUBs), processes upstream of proteasome-mediated protein degradation.

Reversible protein ubiquitination is emerging as a key process for maintaining cell homeostasis, and the enzymes that participate in this process, in particular E3 ubiquitin ligases and deubiquitinases (DUBs), are increasingly being regarded as candidates for drug discovery. Human DUBs are a group of approximately 100 proteins, whose cellular functions and regulatory mechanisms remain, with some exceptions, poorly characterized. Among the DUBs, ubiquitin-specific protease 1 (USP1) has become an attractive anticancer target because of its involvement in regulating DNA damage response pathways.

USP1 is also one of the best-characterized human DUBs which plays an important role in the cellular response to DNA damage. USP1 levels, localization and activity are modulated through several mechanisms, including protein-protein interactions, autocleavage/degradation and phosphorylation, ensuring that USP1 function is carried out in a properly regulated spatio-temporal manner. Importantly, USP1 expression is deregulated in certain types of human cancer, suggesting that USP1 could represent a valid target in cancer therapy. (see Garcia-Santisteban et al. Molecular Cancer 2013, 12:91)

USP1 associates with UAF1 (USP1-associated factor 1), resulting in the heterodimeric USP1/UAF1 complex that is required for deubiquitinase activity. The USP1/UAF1 complex has been shown to regulate the tolerance of DNA damage induced by DNA cross-linking agents through deubiquitination of PCNA (proliferating cell nuclear antigen) and FANCD2 (Fanconi anemia complementation group D2), which are proteins that function in translesion synthesis and the Fanconi anemia pathway, respectively. Williams et al. also recently reported that USP1/UAF1 deubiquitinates and prevents proteasomal degradation of ID (inhibitor of DNA binding) proteins, which have been shown to activate multiple pathways involved in tumor progression, including preservation of the cancer stem cell phenotype. Moreover, evidence for aberrant overexpression of USP1 in several tumor types suggests that inhibitors of USP1 are likely to provide a therapeutic benefit. (see Thomas S. Dexheimer et. al., J. Med. Chem. 2014, 57, 8099-8110)

Various research groups around the world have been working on targeting USP1 and

Patent literature targeting USP1 include the following patents and/or patent applications: WO/2024/208292, WO/2024/153175, WO/2024/153250, WO/2024/146541, WO/2024/131742, WO/2024/094170, WO/2024/086790, WO/2024/083237, WO/2024/078436, WO/2024/064883, WO/2024/046471, WO/2024/041634, WO/2024/032647, WO/2024/022519, WO/2024/022266, WO/2024/006879, WO/2023/250084, WO/2023/216910, WO/2023/208130, WO/2023/196955, WO/2023/148643, WO/2023/155866, WO/2023/143424, WO/2023/066299, WO/2023/083285, WO/2023/083297, WO/2023/083286, WO/2023/147311, WO/2023/030295, WO/2022/216820, WO/2022/199652, WO/2022/253188, WO/2022/214053, WO/2022/174184, WO/2022/197892, WO/2022/228399, WO/2022/233263, WO/2021/163530, WO/2021/247606, WO/2020/132269, WO/2020/139988, WO/2017/087837, WO/2014/105952 and WO/2011/137320

Further review and literature disclosure on USP1 inhibitors have been given by see. Li, X Y. et al. Leukemia 37, 164-177 (2023), Mallard H J. Et al. Mol Cancer Res. 2023 Nov. 1; 21(11):1186-1204; Thomas S. Dexheimer et. al. J. Med. Chem. 2014, 57, 8099-8110; Qin Liang et. al., Nat Chem Biol. 2014 April; 10(4): 298-304; and Iraia Garcia-Santisteban et. al., Garcia-Santisteban et al. Molecular Cancer 2013, 12:91. All of these patents and/or patent applications and literature disclosures are incorporated herein as reference in their entirety for all purposes.

We believe, Cancer in general have very few options of treatment, especially when the cancer is a result of metastasis and is unresectable. On one end there have been several advancements in options of treatment for example use of chemotherapy either alone or in combination with radiation and/or surgery however on the other side there has been a significant amount of challenges with prognosis especially for the cancers such as the breast cancer, ovarian cancer, prostate cancer, lung cancer, gastric cancer, endometrial cancer, colorectal cancer.

Accordingly, there do exists an unmet medical need for treatments for such cancers and the present invention aims to address the same.

### SUMMARY OF THE INVENTION

The present invention relates to a compound of formula (I), or pharmaceutically acceptable salts or compositions and methods of treatment with them, in particular the present invention relates to a compound of formula (I) and their pharmaceutically acceptable salts thereof useful in the treatment, prevention and/or amelioration of diseases or disorders associated with USP1.

In one embodiment of the invention, the present invention is related to a compound of Formula (I):

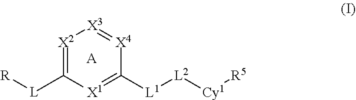

or a tautomer thereof, prodrug thereof, N-oxide thereof, a pharmaceutically acceptable ester thereof, a pharmaceutically acceptable salt thereof, or a stereoisomer thereof,
wherein
X¹ is CR¹ or N;
X² is CR², NR² or N;
X³ is CR³, NR³ or N;
X⁴ is CR⁴, NR⁴ or N;
each occurrence of R¹ is independently selected from hydrogen, hydroxy, halogen, CN (Cyano), substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino or any two R^{a} may be joined to a form a substituted or unsubstituted saturated or unsaturated 3-6 member ring, which may optionally include heteroatoms which may be same or different and are selected from 0, NR^{a} or S.
each occurrence of R², R³ and R⁴ are independently selected from hydrogen, hydroxy, halogen, CN (Cyano), substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino or any two R^{a} may be joined to a form a substituted or unsubstituted saturated or unsaturated 3-6 member ring, which may optionally include heteroatoms which may be same or different and are selected from O, NR^{a} or S or; any two variables of R² and R³ or R³ and R⁴ together with the ring they attached can form a 5 to 8 membered optionally substituted ring, which may optionally include one or more heteroatoms which may be the same or different and are selected from O, NR^{x} or S.
each occurrence of L, L¹ and L² are independently absent or independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₙ—, O, S, —S(═O)ₚ—, —C(═O)—, —NR^{x}—, —S(═O)ₚ—NH—, —NH—S(═O)ₚ—, —CO—NR^{x}— and —NR^{x}—CO—;
Cy¹ is selected from a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl;
each occurrence of R and R⁵ are independently selected from hydrogen, hydroxy, halogen, CN (Cyano), substituted or unsubstituted alkyl, substituted or unsubstituted alkoxy, substituted or unsubstituted haloalkyl, substituted or unsubstituted haloalkenyl, substituted or unsubstituted haloalkynyl, substituted or unsubstituted haloalkoxy, COOH, CONH₂, SO₃H, C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}C(═O)NR^{b}R^{c}, —NR^{b}S(═O)R^{c}, —NWS(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b}, —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring or substituted heterocyclylalkyl ring;
each occurrence of R^{a} is independently selected from hydrogen, hydroxy, halogen, cyano, carboxyl, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{c}OC(═O)R^{c}, —SR^{b}, —SOR^{c}, —SO₂R^{b}, —CR^{b}R^{c}C(═O)R^{b} or —CR^{b}R^{c}C(═S)R^{z};
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted haloalkyl, substituted or unsubstituted haloalkenyl, substituted or unsubstituted haloalkynyl, substituted or unsubstituted haloalkoxy, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{c} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio (═S) or imino (═NR^{x}) group;
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z′)—R^{a}, substituted or unsubstituted alkyl group, substituted or unsubstituted alkenyl, substituted or unsubstituted alkynyl, and substituted or unsubstituted Cycloalkyl; and
each occurrence of
n is 0, 1, 2, 3, 4, 5, 6, 7 or 8;
p, and q are independently 0.1 or 2.
   with the proviso that,
(a) when L is absent, L¹ is —NH— or —O—, then ring A in the compound of formula (I), is not furo[3,2-d]pyrimidine.
(b) when L is absent, L¹ is —NH—, L² is CH₂, Cy¹ is unsubstituted phenyl, X¹═X²═N, X³═CR³, X⁴═CR⁴, wherein R³ and R⁴ together form an unsubstituted phenyl, then
   (i) R in the compound of formula (I) is not 2-trifluoromethyl phenyl
   (ii) R⁵ in the compound of formula (I) is not unsubstituted pyridine-3-yl
(c) when X¹═X²═N, X³═CR³, X⁴═CR⁴, L is absent, L¹ is —NH—, L² is CH₂, Cy¹ is unsubstituted phenyl, R⁵ is unsubstituted pyridin-3-yl then R in the compound of formula (I) is not 2-isopropylphenyl.
(d) the compound of the formula (I) is not
   (i) 2-(2-isopropylphenyl)-N-(4-(1-methyl-4-(trifluoromethyl)-1H-imidazol-2-yl)benzyl)pyrido[3,2-d]pyrimidin-4-amine;
   (ii) 2-(4-cyclopropyl-6-methoxypyrimidin-5-yl)-4-((4-(1-methyl-4-(trifluoromethyl)-1H-imidazol-2-yl)benzyl)oxy)pyrido[3,2-d]pyrimidine; and
   (iii) 2-(2-isopropylphenyl)-N-(4-(pyridin-2-yl)benzyl)-5H-pyrrolo[3,2-d]pyrimidin-4-amine

Further preferred are compounds having the formula (I), wherein X¹ is CR¹ or N.

Further preferred are compounds having the formula (I), wherein X² is CR² or N.

Further preferred are compounds having the formula (I), wherein X³ is CR³ or N.

Further preferred are compounds having the formula (I), wherein X⁴ is CR⁴ or N.

Further preferred are compounds having the formula (I), wherein X² is N or NR².

Further preferred are compounds having the formula (I), wherein X³ is N or NR³.

Further preferred are compounds having the formula (I), wherein X⁴ is N or NR⁴.

Further preferred are compounds having the formula (I), wherein X² is NR² and X³ is CR³.

Further preferred are compounds having the formula (I), wherein X³ is NR³ and X⁴ is CR⁴.

Further preferred are compounds having the formula (I), wherein X² is NR² and X³ is CR³ wherein each of R² and R³ together with the ring they attached form a 5 to 8 membered optionally substituted ring, which may optionally include one or more heteroatoms which may be the same or different and are selected from O, NR^{x} or S.

Further preferred are compounds having the formula (I), wherein X³ is NR³ and X⁴ is CR⁴ wherein each of R³ and R⁴ together with the ring they attached form a 5 to 8 membered optionally substituted ring, which may optionally include one or more heteroatoms which may be the same or different and are selected from O, NR^{x} or S.

Further preferred are compounds having the formula (I), wherein X² is NR² and X³ is CR³ wherein each of R² and R³ together with the ring they attached form a 5 membered optionally substituted ring, which include one or more N atoms.

Further preferred are compounds having the formula (I), wherein X³ is NR³ and X⁴ is CR⁴ wherein each of R³ and R⁴ together with the ring they attached form a 5 membered optionally substituted ring, which include one or more N atoms.

Further preferred are compounds having the formula (I), wherein X² is CR² or N.

Further preferred are compounds having the formula (I), wherein X⁴ is CR⁴ or N.

Further preferred are compounds having the formula (I), wherein X² and X³ are selected from CR² and CR³ respectively, where in each of R² and R³ together with the ring they attached form a 4 to 7 membered optionally substituted ring preferably a 4 to 7 membered optionally substituted cycloalkyl ring.

Further preferred are compounds having the formula (I), wherein X³ and X⁴ are selected from CR³ and CR⁴ respectively, where in each of R³ and R⁴ together with the ring they attached form a 4 to 7 membered optionally substituted ring preferably a 4 to 7 membered optionally substituted cycloalkyl ring.

Further preferred are compounds having the formula (I), wherein X² and X³ are selected from CR² and CR³ respectively, where in each of R² and R³ together with the ring they attached form a Cycloalkyl ring.

Further preferred are compounds having the formula (I), wherein X³ and X⁴ are selected from CR³ and CR⁴ respectively, where in each of R³ and R⁴ together with the ring they attached form a Cycloalkyl ring.

Further preferred are compounds having the formula (I), wherein any two variables of R² and R³ together with the ring they attached can form a 5 to 8 membered optionally substituted ring, which may optionally include one or more heteroatoms which may be the same or different and are selected from O, NR^{x} or S.

Further preferred are compounds having the formula (I), wherein any two variables of R³ and R⁴ together with the ring they attached can form a 5 to 8 membered optionally substituted ring, which may optionally include one or more heteroatoms which may be the same or different and are selected from O, NR^{x} or S.

Further preferred are compounds having the formula (I), wherein R is substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring or substituted heterocyclylalkyl ring;

Further preferred are compounds having the formula (I), wherein R⁵ is substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring or substituted heterocyclylalkyl ring;

In one embodiment of the invention, the present invention is related to a compound of Formula (IA) or (IB):

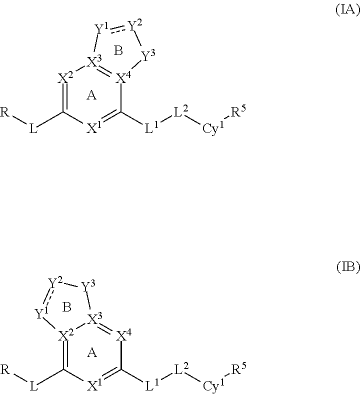

or a tautomer thereof, prodrug thereof, N-oxide thereof, a pharmaceutically acceptable ester thereof, a pharmaceutically acceptable salt thereof, or a stereoisomer thereof,
wherein
Y¹ is —CR^{d}, —NR^{d}, —CR^{d}R^{e}—CR^{d}R^{e}—, —CR^{d}R^{e}—N—, —N—CR^{d}R^{e}—, —NR^{d}—NR^{d}—, —CR^{d}═CR^{d}—, —CR^{d}N—, —N═CR^{d}, —N═N—, —O—, —S— or —N—;
Y² is —CR^{d}, —NR^{d}—, —CR^{d}R^{e}—CR^{d}R^{e}—, —CR^{d}R^{e}—N—, —N—CR^{d}R^{e}—, —NR^{d}—NR^{e}, —CR^{d}═CR^{d}—, —CR^{a}═N—, —N═CR^{a}—, —N═N—, —O—, —S— or —N—;
Y³ is —CR^{d}, —NR^{d}, —CR^{d}R^{e}—CR^{d}R^{e}—, —CR^{d}R^{e}—N—, —N—CR^{d}R^{e}—, —NR^{d}—NR^{d}—, —CR^{d}═CR^{d}—, —CR^{d}═N—, —N═CR^{d}—, —N═N—, —O—, —S— or —N—;
each occurrence of R^{d} or R^{e} are independently selected from hydrogen, hydroxy, halogen, oxo, CN (Cyano), substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino or any two R^{a} may be joined to a form a substituted or unsubstituted saturated or unsaturated 3-6 member ring, which may optionally include heteroatoms which may be same or different and are selected from 0, NR^{a} or S; any two variables of R^{d} and R^{c} together with the ring they attached can form an oxo (═O) or a 3 to 8 membered optionally substituted ring, which may optionally include one or more heteroatoms which may be the same or different and are selected from O, NR^{x} or S.
unless otherwise stated, all the other variables (X¹, X², X³, X⁴, L, L¹, L², R, R⁵, and Cy¹) are independently as defined above in relation to a compound of formula (I);
   with the proviso that,
(a) when L is absent, L¹ is —NH— or —O—, then ring A in the compound of formula (IA) is not furo[3,2-d]pyrimidine.
(b) when L is absent, L¹ is —NH—, L² is CH₂, Cy¹ is unsubstituted phenyl, X¹═X²═N, X³═CR³, X⁴═CR⁴, wherein R³ and R⁴ together form an unsubstituted phenyl, then
   (i) R in the compound of formula (IA) is not 2-trifluoromethyl phenyl
   (ii) R⁵ in the compound of formula (IA) is not unsubstituted pyridine-3-yl
(c) when X¹═X²═N, X³═CR³, X⁴═CR⁴, L is absent, L¹ is —NH—, L² is CH₂, Cy¹ is unsubstituted phenyl, R⁵ is unsubstituted pyridin-3-yl then R in the compound of formula (IA) is not 2-isopropylphenyl.
(d) the compound of the formula (IA) is not
   (i) 2-(2-isopropylphenyl)-N-(4-(1-methyl-4-(trifluoromethyl)-1H-imidazol-2-yl)benzyl)pyrido[3,2-d]pyrimidin-4-amine;
   (ii) 2-(4-cyclopropyl-6-methoxypyrimidin-5-yl)-4-((4-(1-methyl-4-(trifluoromethyl)-1H-imidazol-2-yl)benzyl)oxy)pyrido[3,2-d]pyrimidine; and
   (iii) 2-(2-isopropylphenyl)-N-(4-(pyridin-2-yl)benzyl)-5H-pyrrolo[3,2-d]pyrimidin-4-amine.

In one embodiment of the invention, the present invention is related to a compound of Formula (IA-1), (IA-2), (IB-1) or (IB-2):

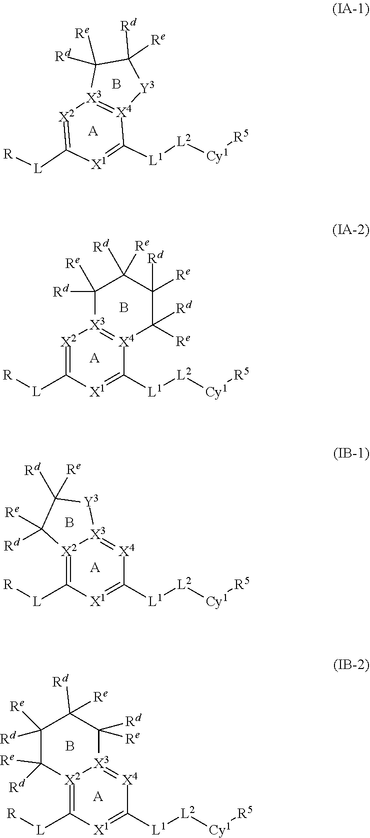

or a tautomer thereof, prodrug thereof, N-oxide thereof, a pharmaceutically acceptable ester thereof, a pharmaceutically acceptable salt thereof, or a stereoisomer thereof, wherein
unless otherwise stated, all the variables (X¹, X², X³, X⁴, Y³, L, L¹, L², R, R⁵, R^{d}, R^{e} and Cy¹) independently are as defined above in relation to a compound of formula (I), (IA) or (IB).

Further preferred are compounds having the formula (I), (IA), (IA-1), (IA-2), (IB-1) or (IB-2), wherein X¹ is CR¹ or N.

Further preferred are compounds having the formula (I), (IA), (IB), (IA-1), (IA-2), (IB-1) or (IB-2), wherein X¹ is CR¹, or N wherein R¹ is H or CN.

Further preferred are compounds having the formula (I), (IA), (IB), (IA-1), (IA-2), (IB-1) or (IB-2), wherein X¹ is N.

Further preferred are compounds having the formula (T), (IA), (IB), (IA-1), (IA-2), (IB-1) or (IB-2), wherein X² is CR² or N.

Further preferred are compounds having the formula (I), (IA), (IB), (IA-1), (IA-2), (IB-1) or (IB-2), wherein X² is CR², or N wherein R² is H or CN.

Further preferred are compounds having the formula (I), (IA), (IB), (IA-1), (IA-2), (IB-1) or (IB-2), wherein X² is N.

Further preferred are compounds having the formula (I), (IA), (IB), (IA-1), (IA-2), (IB-1) or (IB-2), wherein X³ is CR³ or N.

Further preferred are compounds having the formula (I), (IA), (IB), (IA-1), (IA-2), (IB-1) or (IB-2), wherein X³ is CR³, or N wherein R³ is H or CN.

Further preferred are compounds having the formula (I), (IA), (IB), (IA-1), (IA-2), (IB-1) or (IB-2), wherein X³ is CH or N.

Further preferred are compounds having the formula (I), (IA), (IB), (IA-1), (IA-2), (IB-1) or (IB-2), wherein X³ is CH.

Further preferred are compounds having the formula (I), (IA), (IB), (IA-1), (IA-2), (IB-1) or (IB-2), wherein X⁴ is CR⁴ or N.

Further preferred are compounds having the formula (I), (IA), (IB), (IA-1), (IA-2), (IB-1) or (IB-2), wherein X⁴ is CR⁴, or N wherein R⁴ is H or CN.

Further preferred are compounds having the formula (I), (IA), (IB), (IA-1), (IA-2), (IB-1) or (IB-2), wherein X⁴ is CH.

Further preferred are compounds having the formula (I), (IA), (IB), (IA-1), (IA-2), (IB-1) or (IB-2), wherein Y¹ is selected from —CR^{d}, —NR^{d}, —CR^{d}R^{e}—CR^{d}R^{e}—, —CR^{d}R^{e}—N—, —N—CR^{d}R^{e}—, —O—, —S— or —N—.

Further preferred are compounds having the formula (I), (IA), (IB), (IA-1), (IA-2), (IB-1) or (IB-2), wherein Y² is selected from —CR^{d}, —NR^{d}, —CR^{d}R^{e}—CR^{d}R^{e}—, —CR^{d}R^{e}—N—, —N—CR^{d}R^{e}—, —O—, —S— or —N—.

Further preferred are compounds having the formula (I), (IA), (IB), (IA-1), (IA-2), (IB-1) or (IB-2), wherein Y³ is selected from —CR^{d}, —NR^{d}, —CR^{d}R^{e}—CR^{d}R^{e}—, —CR^{d}R^{e}—N—, N—CR^{d}R^{e}—, —O—, —S— or —N—.

Further preferred are compounds having the formula (I), (IA), (IB), (IA-1), (IA-2), (IB-1) or (IB-2), wherein R^{d} is selected from hydrogen, hydroxy, halogen, oxo, CN (Cyano) or substituted or unsubstituted alkyl;

Further preferred are compounds having the formula (I), (IA), (IB), (IA-1), (IA-2), (IB-1) or (IB-2), wherein R^{e} is selected from hydrogen, hydroxy, halogen, oxo, CN (Cyano) or substituted or unsubstituted alkyl;

Further preferred are compounds having the formula (I), (IA), (IB), (IA-1), (IA-2), (IB-1) or (IB-2), wherein
(i) R^{d} is hydrogen and R^{e} is halogen;
(ii) R^{d} is halogen and R^{e} is halogen;
(iii) R^{d} is hydrogen and R^{e} is substituted or unsubstituted alkyl;
(iv) R^{d} is halogen and R^{e} is substituted or unsubstituted alkyl;
(v) R^{d} is substituted or unsubstituted alkyl and R^{e} is substituted or unsubstituted alkyl;
(vi) both R^{d} and R^{e} together form an oxo (═O);
(vii) R^{d} is hydrogen and R^{e} is hydroxy;
(viii) R^{d} is hydroxy and R^{e} is hydroxy;
(ix) R^{d} is hydroxy and R^{e} is halogen; or
(x) R^{d} is hydroxy and R^{e} is substituted or unsubstituted alkyl;

Further preferred are compounds having the formula (I), (IA), (IB), (IA-1), (IA-2), (IB-1) or (IB-2), wherein
(i) R^{d} is hydrogen and R^{e} is fluorine or chlorine
(ii) R^{d} is fluorine or chlorine and R^{e} is fluorine or chlorine
(iii) R^{d} is hydrogen and R^{e} is methyl or ethyl
(iv) R^{d} is fluorine or chlorine and R^{e} is methyl or ethyl
(v) R^{d} is methyl or ethyl and R^{e} is methyl or ethyl
(vi) both R^{d} and R^{e} together form an oxo (═O);
(vii) R^{d} is hydrogen and R^{e} is hydroxy;
(viii) R^{d} is hydroxy and R^{e} is hydroxy;
(ix) R^{d} is hydroxy and R^{e} is fluorine or chlorine; or
(x) R^{d} is hydroxy and R^{e} is methyl or ethyl;

Further preferred are compounds having the formula (I), (IA), (IB), (IA-1), (IA-2), (IB-1) or (IB-2), wherein L is absent or selected from substituted or unsubstituted C₁₋₄ alkyl, 0, S, —S(═O)ₚ or NR.

Further preferred are compounds having the formula (I), (IA), (IB), (IA-1), (IA-2), (IB-1) or (IB-2), wherein L is absent or selected from substituted or unsubstituted C₁₋₄ alkyl.

Further preferred are compounds having the formula (I), (IA), (IB), (IA-1), (IA-2), (IB-1) or (IB-2), wherein L is absent.

Further preferred are compounds having the formula (I), (IA), (IB), (IA-1), (IA-2), (IB-1) or (IB-2), wherein R is selected from substituted or unsubstituted aryl or substituted or unsubstituted heteroaryl.

Further preferred are compounds having the formula (I), (IA), (IB), (IA-1), (IA-2), (IB-1) or (IB-2) wherein ring A including with variables X¹, X², X³ and X⁴ is selected from

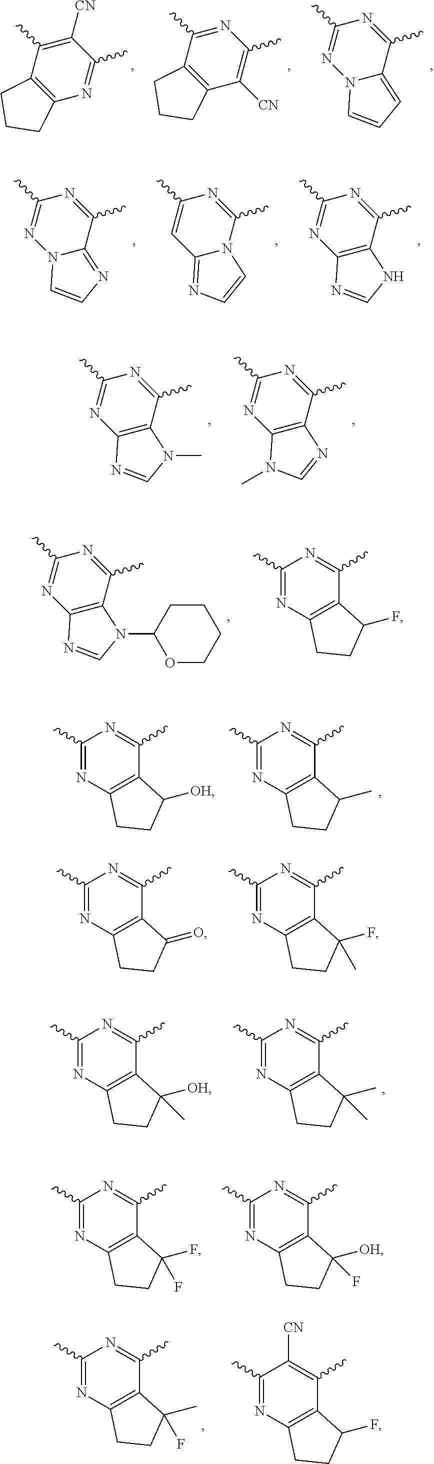

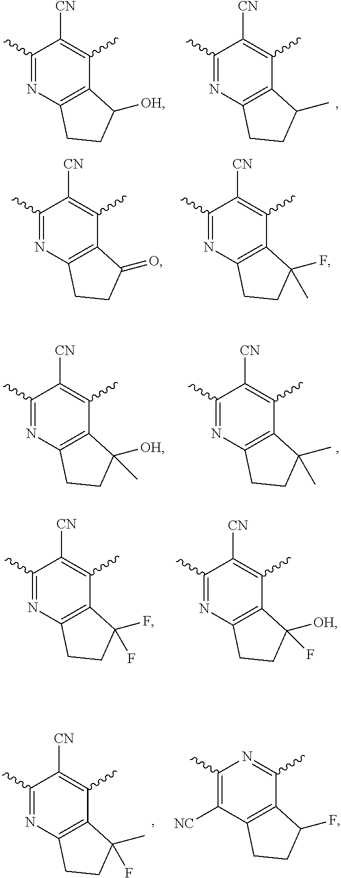

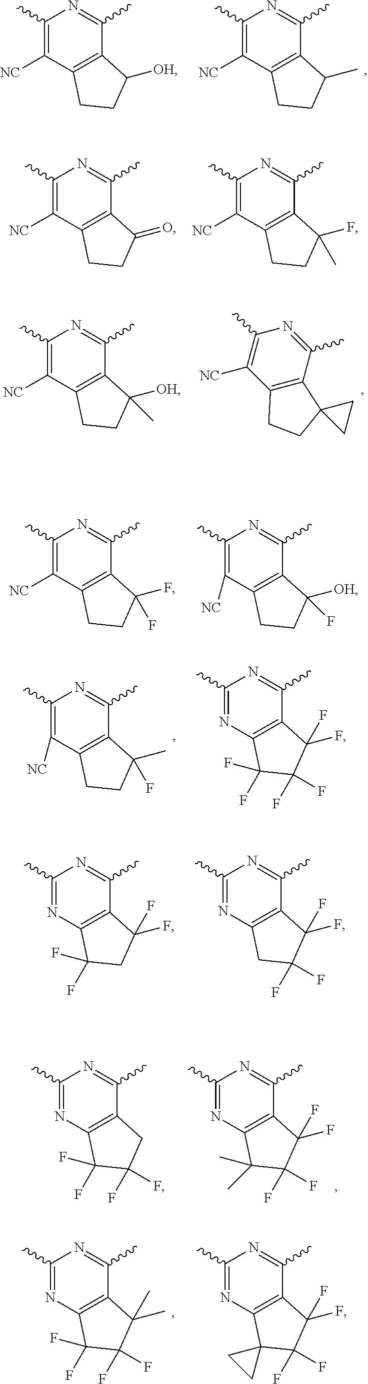

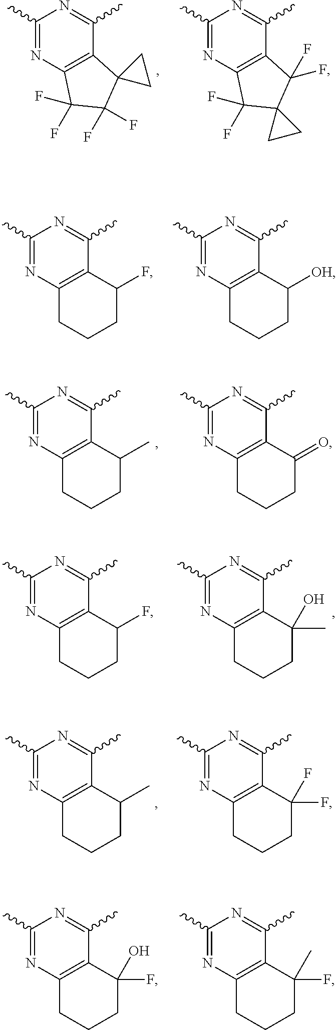

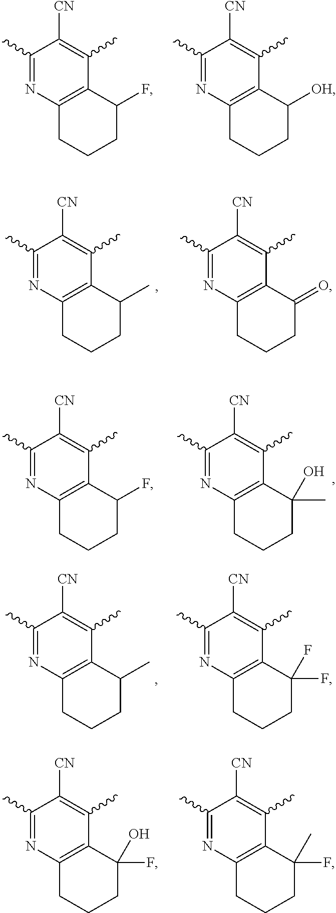

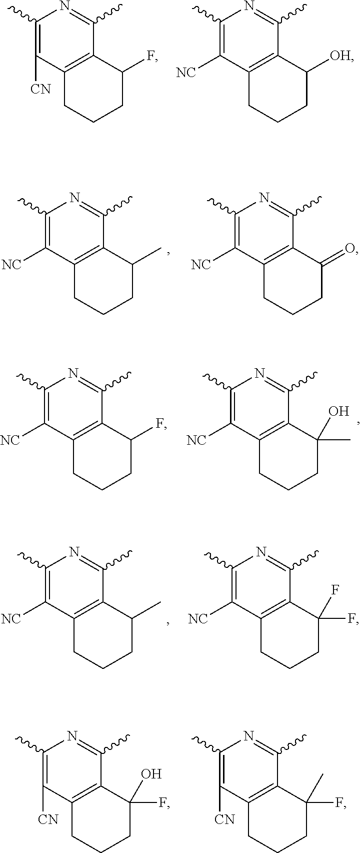

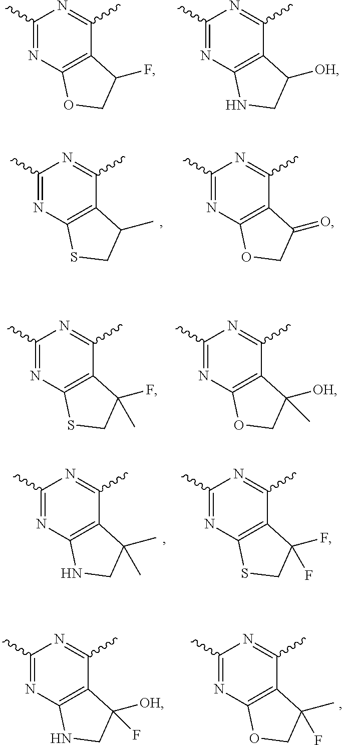

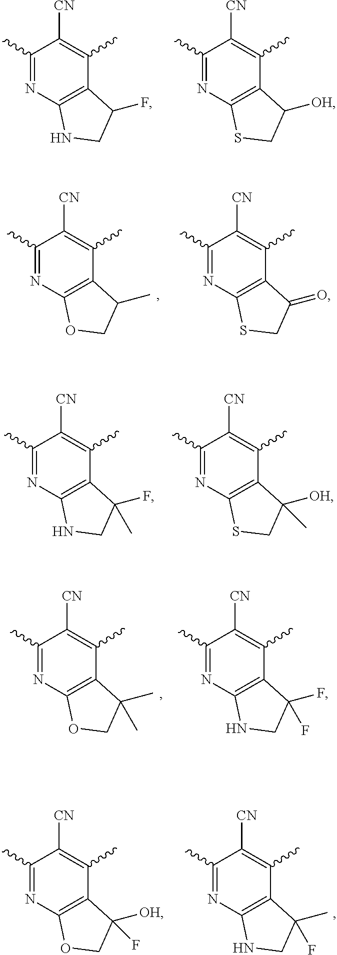

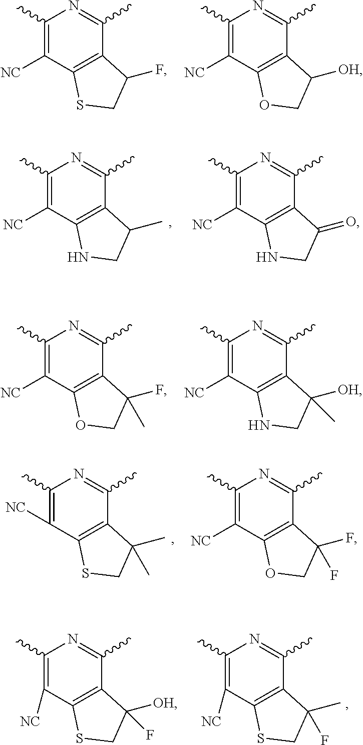

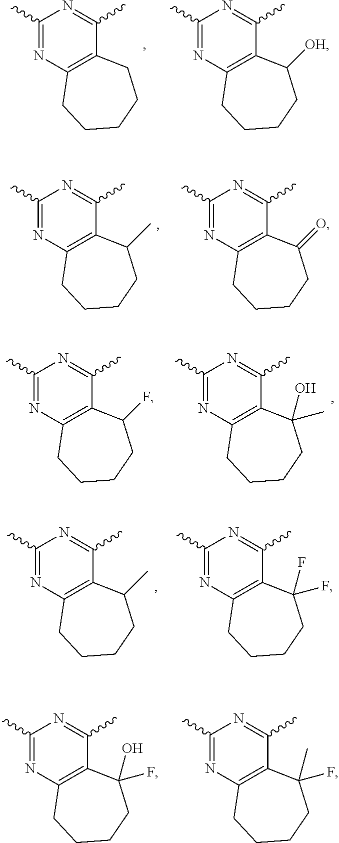

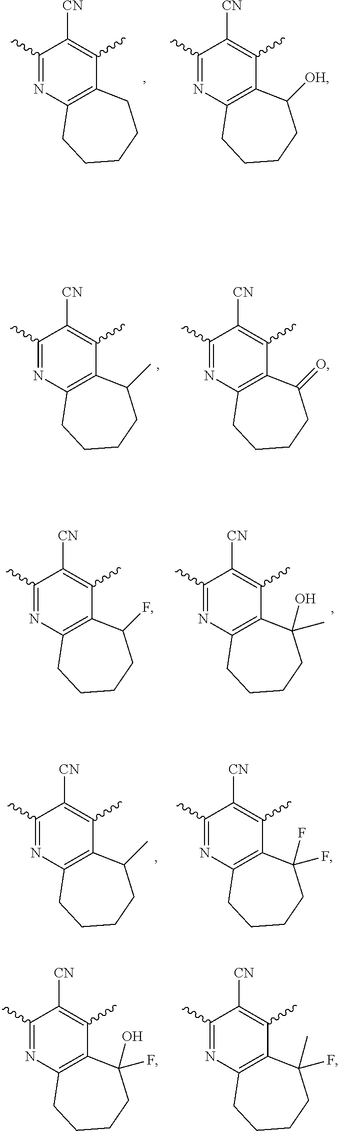

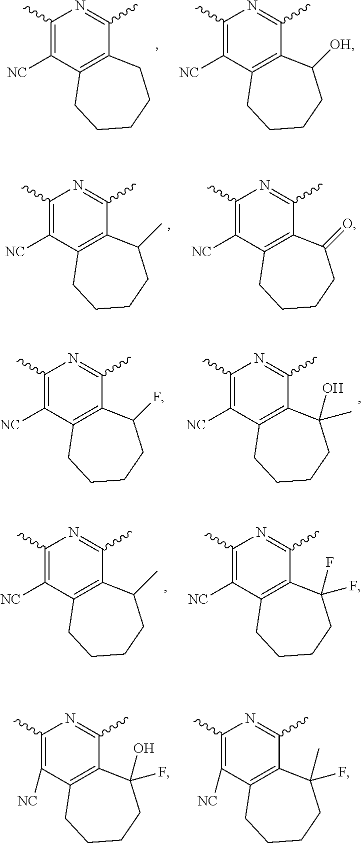

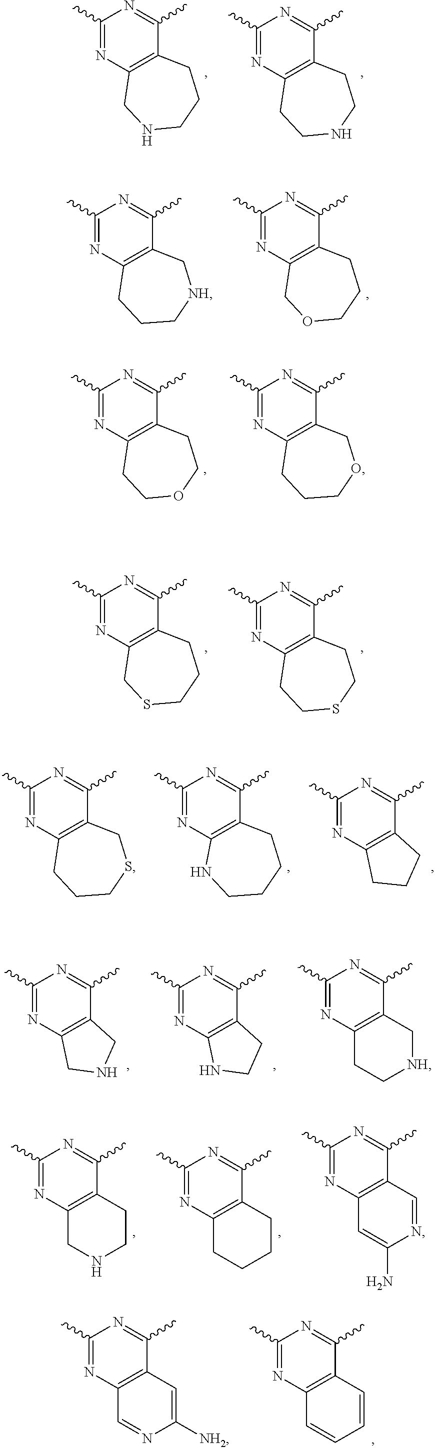

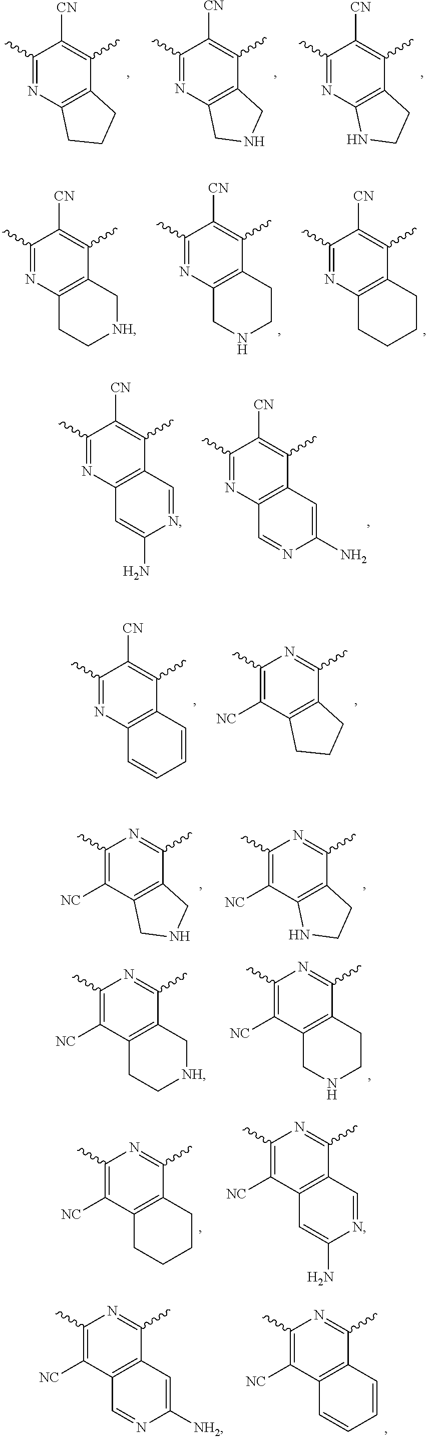

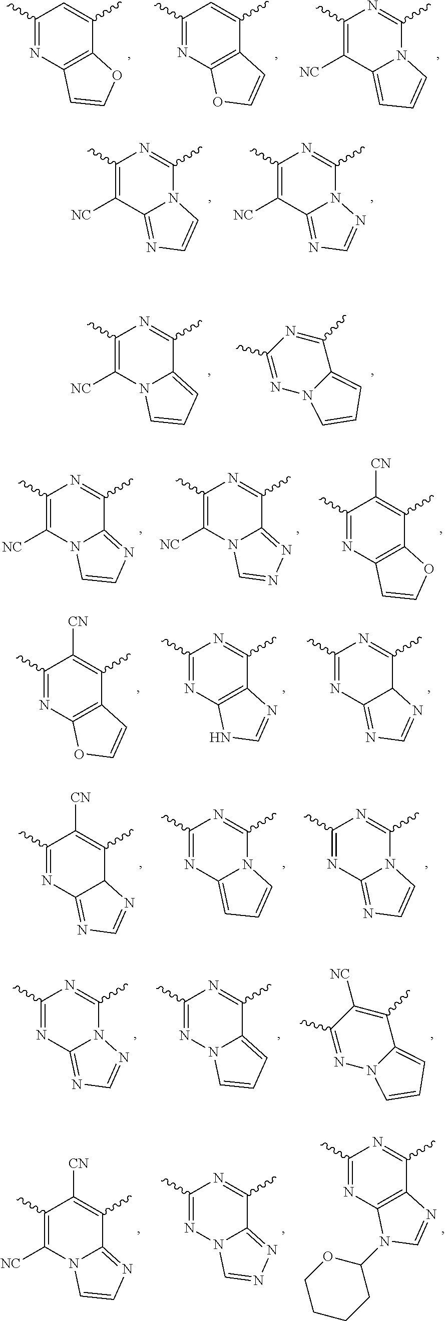

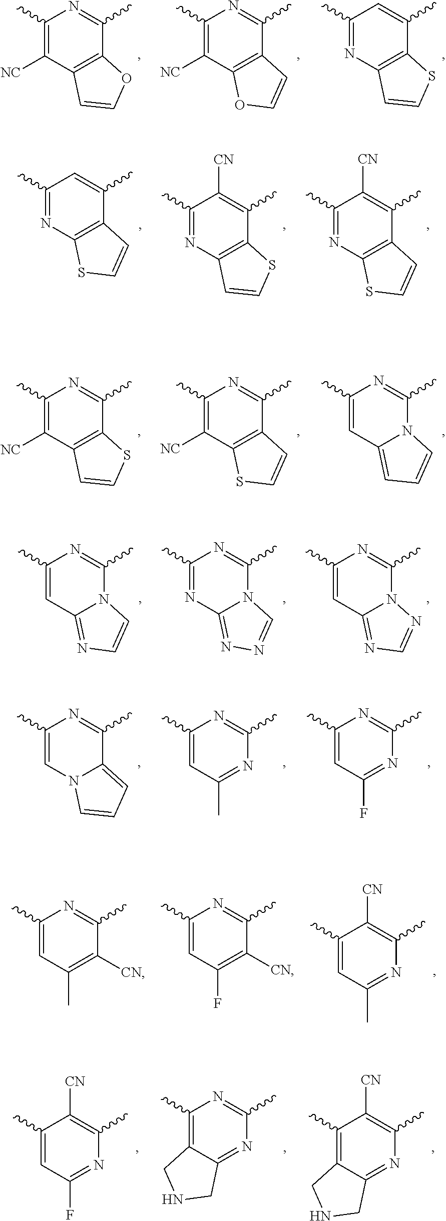

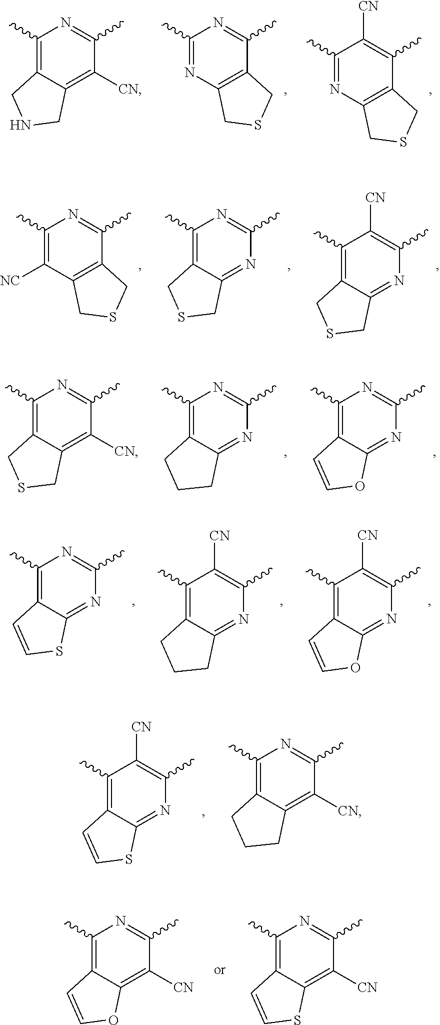

Further preferred are compounds having the formula (I), (IA), (IB), (IA-1), (IA-2), (IB-1) or (IB-2), wherein ring A including with variables X¹, X², X³ and X⁴ is selected from

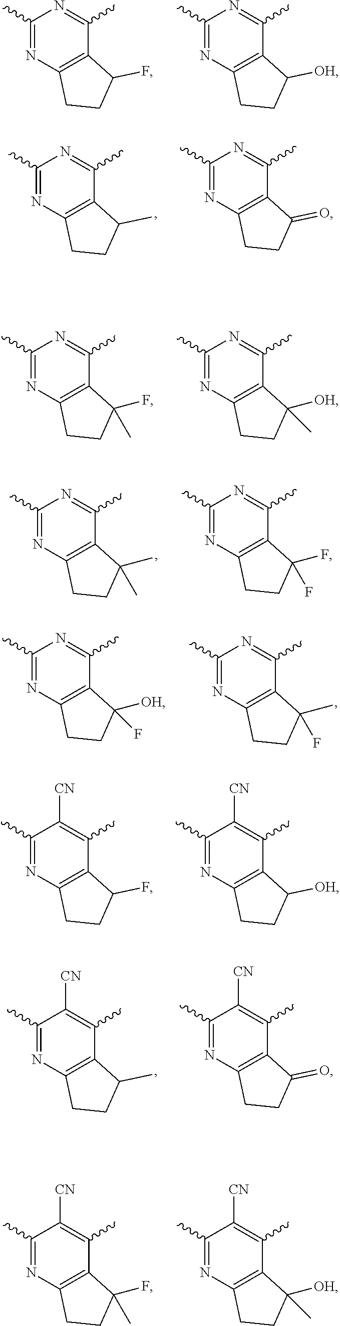

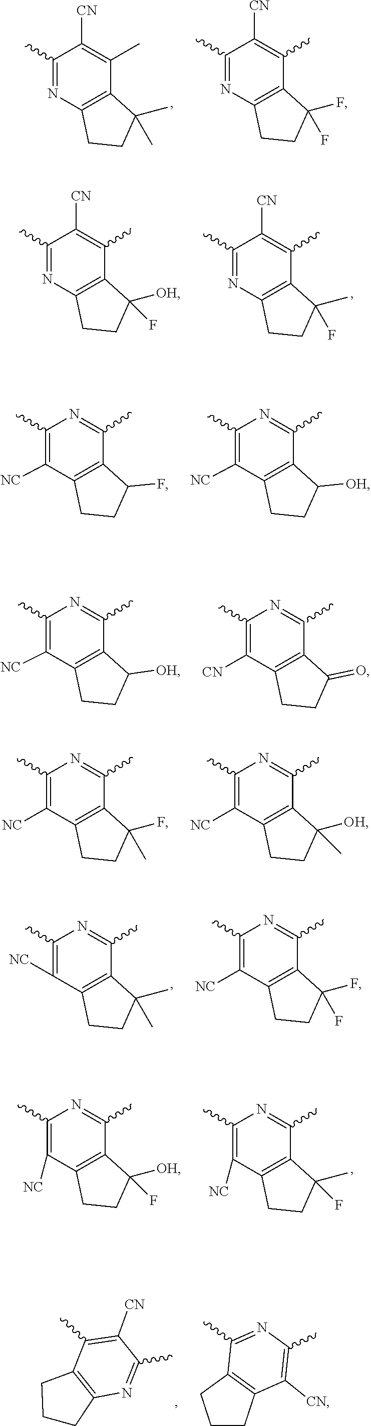

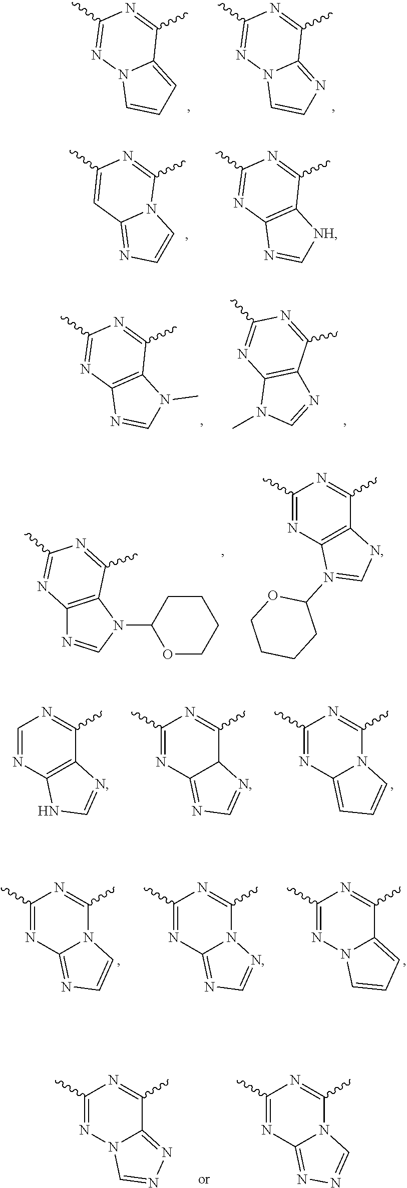

Further preferred are compounds having the formula (I), (IA), (IB), (IA-1), (IA-2), (IB-1) or (IB-2), wherein ring A including with variables X¹, X², X³ and X⁴ is selected from

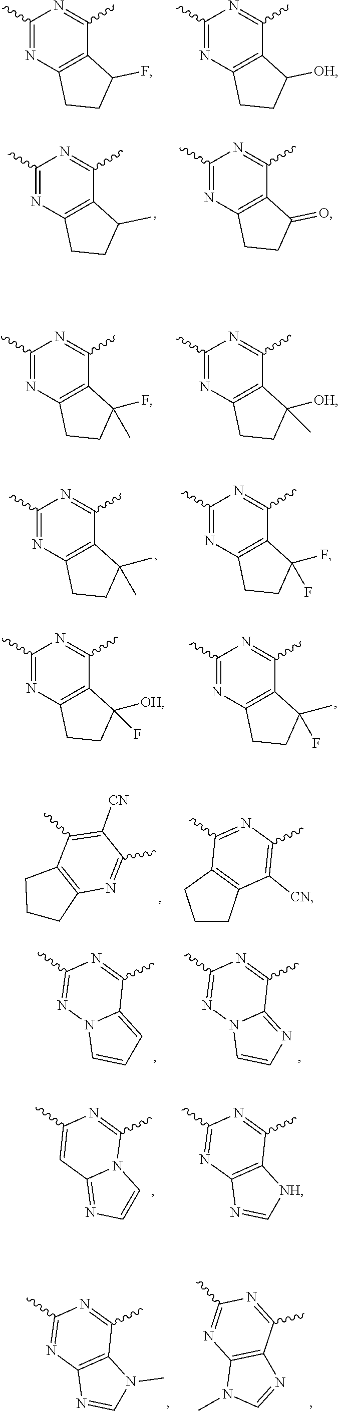

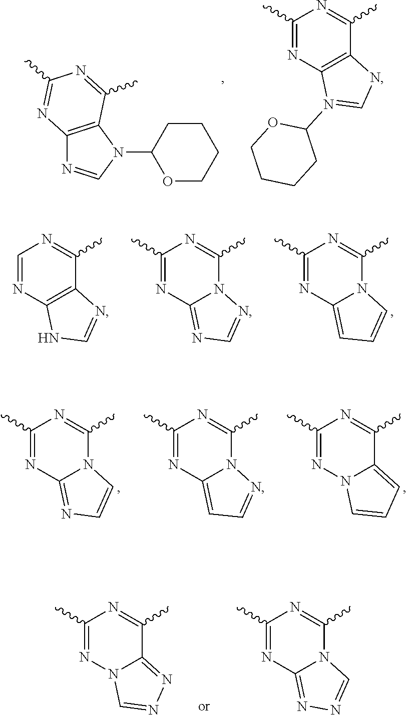

Further preferred are compounds having the formula (I), (IA), (IB), (IA-1), (IA-2), (IB-1) or (IB-2), wherein ring A including with variables X¹, X², X³ and X⁴ is selected from

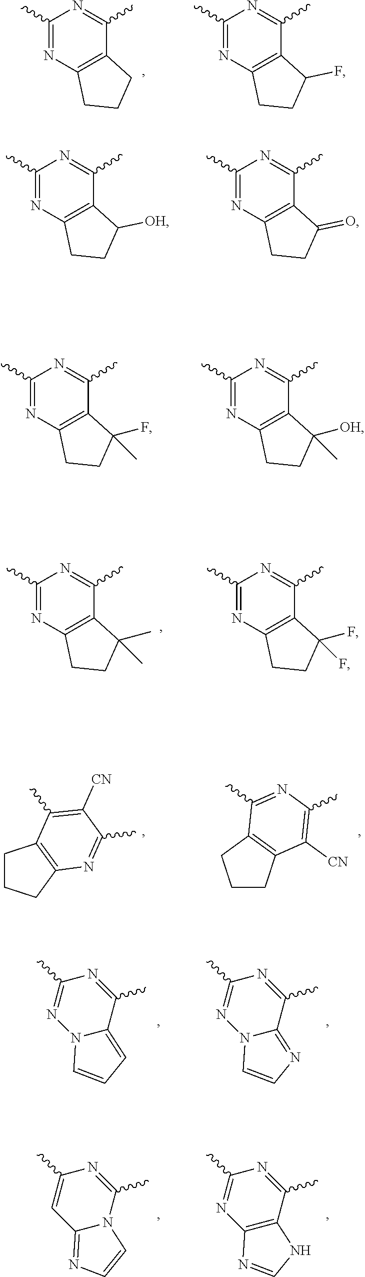

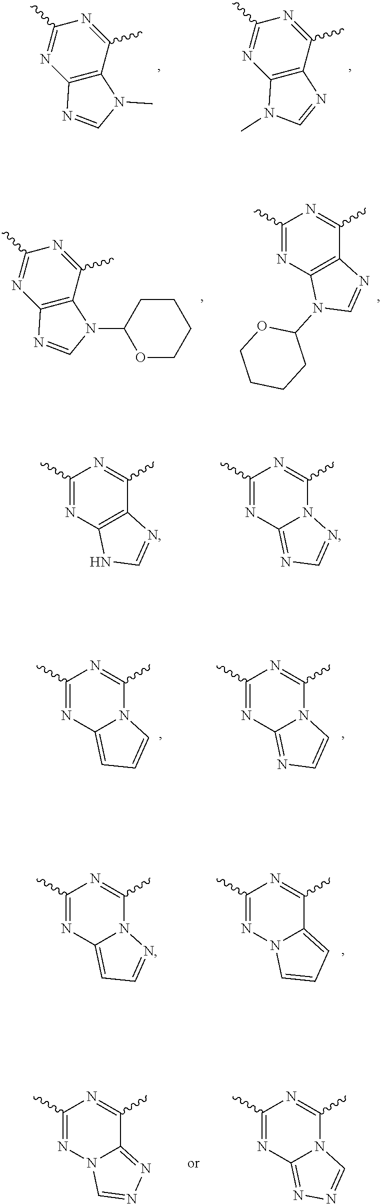

Further preferred are compounds having the formula (I), (IA), (IB), (IA-1), (IA-2), (IB-1) or (IB-2), wherein ring A including with variables X¹, X², X³ and X⁴ is selected from

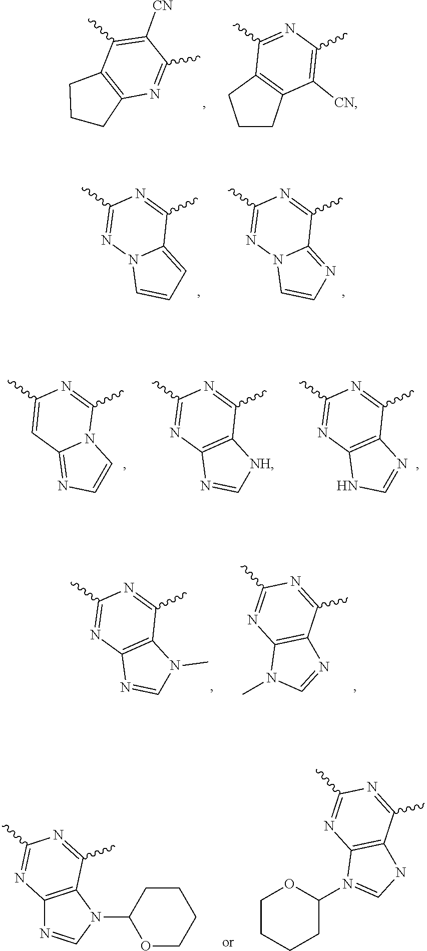

Further preferred are compounds having the formula (I), (IA), (IB), (IA-1), (IA-2), (IB-1) or (IB-2), wherein R is selected from selected from substituted or unsubstituted aryl or substituted or unsubstituted heteroaryl.

Further preferred are compounds having the formula (I), (IA), (IB), (IA-1), (IA-2), (IB-1) or (IB-2), wherein R is selected from

hydrogen, halogen,

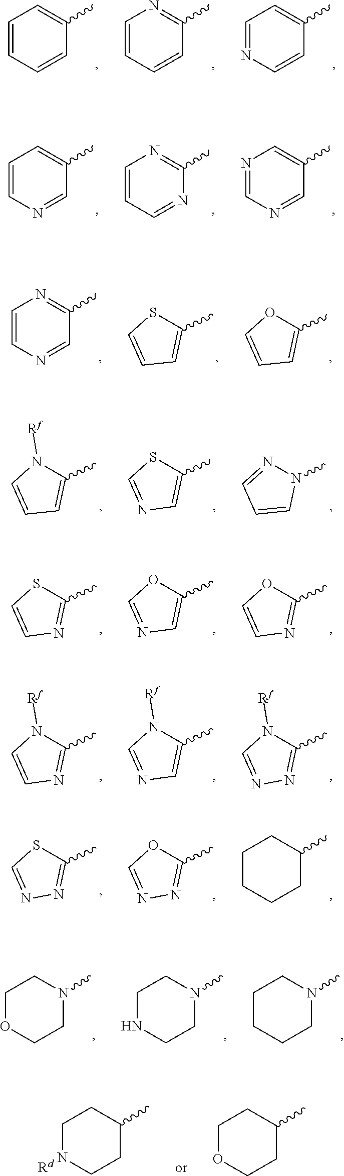

wherein R is optionally substituted with one or more R^{f};
   each occurrence of R^{f} is independently selected from hydrogen, hydroxy, halogen, CN (Cyano), substituted or unsubstituted alkyl, substituted or unsubstituted alkoxy, substituted or unsubstituted haloalkyl, substituted or unsubstituted haloalkenyl, substituted or unsubstituted haloalkynyl, substituted or unsubstituted haloalkoxy, COOH, CONH₂, SO₃H, C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —ROC(═O)R^{c}, —SR^{b}, —SOR^{c}, —SO₂R^{b}, —CR^{b}R^{c}C(═O)R^{b}, —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring or substituted heterocyclylalkyl ring;

Further preferred are compounds having the formula (I), (IA), (IB), (IA-1), (IA-2), (IB-1) or (IB-2), wherein R is selected from hydrogen, halogen,

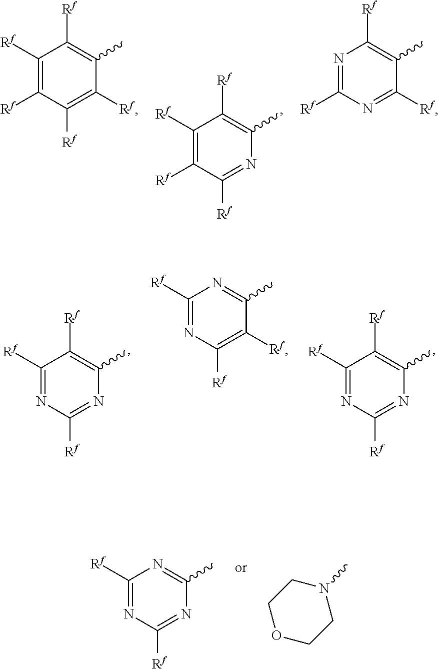

Further preferred are compounds having the formula (I), (IA), (IB), (IA-1), (IA-2), (IB-1) or (IB-2), wherein R is selected from hydrogen, fluorine, chlorine,

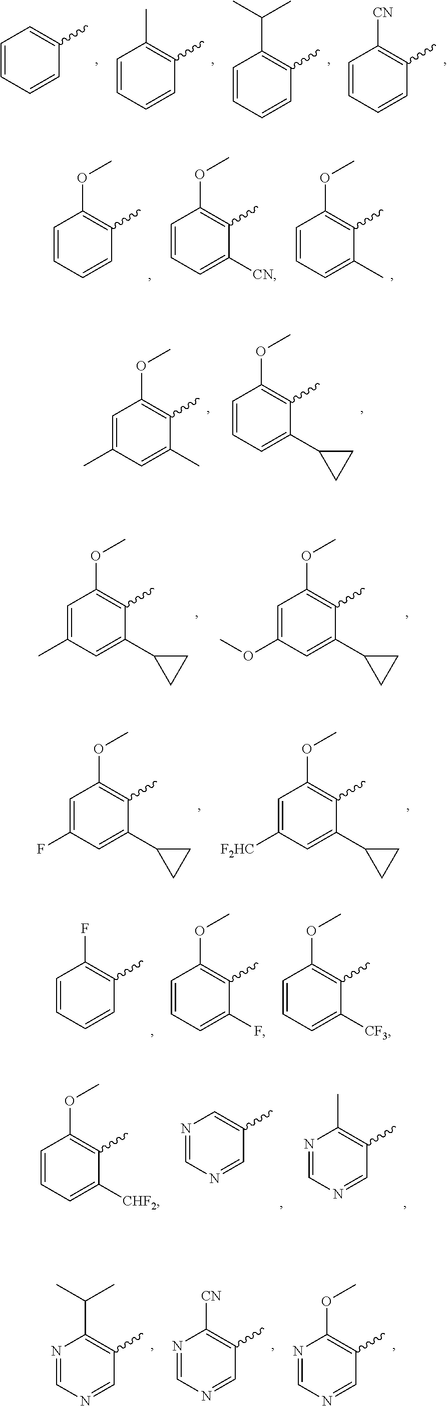

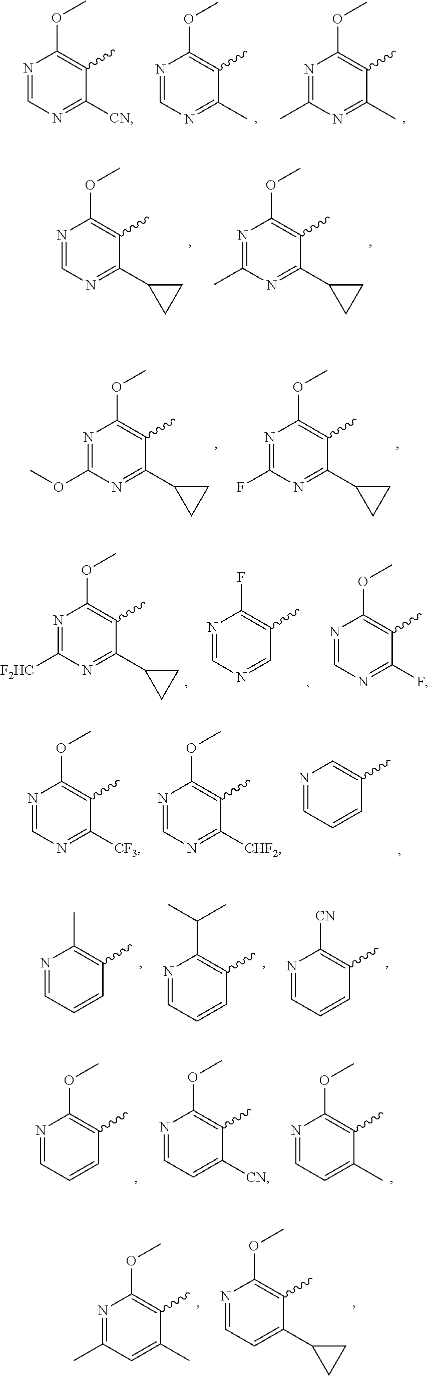

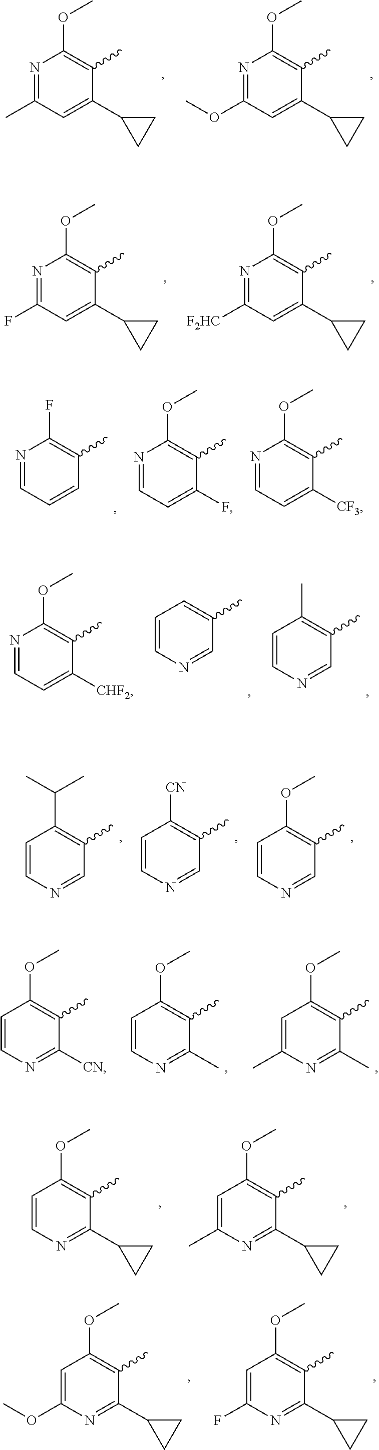

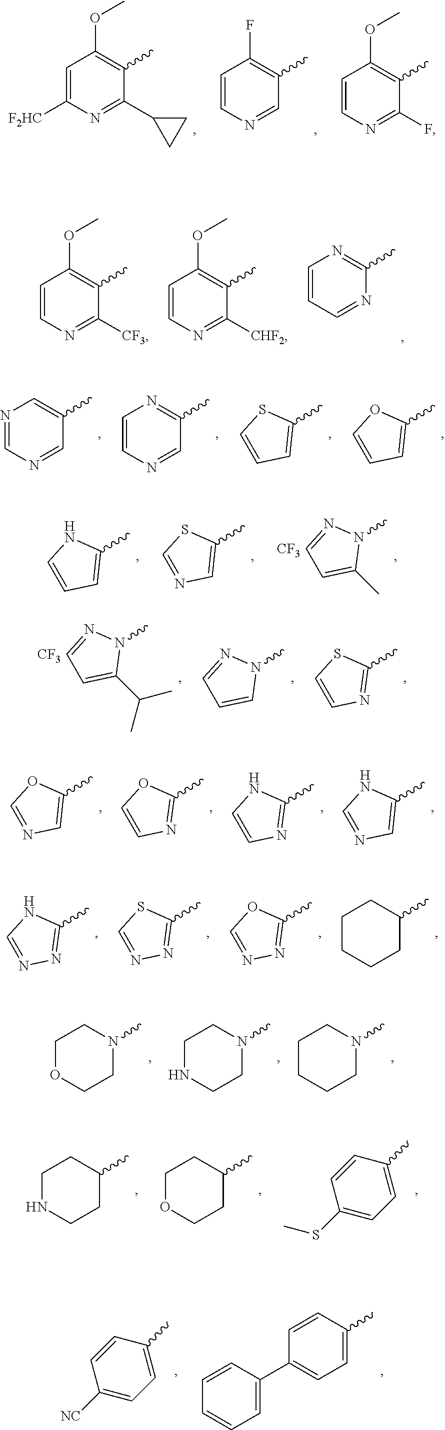

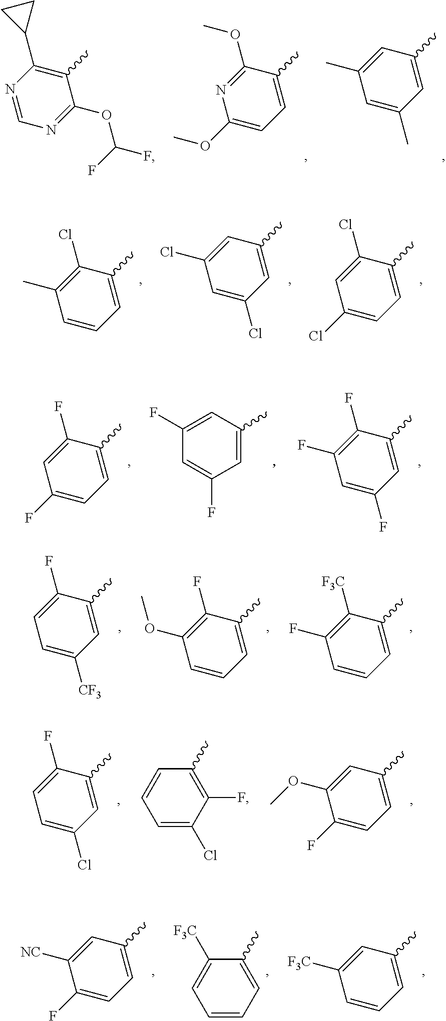

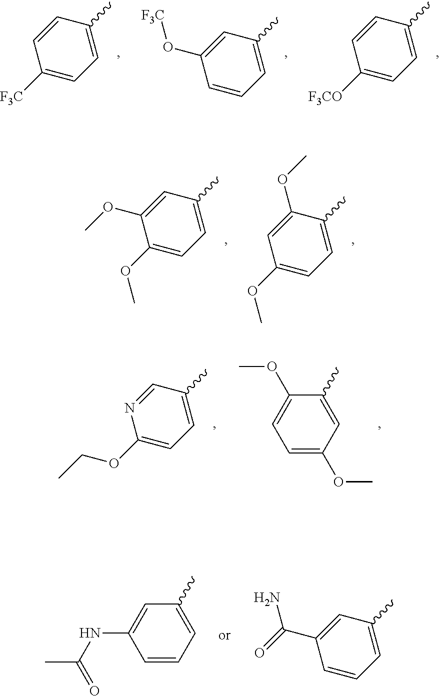

Further preferred are compounds having the formula (I), (IA), (IB), (IA-1), (IA-2), (IB-1) or (IB-2), wherein R is selected from hydrogen, hydroxy, halogen, CN (Cyano), substituted or unsubstituted alkyl, substituted or unsubstituted alkoxy, substituted or unsubstituted haloalkyl, substituted or unsubstituted cycloalkyl, COOH, CONH₂, —OR^{b}, —SR^{b}, —SOR^{c} or —SO₂R^{b}.

Further preferred are compounds having the formula (I), (IA), (IB), (IA-1), (IA-2), (IB-1) or (IB-2), wherein R^{f} is selected from hydrogen, halogen, substituted or unsubstituted alkyl, substituted or unsubstituted alkoxy or substituted or unsubstituted cycloalkyl.

Further preferred are compounds having the formula (I), (IA), (IB), (IA-1), (IA-2), (IB-1) or (IB-2), wherein Cy¹ is selected from substituted or unsubstituted aryl or substituted or unsubstituted heteroaryl.

In one embodiment of the invention, the present invention is related to a compound of Formula (IA-1A), or (IA-1B):

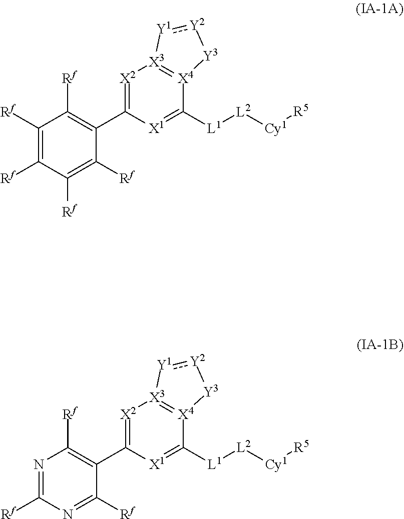

or a tautomer thereof, prodrug thereof, N-oxide thereof, a pharmaceutically acceptable ester thereof, a pharmaceutically acceptable salt thereof, or a stereoisomer thereof,
wherein
Cy¹ is selected from

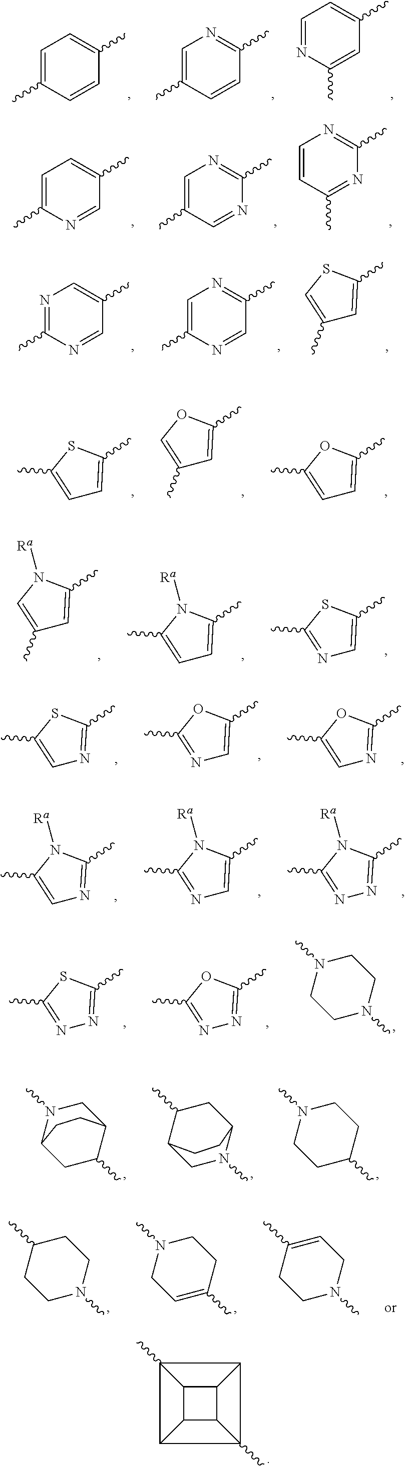

wherein Cy is optionally substituted with one or more R^{a}.
   and unless otherwise stated all the other variables (X¹, X², X³, X⁴, Y¹, Y², Y³, L¹, L², R⁵, R^{a}, R^{d} and R^{f}) independently are as defined above in relation to a compound of formula (I), (IA), (IB), (IA-1), (IA-2), (IB-1) or (IB-2);
      with the proviso that,
(a) when L is absent, L¹ is —NH— or —O—, then ring A in the compound of formula (IA-1A), or (IA-1B) is not furo[3,2-d]pyrimidine.
(b) when L is absent, L¹ is —NH—, L² is CH₂, Cy¹ is unsubstituted phenyl, X¹═X²═N, X³═CR³, X⁴═CR⁴, wherein R³ and R⁴ together form an unsubstituted phenyl, then
   (i) R in the compound of formula (IA-1A), or (IA-1B) is not 2-trifluoromethyl phenyl
   (ii) R⁵ in the compound of formula (IA-1A), or (IA-1B) is not unsubstituted pyridine-3-yl
(c) when X¹═X²═N, X³═CR³, X⁴═CR⁴, L is absent, L¹ is —NH—, L² is CH₂, Cy¹ is unsubstituted phenyl, R⁵ is unsubstituted pyridin-3-yl then R in the compound of formula (IA-1A), or (IA-1B) is not 2-isopropylphenyl.
(d) the compound of the formula (IA-1A), or (IA-1B) is not
   (i) 2-(2-isopropylphenyl)-N-(4-(1-methyl-4-(trifluoromethyl)-1H-imidazol-2-yl)benzyl)pyrido[3,2-d]pyrimidin-4-amine;
   (ii) 2-(4-cyclopropyl-6-methoxypyrimidin-5-yl)-4-((4-(1-methyl-4-(trifluoromethyl)-1H-imidazol-2-yl)benzyl)oxy)pyrido[3,2-d]pyrimidine; and
   (iii) 2-(2-isopropylphenyl)-N-(4-(pyridin-2-yl)benzyl)-5H-pyrrolo[3,2-d]pyrimidin-4-amine

In one embodiment of the invention, the present invention is related to a compound of Formula (IA-1A1), or (IA-1B1):

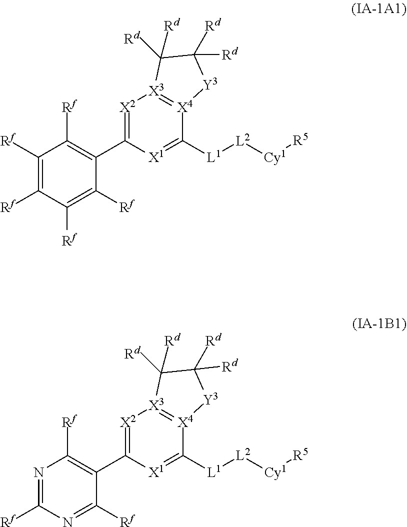

or a tautomer thereof, prodrug thereof, N-oxide thereof, a pharmaceutically acceptable ester thereof, a pharmaceutically acceptable salt thereof, or a stereoisomer thereof,
wherein
Cy¹ is selected from

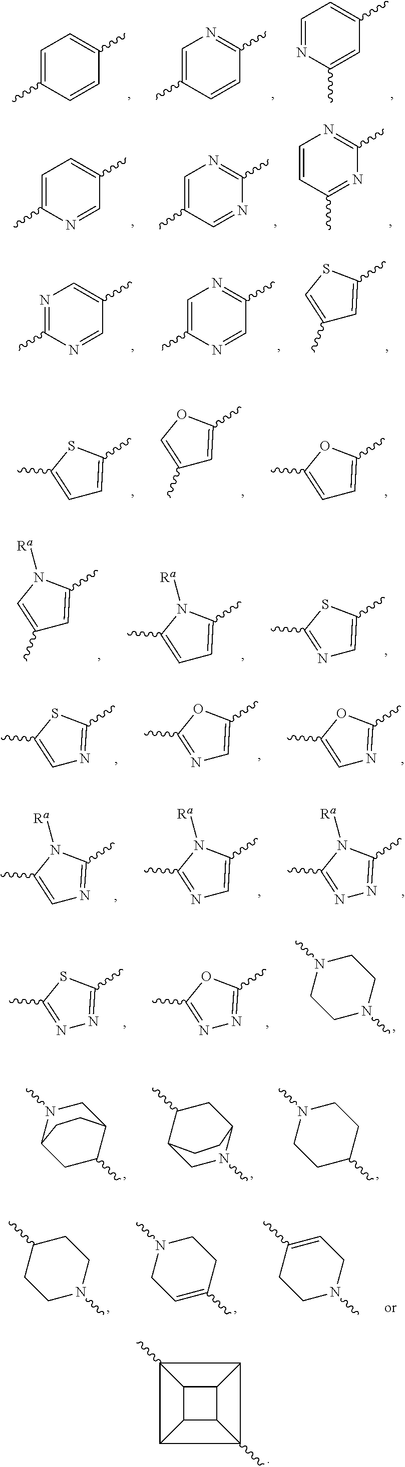

wherein Cy¹ is optionally substituted with one or more R^{a}
   unless otherwise stated all the other variables (X¹, X², X³, X⁴, Y¹, Y², Y³, L¹, L², R⁵, R^{a}, R^{d} and R^{f}) are as defined above in relation to a compound of formula (I), (IA), (IB), (IA-1), (IA-2), (IB-1), (IB-2), (IA-1A), or (IA-1B).

In one embodiment of the invention, the present invention is related to a compound of Formula (IA-1A2) or (IA-1B2).

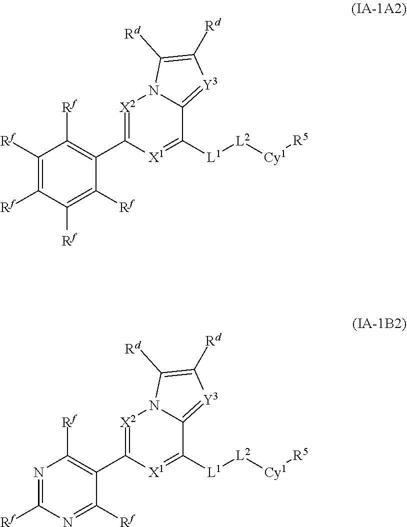

or a tautomer thereof, prodrug thereof, N-oxide thereof, a pharmaceutically acceptable ester thereof, a pharmaceutically acceptable salt thereof, or a stereoisomer thereof,
wherein
Cy¹ is selected from

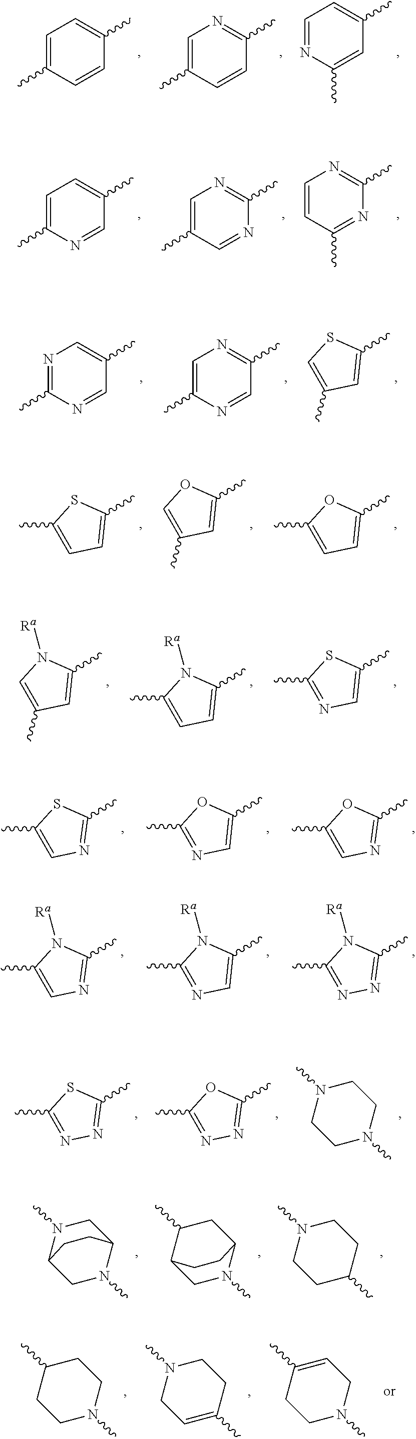

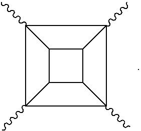

wherein Cy is optionally substituted with one or more R^{a}.
   unless otherwise stated all the other variables (X¹, X², X³, X⁴, Y¹, Y², Y³, L¹, L², R⁵, R^{a}, R^{d} and R^{f}) are as defined above in relation to a compound of formula (I), (IA), (IB), (IA-1), (IA-2), (IB-1), (IB-2), (IA-1A), or (IA-1B).

In one embodiment of the invention, the present invention is related to a compound of Formula (IA-1A3), or (IA-1B3):

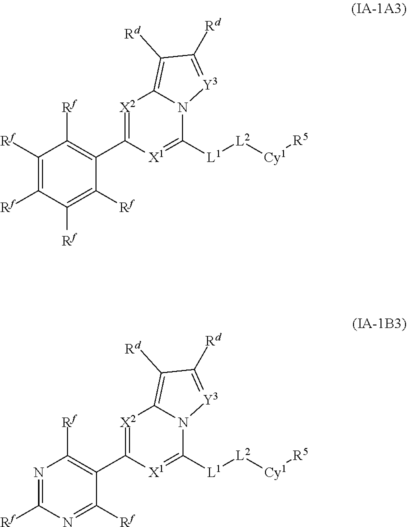

or a tautomer thereof, prodrug thereof, N-oxide thereof, a pharmaceutically acceptable ester thereof, a pharmaceutically acceptable salt thereof, or a stereoisomer thereof,
wherein
Cy¹ is selected from

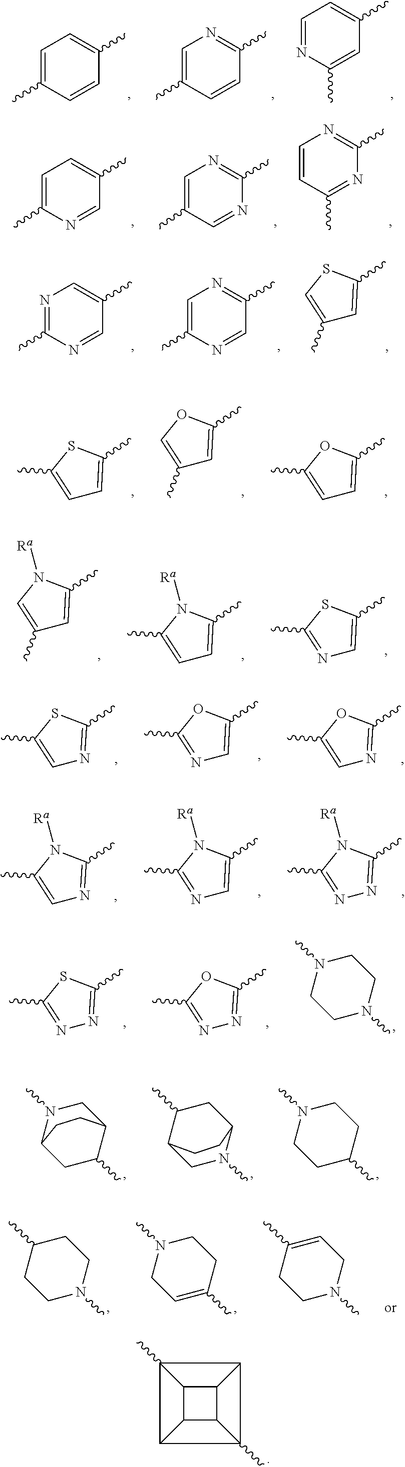

wherein Cy¹ is optionally substituted with one or more R^{a}
   unless otherwise stated all the other variables (X¹, X², X³, X⁴, Y¹, Y², Y³, L¹, L², R⁵, R^{a}, R^{d} and R^{f}) are as defined above in relation to a compound of formula (I), (IA), (IB), (IA-1), (IA-2), (IB-1), (IB-2), (IA-1A), or (IA-1B).

In one embodiment of the invention, the present invention is related to a compound of Formula (IA-1A4), or (IA-1B4):

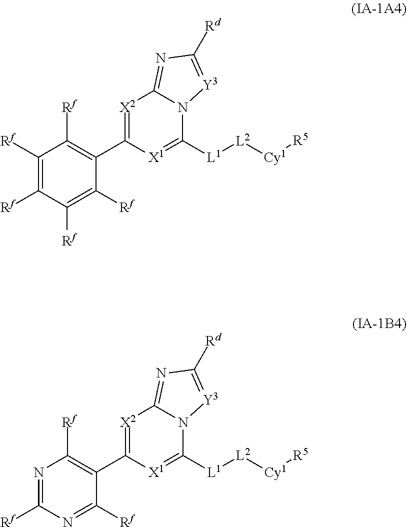

or a tautomer thereof, prodrug thereof, N-oxide thereof, a pharmaceutically acceptable ester thereof, a pharmaceutically acceptable salt thereof, or a stereoisomer thereof,
wherein
Cy¹ is selected from

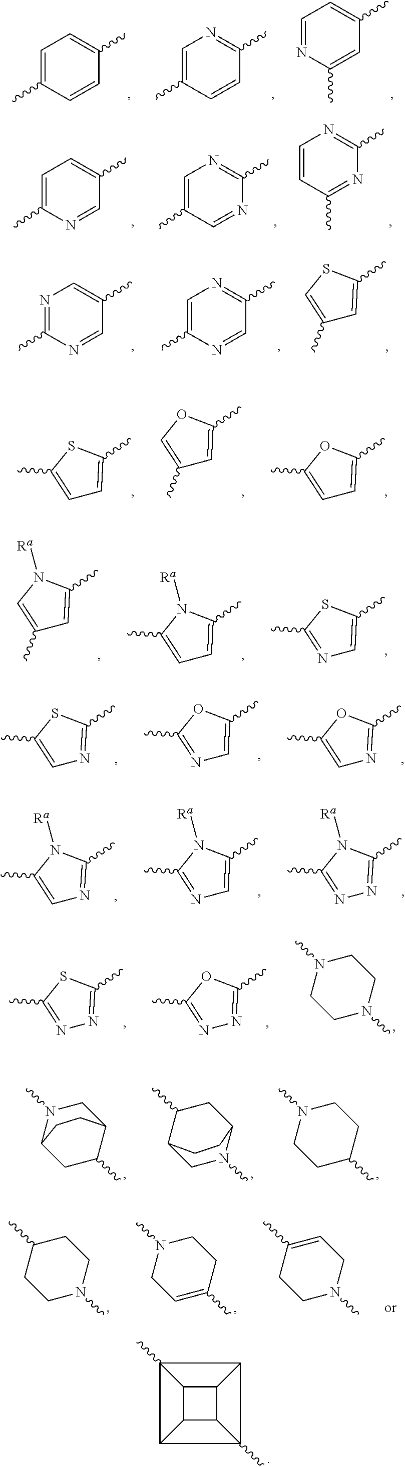

wherein Cy¹ is optionally substituted with one or more R^{a}.
   unless otherwise stated all the other variables (X¹, X², X³, X⁴, Y¹, Y², Y³, L¹, L², R⁵, R^{a}, R^{d} and R^{f}) are as defined above in relation to a compound of formula (I), (IA), (IB), (IA-1), (IA-2), (IB-1), (IB-2), (IA-1A), or (IA-1B).

In one embodiment of the invention, the present invention is related to a compound of Formula (IA-1A5) or (IA-1B5):

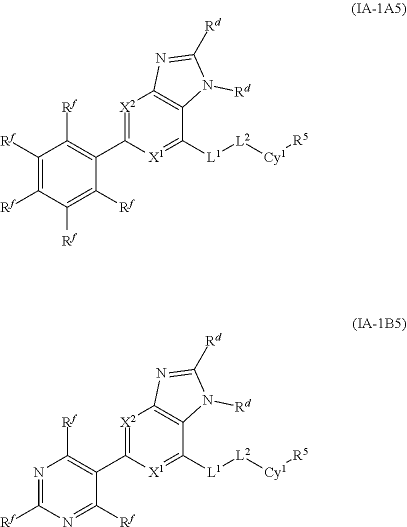

or a tautomer thereof, prodrug thereof, N-oxide thereof, a pharmaceutically acceptable ester thereof, a pharmaceutically acceptable salt thereof, or a stereoisomer thereof,
wherein
Cy¹ is selected from

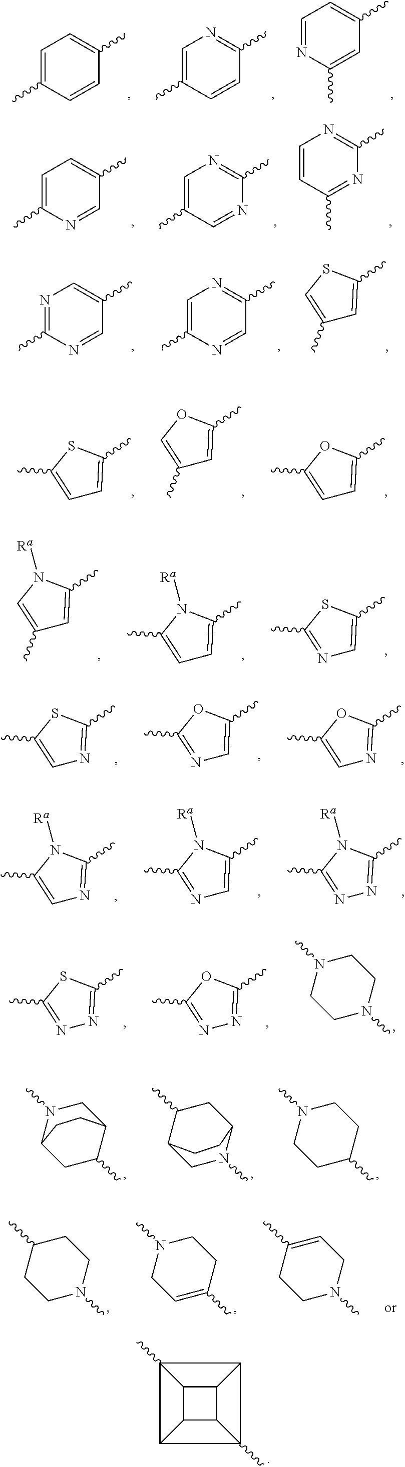

wherein Cy is optionally substituted with one or more R^{a}.
   unless otherwise stated all the other variables (X¹, X², X³, X⁴, Y¹, Y², Y³, L¹, L², R⁵, R^{a}, R^{d} and R^{f}) are as defined above in relation to a compound of formula (I), (IA), (IB), (IA-1), (IA-2), (IB-1), (IB-2), (IA-1A), or (IA-1B).

Further preferred are compounds having the formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and/or (IA-1B5), wherein Cy¹ is selected from

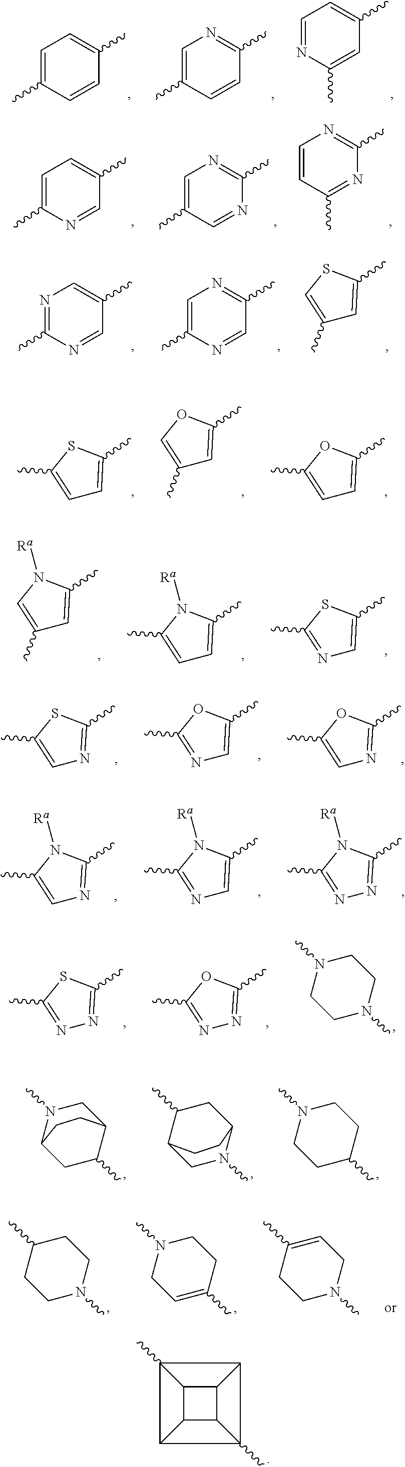

wherein Cy¹ is optionally substituted with one or more R^{a}.

Further preferred are compounds having the formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and/or (IA-1B5), wherein Cy is selected from

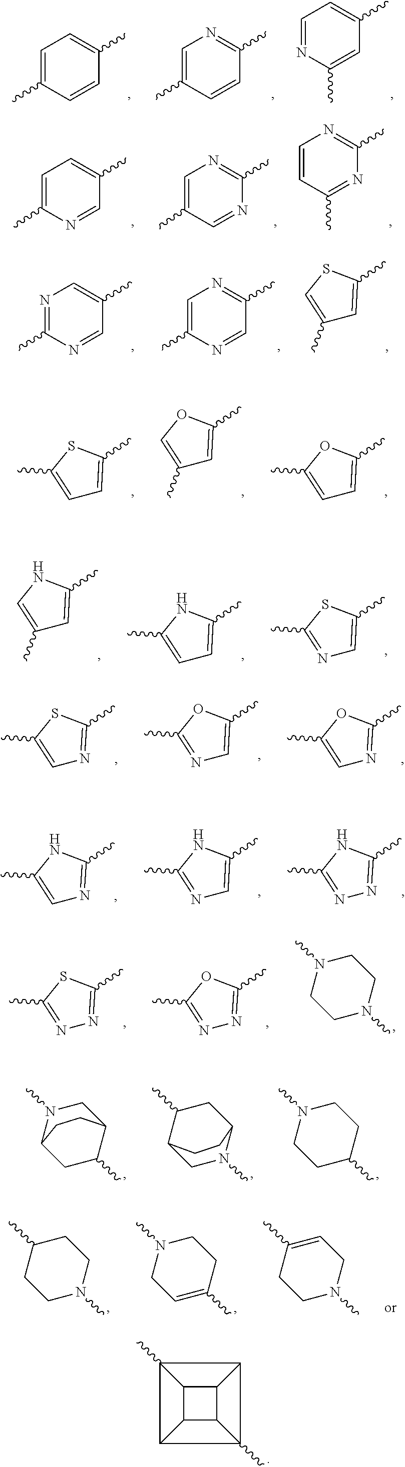

Further preferred are compounds having the formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and/or (IA-1B5), wherein L¹ is absent or selected from substituted or unsubstituted C₁₋₄ alkyl, O, S, —S(═O)ₚ or NR^{x}.

Further preferred are compounds having the formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and/or (IA-1B5), wherein L¹ is NH or O.

Further preferred are compounds having the formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and/or (IA-1B5), wherein L¹ is absent.

Further preferred are compounds having the formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and/or (IA-1B5), wherein L² is selected from substituted or unsubstituted C₁₋₄ alkyl, O, S, —S(═O)ₚ or NR^{x}.

Further preferred are compounds having the formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and/or (IA-1B5), wherein L² is substituted or unsubstituted C₁₋₄ alkyl.

Further preferred are compounds having the formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and/or (IA-1B5), wherein L² is —(CR^{b}R^{c})n-.

Further preferred are compounds having the formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and/or (IA-1B5), wherein each of R^{b} and R^{c} are independently selected form hydrogen, halogen or substituted or unsubstituted alkyl and n is 1-2.

Further preferred are compounds having the formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and/or (IA-1B5), wherein R^{b}═R^{c}═H, R^{b}═H and R^{c}=methyl and n is 1.

Further preferred are compounds having the formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and/or (IA-1B5), wherein L² is —CH₂—, —CH(Me)-.

Further preferred are compounds having the formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and/or (IA-1B5), wherein R⁵ is selected from substituted or unsubstituted aryl or substituted or unsubstituted heteroaryl.

Further preferred are compounds having the formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and/or (IA-1B5), wherein R⁵ is selected from

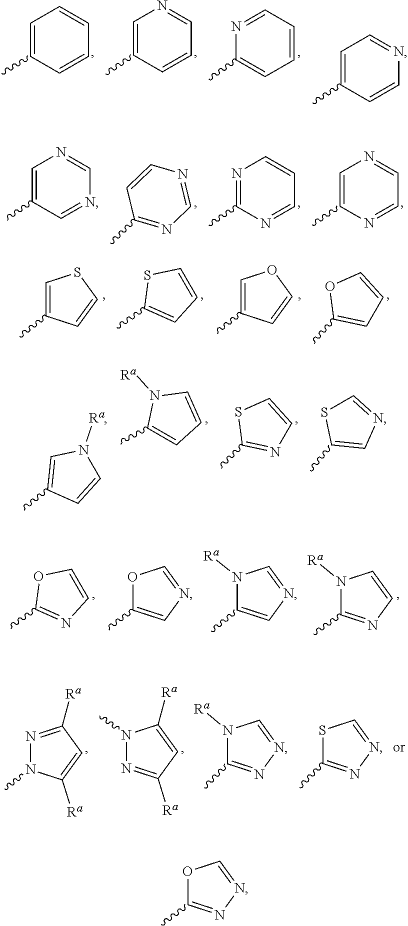

Wherein R⁵ is optionally substituted with one or more R^{a}.

Further preferred are compounds having the formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and/or (IA-1B5), wherein R⁵ is selected from

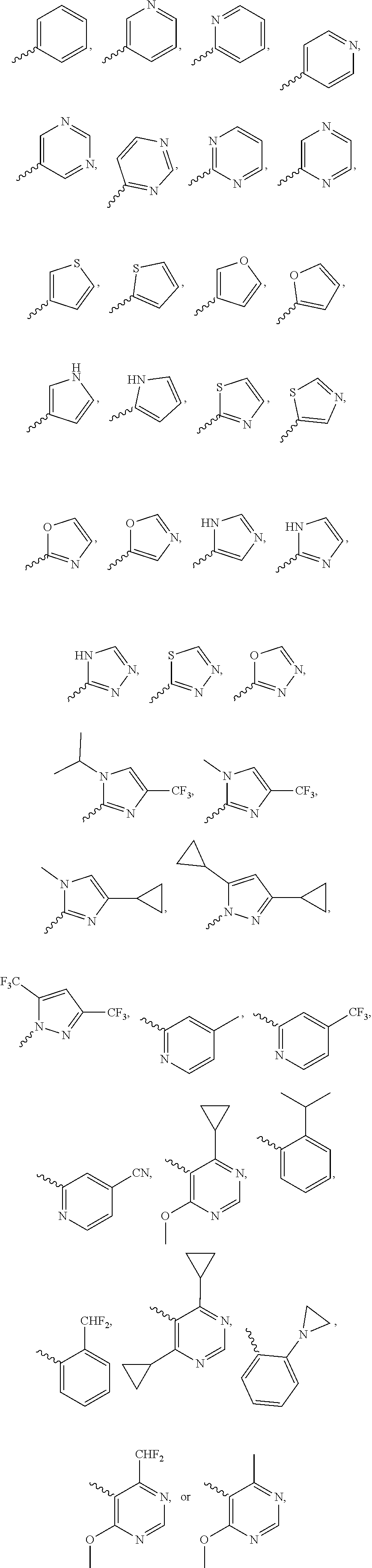

The non-limiting representative compounds of the present invention are as listed herein below and pharmaceutically acceptable salts thereof. The present invention should not be construed to be limited to these compounds.
1. 2-(4-cyclopropyl-6-methoxypyrimidin-5-yl)-N-(4-(1-methyl-4-(trifluoromethyl)-1H-imidazol-2-yl)benzyl)-6,7-dihydro-5H-cyclopenta[d]pyrimidin-4-amine;
2. 3-(4-cyclopropyl-6-methoxypyrimidin-5-yl)-1-((4-(1-methyl-4-(trifluoromethyl)-1H-imidazol-2-yl)benzyl)amino)-6,7-dihydro-5H-cyclopenta[c]pyridine-4-carbonitrile;
3. 2-(2-isopropylphenyl)-N-(4-(1-methyl-4-(trifluoromethyl)-1H-imidazol-2-yl)benzyl)-6,7-dihydro-5H-cyclopenta[d]pyrimidin-4-amine;
4. 3-(2-isopropylphenyl)-1-((4-(1-methyl-4-(trifluoromethyl)-1H-imidazol-2-yl)benzyl)amino)-6,7-dihydro-5H-cyclopenta[c]pyridine-4-carbonitrile;
5. 2-(2-isopropylphenyl)-N-(4-(1-methyl-4-(trifluoromethyl)-1H-imidazol-2-yl)benzyl)pyrrolo[2,1-f][1,2,4]triazin-4-amine;
6. 2-(4-cyclopropyl-6-methoxypyrimidin-5-yl)-N-(4-(1-methyl-4-(trifluoromethyl)-1H-imidazol-2-yl)benzyl)pyrrolo[2,1-f][1,2,4]triazin-4-amine;
7. 7-(2-isopropylphenyl)-N-(4-(1-methyl-4-(trifluoromethyl)-1H-imidazol-2-yl)benzyl)imidazo[1,2-c]pyrimidin-5-amine;
8. 7-(4-cyclopropyl-6-methoxypyrimidin-5-yl)-N-(4-(1-methyl-4-(trifluoromethyl)-1H-imidazol-2-yl)benzyl)imidazo[1,2-c]pyrimidin-5-amine;
9. N-(4-(1-isopropyl-4-(trifluoromethyl)-1H-imidazol-2-yl)benzyl)-2-(2-isopropylphenyl)-6,7-dihydro-5H-cyclopenta[d]pyrimidin-4-amine;
10. 2-(4-cyclopropyl-6-methoxypyrimidin-5-yl)-N-(4-(1-isopropyl-4-(trifluoromethyl)-1H-imidazol-2-yl)benzyl)-6,7-dihydro-5H-cyclopenta[d]pyrimidin-4-amine;
11. 2-(4-cyclopropyl-6-(difluoromethoxy)pyrimidin-5-yl)-N-(4-(1-isopropyl-4-(trifluoromethyl)-1H-imidazol-2-yl)benzyl)-6,7-dihydro-5H-cyclopenta[d]pyrimidin-4-amine;
12. 1-(2-(4-cyclopropyl-6-methoxypyrimidin-5-yl)-6,7-dihydro-5H-cyclopenta[d]pyrimidin-4-yl)-1-(4-(1-methyl-4-(trifluoromethyl)-1H-imidazol-2-yl)benzyl)urea;
13. 4-((4-(1-isopropyl-4-(trifluoromethyl)-1H-imidazol-2-yl)benzyl)oxy)-2-(2-isopropylphenyl)-6,7-dihydro-5H-cyclopenta[d]pyrimidine;
14. 1-(4-(1-isopropyl-4-(trifluoromethyl)-1H-imidazol-2-yl)benzyl)-1-(2-(2-isopropylphenyl)-6,7-dihydro-5H-cyclopenta[d]pyrimidin-4-yl)-3,3-dimethylurea;
15. 2-(2-isopropylphenyl)-N-(4-(1-methyl-4-(trifluoromethyl)-1H-imidazol-2-yl)benzyl)-7H-purin-6-amine;
16. 2-(4-cyclopropyl-6-methoxypyrimidin-5-yl)-N-(4-(1-methyl-4-(trifluoromethyl)-1H-imidazol-2-yl)benzyl)-7H-purin-6-amine
17. 2-chloro-N-(4-(1-methyl-4-(trifluoromethyl)-1H-imidazol-2-yl)benzyl)-9-(tetrahydro-2H-pyran-2-yl)-9H-purin-6-amine;
18. 2-(2-isopropylphenyl)-N-(4-(1-methyl-4-(trifluoromethyl)-1H-imidazol-2-yl)benzyl)-9-(tetrahydro-2H-pyran-2-yl)-9H-purin-6-amine;
19. 2-(4-cyclopropyl-6-methoxypyrimidin-5-yl)-N-(4-(1-methyl-4-(trifluoromethyl)-1H-imidazol-2-yl)benzyl)-9-(tetrahydro-2H-pyran-2-yl)-9H-purin-6-amine;
20. 2-(2-isopropylphenyl)-4-((4-(1-methyl-4-(trifluoromethyl)-1H-imidazol-2-yl)benzyl)amino)-6,7-dihydro-5H-cyclopenta[b]pyridine-3-carbonitrile;
21. 2-(4-cyclopropyl-6-methoxypyrimidin-5-yl)-4-((4-(1-methyl-4-(trifluoromethyl)-1H-imidazol-2-yl)benzyl)amino)-6,7-dihydro-5H-cyclopenta[b]pyridine-3-carbonitriles;
22. 2-(4-cyclopropyl-6-methoxypyrimidin-5-yl)-N-(4-(1-isopropyl-4-(trifluoromethyl)-1H-imidazol-2-yl)benzyl)pyrrolo[2,1-f][1,2,4]triazin-4-amine;
23. 2-(4-cyclopropyl-6-methoxypyrimidin-5-yl)-4-((4-(1-methyl-4-(trifluoromethyl)-1H-imidazol-2-yl)benzyl)oxy)pyrrolo[2,1-f][1,2,4]triazine;
24. 2-(4-cyclopropyl-6-methoxypyrimidin-5-yl)-4-((4-(1-methyl-4-(trifluoromethyl)-1H-imidazol-2-yl)benzyl)oxy)-6,7-dihydro-5H-cyclopenta[d]pyrimidine;
25. 2-(4-cyclopropyl-6-methoxypyrimidin-5-yl)-6-((4-(1-methyl-4-(trifluoromethyl)-1H-imidazol-2-yl)benzyl)oxy)-9-(tetrahydro-2H-pyran-2-yl)-9H-purine;
26. 2-(4-cyclopropyl-6-methoxypyrimidin-5-yl)-6-((4-(1-methyl-4-(trifluoromethyl)-1H-imidazol-2-yl)benzyl)oxy)-7H-purine;
27. 2-(2-fluoro-6-methoxyphenyl)-6-((4-(1-methyl-4-(trifluoromethyl)-1H-imidazol-2-yl)benzyl)oxy)-9-(tetrahydro-2H-pyran-2-yl)-9H-purine;
28. 2-(2-fluoro-6-methoxyphenyl)-6-((4-(1-methyl-4-(trifluoromethyl)-1H-imidazol-2-yl)benzyl)oxy)-7H-purine;
29. 2-(2-fluoro-6-methoxyphenyl)-N-(4-(1-methyl-4-(trifluoromethyl)-1H-imidazol-2-yl)benzyl)-9-(tetrahydro-2H-pyran-2-yl)-9H-purin-6-amine;
30. 2-(2-fluoro-6-methoxyphenyl)-N-(4-(1-methyl-4-(trifluoromethyl)-1H-imidazol-2-yl)benzyl)-7H-purin-6-amine;
31. 2-(2-methoxypyridin-3-yl)-N-(4-(1-methyl-4-(trifluoromethyl)-1H-imidazol-2-yl)benzyl)-9-(tetrahydro-2H-pyran-2-yl)-9H-purin-6-amine;
32. 2-(2-methoxypyridin-3-yl)-N-(4-(1-methyl-4-(trifluoromethyl)-1H-imidazol-2-yl)benzyl)-7H-purin-6-amine;
33. 2-(4-methoxypyridin-3-yl)-N-(4-(1-methyl-4-(trifluoromethyl)-1H-imidazol-2-yl)benzyl)-9-(tetrahydro-2H-pyran-2-yl)-9H-purin-6-amine;
34. 2-(4-methoxypyridin-3-yl)-N-(4-(1-methyl-4-(trifluoromethyl)-1H-imidazol-2-yl)benzyl)-7H-purin-6-amine;
35. N-(4-(1-methyl-4-(trifluoromethyl)-1H-imidazol-2-yl)benzyl)-2-morpholino-9-(tetrahydro-2H-pyran-2-yl)-9H-purin-6-amine;
36. N-(4-(1-methyl-4-(trifluoromethyl)-1H-imidazol-2-yl)benzyl)-2-morpholino-7H-purin-6-amine;
37. 2-(4-cyclopropyl-6-methoxypyrimidin-5-yl)-9-methyl-N-(4-(1-methyl-4-(trifluoromethyl)-1H-imidazol-2-yl)benzyl)-9H-purin-6-amine
38. 2-(2-chloro-3-methylphenyl)-N-(4-(1-methyl-4-(trifluoromethyl)-1H-imidazol-2-yl)benzyl)-7H-purin-6-amine
39. 2-(3,5-dichlorophenyl)-N-(4-(1-methyl-4-(trifluoromethyl)-1H-imidazol-2-yl)benzyl)-7H-purin-6-amine
40. 2-(2,4-difluorophenyl)-N-(4-(1-methyl-4-(trifluoromethyl)-1H-imidazol-2-yl)benzyl)-7H-purin-6-amine
41. 2-(3,5-difluorophenyl)-N-(4-(1-methyl-4-(trifluoromethyl)-1H-imidazol-2-yl)benzyl)-7H-purin-6-amine
42. 2-(2,4-dichlorophenyl)-N-(4-(1-methyl-4-(trifluoromethyl)-1H-imidazol-2-yl)benzyl)-7H-purin-6-amine
43. 2-(2,6-dimethoxypyridin-3-yl)-N-(4-(1-methyl-4-(trifluoromethyl)-1H-imidazol-2-yl)benzyl)-7H-purin-6-amine
44. 2-(2-fluoro-5-(trifluoromethyl)phenyl)-N-(4-(1-methyl-4-(trifluoromethyl)-1H-imidazol-2-yl)benzyl)-7H-purin-6-amine
45. N-(4-(1-methyl-4-(trifluoromethyl)-1H-imidazol-2-yl)benzyl)-2-(2-(trifluoromethyl)phenyl)-7H-purin-6-amine
46. N-(4-(1-methyl-4-(trifluoromethyl)-1H-imidazol-2-yl)benzyl)-2-(3-(trifluoromethoxy)phenyl)-7H-purin-6-amine
47. N-(4-(1-methyl-4-(trifluoromethyl)-1H-imidazol-2-yl)benzyl)-2-(4-(trifluoromethoxy)phenyl)-7H-purin-6-amine
48. 2-(2-fluoro-3-methoxyphenyl)-N-(4-(1-methyl-4-(trifluoromethyl)-1H-imidazol-2-yl)benzyl)-7H-purin-6-amine
49. 2-fluoro-5-(6-((4-(1-methyl-4-(trifluoromethyl)-1H-imidazol-2-yl)benzyl)amino)-7H-purin-2-yl)benzonitrile
50. 2-(3,5-dimethylphenyl)-N-(4-(1-methyl-4-(trifluoromethyl)-1H-imidazol-2-yl)benzyl)-7H-purin-6-amine
51. 2-(3,4-dimethoxyphenyl)-N-(4-(1-methyl-4-(trifluoromethyl)-1H-imidazol-2-yl)benzyl)-7H-purin-6-amine
52. 2-(4-fluoro-3-methoxyphenyl)-N-(4-(1-methyl-4-(trifluoromethyl)-1H-imidazol-2-yl)benzyl)-7H-purin-6-amine
53. 2-(2,4-dimethoxyphenyl)-N-(4-(1-methyl-4-(trifluoromethyl)-1H-imidazol-2-yl)benzyl)-7H-purin-6-amine
54. 2-(3-fluoro-2-(trifluoromethyl)phenyl)-N-(4-(1-methyl-4-(trifluoromethyl)-1H-imidazol-2-yl)benzyl)-7H-purin-6-amine
55. 3-(6-((4-(1-methyl-4-(trifluoromethyl)-1H-imidazol-2-yl)benzyl)amino)-7H-purin-2-yl)benzamide
56. N-(4-(1-methyl-4-(trifluoromethyl)-1H-imidazol-2-yl)benzyl)-2-(4-(methylthio)phenyl)-7H-purin-6-amine
57. N-(4-(1-methyl-4-(trifluoromethyl)-1H-imidazol-2-yl)benzyl)-2-(4-(trifluoromethyl)phenyl)-7H-purin-6-amine
58. N-(4-(1-methyl-4-(trifluoromethyl)-1H-imidazol-2-yl)benzyl)-2-(3-(trifluoromethyl)phenyl)-7H-purin-6-amine
59. N-(3-(4-((4-(1-methyl-4-(trifluoromethyl)-1H-imidazol-2-yl)benzyl)amino)-6,7-dihydro-5H-cyclopenta[d]pyrimidin-2-yl)phenyl)acetamide
60. 2-fluoro-5-(4-((4-(1-methyl-4-(trifluoromethyl)-1H-imidazol-2-yl)benzyl)amino)-6,7-dihydro-5H-cyclopenta[d]pyrimidin-2-yl)benzonitrile
61. 2-(5-chloro-2-fluorophenyl)-N-(4-(1-methyl-4-(trifluoromethyl)-1H-imidazol-2-yl)benzyl)-6,7-dihydro-5H-cyclopenta[d]pyrimidin-4-amine
62. 2-(6-ethoxypyridin-3-yl)-N-(4-(1-methyl-4-(trifluoromethyl)-1H-imidazol-2-yl)benzyl)-6,7-dihydro-5H-cyclopenta[d]pyrimidin-4-amine
63. 4-(4-((4-(1-methyl-4-(trifluoromethyl)-1H-imidazol-2-yl)benzyl)amino)-6,7-dihydro-5H-cyclopenta[d]pyrimidin-2-yl)benzonitrile
64. 2-([1,1′-biphenyl]-4-yl)-N-(4-(1-methyl-4-(trifluoromethyl)-1H-imidazol-2-yl)benzyl)-6,7-dihydro-5H-cyclopenta[d]pyrimidin-4-amine
65. 2-(2,6-dimethoxypyridin-3-yl)-N-(4-(1-methyl-4-(trifluoromethyl)-1H-imidazol-2-yl)benzyl)-6,7-dihydro-5H-cyclopenta[d]pyrimidin-4-amine
66. 2-(2,5-dimethoxyphenyl)-N-(4-(1-methyl-4-(trifluoromethyl)-1H-imidazol-2-yl)benzyl)-6,7-dihydro-5H-cyclopenta[d]pyrimidin-4-amine
67. 2-(3-chloro-2-fluorophenyl)-N-(4-(1-methyl-4-(trifluoromethyl)-1H-imidazol-2-yl)benzyl)-6,7-dihydro-5H-cyclopenta[d]pyrimidin-4-amine
68. 2-(2,4-dimethoxyphenyl)-N-(4-(1-methyl-4-(trifluoromethyl)-1H-imidazol-2-yl)benzyl)-6,7-dihydro-5H-cyclopenta[d]pyrimidin-4-amine
69. 2-(3,4-dimethoxyphenyl)-N-(4-(1-methyl-4-(trifluoromethyl)-1H-imidazol-2-yl)benzyl)-6,7-dihydro-5H-cyclopenta[d]pyrimidin-4-amine
70. N-(4-(1-methyl-4-(trifluoromethyl)-1H-imidazol-2-yl)benzyl)-2-(2-(trifluoromethyl)phenyl)-6,7-dihydro-5H-cyclopenta[d]pyrimidin-4-amine
71. N-(4-(1-methyl-4-(trifluoromethyl)-1H-imidazol-2-yl)benzyl)-2-(4-(trifluoromethyl)phenyl)-6,7-dihydro-5H-cyclopenta[d]pyrimidin-4-amine
72. 3-(4-((4-(1-methyl-4-(trifluoromethyl)-1H-imidazol-2-yl)benzyl)amino)-6,7-dihydro-5H-cyclopenta[d]pyrimidin-2-yl)benzamide
73. 2-(3,5-dichlorophenyl)-N-(4-(1-methyl-4-(trifluoromethyl)-1H-imidazol-2-yl)benzyl)-6,7-dihydro-5H-cyclopenta[d]pyrimidin-4-amine
74. N-(4-(1-methyl-4-(trifluoromethyl)-1H-imidazol-2-yl)benzyl)-2-(2,3,5-trifluorophenyl)-6,7-dihydro-5H-cyclopenta[d]pyrimidin-4-amine
75. 2-(4-cyclopropyl-6-methoxypyrimidin-5-yl)-N-(1-(4-(1-methyl-4-(trifluoromethyl)-1H-imidazol-2-yl)phenyl)ethyl)-7H-purin-6-amine
76. 2-(4-cyclopropyl-6-methoxypyrimidin-5-yl)-N-(4-(pyridin-3-yl)benzyl)-7H-purin-6-amine 77. 2-(4-cyclopropyl-6-methoxypyrimidin-5-yl)-N-(4-(5-fluoropyridin-2-yl)benzyl)-7H-purin-6-amine and pharmaceutically acceptable salts thereof.

Further preferred is a compound selected from
2-(4-cyclopropyl-6-methoxypyrimidin-5-yl)-N-(4-(1-methyl-4-(trifluoromethyl)-1H-imidazol-2-yl)benzyl)-6,7-dihydro-5H-cyclopenta[d]pyrimidin-4-amine;
3-(4-cyclopropyl-6-methoxypyrimidin-5-yl)-1-((4-(1-methyl-4-(trifluoromethyl)-1H-imidazol-2-yl)benzyl)amino)-6,7-dihydro-5H-cyclopenta[c]pyridine-4-carbonitrile;
2-(2-isopropylphenyl)-N-(4-(1-methyl-4-(trifluoromethyl)-1H-imidazol-2-yl)benzyl)-6,7-dihydro-5H-cyclopenta[d]pyrimidin-4-amine;
3-(2-isopropylphenyl)-1-((4-(1-methyl-4-(trifluoromethyl)-1H-imidazol-2-yl)benzyl)amino)-6,7-dihydro-5H-cyclopenta[c]pyridine-4-carbonitrile;
N-(4-(1-isopropyl-4-(trifluoromethyl)-1H-imidazol-2-yl)benzyl)-2-(2-isopropylphenyl)-6,7-dihydro-5H-cyclopenta[d]pyrimidin-4-amine;
2-(4-cyclopropyl-6-methoxypyrimidin-5-yl)-N-(4-(1-isopropyl-4-(trifluoromethyl)-1H-imidazol-2-yl)benzyl)-6,7-dihydro-5H-cyclopenta[d]pyrimidin-4-amine;
2-(4-cyclopropyl-6-(difluoromethoxy)pyrimidin-5-yl)-N-(4-(1-isopropyl-4-(trifluoromethyl)-1H-imidazol-2-yl)benzyl)-6,7-dihydro-5H-cyclopenta[d]pyrimidin-4-amine;
1-(2-(4-cyclopropyl-6-methoxypyrimidin-5-yl)-6,7-dihydro-5H-cyclopenta[d]pyrimidin-4-yl)-1-(4-(1-methyl-4-(trifluoromethyl)-1H-imidazol-2-yl)benzyl)urea;
4-((4-(1-isopropyl-4-(trifluoromethyl)-1H-imidazol-2-yl)benzyl)oxy)-2-(2-isopropylphenyl)-6,7-dihydro-5H-cyclopenta[d]pyrimidine;
1-(4-(1-isopropyl-4-(trifluoromethyl)-1H-imidazol-2-yl)benzyl)-1-(2-(2-isopropylphenyl)-6,7-dihydro-5H-cyclopenta[d]pyrimidin-4-yl)-3,3-dimethylurea;
2-(2-isopropylphenyl)-4-((4-(1-methyl-4-(trifluoromethyl)-1H-imidazol-2-yl)benzyl)amino)-6,7-dihydro-5H-cyclopenta[b]pyridine-3-carbonitrile;
2-(4-cyclopropyl-6-methoxypyrimidin-5-yl)-4-((4-(1-methyl-4-(trifluoromethyl)-1H-imidazol-2-yl)benzyl)amino)-6,7-dihydro-5H-cyclopenta[b]pyridine-3-carbonitriles;
2-(4-cyclopropyl-6-methoxypyrimidin-5-yl)-4-((4-(1-methyl-4-(trifluoromethyl)-1H-imidazol-2-yl)benzyl)oxy)-6,7-dihydro-5H-cyclopenta[d]pyrimidine;
N-(3-(4-((4-(1-methyl-4-(trifluoromethyl)-1H-imidazol-2-yl)benzyl)amino)-6,7-dihydro-5H-cyclopenta[d]pyrimidin-2-yl)phenyl)acetamide
2-fluoro-5-(4-((4-(1-methyl-4-(trifluoromethyl)-1H-imidazol-2-yl)benzyl)amino)-6,7-dihydro-5H-cyclopenta[d]pyrimidin-2-yl)benzonitrile
2-(5-chloro-2-fluorophenyl)-N-(4-(1-methyl-4-(trifluoromethyl)-1H-imidazol-2-yl)benzyl)-6,7-dihydro-5H-cyclopenta[d]pyrimidin-4-amine
2-(6-ethoxypyridin-3-yl)-N-(4-(1-methyl-4-(trifluoromethyl)-1H-imidazol-2-yl)benzyl)-6,7-dihydro-5H-cyclopenta[d]pyrimidin-4-amine
4-(4-((4-(1-methyl-4-(trifluoromethyl)-1H-imidazol-2-yl)benzyl)amino)-6,7-dihydro-5H-cyclopenta[d]pyrimidin-2-yl)benzonitrile
2-([1,1′-biphenyl]-4-yl)-N-(4-(1-methyl-4-(trifluoromethyl)-1H-imidazol-2-yl)benzyl)-6,7-dihydro-5H-cyclopenta[d]pyrimidin-4-amine
2-(2,6-dimethoxypyridin-3-yl)-N-(4-(1-methyl-4-(trifluoromethyl)-1H-imidazol-2-yl)benzyl)-6,7-dihydro-5H-cyclopenta[d]pyrimidin-4-amine
2-(2,5-dimethoxyphenyl)-N-(4-(1-methyl-4-(trifluoromethyl)-1H-imidazol-2-yl)benzyl)-6,7-dihydro-5H-cyclopenta[d]pyrimidin-4-amine
2-(3-chloro-2-fluorophenyl)-N-(4-(1-methyl-4-(trifluoromethyl)-1H-imidazol-2-yl)benzyl)-6,7-dihydro-5H-cyclopenta[d]pyrimidin-4-amine
2-(2,4-dimethoxyphenyl)-N-(4-(1-methyl-4-(trifluoromethyl)-1H-imidazol-2-yl)benzyl)-6,7-dihydro-5H-cyclopenta[d]pyrimidin-4-amine
2-(3,4-dimethoxyphenyl)-N-(4-(1-methyl-4-(trifluoromethyl)-1H-imidazol-2-yl)benzyl)-6,7-dihydro-5H-cyclopenta[d]pyrimidin-4-amine
N-(4-(1-methyl-4-(trifluoromethyl)-1H-imidazol-2-yl)benzyl)-2-(2-(trifluoromethyl)phenyl)-6,7-dihydro-5H-cyclopenta[d]pyrimidin-4-amine
N-(4-(1-methyl-4-(trifluoromethyl)-1H-imidazol-2-yl)benzyl)-2-(4-(trifluoromethyl)phenyl)-6,7-dihydro-5H-cyclopenta[d]pyrimidin-4-amine
3-(4-((4-(1-methyl-4-(trifluoromethyl)-1H-imidazol-2-yl)benzyl)amino)-6,7-dihydro-5H-cyclopenta[d]pyrimidin-2-yl)benzamide
2-(3,5-dichlorophenyl)-N-(4-(1-methyl-4-(trifluoromethyl)-1H-imidazol-2-yl)benzyl)-6,7-dihydro-5H-cyclopenta[d]pyrimidin-4-amine
N-(4-(1-methyl-4-(trifluoromethyl)-1H-imidazol-2-yl)benzyl)-2-(2,3,5-trifluorophenyl)-6,7-dihydro-5H-cyclopenta[d]pyrimidin-4-amine and pharmaceutically acceptable salts thereof.

Further preferred is a compound selected from
2-(2-isopropylphenyl)-N-(4-(1-methyl-4-(trifluoromethyl)-1H-imidazol-2-yl)benzyl)pyrrolo[2,1-f][1,2,4]triazin-4-amine;
2-(4-cyclopropyl-6-methoxypyrimidin-5-yl)-N-(4-(1-methyl-4-(trifluoromethyl)-1H-imidazol-2-yl)benzyl)pyrrolo[2,1-f][1,2,4]triazin-4-amine;
7-(2-isopropylphenyl)-N-(4-(1-methyl-4-(trifluoromethyl)-1H-imidazol-2-yl)benzyl)imidazo[1,2-c]pyrimidin-5-amine;
7-(4-cyclopropyl-6-methoxypyrimidin-5-yl)-N-(4-(1-methyl-4-(trifluoromethyl)-1H-imidazol-2-yl)benzyl)imidazo[1,2-c]pyrimidin-5-amine;
2-(4-cyclopropyl-6-methoxypyrimidin-5-yl)-N-(4-(1-isopropyl-4-(trifluoromethyl)-1H-imidazol-2-yl)benzyl)pyrrolo[2,1-f][1,2,4]triazin-4-amine;
2-(4-cyclopropyl-6-methoxypyrimidin-5-yl)-4-((4-(1-methyl-4-(trifluoromethyl)-1H-imidazol-2-yl)benzyl)oxy)pyrrolo[2,1-f][1,2,4]triazine; and pharmaceutically acceptable salts thereof.

Further preferred is a compound selected from 2-(2-isopropylphenyl)-N-(4-(1-methyl-4-(trifluoromethyl)-1H-imidazol-2-yl)benzyl)-7H-purin-6-amine;
2-(4-cyclopropyl-6-methoxypyrimidin-5-yl)-N-(4-(1-methyl-4-(trifluoromethyl)-1H-imidazol-2-yl)benzyl)-7H-purin-6-amine
2-chloro-N-(4-(1-methyl-4-(trifluoromethyl)-1H-imidazol-2-yl)benzyl)-9-(tetrahydro-2H-pyran-2-yl)-9H-purin-6-amine;
2-(2-isopropylphenyl)-N-(4-(1-methyl-4-(trifluoromethyl)-1H-imidazol-2-yl)benzyl)-9-(tetrahydro-2H-pyran-2-yl)-9H-purin-6-amine;
2-(4-cyclopropyl-6-methoxypyrimidin-5-yl)-N-(4-(1-methyl-4-(trifluoromethyl)-1H-imidazol-2-yl)benzyl)-9-(tetrahydro-2H-pyran-2-yl)-9H-purin-6-amine;
2-(4-cyclopropyl-6-methoxypyrimidin-5-yl)-6-((4-(1-methyl-4-(trifluoromethyl)-1H-imidazol-2-yl)benzyl)oxy)-9-(tetrahydro-2H-pyran-2-yl)-9H-purine;
2-(4-cyclopropyl-6-methoxypyrimidin-5-yl)-6-((4-(1-methyl-4-(trifluoromethyl)-1H-imidazol-2-yl)benzyl)oxy)-7H-purine;
2-(2-fluoro-6-methoxyphenyl)-6-((4-(1-methyl-4-(trifluoromethyl)-1H-imidazol-2-yl)benzyl)oxy)-9-(tetrahydro-2H-pyran-2-yl)-9H-purine;
2-(2-fluoro-6-methoxyphenyl)-6-((4-(1-methyl-4-(trifluoromethyl)-1H-imidazol-2-yl)benzyl)oxy)-7H-purine;
2-(2-fluoro-6-methoxyphenyl)-N-(4-(1-methyl-4-(trifluoromethyl)-1H-imidazol-2-yl)benzyl)-9-(tetrahydro-2H-pyran-2-yl)-9H-purin-6-amine;
2-(2-fluoro-6-methoxyphenyl)-N-(4-(1-methyl-4-(trifluoromethyl)-1H-imidazol-2-yl)benzyl)-7H-purin-6-amine;
2-(2-methoxypyridin-3-yl)-N-(4-(1-methyl-4-(trifluoromethyl)-1H-imidazol-2-yl)benzyl)-9-(tetrahydro-2H-pyran-2-yl)-9H-purin-6-amine;
2-(2-methoxypyridin-3-yl)-N-(4-(1-methyl-4-(trifluoromethyl)-1H-imidazol-2-yl)benzyl)-7H-purin-6-amine;
2-(4-methoxypyridin-3-yl)-N-(4-(1-methyl-4-(trifluoromethyl)-1H-imidazol-2-yl)benzyl)-9-(tetrahydro-2H-pyran-2-yl)-9H-purin-6-amine;
2-(4-methoxypyridin-3-yl)-N-(4-(1-methyl-4-(trifluoromethyl)-1H-imidazol-2-yl)benzyl)-7H-purin-6-amine;
N-(4-(1-methyl-4-(trifluoromethyl)-1H-imidazol-2-yl)benzyl)-2-morpholino-9-(tetrahydro-2H-pyran-2-yl)-9H-purin-6-amine;
N-(4-(1-methyl-4-(trifluoromethyl)-1H-imidazol-2-yl)benzyl)-2-morpholino-7H-purin-6-amine;
2-(4-cyclopropyl-6-methoxypyrimidin-5-yl)-9-methyl-N-(4-(1-methyl-4-(trifluoromethyl)-1H-imidazol-2-yl)benzyl)-9H-purin-6-amine
2-(2-chloro-3-methylphenyl)-N-(4-(1-methyl-4-(trifluoromethyl)-1H-imidazol-2-yl)benzyl)-7H-purin-6-amine
2-(3,5-dichlorophenyl)-N-(4-(1-methyl-4-(trifluoromethyl)-1H-imidazol-2-yl)benzyl)-7H-purin-6-amine
2-(2,4-difluorophenyl)-N-(4-(1-methyl-4-(trifluoromethyl)-1H-imidazol-2-yl)benzyl)-7H-purin-6-amine
2-(3,5-difluorophenyl)-N-(4-(1-methyl-4-(trifluoromethyl)-1H-imidazol-2-yl)benzyl)-7H-purin-6-amine
2-(2,4-dichlorophenyl)-N-(4-(1-methyl-4-(trifluoromethyl)-1H-imidazol-2-yl)benzyl)-7H-purin-6-amine
2-(2,6-dimethoxypyridin-3-yl)-N-(4-(1-methyl-4-(trifluoromethyl)-1H-imidazol-2-yl)benzyl)-7H-purin-6-amine
2-(2-fluoro-5-(trifluoromethyl)phenyl)-N-(4-(1-methyl-4-(trifluoromethyl)-1H-imidazol-2-yl)benzyl)-7H-purin-6-amine
N-(4-(1-methyl-4-(trifluoromethyl)-1H-imidazol-2-yl)benzyl)-2-(2-(trifluoromethyl)phenyl)-7H-purin-6-amine
N-(4-(1-methyl-4-(trifluoromethyl)-1H-imidazol-2-yl)benzyl)-2-(3-(trifluoromethoxy)phenyl)-7H-purin-6-amine
N-(4-(1-methyl-4-(trifluoromethyl)-1H-imidazol-2-yl)benzyl)-2-(4-(trifluoromethoxy)phenyl)-7H-purin-6-amine
2-(2-fluoro-3-methoxyphenyl)-N-(4-(1-methyl-4-(trifluoromethyl)-1H-imidazol-2-yl)benzyl)-7H-purin-6-amine
2-fluoro-5-(6-((4-(1-methyl-4-(trifluoromethyl)-1H-imidazol-2-yl)benzyl)amino)-7H-purin-2-yl)benzonitrile
2-(3,5-dimethylphenyl)-N-(4-(1-methyl-4-(trifluoromethyl)-1H-imidazol-2-yl)benzyl)-7H-purin-6-amine
2-(3,4-dimethoxyphenyl)-N-(4-(1-methyl-4-(trifluoromethyl)-1H-imidazol-2-yl)benzyl)-7H-purin-6-amine
2-(4-fluoro-3-methoxyphenyl)-N-(4-(1-methyl-4-(trifluoromethyl)-1H-imidazol-2-yl)benzyl)-7H-purin-6-amine
2-(2,4-dimethoxyphenyl)-N-(4-(1-methyl-4-(trifluoromethyl)-1H-imidazol-2-yl)benzyl)-7H-purin-6-amine
2-(3-fluoro-2-(trifluoromethyl)phenyl)-N-(4-(1-methyl-4-(trifluoromethyl)-1H-imidazol-2-yl)benzyl)-7H-purin-6-amine
3-(6-((4-(1-methyl-4-(trifluoromethyl)-1H-imidazol-2-yl)benzyl)amino)-7H-purin-2-yl)benzamide
N-(4-(1-methyl-4-(trifluoromethyl)-1H-imidazol-2-yl)benzyl)-2-(4-(methylthio)phenyl)-7H-purin-6-amine
N-(4-(1-methyl-4-(trifluoromethyl)-1H-imidazol-2-yl)benzyl)-2-(4-(trifluoromethyl)phenyl)-7H-purin-6-amine
N-(4-(1-methyl-4-(trifluoromethyl)-1H-imidazol-2-yl)benzyl)-2-(3-(trifluoromethyl)phenyl)-7H-purin-6-amine
2-(4-cyclopropyl-6-methoxypyrimidin-5-yl)-N-(1-(4-(1-methyl-4-(trifluoromethyl)-1H-imidazol-2-yl)phenyl)ethyl)-7H-purin-6-amine
2-(4-cyclopropyl-6-methoxypyrimidin-5-yl)-N-(4-(pyridin-3-yl)benzyl)-7H-purin-6-amine
2-(4-cyclopropyl-6-methoxypyrimidin-5-yl)-N-(4-(5-fluoropyridin-2-yl)benzyl)-7H-purin-6-amine and pharmaceutically acceptable salts thereof.

Further preferred is a compound selected from
2-(4-cyclopropyl-6-methoxypyrimidin-5-yl)-N-(4-(1-methyl-4-(trifluoromethyl)-1H-imidazol-2-yl)benzyl)-6,7-dihydro-5H-cyclopenta[d]pyrimidin-4-amine;
2-(4-cyclopropyl-6-methoxypyrimidin-5-yl)-N-(4-(1-methyl-4-(trifluoromethyl)-1H-imidazol-2-yl)benzyl)pyrrolo[2,1-f][1,2,4]triazin-4-amine;
2-(4-cyclopropyl-6-methoxypyrimidin-5-yl)-N-(4-(1-isopropyl-4-(trifluoromethyl)-1H-imidazol-2-yl)benzyl)-6,7-dihydro-5H-cyclopenta[d]pyrimidin-4-amine;
2-(4-cyclopropyl-6-methoxypyrimidin-5-yl)-N-(4-(1-methyl-4-(trifluoromethyl)-1H-imidazol-2-yl)benzyl)-7H-purin-6-amine;
2-(4-cyclopropyl-6-methoxypyrimidin-5-yl)-N-(4-(1-isopropyl-4-(trifluoromethyl)-1H-imidazol-2-yl)benzyl)pyrrolo[2,1-f][1,2,4]triazin-4-amine;
2-(2-methoxypyridin-3-yl)-N-(4-(1-methyl-4-(trifluoromethyl)-1H-imidazol-2-yl)benzyl)-7H-purin-6-amine;
2-(4-methoxypyridin-3-yl)-N-(4-(1-methyl-4-(trifluoromethyl)-1H-imidazol-2-yl)benzyl)-7H-purin-6-amine; and pharmaceutically acceptable salts thereof.

Further preferred is a compound, 2-(4-cyclopropyl-6-methoxypyrimidin-5-yl)-N-(4-(1-methyl-4-(trifluoromethyl)-1H-imidazol-2-yl)benzyl)-6,7-dihydro-5H-cyclopenta[d]pyrimidin-4-amine;

and pharmaceutically acceptable salts thereof.

Further preferred is a compound, 2-(4-cyclopropyl-6-methoxypyrimidin-5-yl)-N-(4-(1-methyl-4-(trifluoromethyl)-1H-imidazol-2-yl)benzyl)pyrrolo[2,1-f][1,2,4]triazin-4-amine;

and pharmaceutically acceptable salts thereof.

Further preferred is a compound, 2-(4-cyclopropyl-6-methoxypyrimidin-5-yl)-N-(4-(1-isopropyl-4-(trifluoromethyl)-1H-imidazol-2-yl)benzyl)-6,7-dihydro-5H-cyclopenta[d]pyrimidin-4-amine and pharmaceutically acceptable salts thereof.

Further preferred is a compound, 2-(4-cyclopropyl-6-methoxypyrimidin-5-yl)-N-(4-(1-methyl-4-(trifluoromethyl)-1H-imidazol-2-yl)benzyl)-7H-purin-6-amine and pharmaceutically acceptable salts thereof.

Further preferred is a compound, 2-(4-cyclopropyl-6-methoxypyrimidin-5-yl)-N-(4-(1-isopropyl-4-(trifluoromethyl)-1H-imidazol-2-yl)benzyl)pyrrolo[2,1-f][1,2,4]triazin-4-amine and pharmaceutically acceptable salts thereof.

Further preferred is a compound, 2-(2-methoxypyridin-3-yl)-N-(4-(1-methyl-4-(trifluoromethyl)-1H-imidazol-2-yl)benzyl)-7H-purin-6-amine and pharmaceutically acceptable salts thereof.

Further preferred is a compound, 2-(4-methoxypyridin-3-yl)-N-(4-(1-methyl-4-(trifluoromethyl)-1H-imidazol-2-yl)benzyl)-7H-purin-6-amine and pharmaceutically acceptable salts thereof.

TABLE-1

| Ex. | Structure |
|---|---|
| 1 | 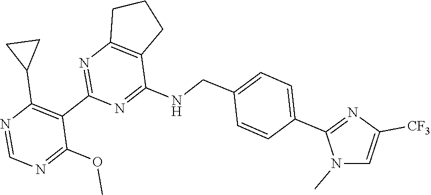 [SMILES: COc1ncnc(C2CC2)c1-c1nc2c(c(NCc3ccc(-c4nc(C(F)(F)F)cn4C)cc3)n1)CCC2] |
| 2 | 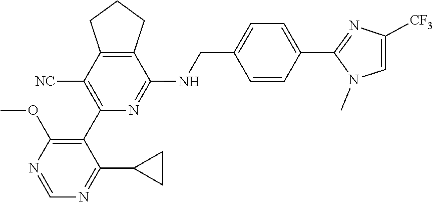 [SMILES: COc1ncnc(C2CC2)c1-c1nc(NCc2ccc(-c3nc(C(F)(F)F)cn3C)cc2)c2c(c1C#N)CCC2] |
| 3 | 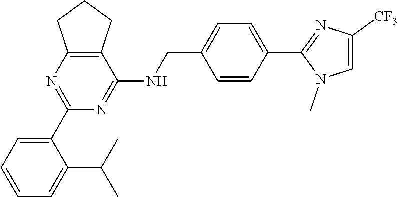 [SMILES: CC(C)c1ccccc1-c1nc2c(c(NCc3ccc(-c4nc(C(F)(F)F)cn4C)cc3)n1)CCC2] |
| 4 | 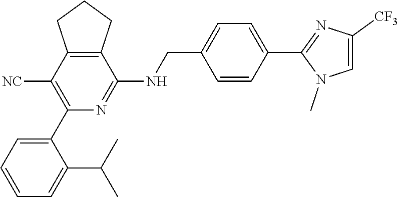 [SMILES: CC(C)c1ccccc1-c1nc(NCc2ccc(-c3nc(C(F)(F)F)cn3C)cc2)c2c(c1C#N)CCC2] |
| 5 | 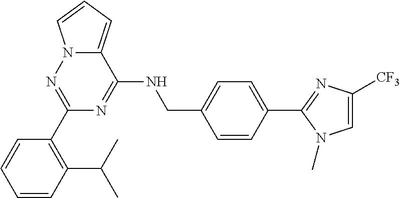 [SMILES: CC(C)c1ccccc1-c1nc(NCc2ccc(-c3nc(C(F)(F)F)cn3C)cc2)c2cccn2n1] |
| 6 | 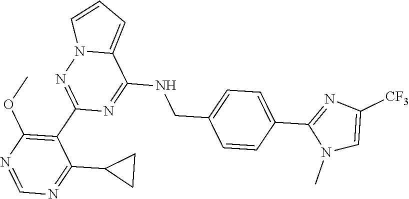 [SMILES: COc1ncnc(C2CC2)c1-c1nc(NCc2ccc(-c3nc(C(F)(F)F)cn3C)cc2)c2cccn2n1] |
| 7 | 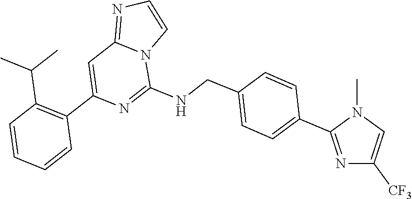 [SMILES: CC(C)c1ccccc1-c1cc2nccn2c(NCc2ccc(-c3nc(C(F)(F)F)cn3C)cc2)n1] |
| 8 | 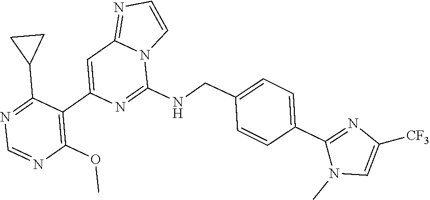 [SMILES: COc1ncnc(C2CC2)c1-c1cc2nccn2c(NCc2ccc(-c3nc(C(F)(F)F)cn3C)cc2)n1] |
| 9 | 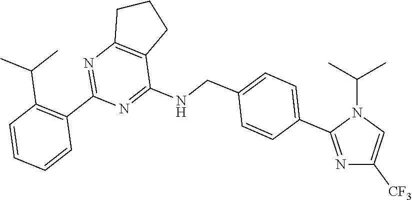 [SMILES: CC(C)c1ccccc1-c1nc2c(c(NCc3ccc(-c4nc(C(F)(F)F)cn4C(C)C)cc3)n1)CCC2] |
| 10 | 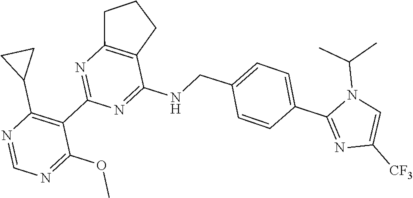 [SMILES: COc1ncnc(C2CC2)c1-c1nc2c(c(NCc3ccc(-c4nc(C(F)(F)F)cn4C(C)C)cc3)n1)CCC2] |
| 11 | 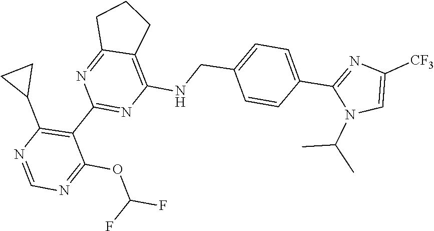 [SMILES: CC(C)n1cc(C(F)(F)F)nc1-c1ccc(CNc2nc(-c3c(OC(F)F)ncnc3C3CC3)nc3c2CCC3)cc1] |
| 12 | 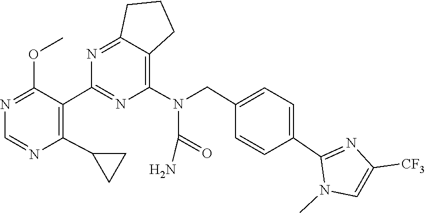 [SMILES: COc1ncnc(C2CC2)c1-c1nc2c(c(N(Cc3ccc(-c4nc(C(F)(F)F)cn4C)cc3)C(N)=O)n1)CCC2] |
| 13 | 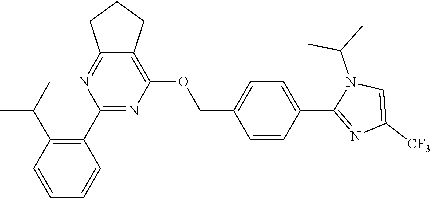 [SMILES: CC(C)c1ccccc1-c1nc2c(c(OCc3ccc(-c4nc(C(F)(F)F)cn4C(C)C)cc3)n1)CCC2] |
| 14 | 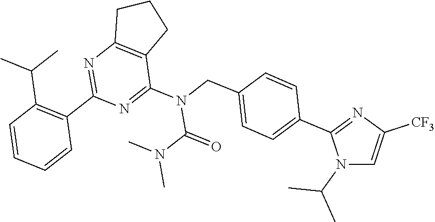 [SMILES: CC(C)c1ccccc1-c1nc2c(c(N(Cc3ccc(-c4nc(C(F)(F)F)cn4C(C)C)cc3)C(=O)N(C)C)n1)CCC2] |
| 15 | 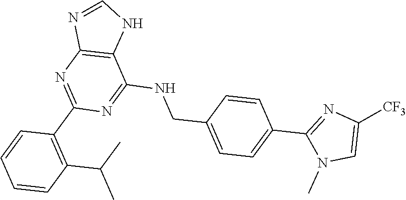 [SMILES: CC(C)c1ccccc1-c1nc(NCc2ccc(-c3nc(C(F)(F)F)cn3C)cc2)c2[nH]cnc2n1] |
| 16 | 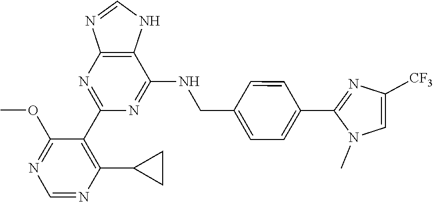 [SMILES: COc1ncnc(C2CC2)c1-c1nc(NCc2ccc(-c3nc(C(F)(F)F)cn3C)cc2)c2[nH]cnc2n1] |
| 17 | 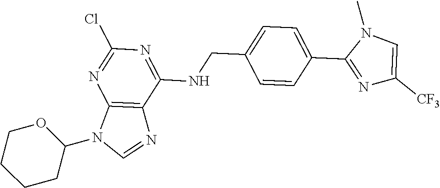 [SMILES: Cn1cc(C(F)(F)F)nc1-c1ccc(CNc2nc(Cl)nc3c2ncn3C2CCCCO2)cc1] |
| 18 | 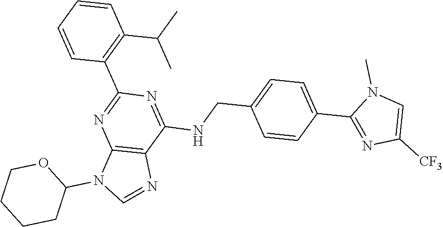 [SMILES: CC(C)c1ccccc1-c1nc(NCc2ccc(-c3nc(C(F)(F)F)cn3C)cc2)c2ncn(C3CCCCO3)c2n1] |
| 19 | 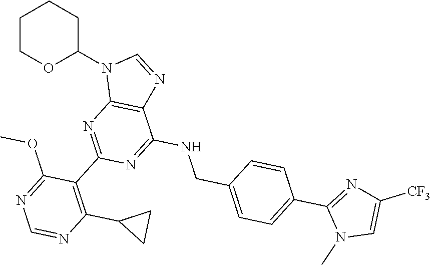 [SMILES: COc1ncnc(C2CC2)c1-c1nc(NCc2ccc(-c3nc(C(F)(F)F)cn3C)cc2)c2ncn(C3CCCCO3)c2n1] |
| 20 | 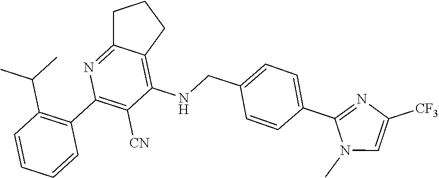 [SMILES: CC(C)c1ccccc1-c1nc2c(c(NCc3ccc(-c4nc(C(F)(F)F)cn4C)cc3)c1C#N)CCC2] |
| 21 | 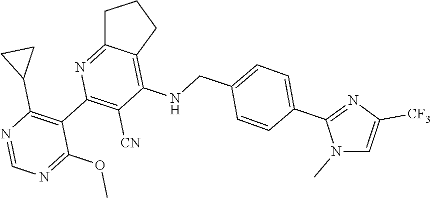 [SMILES: COc1ncnc(C2CC2)c1-c1nc2c(c(NCc3ccc(-c4nc(C(F)(F)F)cn4C)cc3)c1C#N)CCC2] |
| 22 | 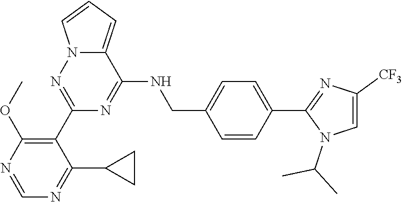 [SMILES: COc1ncnc(C2CC2)c1-c1nc(NCc2ccc(-c3nc(C(F)(F)F)cn3C(C)C)cc2)c2cccn2n1] |
| 23 | 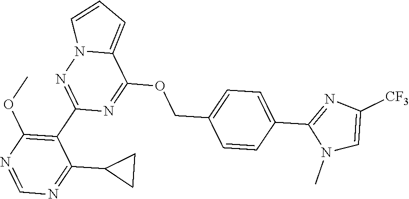 [SMILES: COc1ncnc(C2CC2)c1-c1nc(OCc2ccc(-c3nc(C(F)(F)F)cn3C)cc2)c2cccn2n1] |
| 24 | 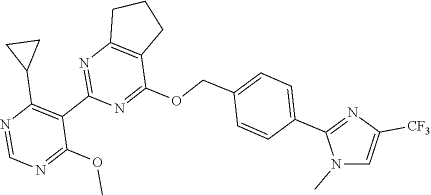 [SMILES: COc1ncnc(C2CC2)c1-c1nc2c(c(OCc3ccc(-c4nc(C(F)(F)F)cn4C)cc3)n1)CCC2] |
| 25 | 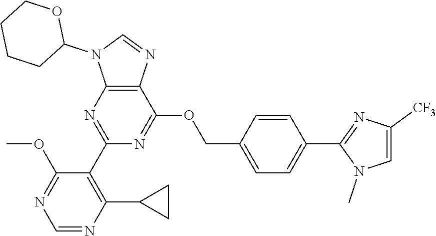 [SMILES: COc1ncnc(C2CC2)c1-c1nc(OCc2ccc(-c3nc(C(F)(F)F)cn3C)cc2)c2ncn(C3CCCCO3)c2n1] |
| 26 | 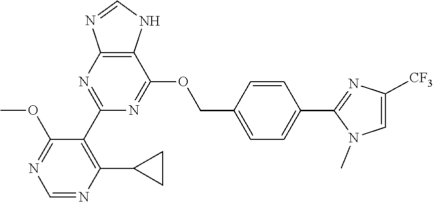 [SMILES: COc1ncnc(C2CC2)c1-c1nc(OCc2ccc(-c3nc(C(F)(F)F)cn3C)cc2)c2[nH]cnc2n1] |
| 27 | 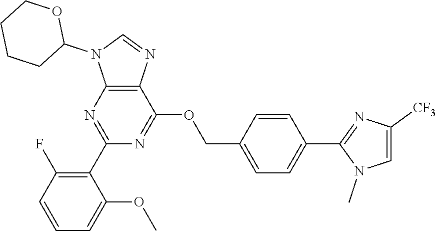 [SMILES: COc1cccc(F)c1-c1nc(OCc2ccc(-c3nc(C(F)(F)F)cn3C)cc2)c2ncn(C3CCCCO3)c2n1] |
| 28 | 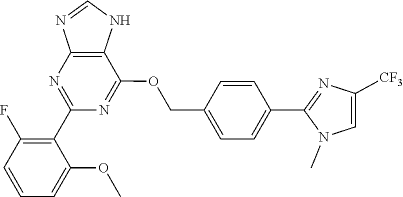 [SMILES: COc1cccc(F)c1-c1nc(OCc2ccc(-c3nc(C(F)(F)F)cn3C)cc2)c2[nH]cnc2n1] |
| 29 | 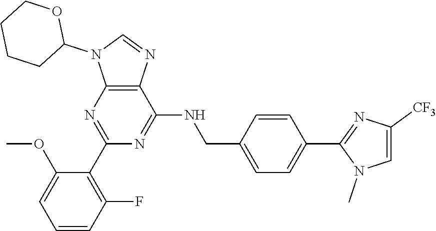 [SMILES: COc1cccc(F)c1-c1nc(NCc2ccc(-c3nc(C(F)(F)F)cn3C)cc2)c2ncn(C3CCCCO3)c2n1] |
| 30 | 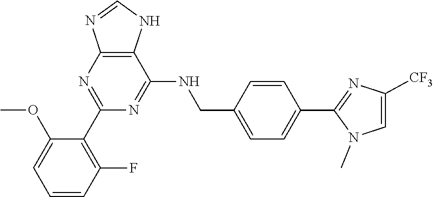 [SMILES: COc1cccc(F)c1-c1nc(NCc2ccc(-c3nc(C(F)(F)F)cn3C)cc2)c2[nH]cnc2n1] |
| 31 | 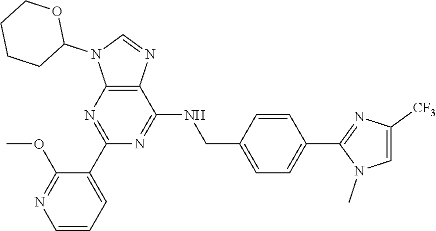 [SMILES: COc1ncccc1-c1nc(NCc2ccc(-c3nc(C(F)(F)F)cn3C)cc2)c2ncn(C3CCCCO3)c2n1] |
| 32 | 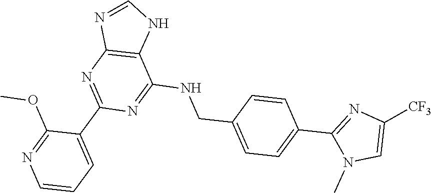 [SMILES: COc1ncccc1-c1nc(NCc2ccc(-c3nc(C(F)(F)F)cn3C)cc2)c2[nH]cnc2n1] |
| 33 | 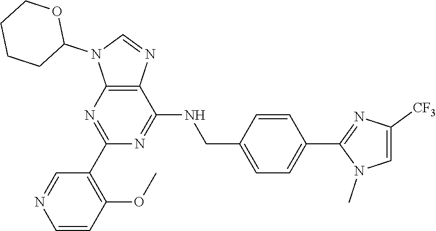 [SMILES: COc1ccncc1-c1nc(NCc2ccc(-c3nc(C(F)(F)F)cn3C)cc2)c2ncn(C3CCCCO3)c2n1] |
| 34 | 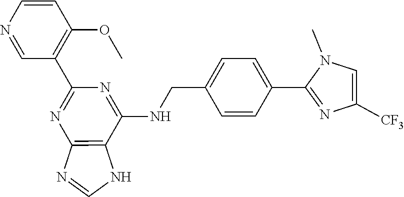 [SMILES: COc1ccncc1-c1nc(NCc2ccc(-c3nc(C(F)(F)F)cn3C)cc2)c2[nH]cnc2n1] |
| 35 | 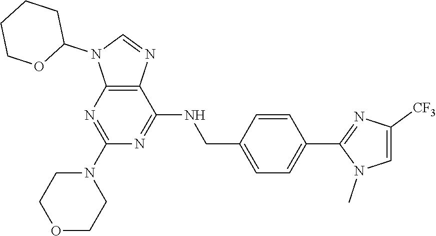 [SMILES: Cn1cc(C(F)(F)F)nc1-c1ccc(CNc2nc(N3CCOCC3)nc3c2ncn3C2CCCCO2)cc1] |
| 36 | 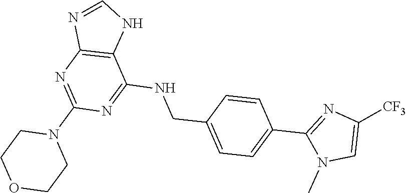 [SMILES: Cn1cc(C(F)(F)F)nc1-c1ccc(CNc2nc(N3CCOCC3)nc3nc[nH]c23)cc1] |
| 37 | 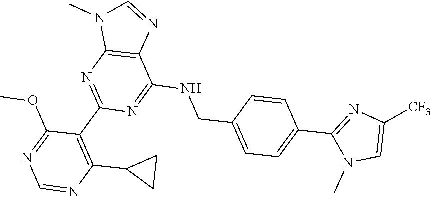 [SMILES: COc1ncnc(C2CC2)c1-c1nc(NCc2ccc(-c3nc(C(F)(F)F)cn3C)cc2)c2ncn(C)c2n1] |
| 38 | 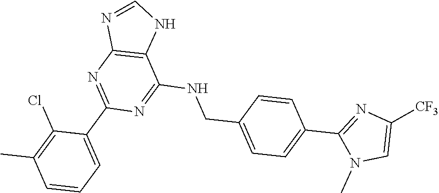 [SMILES: Cc1cccc(-c2nc(NCc3ccc(-c4nc(C(F)(F)F)cn4C)cc3)c3[nH]cnc3n2)c1Cl] |
| 39 | 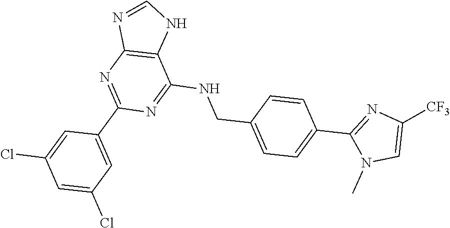 [SMILES: Cn1cc(C(F)(F)F)nc1-c1ccc(CNc2nc(-c3cc(Cl)cc(Cl)c3)nc3nc[nH]c23)cc1] |
| 40 | 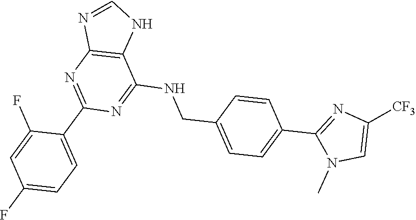 [SMILES: Cn1cc(C(F)(F)F)nc1-c1ccc(CNc2nc(-c3ccc(F)cc3F)nc3nc[nH]c23)cc1] |
| 41 | 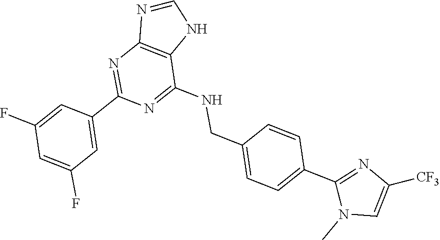 [SMILES: Cn1cc(C(F)(F)F)nc1-c1ccc(CNc2nc(-c3cc(F)cc(F)c3)nc3nc[nH]c23)cc1] |
| 42 | 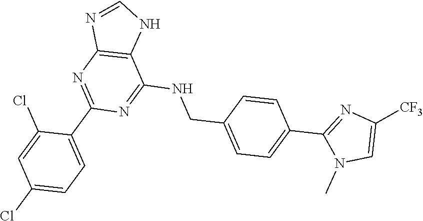 [SMILES: Cn1cc(C(F)(F)F)nc1-c1ccc(CNc2nc(-c3ccc(Cl)cc3Cl)nc3nc[nH]c23)cc1] |
| 43 | 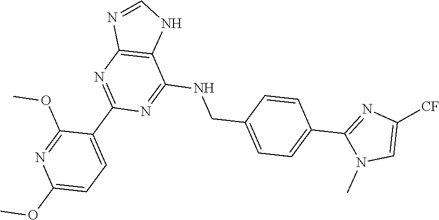 |
| 44 | 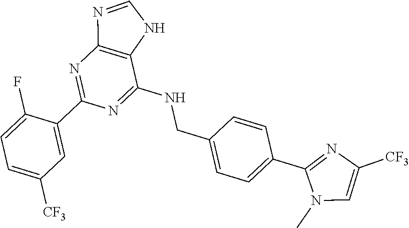 [SMILES: Cn1cc(C(F)(F)F)nc1-c1ccc(CNc2nc(-c3cc(C(F)(F)F)ccc3F)nc3nc[nH]c23)cc1] |
| 45 | 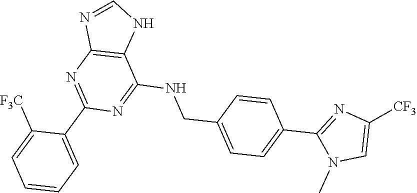 [SMILES: Cn1cc(C(F)(F)F)nc1-c1ccc(CNc2nc(-c3ccccc3C(F)(F)F)nc3nc[nH]c23)cc1] |
| 46 | 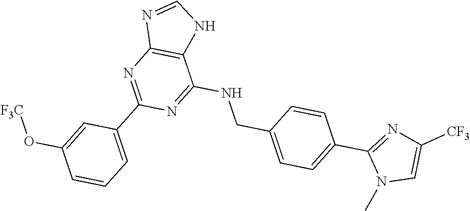 [SMILES: Cn1cc(C(F)(F)F)nc1-c1ccc(CNc2nc(-c3cccc(OC(F)(F)F)c3)nc3nc[nH]c23)cc1] |
| 47 | 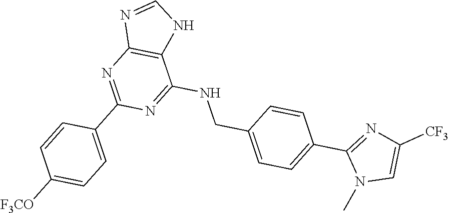 [SMILES: Cn1cc(C(F)(F)F)nc1-c1ccc(CNc2nc(-c3ccc(OC(F)(F)F)cc3)nc3nc[nH]c23)cc1] |
| 48 | 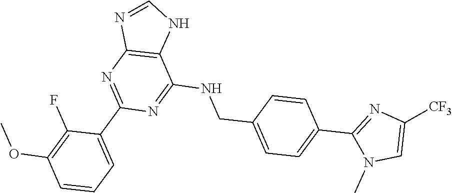 [SMILES: COc1cccc(-c2nc(NCc3ccc(-c4nc(C(F)(F)F)cn4C)cc3)c3[nH]cnc3n2)c1F] |
| 49 | 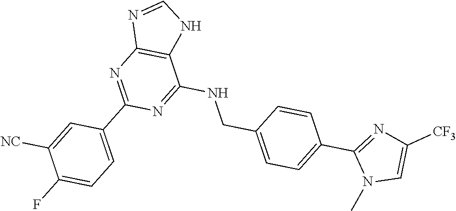 [SMILES: Cn1cc(C(F)(F)F)nc1-c1ccc(CNc2nc(-c3ccc(F)c(C#N)c3)nc3nc[nH]c23)cc1] |
| 50 | 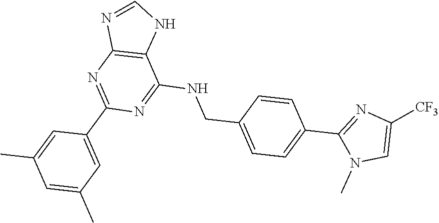 [SMILES: Cc1cc(C)cc(-c2nc(NCc3ccc(-c4nc(C(F)(F)F)cn4C)cc3)c3[nH]cnc3n2)c1] |
| 51 | 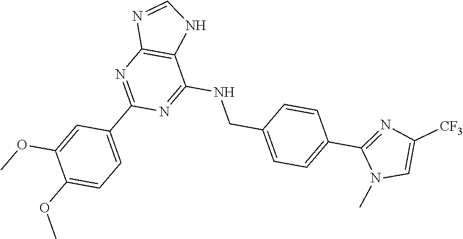 [SMILES: COc1ccc(-c2nc(NCc3ccc(-c4nc(C(F)(F)F)cn4C)cc3)c3[nH]cnc3n2)cc1OC] |
| 52 | 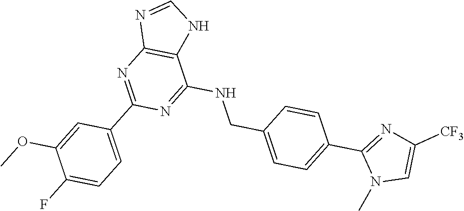 [SMILES: COc1cc(-c2nc(NCc3ccc(-c4nc(C(F)(F)F)cn4C)cc3)c3[nH]cnc3n2)ccc1F] |
| 53 | 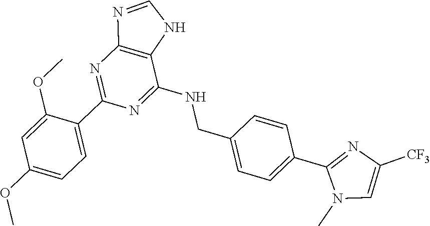 [SMILES: COc1ccc(-c2nc(NCc3ccc(-c4nc(C(F)(F)F)cn4C)cc3)c3[nH]cnc3n2)c(OC)c1] |
| 54 | 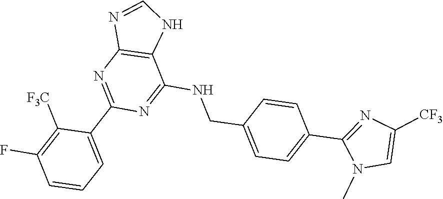 [SMILES: Cn1cc(C(F)(F)F)nc1-c1ccc(CNc2nc(-c3cccc(F)c3C(F)(F)F)nc3nc[nH]c23)cc1] |
| 55 | 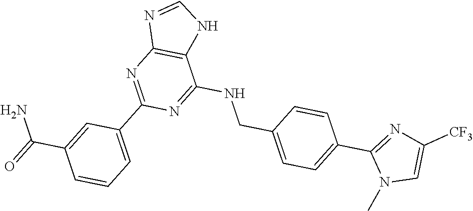 [SMILES: Cn1cc(C(F)(F)F)nc1-c1ccc(CNc2nc(-c3cccc(C(N)=O)c3)nc3nc[nH]c23)cc1] |
| 56 | 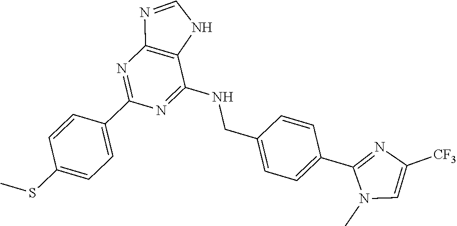 [SMILES: CSc1ccc(-c2nc(NCc3ccc(-c4nc(C(F)(F)F)cn4C)cc3)c3[nH]cnc3n2)cc1] |
| 57 | 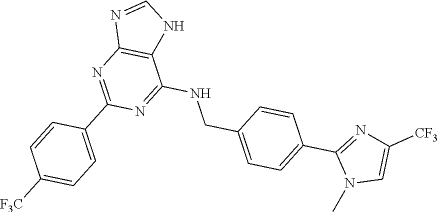 [SMILES: Cn1cc(C(F)(F)F)nc1-c1ccc(CNc2nc(-c3ccc(C(F)(F)F)cc3)nc3nc[nH]c23)cc1] |
| 58 | 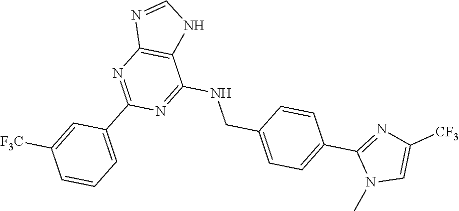 [SMILES: Cn1cc(C(F)(F)F)nc1-c1ccc(CNc2nc(-c3cccc(C(F)(F)F)c3)nc3nc[nH]c23)cc1] |
| 59 | 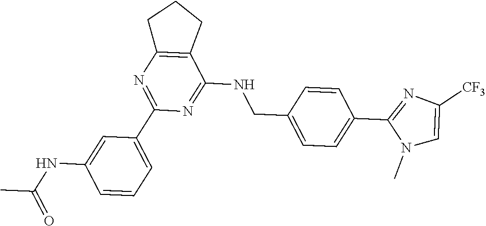 [SMILES: CC(=O)Nc1cccc(-c2nc3c(c(NCc4ccc(-c5nc(C(F)(F)F)cn5C)cc4)n2)CCC3)c1] |
| 60 | 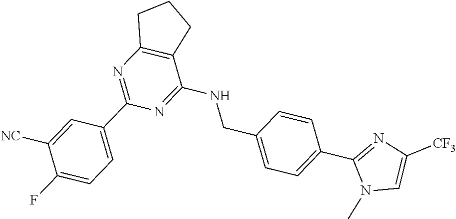 [SMILES: Cn1cc(C(F)(F)F)nc1-c1ccc(CNc2nc(-c3ccc(F)c(C#N)c3)nc3c2CCC3)cc1] |
| 61 | 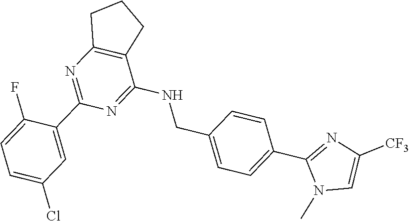 [SMILES: Cn1cc(C(F)(F)F)nc1-c1ccc(CNc2nc(-c3cc(Cl)ccc3F)nc3c2CCC3)cc1] |
| 62 | 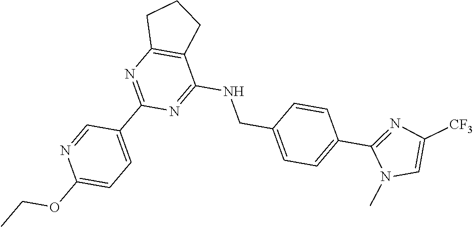 [SMILES: CCOc1ccc(-c2nc3c(c(NCc4ccc(-c5nc(C(F)(F)F)cn5C)cc4)n2)CCC3)cn1] |
| 63 | 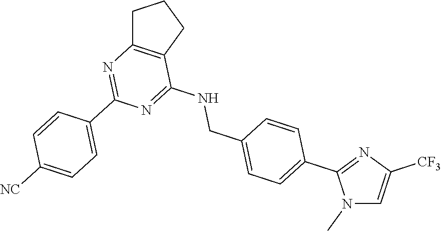 [SMILES: Cn1cc(C(F)(F)F)nc1-c1ccc(CNc2nc(-c3ccc(C#N)cc3)nc3c2CCC3)cc1] |
| 64 | 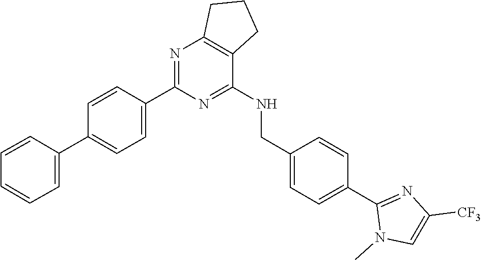 [SMILES: Cn1cc(C(F)(F)F)nc1-c1ccc(CNc2nc(-c3ccc(-c4ccccc4)cc3)nc3c2CCC3)cc1] |
| 65 | 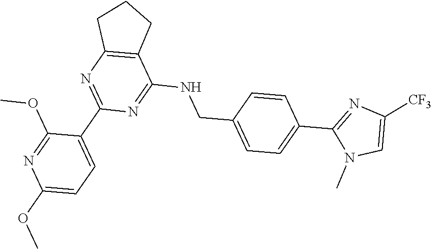 [SMILES: COc1ccc(-c2nc3c(c(NCc4ccc(-c5nc(C(F)(F)F)cn5C)cc4)n2)CCC3)c(OC)n1] |
| 66 | 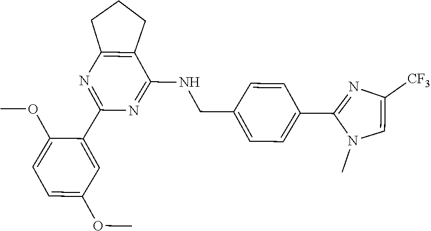 [SMILES: COc1ccc(OC)c(-c2nc3c(c(NCc4ccc(-c5nc(C(F)(F)F)cn5C)cc4)n2)CCC3)c1] |
| 67 | 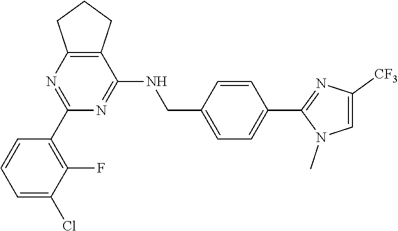 [SMILES: Cn1cc(C(F)(F)F)nc1-c1ccc(CNc2nc(-c3cccc(Cl)c3F)nc3c2CCC3)cc1] |
| 68 | 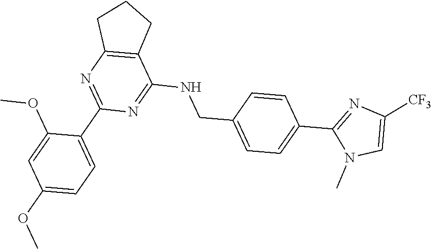 [SMILES: COc1ccc(-c2nc3c(c(NCc4ccc(-c5nc(C(F)(F)F)cn5C)cc4)n2)CCC3)c(OC)c1] |
| 69 | 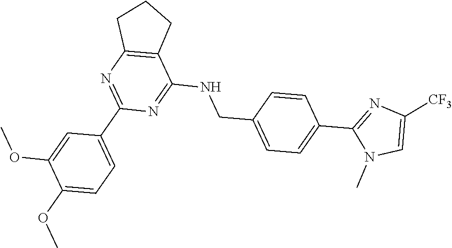 [SMILES: COc1ccc(-c2nc3c(c(NCc4ccc(-c5nc(C(F)(F)F)cn5C)cc4)n2)CCC3)cc1OC] |
| 70 | 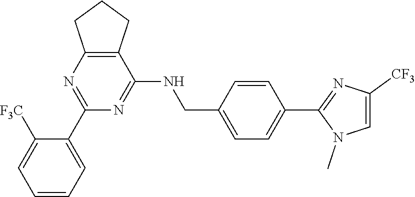 [SMILES: Cn1cc(C(F)(F)F)nc1-c1ccc(CNc2nc(-c3ccccc3C(F)(F)F)nc3c2CCC3)cc1] |
| 71 | 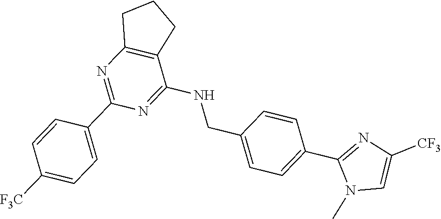 [SMILES: Cn1cc(C(F)(F)F)nc1-c1ccc(CNc2nc(-c3ccc(C(F)(F)F)cc3)nc3c2CCC3)cc1] |
| 72 | 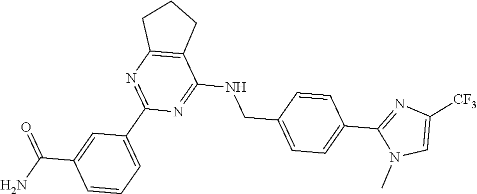 [SMILES: Cn1cc(C(F)(F)F)nc1-c1ccc(CNc2nc(-c3cccc(C(N)=O)c3)nc3c2CCC3)cc1] |
| 73 | 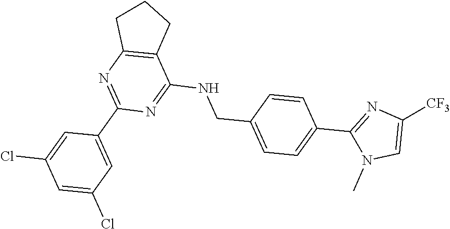 [SMILES: Cn1cc(C(F)(F)F)nc1-c1ccc(CNc2nc(-c3cc(Cl)cc(Cl)c3)nc3c2CCC3)cc1] |
| 74 | 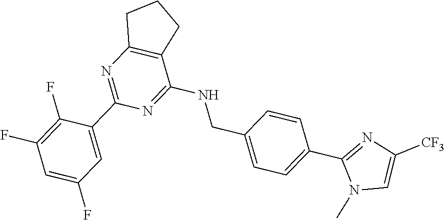 [SMILES: Cn1cc(C(F)(F)F)nc1-c1ccc(CNc2nc(-c3cc(F)cc(F)c3F)nc3c2CCC3)cc1] |
| 75 | 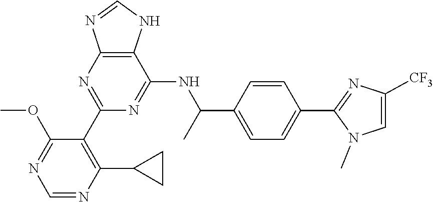 [SMILES: COc1ncnc(C2CC2)c1-c1nc(NC(C)c2ccc(-c3nc(C(F)(F)F)cn3C)cc2)c2[nH]cnc2n1] |
| 76 | 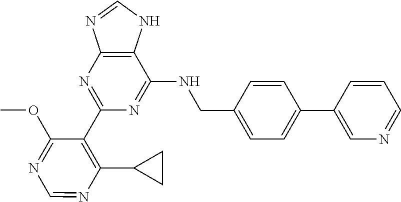 [SMILES: COc1ncnc(C2CC2)c1-c1nc(NCc2ccc(-c3cccnc3)cc2)c2[nH]cnc2n1] |
| 77 | 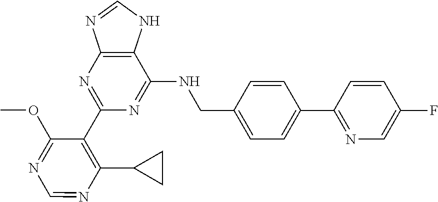 [SMILES: COc1ncnc(C2CC2)c1-c1nc(NCc2ccc(-c3ccc(F)cn3)cc2)c2[nH]cnc2n1] |

The non-limiting representative compounds of the present invention as listed herein below, and pharmaceutically acceptable salts thereof are

TABLE-2

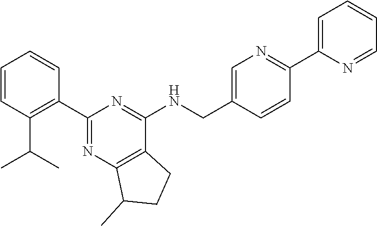 [SMILES: CC(C)c1ccccc1-c1nc(NCc2ccc(-c3ccccn3)nc2)c2c(n1)C(C)CC2]

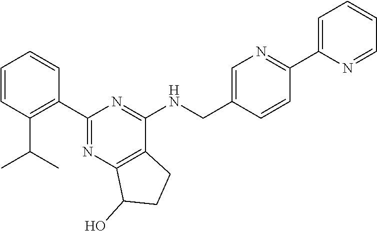 [SMILES: CC(C)c1ccccc1-c1nc(NCc2ccc(-c3ccccn3)nc2)c2c(n1)C(O)CC2]

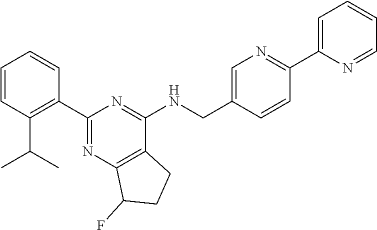 [SMILES: CC(C)c1ccccc1-c1nc(NCc2ccc(-c3ccccn3)nc2)c2c(n1)C(F)CC2]

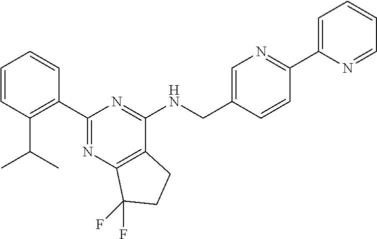 [SMILES: CC(C)c1ccccc1-c1nc(NCc2ccc(-c3ccccn3)nc2)c2c(n1)C(F)(F)CC2]

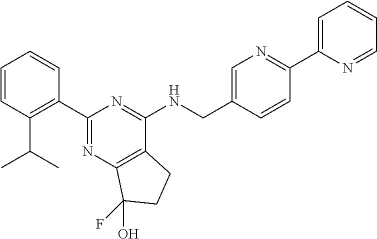 [SMILES: CC(C)c1ccccc1-c1nc(NCc2ccc(-c3ccccn3)nc2)c2c(n1)C(O)(F)CC2]

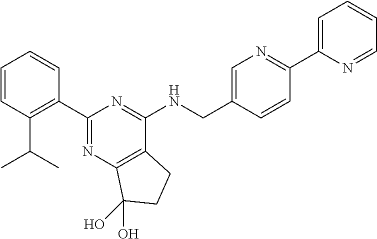 [SMILES: CC(C)c1ccccc1-c1nc(NCc2ccc(-c3ccccn3)nc2)c2c(n1)C(O)(O)CC2]

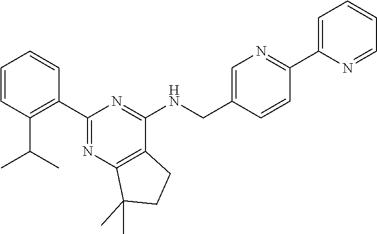 [SMILES: CC(C)c1ccccc1-c1nc(NCc2ccc(-c3ccccn3)nc2)c2c(n1)C(C)(C)CC2]

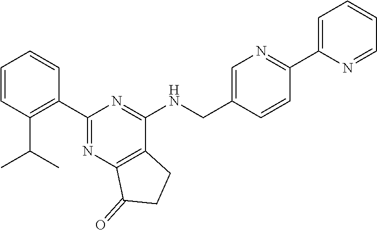 [SMILES: CC(C)c1ccccc1-c1nc(NCc2ccc(-c3ccccn3)nc2)c2c(n1)C(=O)CC2]

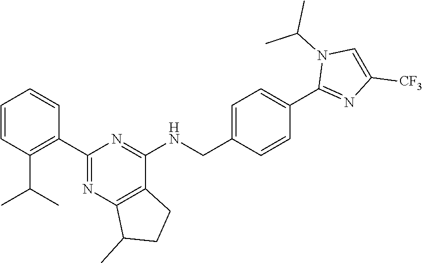 [SMILES: CC(C)c1ccccc1-c1nc(NCc2ccc(-c3nc(C(F)(F)F)cn3C(C)C)cc2)c2c(n1)C(C)CC2]

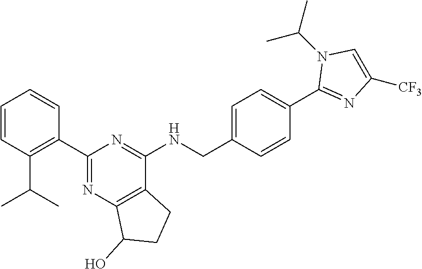 [SMILES: CC(C)c1ccccc1-c1nc(NCc2ccc(-c3nc(C(F)(F)F)cn3C(C)C)cc2)c2c(n1)C(O)CC2]

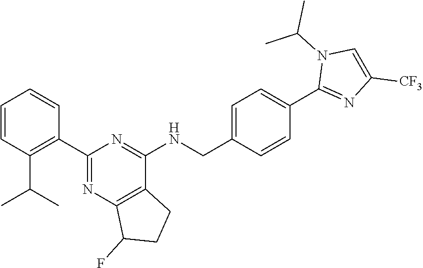 [SMILES: CC(C)c1ccccc1-c1nc(NCc2ccc(-c3nc(C(F)(F)F)cn3C(C)C)cc2)c2c(n1)C(F)CC2]

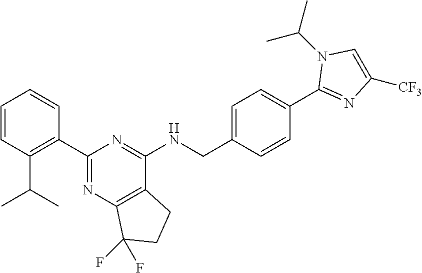 [SMILES: CC(C)c1ccccc1-c1nc(NCc2ccc(-c3nc(C(F)(F)F)cn3C(C)C)cc2)c2c(n1)C(F)(F)CC2]

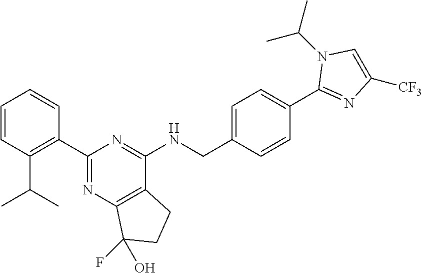 [SMILES: CC(C)c1ccccc1-c1nc(NCc2ccc(-c3nc(C(F)(F)F)cn3C(C)C)cc2)c2c(n1)C(O)(F)CC2]

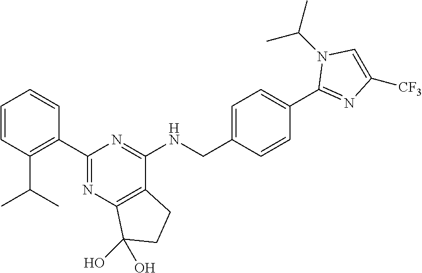 [SMILES: CC(C)c1ccccc1-c1nc(NCc2ccc(-c3nc(C(F)(F)F)cn3C(C)C)cc2)c2c(n1)C(O)(O)CC2]

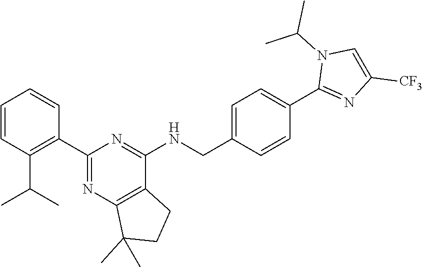 [SMILES: CC(C)c1ccccc1-c1nc(NCc2ccc(-c3nc(C(F)(F)F)cn3C(C)C)cc2)c2c(n1)C(C)(C)CC2]

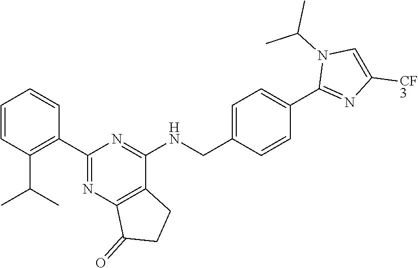 [SMILES: CC(C)c1ccccc1-c1nc(NCc2ccc(-c3nc(C(F)(F)F)cn3C(C)C)cc2)c2c(n1)C(=O)CC2]

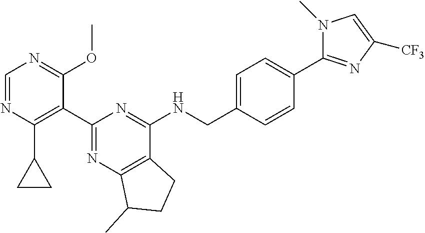 [SMILES: COc1ncnc(C2CC2)c1-c1nc(NCc2ccc(-c3nc(C(F)(F)F)cn3C)cc2)c2c(n1)C(C)CC2]

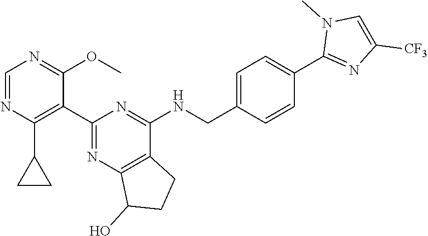 [SMILES: COc1ncnc(C2CC2)c1-c1nc(NCc2ccc(-c3nc(C(F)(F)F)cn3C)cc2)c2c(n1)C(O)CC2]

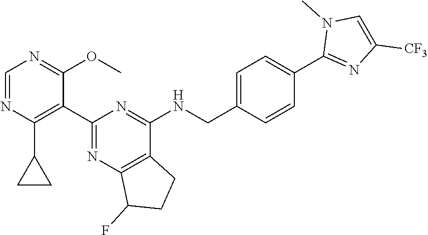 [SMILES: COc1ncnc(C2CC2)c1-c1nc(NCc2ccc(-c3nc(C(F)(F)F)cn3C)cc2)c2c(n1)C(F)CC2]

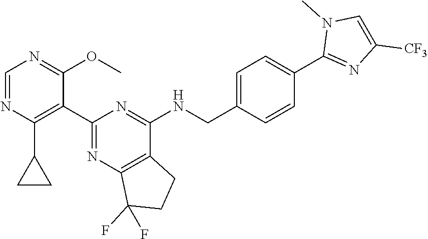 [SMILES: COc1ncnc(C2CC2)c1-c1nc(NCc2ccc(-c3nc(C(F)(F)F)cn3C)cc2)c2c(n1)C(F)(F)CC2]

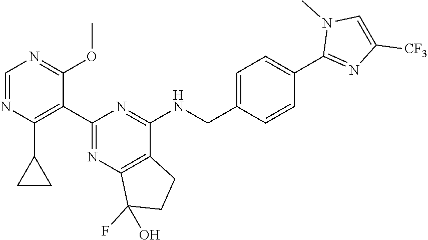 [SMILES: COc1ncnc(C2CC2)c1-c1nc(NCc2ccc(-c3nc(C(F)(F)F)cn3C)cc2)c2c(n1)C(O)(F)CC2]

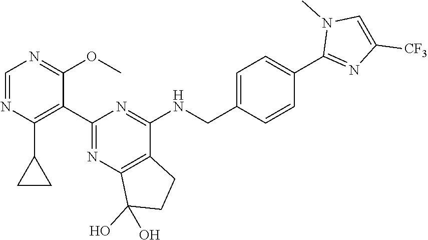 [SMILES: COc1ncnc(C2CC2)c1-c1nc(NCc2ccc(-c3nc(C(F)(F)F)cn3C)cc2)c2c(n1)C(O)(O)CC2]

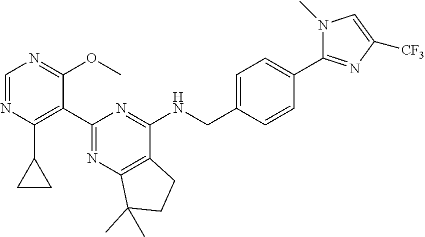 [SMILES: COc1ncnc(C2CC2)c1-c1nc(NCc2ccc(-c3nc(C(F)(F)F)cn3C)cc2)c2c(n1)C(C)(C)CC2]

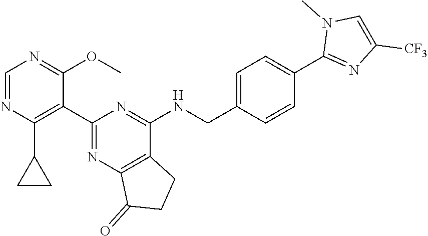 [SMILES: COc1ncnc(C2CC2)c1-c1nc(NCc2ccc(-c3nc(C(F)(F)F)cn3C)cc2)c2c(n1)C(=O)CC2]

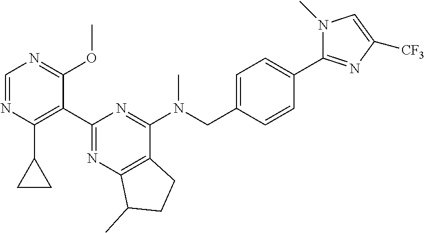 [SMILES: COc1ncnc(C2CC2)c1-c1nc2c(c(N(C)Cc3ccc(-c4nc(C(F)(F)F)cn4C)cc3)n1)CCC2C]

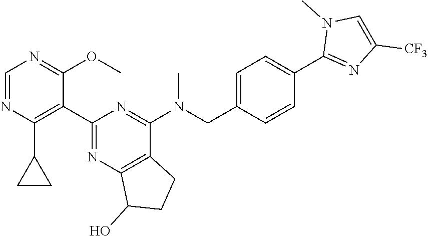 [SMILES: COc1ncnc(C2CC2)c1-c1nc2c(c(N(C)Cc3ccc(-c4nc(C(F)(F)F)cn4C)cc3)n1)CCC2O]

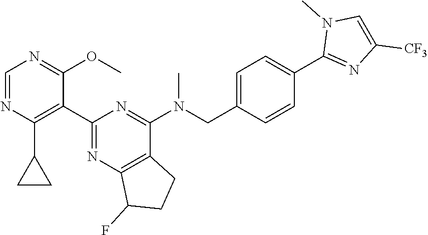 [SMILES: COc1ncnc(C2CC2)c1-c1nc2c(c(N(C)Cc3ccc(-c4nc(C(F)(F)F)cn4C)cc3)n1)CCC2F]

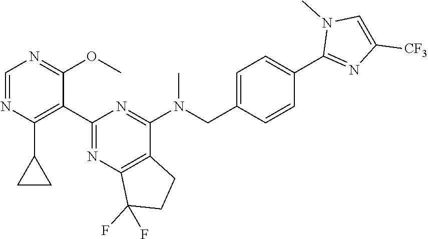 [SMILES: COc1ncnc(C2CC2)c1-c1nc(N(C)Cc2ccc(-c3nc(C(F)(F)F)cn3C)cc2)c2c(n1)C(F)(F)CC2]

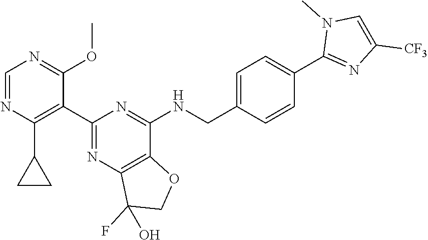 [SMILES: COc1ncnc(C2CC2)c1-c1nc(NCc2ccc(-c3nc(C(F)(F)F)cn3C)cc2)c2c(n1)C(O)(F)CO2]

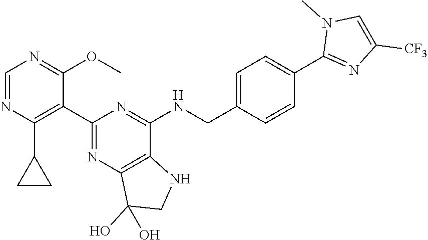 [SMILES: COc1ncnc(C2CC2)c1-c1nc(NCc2ccc(-c3nc(C(F)(F)F)cn3C)cc2)c2c(n1)C(O)(O)CN2]

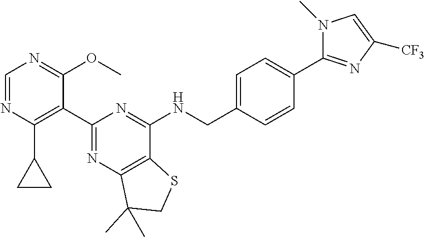 [SMILES: COc1ncnc(C2CC2)c1-c1nc(NCc2ccc(-c3nc(C(F)(F)F)cn3C)cc2)c2c(n1)C(C)(C)CS2]

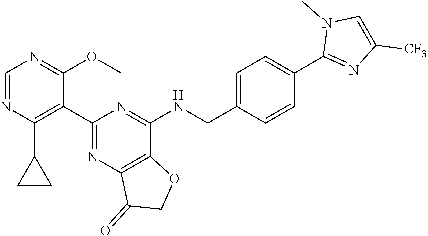 [SMILES: COc1ncnc(C2CC2)c1-c1nc(NCc2ccc(-c3nc(C(F)(F)F)cn3C)cc2)c2c(n1)C(=O)CO2]

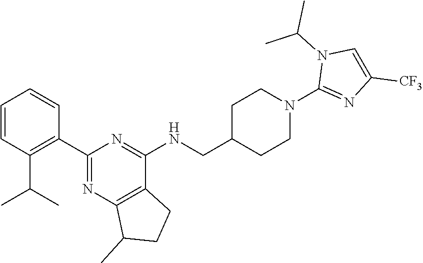 [SMILES: CC(C)c1ccccc1-c1nc(NCC2CCN(c3nc(C(F)(F)F)cn3C(C)C)CC2)c2c(n1)C(C)CC2]

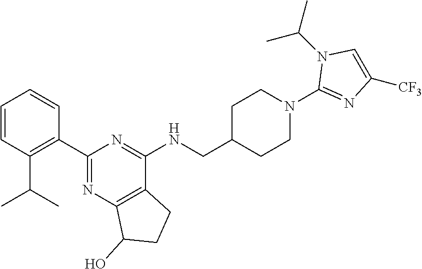 [SMILES: CC(C)c1ccccc1-c1nc(NCC2CCN(c3nc(C(F)(F)F)cn3C(C)C)CC2)c2c(n1)C(O)CC2]

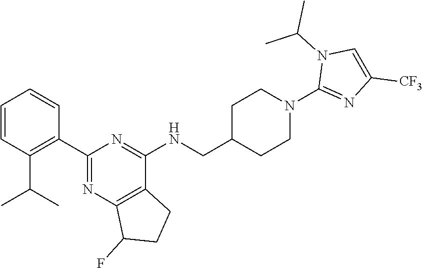 [SMILES: CC(C)c1ccccc1-c1nc(NCC2CCN(c3nc(C(F)(F)F)cn3C(C)C)CC2)c2c(n1)C(F)CC2]

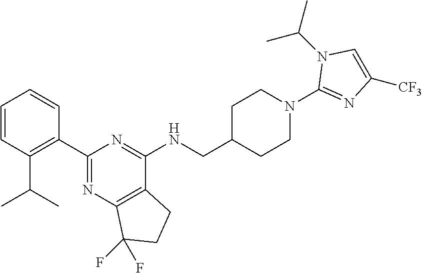 [SMILES: CC(C)c1ccccc1-c1nc(NCC2CCN(c3nc(C(F)(F)F)cn3C(C)C)CC2)c2c(n1)C(F)(F)CC2]

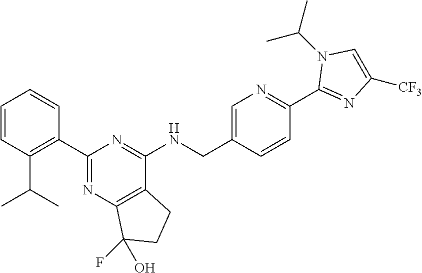 [SMILES: CC(C)c1ccccc1-c1nc(NCc2ccc(-c3nc(C(F)(F)F)cn3C(C)C)nc2)c2c(n1)C(O)(F)CC2]

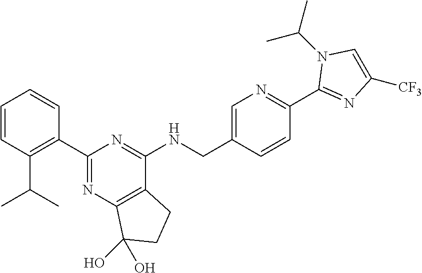 [SMILES: CC(C)c1ccccc1-c1nc(NCc2ccc(-c3nc(C(F)(F)F)cn3C(C)C)nc2)c2c(n1)C(O)(O)CC2]

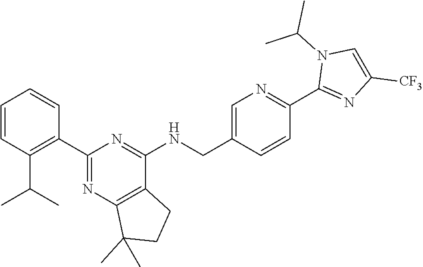 [SMILES: CC(C)c1ccccc1-c1nc(NCc2ccc(-c3nc(C(F)(F)F)cn3C(C)C)nc2)c2c(n1)C(C)(C)CC2]

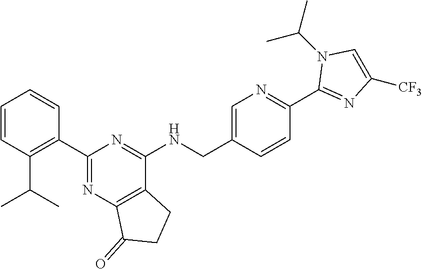 [SMILES: CC(C)c1ccccc1-c1nc(NCc2ccc(-c3nc(C(F)(F)F)cn3C(C)C)nc2)c2c(n1)C(=O)CC2]

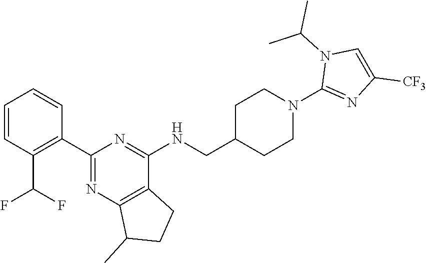 [SMILES: CC1CCc2c(NCC3CCN(c4nc(C(F)(F)F)cn4C(C)C)CC3)nc(-c3ccccc3C(F)F)nc21]

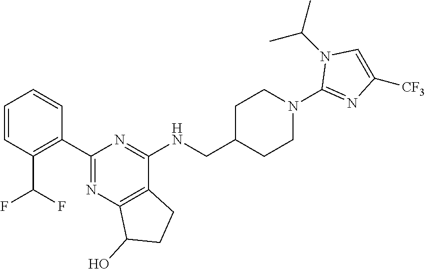 [SMILES: CC(C)n1cc(C(F)(F)F)nc1N1CCC(CNc2nc(-c3ccccc3C(F)F)nc3c2CCC3O)CC1]

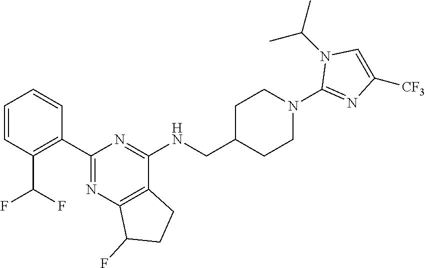 [SMILES: CC(C)n1cc(C(F)(F)F)nc1N1CCC(CNc2nc(-c3ccccc3C(F)F)nc3c2CCC3F)CC1]

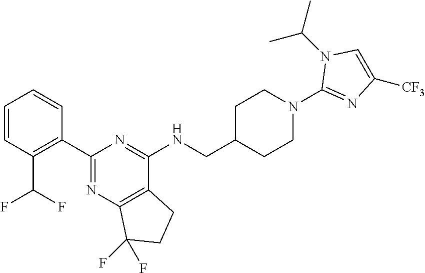 [SMILES: CC(C)n1cc(C(F)(F)F)nc1N1CCC(CNc2nc(-c3ccccc3C(F)F)nc3c2CCC3(F)F)CC1]

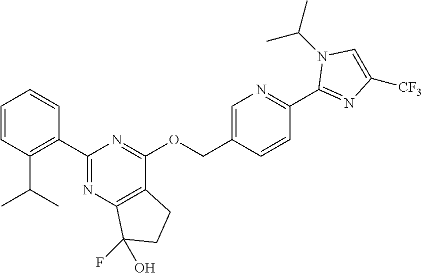 [SMILES: CC(C)c1ccccc1-c1nc(OCc2ccc(-c3nc(C(F)(F)F)cn3C(C)C)nc2)c2c(n1)C(O)(F)CC2]

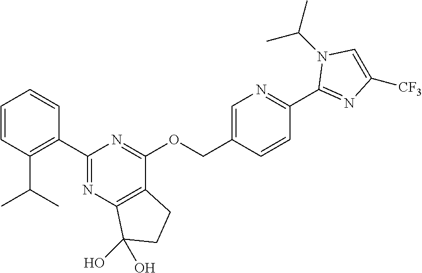 [SMILES: CC(C)c1ccccc1-c1nc(OCc2ccc(-c3nc(C(F)(F)F)cn3C(C)C)nc2)c2c(n1)C(O)(O)CC2]

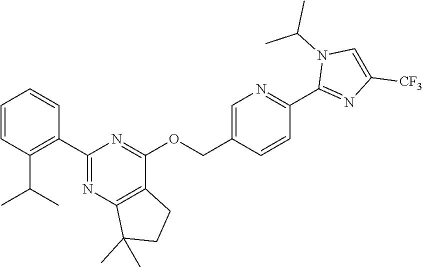 [SMILES: CC(C)c1ccccc1-c1nc(OCc2ccc(-c3nc(C(F)(F)F)cn3C(C)C)nc2)c2c(n1)C(C)(C)CC2]

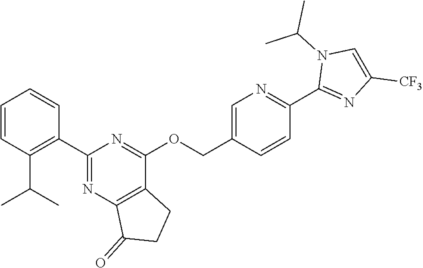 [SMILES: CC(C)c1ccccc1-c1nc(OCc2ccc(-c3nc(C(F)(F)F)cn3C(C)C)nc2)c2c(n1)C(=O)CC2]

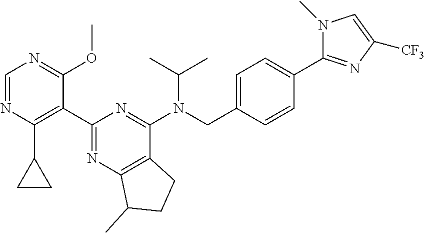 [SMILES: COc1ncnc(C2CC2)c1-c1nc2c(c(N(Cc3ccc(-c4nc(C(F)(F)F)cn4C)cc3)C(C)C)n1)CCC2C]

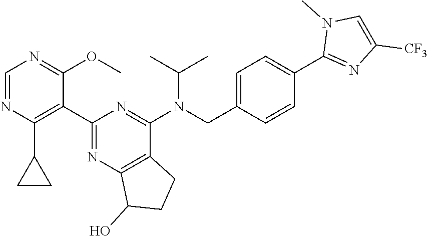 [SMILES: COc1ncnc(C2CC2)c1-c1nc2c(c(N(Cc3ccc(-c4nc(C(F)(F)F)cn4C)cc3)C(C)C)n1)CCC2O]

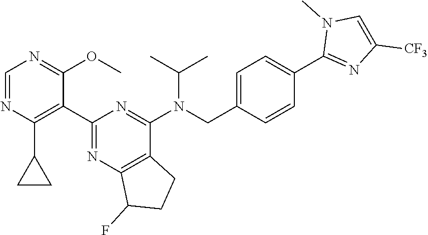 [SMILES: COc1ncnc(C2CC2)c1-c1nc2c(c(N(Cc3ccc(-c4nc(C(F)(F)F)cn4C)cc3)C(C)C)n1)CCC2F]

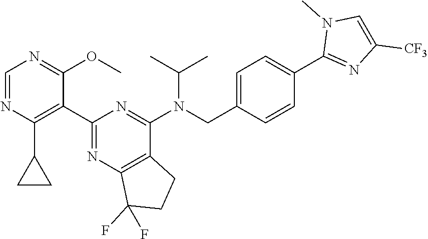 [SMILES: COc1ncnc(C2CC2)c1-c1nc(N(Cc2ccc(-c3nc(C(F)(F)F)cn3C)cc2)C(C)C)c2c(n1)C(F)(F)CC2]

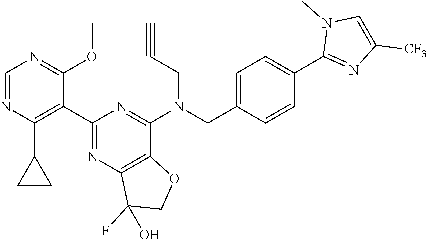 [SMILES: C#CCN(Cc1ccc(-c2nc(C(F)(F)F)cn2C)cc1)c1nc(-c2c(OC)ncnc2C2CC2)nc2c1OCC2(O)F]

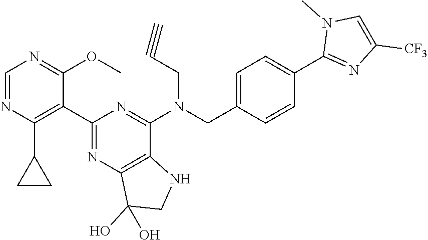 [SMILES: C#CCN(Cc1ccc(-c2nc(C(F)(F)F)cn2C)cc1)c1nc(-c2c(OC)ncnc2C2CC2)nc2c1NCC2(O)O]

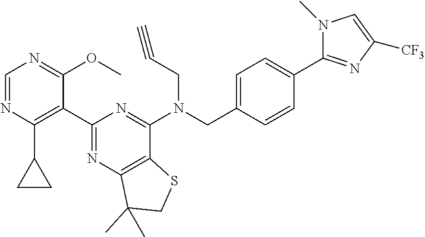 [SMILES: C#CCN(Cc1ccc(-c2nc(C(F)(F)F)cn2C)cc1)c1nc(-c2c(OC)ncnc2C2CC2)nc2c1SCC2(C)C]

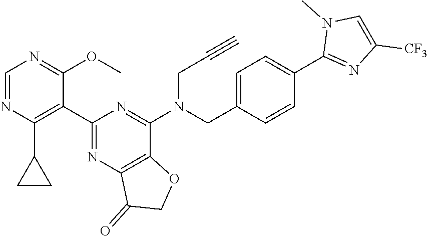 [SMILES: C#CCN(Cc1ccc(-c2nc(C(F)(F)F)cn2C)cc1)c1nc(-c2c(OC)ncnc2C2CC2)nc2c1OCC2=O]

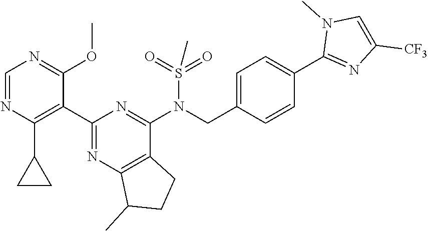 [SMILES: COc1ncnc(C2CC2)c1-c1nc2c(c(N(Cc3ccc(-c4nc(C(F)(F)F)cn4C)cc3)S(C)(=O)=O)n1)CCC2C]

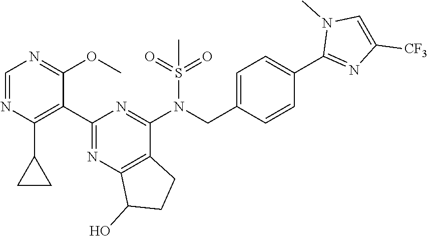 [SMILES: COc1ncnc(C2CC2)c1-c1nc2c(c(N(Cc3ccc(-c4nc(C(F)(F)F)cn4C)cc3)S(C)(=O)=O)n1)CCC2O]

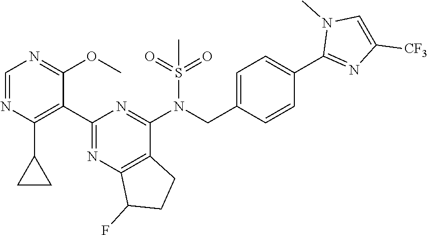 [SMILES: COc1ncnc(C2CC2)c1-c1nc2c(c(N(Cc3ccc(-c4nc(C(F)(F)F)cn4C)cc3)S(C)(=O)=O)n1)CCC2F]

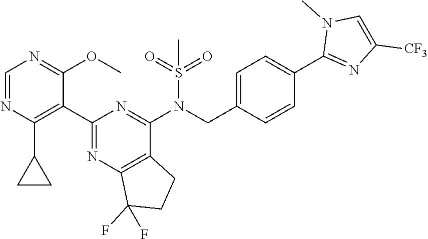 [SMILES: COc1ncnc(C2CC2)c1-c1nc(N(Cc2ccc(-c3nc(C(F)(F)F)cn3C)cc2)S(C)(=O)=O)c2c(n1)C(F)(F)CC2]

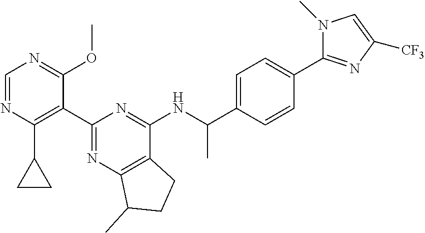 [SMILES: COc1ncnc(C2CC2)c1-c1nc(NC(C)c2ccc(-c3nc(C(F)(F)F)cn3C)cc2)c2c(n1)C(C)CC2]

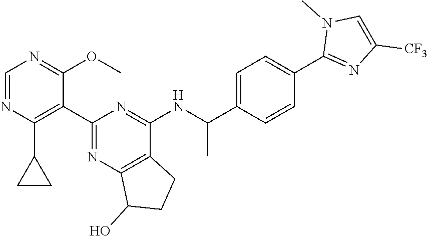 [SMILES: COc1ncnc(C2CC2)c1-c1nc(NC(C)c2ccc(-c3nc(C(F)(F)F)cn3C)cc2)c2c(n1)C(O)CC2]

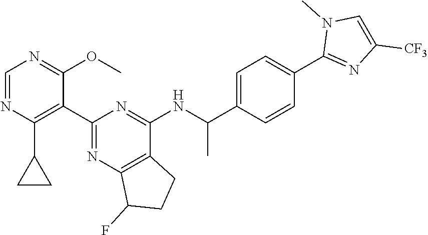 [SMILES: COc1ncnc(C2CC2)c1-c1nc(NC(C)c2ccc(-c3nc(C(F)(F)F)cn3C)cc2)c2c(n1)C(F)CC2]

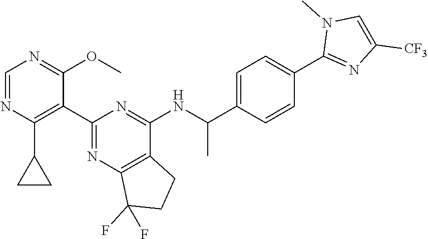 [SMILES: COc1ncnc(C2CC2)c1-c1nc(NC(C)c2ccc(-c3nc(C(F)(F)F)cn3C)cc2)c2c(n1)C(F)(F)CC2]

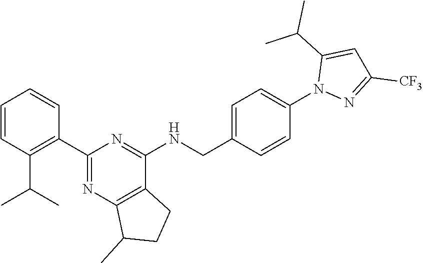 [SMILES: CC(C)c1ccccc1-c1nc(NCc2ccc(-n3nc(C(F)(F)F)cc3C(C)C)cc2)c2c(n1)C(C)CC2]

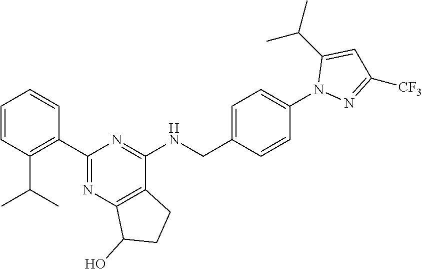 [SMILES: CC(C)c1ccccc1-c1nc(NCc2ccc(-n3nc(C(F)(F)F)cc3C(C)C)cc2)c2c(n1)C(O)CC2]

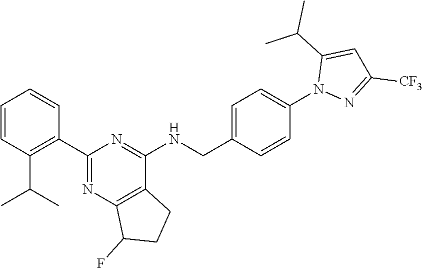 [SMILES: CC(C)c1ccccc1-c1nc(NCc2ccc(-n3nc(C(F)(F)F)cc3C(C)C)cc2)c2c(n1)C(F)CC2]

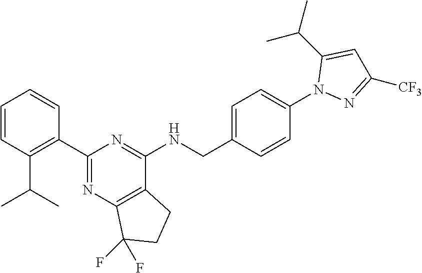 [SMILES: CC(C)c1ccccc1-c1nc(NCc2ccc(-n3nc(C(F)(F)F)cc3C(C)C)cc2)c2c(n1)C(F)(F)CC2]

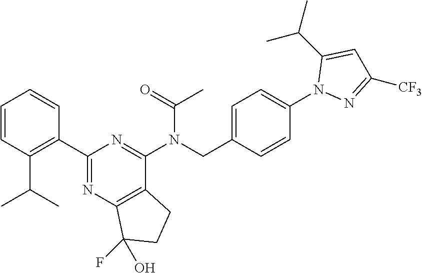 [SMILES: CC(=O)N(Cc1ccc(-n2nc(C(F)(F)F)cc2C(C)C)cc1)c1nc(-c2ccccc2C(C)C)nc2c1CCC2(O)F]

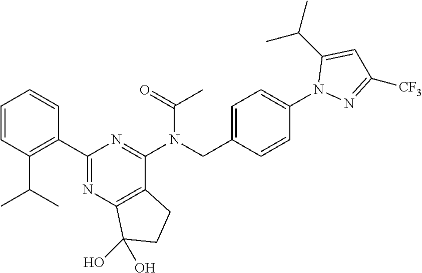 [SMILES: CC(=O)N(Cc1ccc(-n2nc(C(F)(F)F)cc2C(C)C)cc1)c1nc(-c2ccccc2C(C)C)nc2c1CCC2(O)O]

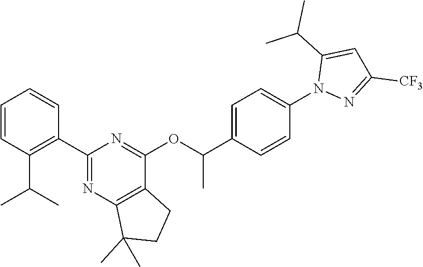 [SMILES: CC(C)c1ccccc1-c1nc(OC(C)c2ccc(-n3nc(C(F)(F)F)cc3C(C)C)cc2)c2c(n1)C(C)(C)CC2]

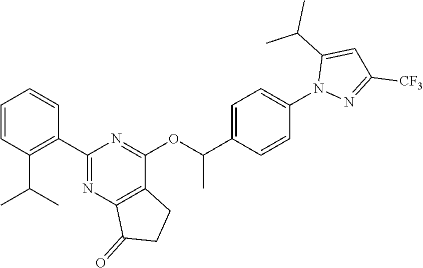 [SMILES: CC(C)c1ccccc1-c1nc(OC(C)c2ccc(-n3nc(C(F)(F)F)cc3C(C)C)cc2)c2c(n1)C(=O)CC2]

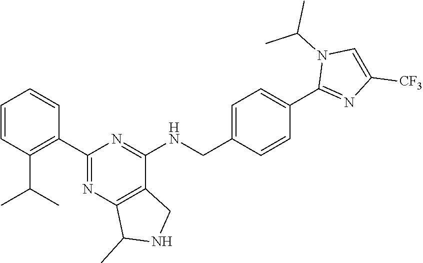 [SMILES: CC(C)c1ccccc1-c1nc(NCc2ccc(-c3nc(C(F)(F)F)cn3C(C)C)cc2)c2c(n1)C(C)NC2]

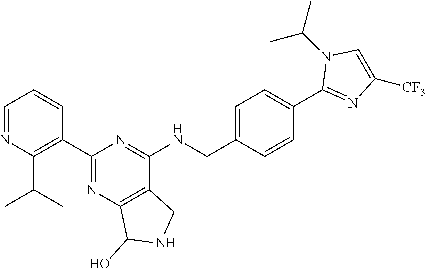 [SMILES: CC(C)c1ncccc1-c1nc(NCc2ccc(-c3nc(C(F)(F)F)cn3C(C)C)cc2)c2c(n1)C(O)NC2]

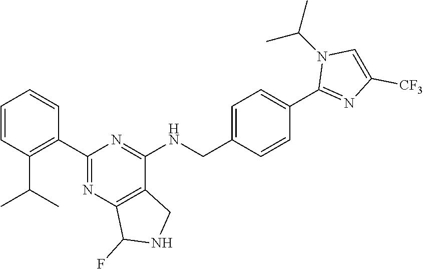 [SMILES: CC(C)c1ccccc1-c1nc(NCc2ccc(-c3nc(C(F)(F)F)cn3C(C)C)cc2)c2c(n1)C(F)NC2]

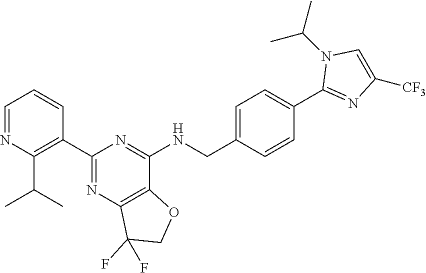 [SMILES: CC(C)c1ncccc1-c1nc(NCc2ccc(-c3nc(C(F)(F)F)cn3C(C)C)cc2)c2c(n1)C(F)(F)CO2]

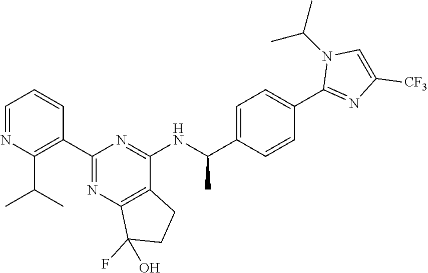 [SMILES: CC(C)c1ncccc1-c1nc(N[C@H](C)c2ccc(-c3nc(C(F)(F)F)cn3C(C)C)cc2)c2c(n1)C(O)(F)CC2]

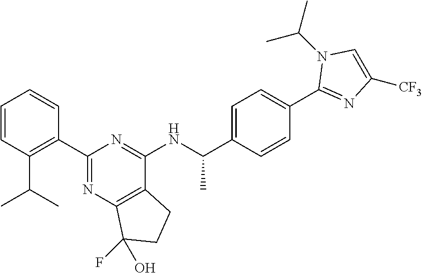 [SMILES: CC(C)c1ccccc1-c1nc(N[C@@H](C)c2ccc(-c3nc(C(F)(F)F)cn3C(C)C)cc2)c2c(n1)C(O)(F)CC2]

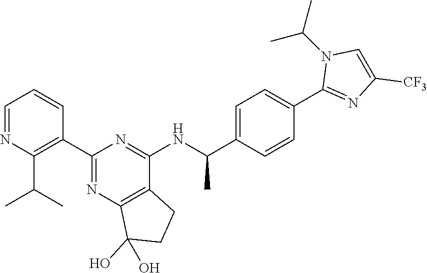 [SMILES: CC(C)c1ncccc1-c1nc(N[C@H](C)c2ccc(-c3nc(C(F)(F)F)cn3C(C)C)cc2)c2c(n1)C(O)(O)CC2]

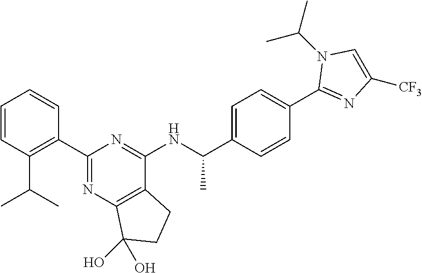 [SMILES: CC(C)c1ccccc1-c1nc(N[C@@H](C)c2ccc(-c3nc(C(F)(F)F)cn3C(C)C)cc2)c2c(n1)C(O)(O)CC2]

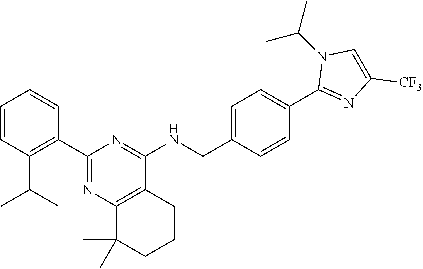 [SMILES: CC(C)c1ccccc1-c1nc(NCc2ccc(-c3nc(C(F)(F)F)cn3C(C)C)cc2)c2c(n1)C(C)(C)CCC2]

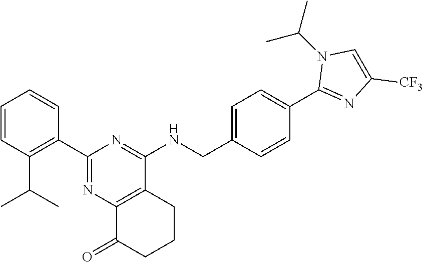 [SMILES: CC(C)c1ccccc1-c1nc(NCc2ccc(-c3nc(C(F)(F)F)cn3C(C)C)cc2)c2c(n1)C(=O)CCC2]

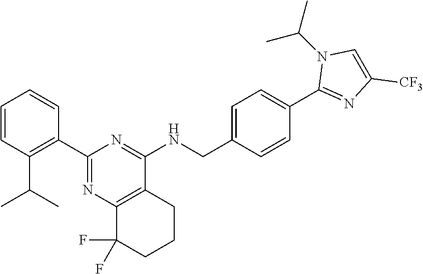 [SMILES: CC(C)c1ccccc1-c1nc(NCc2ccc(-c3nc(C(F)(F)F)cn3C(C)C)cc2)c2c(n1)C(F)(F)CCC2]

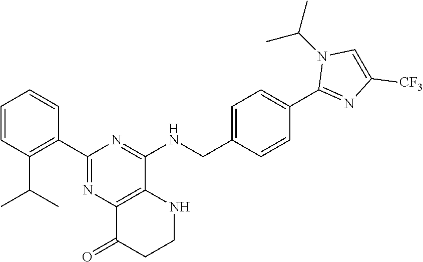 [SMILES: CC(C)c1ccccc1-c1nc(NCc2ccc(-c3nc(C(F)(F)F)cn3C(C)C)cc2)c2c(n1)C(=O)CCN2]

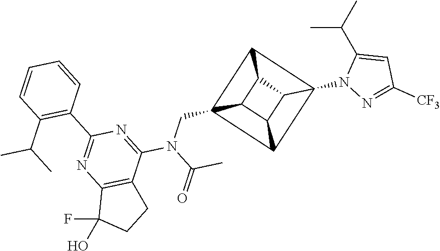

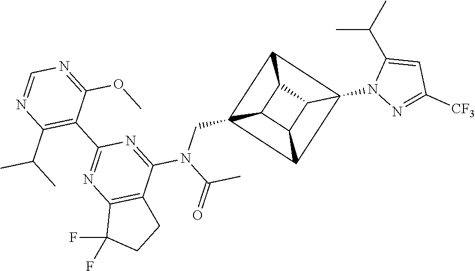

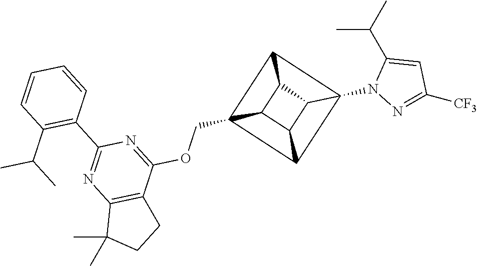

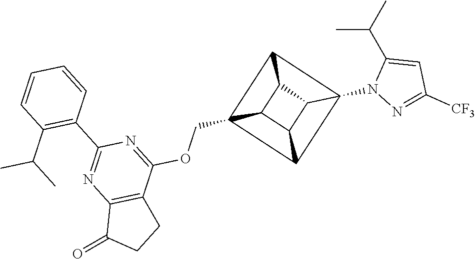

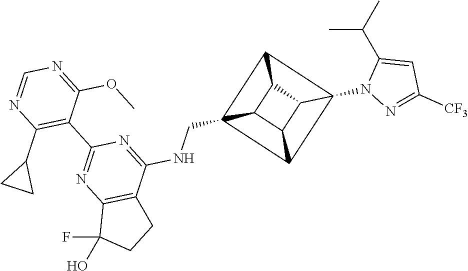

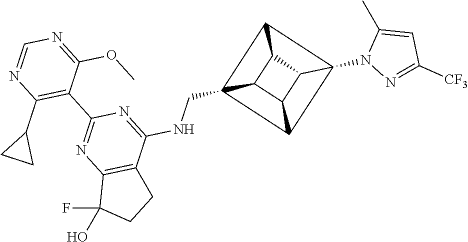

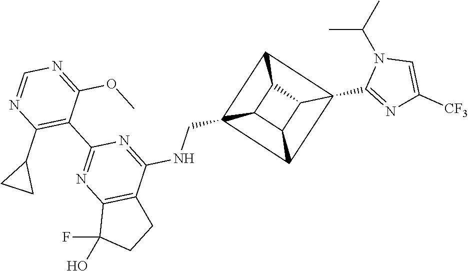

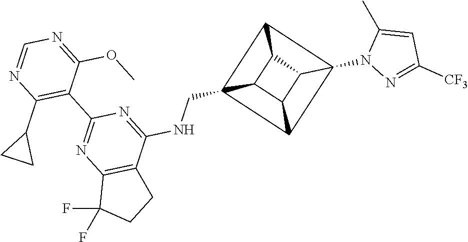

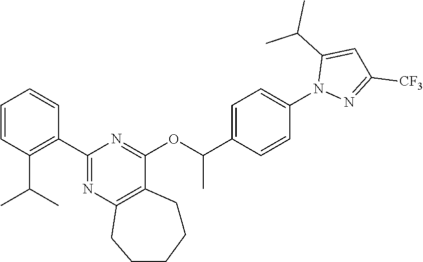 [SMILES: CC(C)c1ccccc1-c1nc2c(c(OC(C)c3ccc(-n4nc(C(F)(F)F)cc4C(C)C)cc3)n1)CCCCC2]

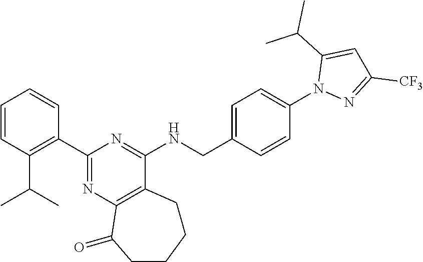 [SMILES: CC(C)c1ccccc1-c1nc(NCc2ccc(-n3nc(C(F)(F)F)cc3C(C)C)cc2)c2c(n1)C(=O)CCCC2]

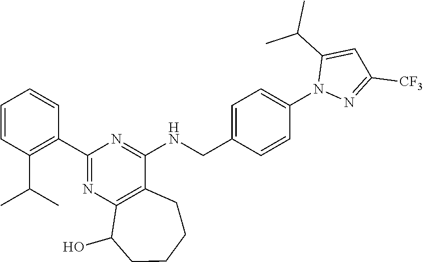 [SMILES: CC(C)c1ccccc1-c1nc(NCc2ccc(-n3nc(C(F)(F)F)cc3C(C)C)cc2)c2c(n1)C(O)CCCC2]

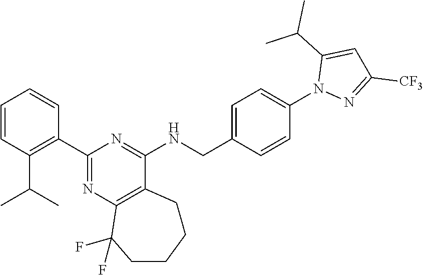 [SMILES: CC(C)c1ccccc1-c1nc(NCc2ccc(-n3nc(C(F)(F)F)cc3C(C)C)cc2)c2c(n1)C(F)(F)CCCC2]

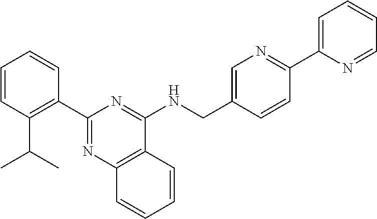 [SMILES: CC(C)c1ccccc1-c1nc(NCc2ccc(-c3ccccn3)nc2)c2ccccc2n1]

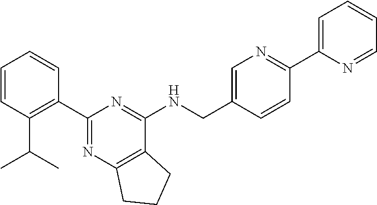 [SMILES: CC(C)c1ccccc1-c1nc2c(c(NCc3ccc(-c4ccccn4)nc3)n1)CCC2]

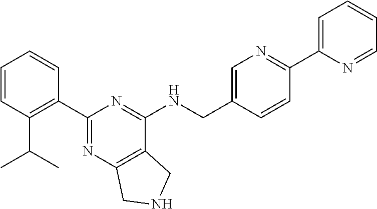 [SMILES: CC(C)c1ccccc1-c1nc2c(c(NCc3ccc(-c4ccccn4)nc3)n1)CNC2]

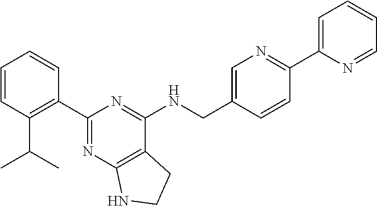 [SMILES: CC(C)c1ccccc1-c1nc2c(c(NCc3ccc(-c4ccccn4)nc3)n1)CCN2]

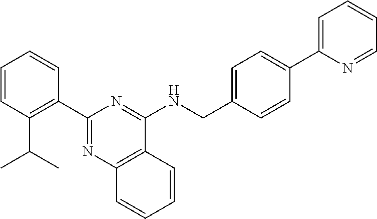 [SMILES: CC(C)c1ccccc1-c1nc(NCc2ccc(-c3ccccn3)cc2)c2ccccc2n1]

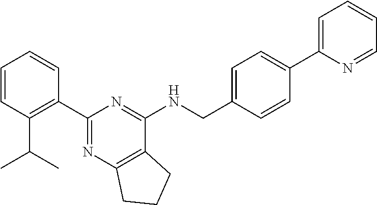 [SMILES: CC(C)c1ccccc1-c1nc2c(c(NCc3ccc(-c4ccccn4)cc3)n1)CCC2]

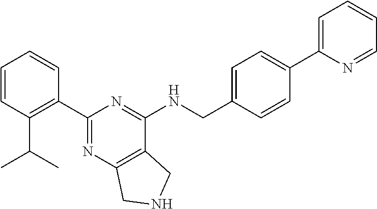 [SMILES: CC(C)c1ccccc1-c1nc2c(c(NCc3ccc(-c4ccccn4)cc3)n1)CNC2]

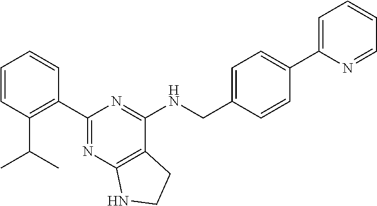 [SMILES: CC(C)c1ccccc1-c1nc2c(c(NCc3ccc(-c4ccccn4)cc3)n1)CCN2]

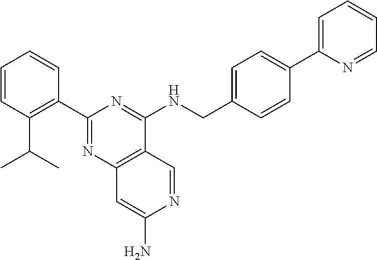 [SMILES: CC(C)c1ccccc1-c1nc(NCc2ccc(-c3ccccn3)cc2)c2cnc(N)cc2n1]

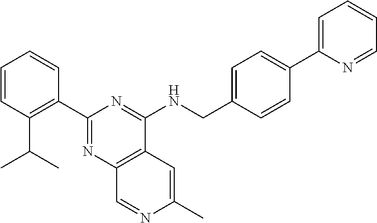 [SMILES: Cc1cc2c(NCc3ccc(-c4ccccn4)cc3)nc(-c3ccccc3C(C)C)nc2cn1]

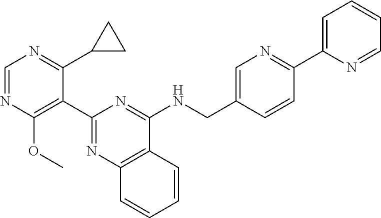 [SMILES: COc1ncnc(C2CC2)c1-c1nc(NCc2ccc(-c3ccccn3)nc2)c2ccccc2n1]

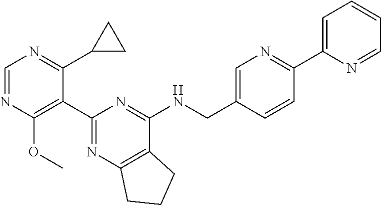 [SMILES: COc1ncnc(C2CC2)c1-c1nc2c(c(NCc3ccc(-c4ccccn4)nc3)n1)CCC2]

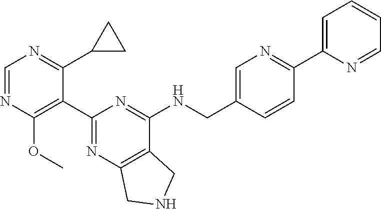 [SMILES: COc1ncnc(C2CC2)c1-c1nc2c(c(NCc3ccc(-c4ccccn4)nc3)n1)CNC2]

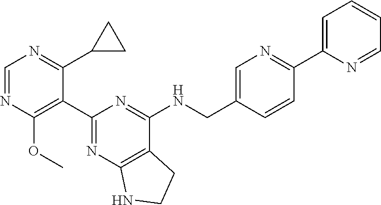 [SMILES: COc1ncnc(C2CC2)c1-c1nc2c(c(NCc3ccc(-c4ccccn4)nc3)n1)CCN2]

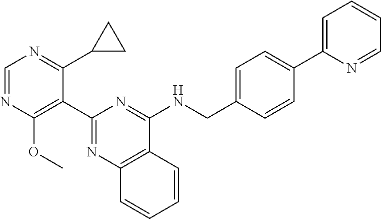 [SMILES: COc1ncnc(C2CC2)c1-c1nc(NCc2ccc(-c3ccccn3)cc2)c2ccccc2n1]

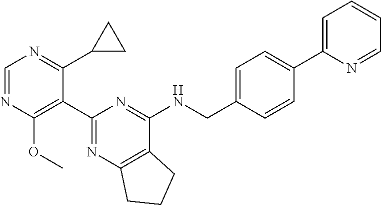 [SMILES: COc1ncnc(C2CC2)c1-c1nc2c(c(NCc3ccc(-c4ccccn4)cc3)n1)CCC2]

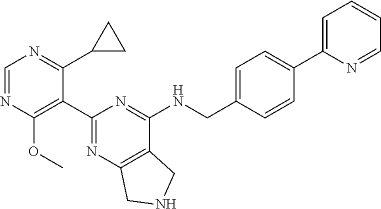 [SMILES: COc1ncnc(C2CC2)c1-c1nc2c(c(NCc3ccc(-c4ccccn4)cc3)n1)CNC2]

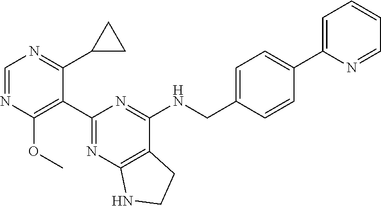 [SMILES: COc1ncnc(C2CC2)c1-c1nc2c(c(NCc3ccc(-c4ccccn4)cc3)n1)CCN2]

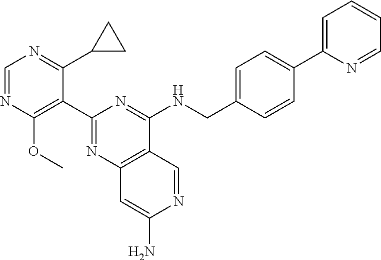 [SMILES: COc1ncnc(C2CC2)c1-c1nc(NCc2ccc(-c3ccccn3)cc2)c2cnc(N)cc2n1]

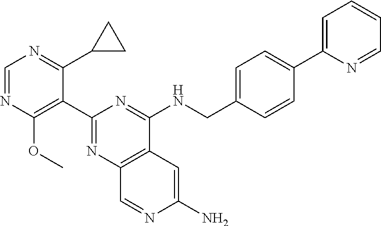 [SMILES: COc1ncnc(C2CC2)c1-c1nc(NCc2ccc(-c3ccccn3)cc2)c2cc(N)ncc2n1]

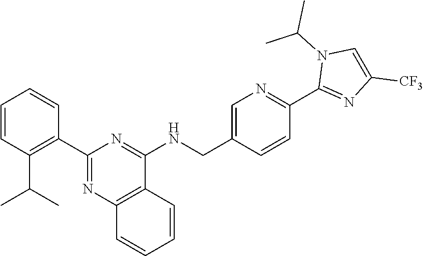 [SMILES: CC(C)c1ccccc1-c1nc(NCc2ccc(-c3nc(C(F)(F)F)cn3C(C)C)nc2)c2ccccc2n1]

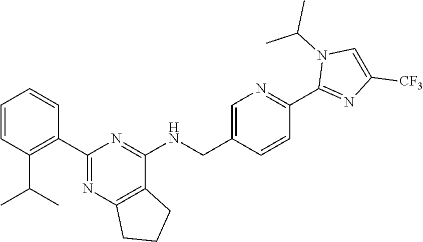 [SMILES: CC(C)c1ccccc1-c1nc2c(c(NCc3ccc(-c4nc(C(F)(F)F)cn4C(C)C)nc3)n1)CCC2]

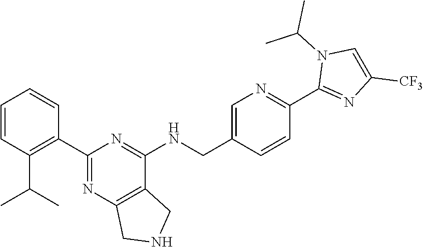 [SMILES: CC(C)c1ccccc1-c1nc2c(c(NCc3ccc(-c4nc(C(F)(F)F)cn4C(C)C)nc3)n1)CNC2]

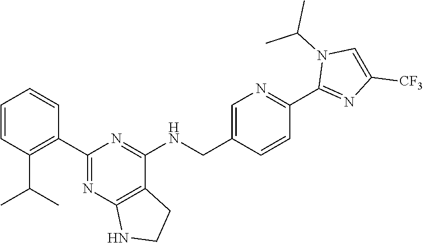 [SMILES: CC(C)c1ccccc1-c1nc2c(c(NCc3ccc(-c4nc(C(F)(F)F)cn4C(C)C)nc3)n1)CCN2]

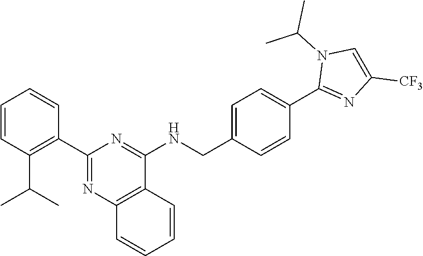 [SMILES: CC(C)c1ccccc1-c1nc(NCc2ccc(-c3nc(C(F)(F)F)cn3C(C)C)cc2)c2ccccc2n1]

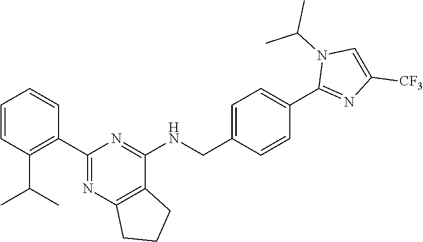 [SMILES: CC(C)c1ccccc1-c1nc2c(c(NCc3ccc(-c4nc(C(F)(F)F)cn4C(C)C)cc3)n1)CCC2]

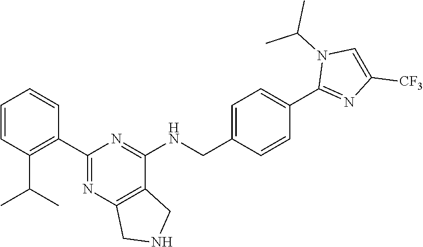 [SMILES: CC(C)c1ccccc1-c1nc2c(c(NCc3ccc(-c4nc(C(F)(F)F)cn4C(C)C)cc3)n1)CNC2]

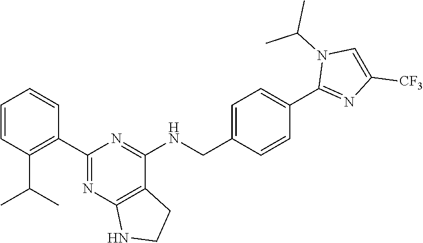 [SMILES: CC(C)c1ccccc1-c1nc2c(c(NCc3ccc(-c4nc(C(F)(F)F)cn4C(C)C)cc3)n1)CCN2]

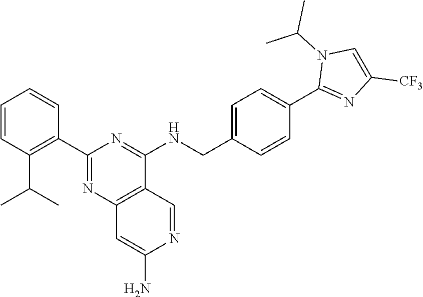 [SMILES: CC(C)c1ccccc1-c1nc(NCc2ccc(-c3nc(C(F)(F)F)cn3C(C)C)cc2)c2cnc(N)cc2n1]

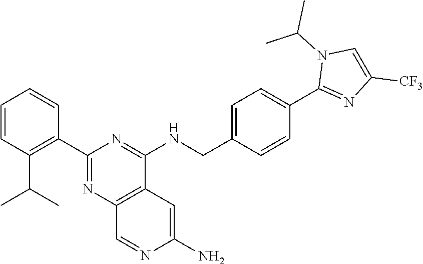 [SMILES: CC(C)c1ccccc1-c1nc(NCc2ccc(-c3nc(C(F)(F)F)cn3C(C)C)cc2)c2cc(N)ncc2n1]

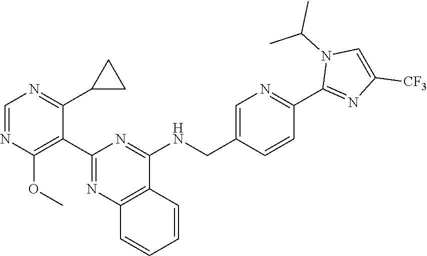 [SMILES: COc1ncnc(C2CC2)c1-c1nc(NCc2ccc(-c3nc(C(F)(F)F)cn3C(C)C)nc2)c2ccccc2n1]

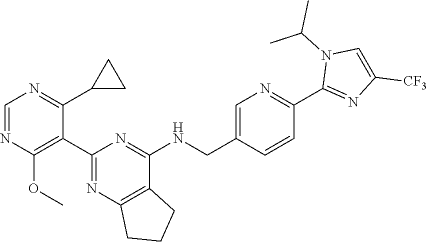 [SMILES: COc1ncnc(C2CC2)c1-c1nc2c(c(NCc3ccc(-c4nc(C(F)(F)F)cn4C(C)C)nc3)n1)CCC2]

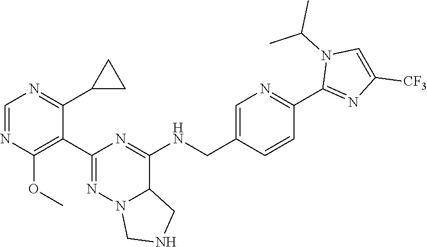 [SMILES: COc1ncnc(C2CC2)c1C1=NN2CNCC2C(NCc2ccc(-c3nc(C(F)(F)F)cn3C(C)C)nc2)=N1]

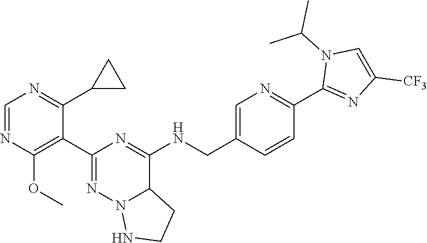 [SMILES: COc1ncnc(C2CC2)c1C1=NN2NCCC2C(NCc2ccc(-c3nc(C(F)(F)F)cn3C(C)C)nc2)=N1]

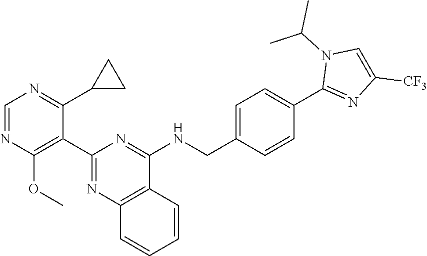 [SMILES: COc1ncnc(C2CC2)c1-c1nc(NCc2ccc(-c3nc(C(F)(F)F)cn3C(C)C)cc2)c2ccccc2n1]

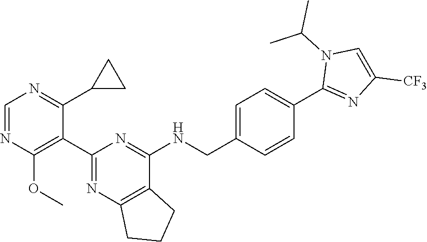 [SMILES: COc1ncnc(C2CC2)c1-c1nc2c(c(NCc3ccc(-c4nc(C(F)(F)F)cn4C(C)C)cc3)n1)CCC2]

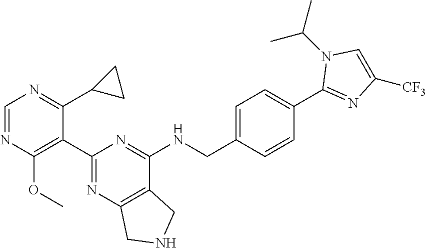 [SMILES: COc1ncnc(C2CC2)c1-c1nc2c(c(NCc3ccc(-c4nc(C(F)(F)F)cn4C(C)C)cc3)n1)CNC2]

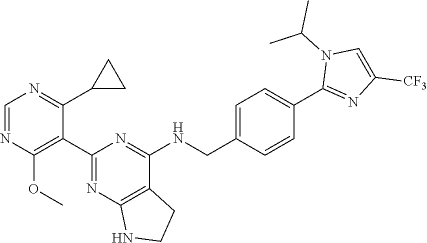 [SMILES: COc1ncnc(C2CC2)c1-c1nc2c(c(NCc3ccc(-c4nc(C(F)(F)F)cn4C(C)C)cc3)n1)CCN2]

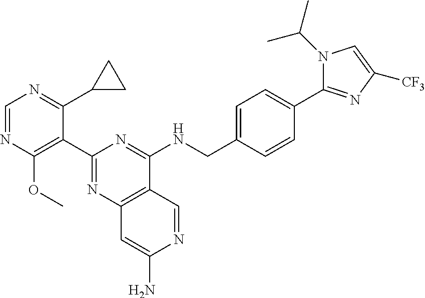 [SMILES: COc1ncnc(C2CC2)c1-c1nc(NCc2ccc(-c3nc(C(F)(F)F)cn3C(C)C)cc2)c2cnc(N)cc2n1]

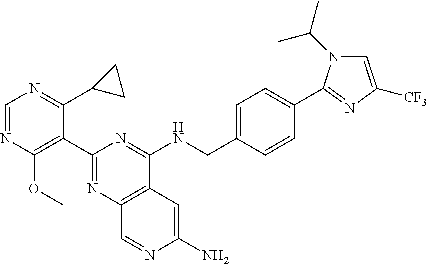 [SMILES: COc1ncnc(C2CC2)c1-c1nc(NCc2ccc(-c3nc(C(F)(F)F)cn3C(C)C)cc2)c2cc(N)ncc2n1]

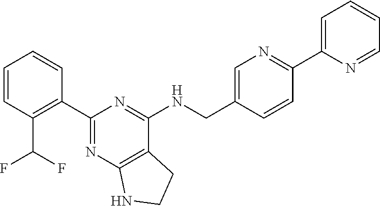 [SMILES: FC(F)c1ccccc1-c1nc2c(c(NCc3ccc(-c4ccccn4)nc3)n1)CCN2]

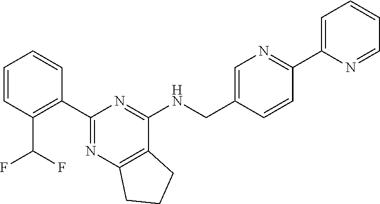 [SMILES: FC(F)c1ccccc1-c1nc2c(c(NCc3ccc(-c4ccccn4)nc3)n1)CCC2]

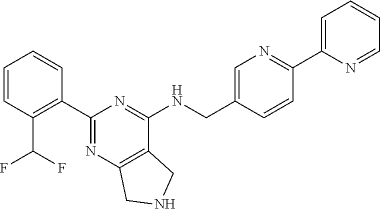 [SMILES: FC(F)c1ccccc1-c1nc2c(c(NCc3ccc(-c4ccccn4)nc3)n1)CNC2]

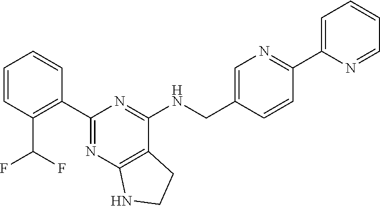 [SMILES: FC(F)c1ccccc1-c1nc2c(c(NCc3ccc(-c4ccccn4)nc3)n1)CCN2]

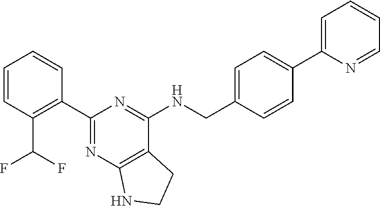 [SMILES: FC(F)c1ccccc1-c1nc2c(c(NCc3ccc(-c4ccccn4)cc3)n1)CCN2]

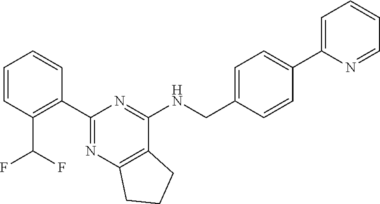 [SMILES: FC(F)c1ccccc1-c1nc2c(c(NCc3ccc(-c4ccccn4)cc3)n1)CCC2]

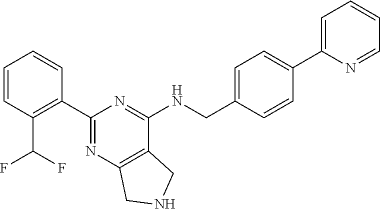 [SMILES: FC(F)c1ccccc1-c1nc2c(c(NCc3ccc(-c4ccccn4)cc3)n1)CNC2]

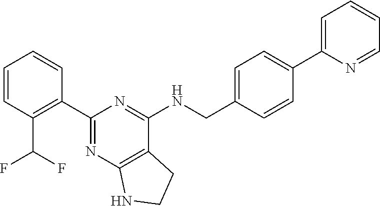 [SMILES: FC(F)c1ccccc1-c1nc2c(c(NCc3ccc(-c4ccccn4)cc3)n1)CCN2]

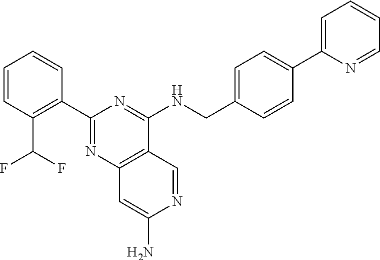 [SMILES: Nc1cc2nc(-c3ccccc3C(F)F)nc(NCc3ccc(-c4ccccn4)cc3)c2cn1]

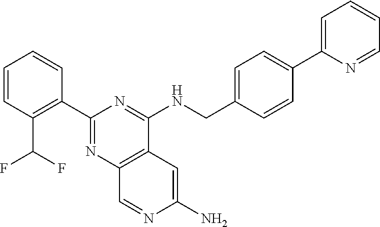 [SMILES: Nc1cc2c(NCc3ccc(-c4ccccn4)cc3)nc(-c3ccccc3C(F)F)nc2cn1]

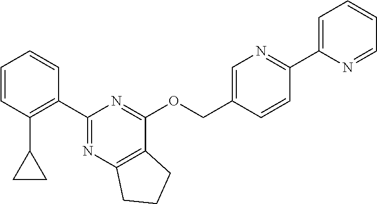 [SMILES: c1ccc(-c2ccc(COc3nc(-c4ccccc4C4CC4)nc4c3CCC4)cn2)nc1]

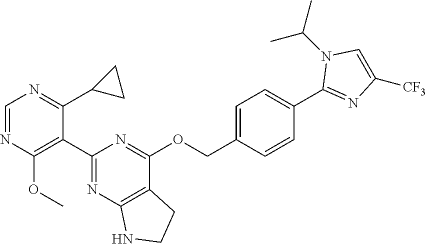 [SMILES: COc1ncnc(C2CC2)c1-c1nc2c(c(OCc3ccc(-c4nc(C(F)(F)F)cn4C(C)C)cc3)n1)CCN2]

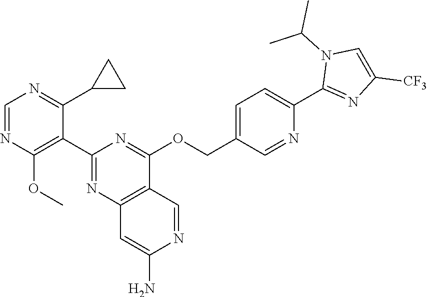 [SMILES: COc1ncnc(C2CC2)c1-c1nc(OCc2ccc(-c3nc(C(F)(F)F)cn3C(C)C)nc2)c2cnc(N)cc2n1]

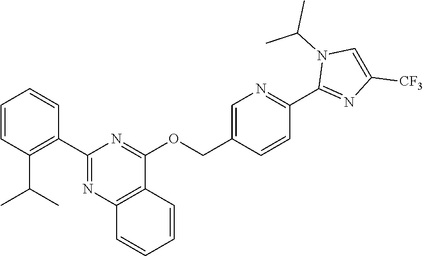 [SMILES: CC(C)c1ccccc1-c1nc(OCc2ccc(-c3nc(C(F)(F)F)cn3C(C)C)nc2)c2ccccc2n1]

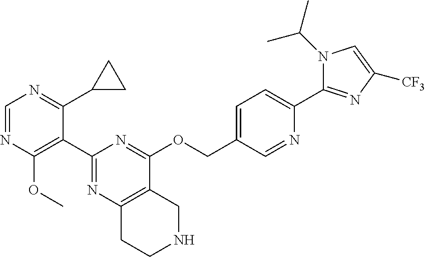 [SMILES: COc1ncnc(C2CC2)c1-c1nc2c(c(OCc3ccc(-c4nc(C(F)(F)F)cn4C(C)C)nc3)n1)CNCC2]

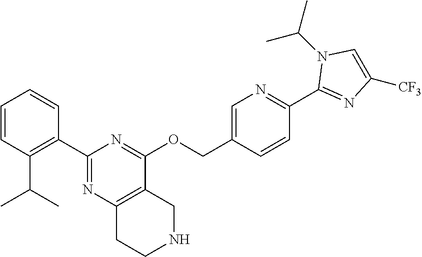 [SMILES: CC(C)c1ccccc1-c1nc2c(c(OCc3ccc(-c4nc(C(F)(F)F)cn4C(C)C)nc3)n1)CNCC2]

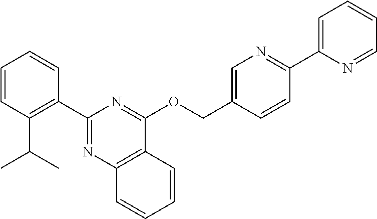 [SMILES: CC(C)c1ccccc1-c1nc(OCc2ccc(-c3ccccn3)nc2)c2ccccc2n1]

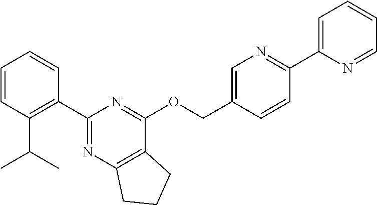 [SMILES: CC(C)c1ccccc1-c1nc2c(c(OCc3ccc(-c4ccccn4)nc3)n1)CCC2]

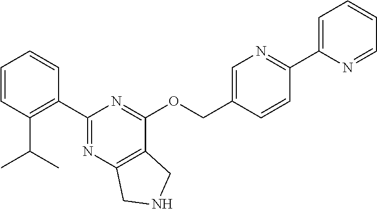 [SMILES: CC(C)c1ccccc1-c1nc2c(c(OCc3ccc(-c4ccccn4)nc3)n1)CNC2]

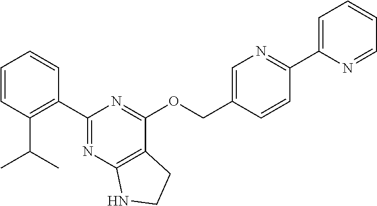 [SMILES: CC(C)c1ccccc1-c1nc2c(c(OCc3ccc(-c4ccccn4)nc3)n1)CCN2]

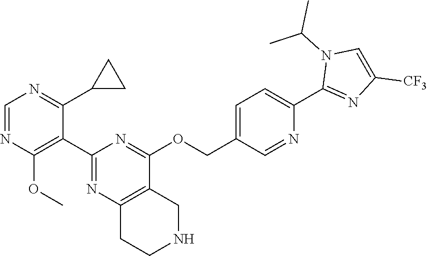 [SMILES: COc1ncnc(C2CC2)c1-c1nc2c(c(OCc3ccc(-c4nc(C(F)(F)F)cn4C(C)C)nc3)n1)CNCC2]

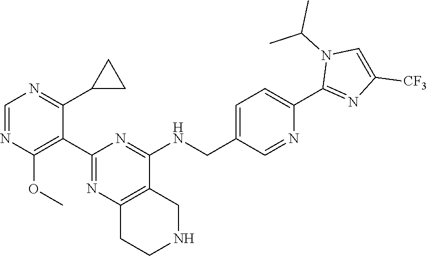 [SMILES: COc1ncnc(C2CC2)c1-c1nc2c(c(NCc3ccc(-c4nc(C(F)(F)F)cn4C(C)C)nc3)n1)CNCC2]

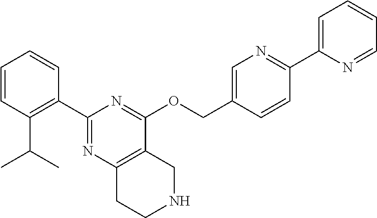 [SMILES: CC(C)c1ccccc1-c1nc2c(c(OCc3ccc(-c4ccccn4)nc3)n1)CNCC2]

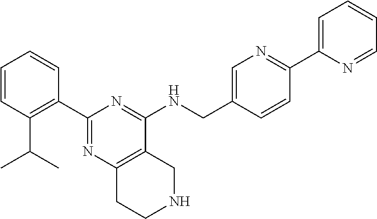 [SMILES: CC(C)c1ccccc1-c1nc2c(c(NCc3ccc(-c4ccccn4)nc3)n1)CNCC2]

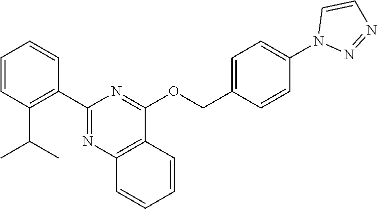 [SMILES: CC(C)c1ccccc1-c1nc(OCc2ccc(-n3ccnn3)cc2)c2ccccc2n1]

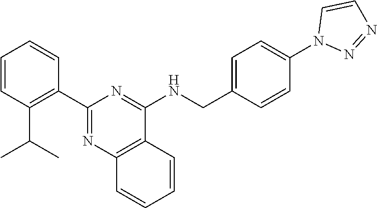 [SMILES: CC(C)c1ccccc1-c1nc(NCc2ccc(-n3ccnn3)cc2)c2ccccc2n1]

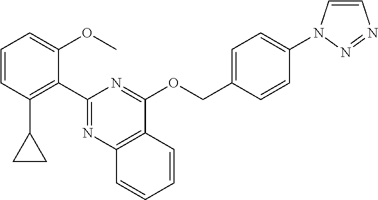 [SMILES: COc1cccc(C2CC2)c1-c1nc(OCc2ccc(-n3ccnn3)cc2)c2ccccc2n1]

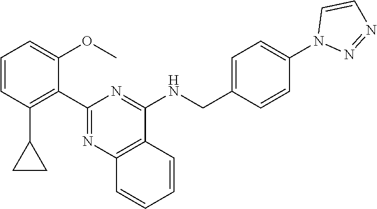 [SMILES: COc1cccc(C2CC2)c1-c1nc(NCc2ccc(-n3ccnn3)cc2)c2ccccc2n1]

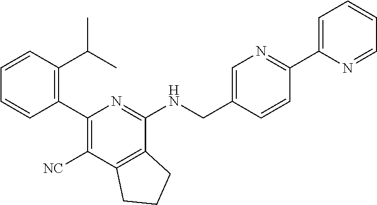 [SMILES: CC(C)c1ccccc1-c1nc(NCc2ccc(-c3ccccn3)nc2)c2c(c1C#N)CCC2]

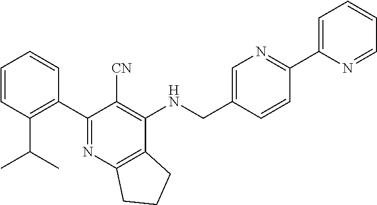 [SMILES: CC(C)c1ccccc1-c1nc2c(c(NCc3ccc(-c4ccccn4)nc3)c1C#N)CCC2]

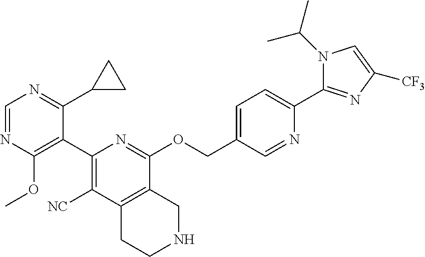 [SMILES: COc1ncnc(C2CC2)c1-c1nc(OCc2ccc(-c3nc(C(F)(F)F)cn3C(C)C)nc2)c2c(c1C#N)CCNC2]

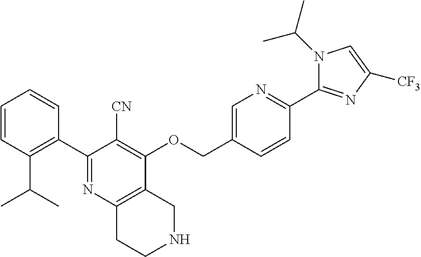 [SMILES: CC(C)c1ccccc1-c1nc2c(c(OCc3ccc(-c4nc(C(F)(F)F)cn4C(C)C)nc3)c1C#N)CNCC2]

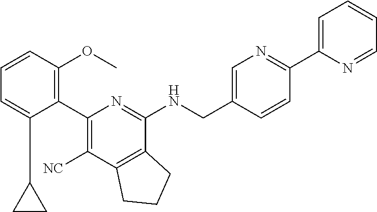 [SMILES: COc1cccc(C2CC2)c1-c1nc(NCc2ccc(-c3ccccn3)nc2)c2c(c1C#N)CCC2]

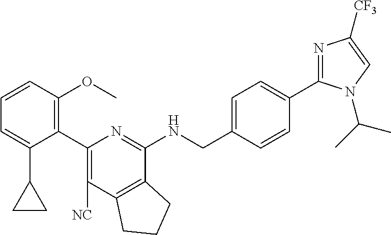 [SMILES: COc1cccc(C2CC2)c1-c1nc(NCc2ccc(-c3nc(C(F)(F)F)cn3C(C)C)cc2)c2c(c1C#N)CCC2]

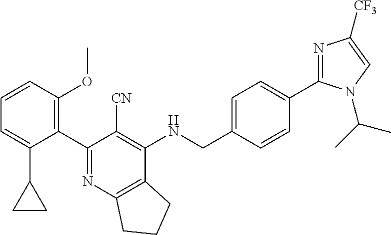 [SMILES: COc1cccc(C2CC2)c1-c1nc2c(c(NCc3ccc(-c4nc(C(F)(F)F)cn4C(C)C)cc3)c1C#N)CCC2]

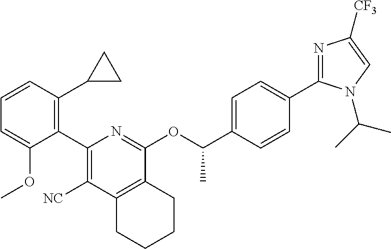 [SMILES: COc1cccc(C2CC2)c1-c1nc(O[C@@H](C)c2ccc(-c3nc(C(F)(F)F)cn3C(C)C)cc2)c2c(c1C#N)CCCC2]

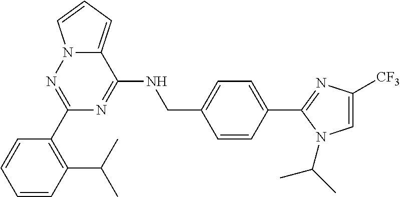 [SMILES: CC(C)c1ccccc1-c1nc(NCc2ccc(-c3nc(C(F)(F)F)cn3C(C)C)cc2)c2cccn2n1]

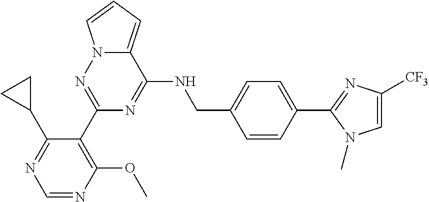 [SMILES: COc1ncnc(C2CC2)c1-c1nc(NCc2ccc(-c3nc(C(F)(F)F)cn3C)cc2)c2cccn2n1]

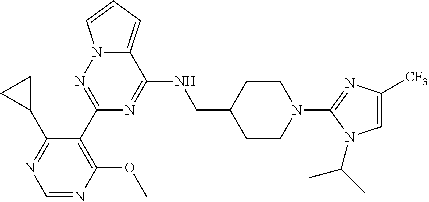 [SMILES: COc1ncnc(C2CC2)c1-c1nc(NCC2CCN(c3nc(C(F)(F)F)cn3C(C)C)CC2)c2cccn2n1]

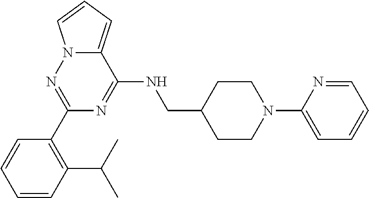 [SMILES: CC(C)c1ccccc1-c1nc(NCC2CCN(c3ccccn3)CC2)c2cccn2n1]

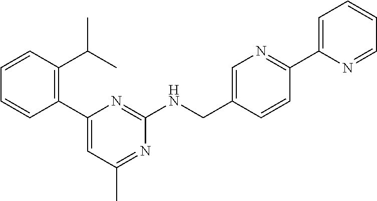 [SMILES: Cc1cc(-c2ccccc2C(C)C)nc(NCc2ccc(-c3ccccn3)nc2)n1]

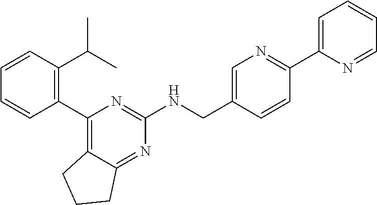 [SMILES: CC(C)c1ccccc1-c1nc(NCc2ccc(-c3ccccn3)nc2)nc2c1CCC2]

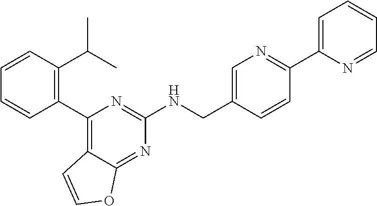 [SMILES: CC(C)c1ccccc1-c1nc(NCc2ccc(-c3ccccn3)nc2)nc2occc12]

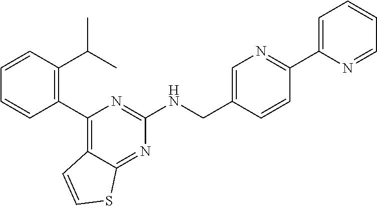 [SMILES: CC(C)c1ccccc1-c1nc(NCc2ccc(-c3ccccn3)nc2)nc2sccc12]

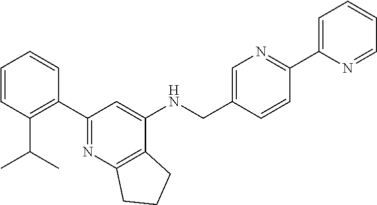 [SMILES: CC(C)c1ccccc1-c1cc(NCc2ccc(-c3ccccn3)nc2)c2c(n1)CCC2]

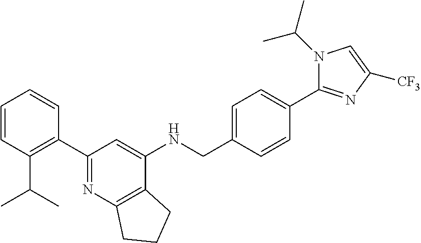 [SMILES: CC(C)c1ccccc1-c1cc(NCc2ccc(-c3nc(C(F)(F)F)cn3C(C)C)cc2)c2c(n1)CCC2]

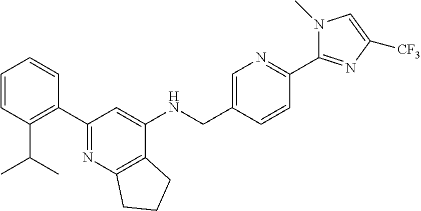 [SMILES: CC(C)c1ccccc1-c1cc(NCc2ccc(-c3nc(C(F)(F)F)cn3C)nc2)c2c(n1)CCC2]

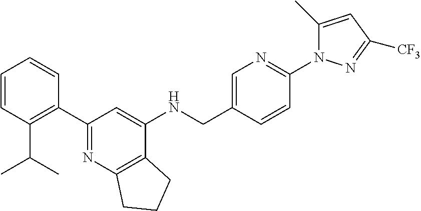 [SMILES: Cc1cc(C(F)(F)F)nn1-c1ccc(CNc2cc(-c3ccccc3C(C)C)nc3c2CCC3)cn1]

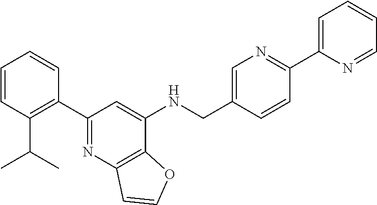 [SMILES: CC(C)c1ccccc1-c1cc(NCc2ccc(-c3ccccn3)nc2)c2occc2n1]

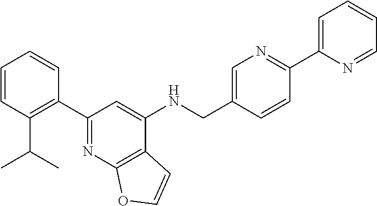 [SMILES: CC(C)c1ccccc1-c1cc(NCc2ccc(-c3ccccn3)nc2)c2ccoc2n1]

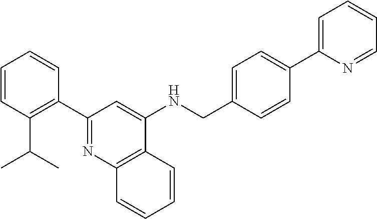 [SMILES: CC(C)c1ccccc1-c1cc(NCc2ccc(-c3ccccn3)cc2)c2ccccc2n1]

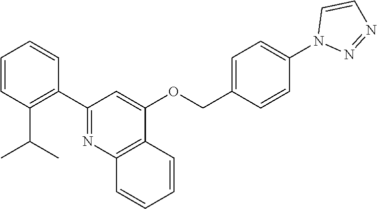 [SMILES: CC(C)c1ccccc1-c1cc(OCc2ccc(-n3ccnn3)cc2)c2ccccc2n1]

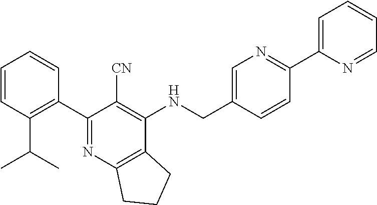 [SMILES: CC(C)c1ccccc1-c1nc2c(c(NCc3ccc(-c4ccccn4)nc3)c1C#N)CCC2]

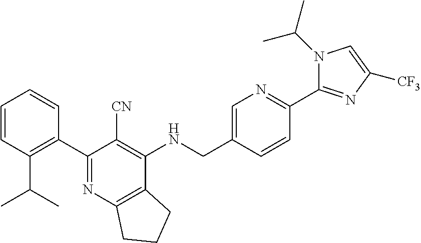 [SMILES: CC(C)c1ccccc1-c1nc2c(c(NCc3ccc(-c4nc(C(F)(F)F)cn4C(C)C)nc3)c1C#N)CCC2]

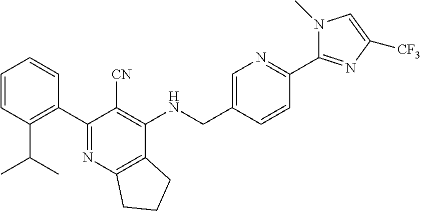 [SMILES: CC(C)c1ccccc1-c1nc2c(c(NCc3ccc(-c4nc(C(F)(F)F)cn4C)nc3)c1C#N)CCC2]

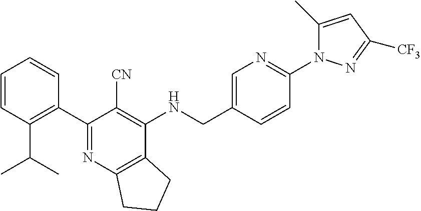 [SMILES: Cc1cc(C(F)(F)F)nn1-c1ccc(CNc2c(C#N)c(-c3ccccc3C(C)C)nc3c2CCC3)cn1]

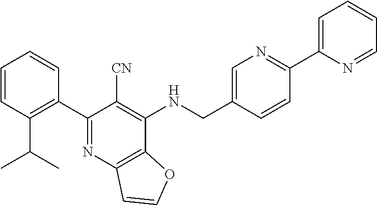 [SMILES: CC(C)c1ccccc1-c1nc2ccoc2c(NCc2ccc(-c3ccccn3)nc2)c1C#N]

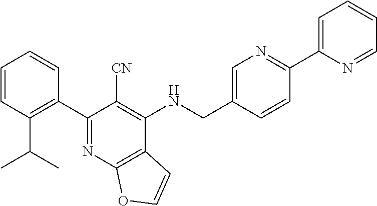 [SMILES: CC(C)c1ccccc1-c1nc2occc2c(NCc2ccc(-c3ccccn3)nc2)c1C#N]

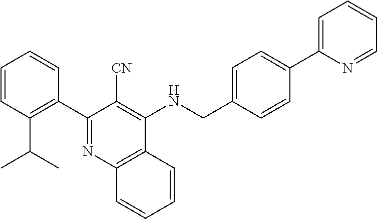 [SMILES: CC(C)c1ccccc1-c1nc2ccccc2c(NCc2ccc(-c3ccccn3)cc2)c1C#N]

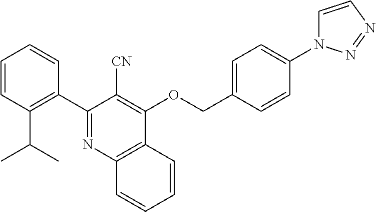 [SMILES: CC(C)c1ccccc1-c1nc2ccccc2c(OCc2ccc(-n3ccnn3)cc2)c1C#N]

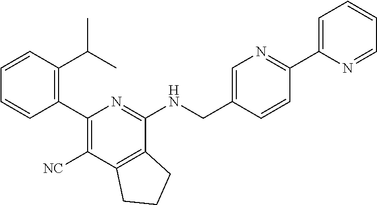 [SMILES: CC(C)c1ccccc1-c1nc(NCc2ccc(-c3ccccn3)nc2)c2c(c1C#N)CCC2]

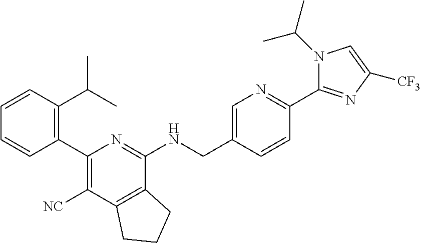 [SMILES: CC(C)c1ccccc1-c1nc(NCc2ccc(-c3nc(C(F)(F)F)cn3C(C)C)nc2)c2c(c1C#N)CCC2]

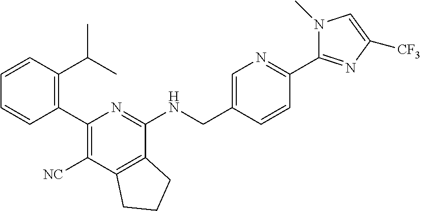 [SMILES: CC(C)c1ccccc1-c1nc(NCc2ccc(-c3nc(C(F)(F)F)cn3C)nc2)c2c(c1C#N)CCC2]

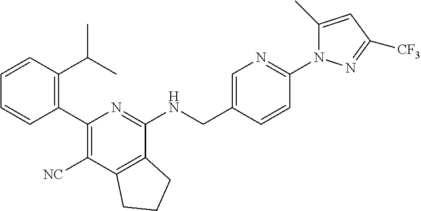 [SMILES: Cc1cc(C(F)(F)F)nn1-c1ccc(CNc2nc(-c3ccccc3C(C)C)c(C#N)c3c2CCC3)cn1]

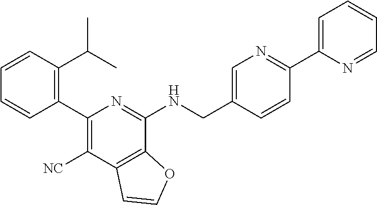 [SMILES: CC(C)c1ccccc1-c1nc(NCc2ccc(-c3ccccn3)nc2)c2occc2c1C#N]

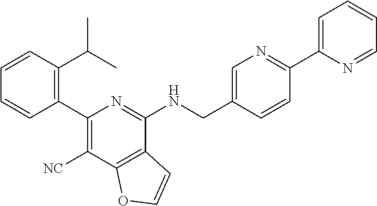 [SMILES: CC(C)c1ccccc1-c1nc(NCc2ccc(-c3ccccn3)nc2)c2ccoc2c1C#N]

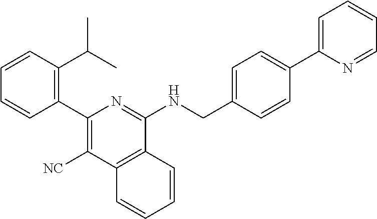 [SMILES: CC(C)c1ccccc1-c1nc(NCc2ccc(-c3ccccn3)cc2)c2ccccc2c1C#N]

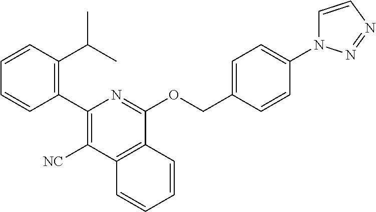 [SMILES: CC(C)c1ccccc1-c1nc(OCc2ccc(-n3ccnn3)cc2)c2ccccc2c1C#N]

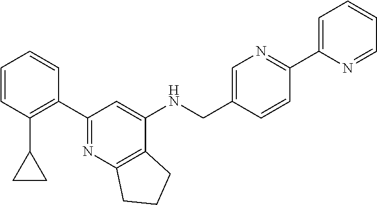 [SMILES: c1ccc(-c2ccc(CNc3cc(-c4ccccc4C4CC4)nc4c3CCC4)cn2)nc1]

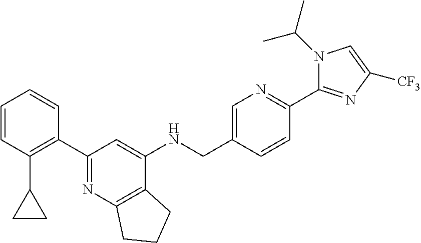 [SMILES: CC(C)n1cc(C(F)(F)F)nc1-c1ccc(CNc2cc(-c3ccccc3C3CC3)nc3c2CCC3)cn1]

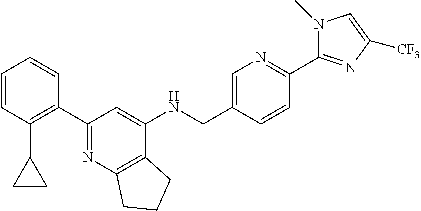 [SMILES: Cn1cc(C(F)(F)F)nc1-c1ccc(CNc2cc(-c3ccccc3C3CC3)nc3c2CCC3)cn1]

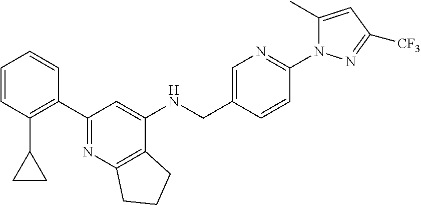 [SMILES: Cc1cc(C(F)(F)F)nn1-c1ccc(CNc2cc(-c3ccccc3C3CC3)nc3c2CCC3)cn1]

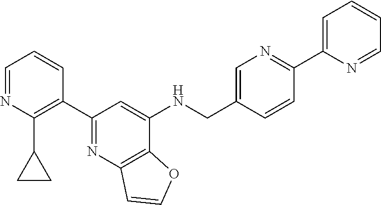 [SMILES: c1ccc(-c2ccc(CNc3cc(-c4cccnc4C4CC4)nc4ccoc34)cn2)nc1]

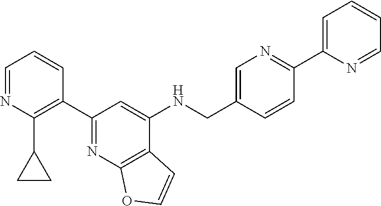 [SMILES: c1ccc(-c2ccc(CNc3cc(-c4cccnc4C4CC4)nc4occc34)cn2)nc1]

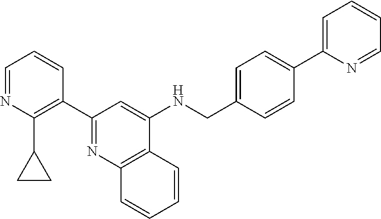 [SMILES: c1ccc(-c2ccc(CNc3cc(-c4cccnc4C4CC4)nc4ccccc34)cc2)nc1]

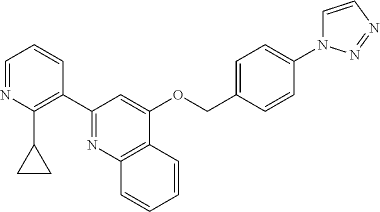 [SMILES: c1cnc(C2CC2)c(-c2cc(OCc3ccc(-n4ccnn4)cc3)c3ccccc3n2)c1]

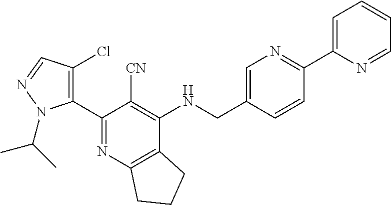 [SMILES: CC(C)n1ncc(Cl)c1-c1nc2c(c(NCc3ccc(-c4ccccn4)nc3)c1C#N)CCC2]

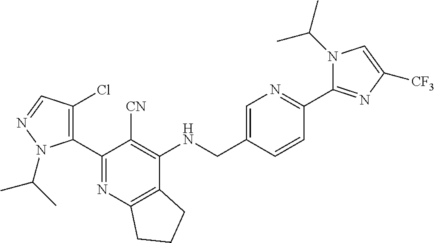 [SMILES: CC(C)n1cc(C(F)(F)F)nc1-c1ccc(CNc2c(C#N)c(-c3c(Cl)cnn3C(C)C)nc3c2CCC3)cn1]

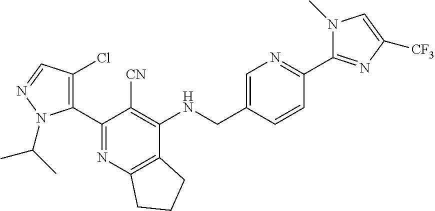 [SMILES: CC(C)n1ncc(Cl)c1-c1nc2c(c(NCc3ccc(-c4nc(C(F)(F)F)cn4C)nc3)c1C#N)CCC2]

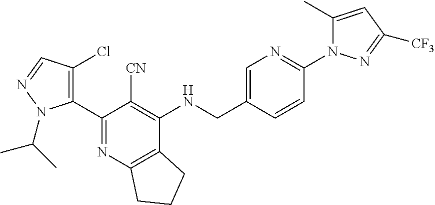 [SMILES: Cc1cc(C(F)(F)F)nn1-c1ccc(CNc2c(C#N)c(-c3c(Cl)cnn3C(C)C)nc3c2CCC3)cn1]

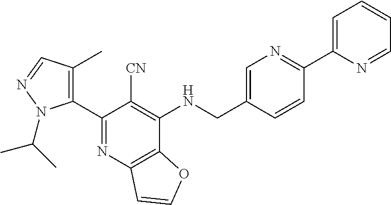 [SMILES: Cc1cnn(C(C)C)c1-c1nc2ccoc2c(NCc2ccc(-c3ccccn3)nc2)c1C#N]

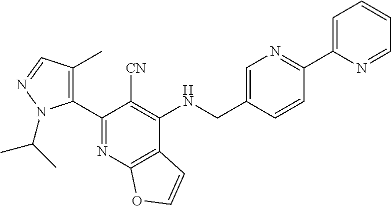 [SMILES: Cc1cnn(C(C)C)c1-c1nc2occc2c(NCc2ccc(-c3ccccn3)nc2)c1C#N]

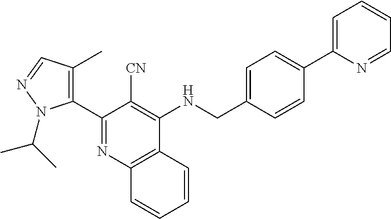 [SMILES: Cc1cnn(C(C)C)c1-c1nc2ccccc2c(NCc2ccc(-c3ccccn3)cc2)c1C#N]

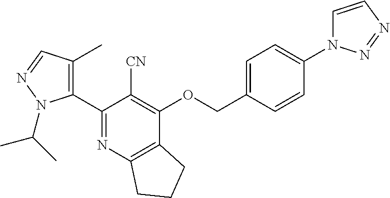 [SMILES: Cc1cnn(C(C)C)c1-c1nc2c(c(OCc3ccc(-n4ccnn4)cc3)c1C#N)CCC2]

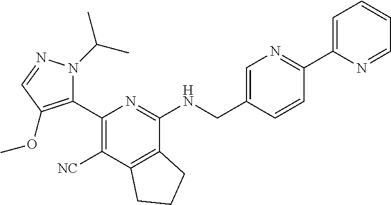 [SMILES: COc1cnn(C(C)C)c1-c1nc(NCc2ccc(-c3ccccn3)nc2)c2c(c1C#N)CCC2]

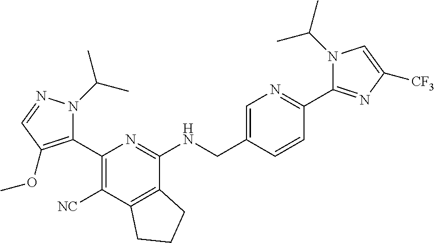 [SMILES: COc1cnn(C(C)C)c1-c1nc(NCc2ccc(-c3nc(C(F)(F)F)cn3C(C)C)nc2)c2c(c1C#N)CCC2]

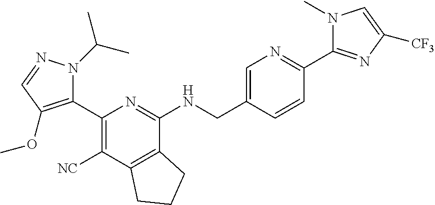 [SMILES: COc1cnn(C(C)C)c1-c1nc(NCc2ccc(-c3nc(C(F)(F)F)cn3C)nc2)c2c(c1C#N)CCC2]

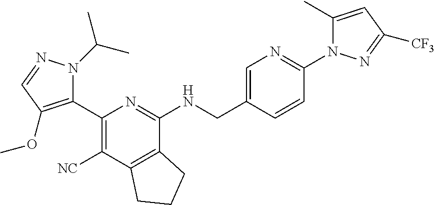 [SMILES: COc1cnn(C(C)C)c1-c1nc(NCc2ccc(-n3nc(C(F)(F)F)cc3C)nc2)c2c(c1C#N)CCC2]

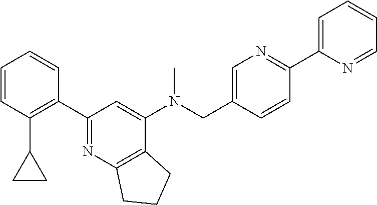 [SMILES: CN(Cc1ccc(-c2ccccn2)nc1)c1cc(-c2ccccc2C2CC2)nc2c1CCC2]

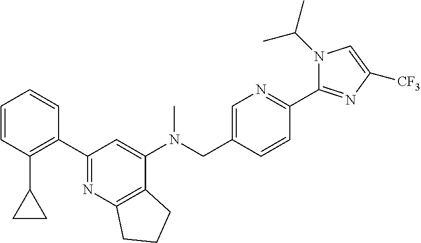 [SMILES: CC(C)n1cc(C(F)(F)F)nc1-c1ccc(CN(C)c2cc(-c3ccccc3C3CC3)nc3c2CCC3)cn1]

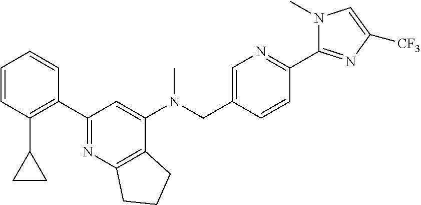 [SMILES: CN(Cc1ccc(-c2nc(C(F)(F)F)cn2C)nc1)c1cc(-c2ccccc2C2CC2)nc2c1CCC2]

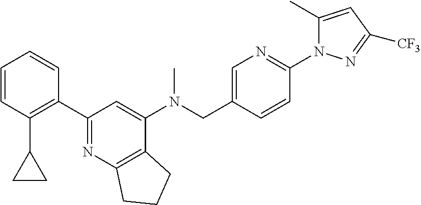 [SMILES: Cc1cc(C(F)(F)F)nn1-c1ccc(CN(C)c2cc(-c3ccccc3C3CC3)nc3c2CCC3)cn1]

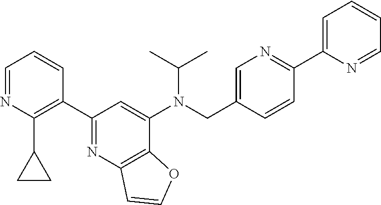 [SMILES: CC(C)N(Cc1ccc(-c2ccccn2)nc1)c1cc(-c2cccnc2C2CC2)nc2ccoc12]

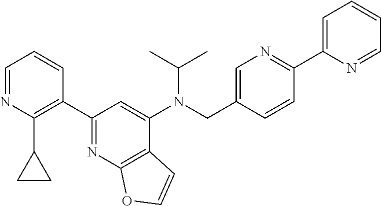 [SMILES: CC(C)N(Cc1ccc(-c2ccccn2)nc1)c1cc(-c2cccnc2C2CC2)nc2occc12]

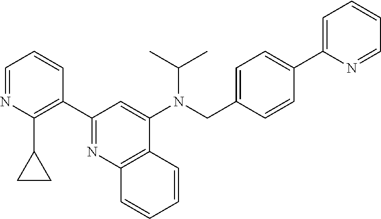 [SMILES: CC(C)N(Cc1ccc(-c2ccccn2)cc1)c1cc(-c2cccnc2C2CC2)nc2ccccc12]

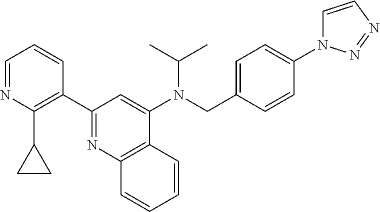 [SMILES: CC(C)N(Cc1ccc(-n2ccnn2)cc1)c1cc(-c2cccnc2C2CC2)nc2ccccc12]

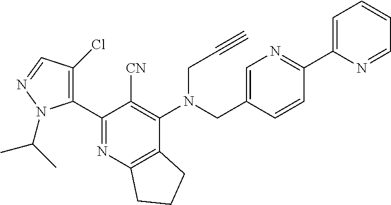 [SMILES: C#CCN(Cc1ccc(-c2ccccn2)nc1)c1c(C#N)c(-c2c(Cl)cnn2C(C)C)nc2c1CCC2]

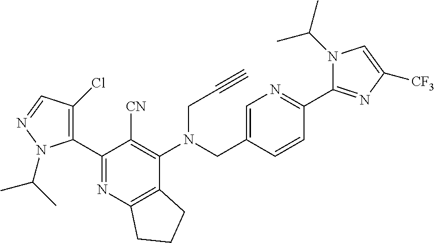 [SMILES: C#CCN(Cc1ccc(-c2nc(C(F)(F)F)cn2C(C)C)nc1)c1c(C#N)c(-c2c(Cl)cnn2C(C)C)nc2c1CCC2]

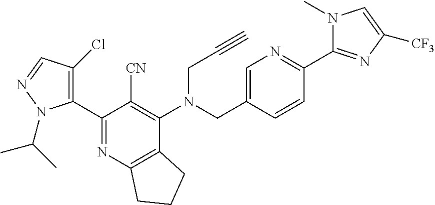 [SMILES: C#CCN(Cc1ccc(-c2nc(C(F)(F)F)cn2C)nc1)c1c(C#N)c(-c2c(Cl)cnn2C(C)C)nc2c1CCC2]

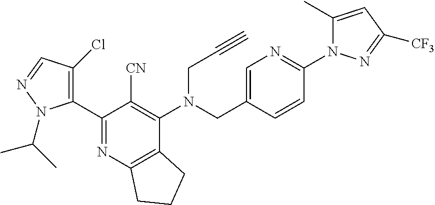 [SMILES: C#CCN(Cc1ccc(-n2nc(C(F)(F)F)cc2C)nc1)c1c(C#N)c(-c2c(Cl)cnn2C(C)C)nc2c1CCC2]

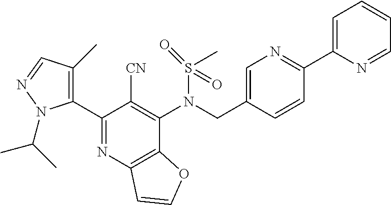 [SMILES: Cc1cnn(C(C)C)c1-c1nc2ccoc2c(N(Cc2ccc(-c3ccccn3)nc2)S(C)(=O)=O)c1C#N]

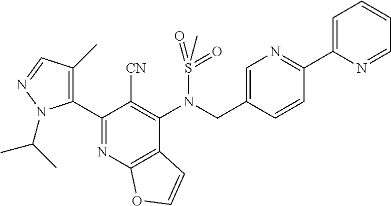 [SMILES: Cc1cnn(C(C)C)c1-c1nc2occc2c(N(Cc2ccc(-c3ccccn3)nc2)S(C)(=O)=O)c1C#N]

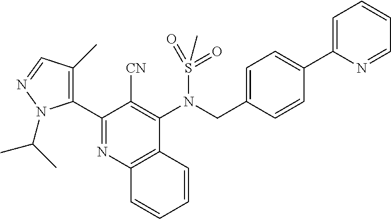 [SMILES: Cc1cnn(C(C)C)c1-c1nc2ccccc2c(N(Cc2ccc(-c3ccccn3)cc2)S(C)(=O)=O)c1C#N]

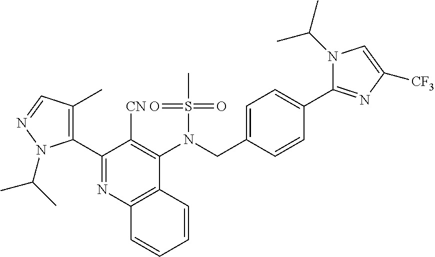 [SMILES: Cc1cnn(C(C)C)c1-c1nc2ccccc2c(N(Cc2ccc(-c3nc(C(F)(F)F)cn3C(C)C)cc2)S(C)(=O)=O)c1C#N]

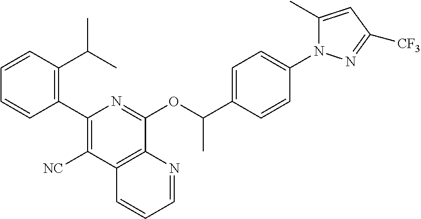 [SMILES: Cc1cc(C(F)(F)F)nn1-c1ccc(C(C)Oc2nc(-c3ccccc3C(C)C)c(C#N)c3cccnc23)cc1]

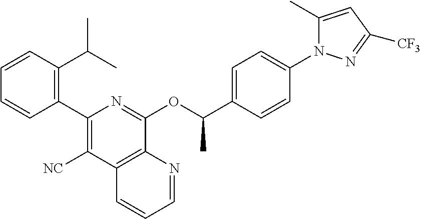 [SMILES: Cc1cc(C(F)(F)F)nn1-c1ccc([C@@H](C)Oc2nc(-c3ccccc3C(C)C)c(C#N)c3cccnc23)cc1]

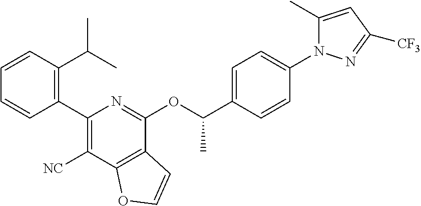 [SMILES: Cc1cc(C(F)(F)F)nn1-c1ccc([C@H](C)Oc2nc(-c3ccccc3C(C)C)c(C#N)c3occc23)cc1]

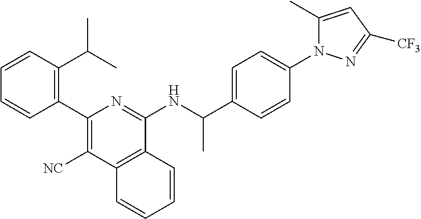 [SMILES: Cc1cc(C(F)(F)F)nn1-c1ccc(C(C)Nc2nc(-c3ccccc3C(C)C)c(C#N)c3ccccc23)cc1]

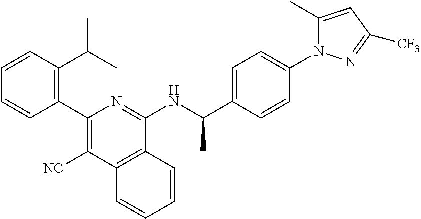 [SMILES: Cc1cc(C(F)(F)F)nn1-c1ccc([C@@H](C)Nc2nc(-c3ccccc3C(C)C)c(C#N)c3ccccc23)cc1]

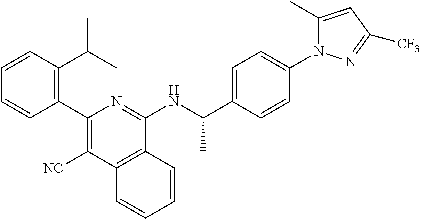 [SMILES: Cc1cc(C(F)(F)F)nn1-c1ccc([C@H](C)Nc2nc(-c3ccccc3C(C)C)c(C#N)c3ccccc23)cc1]

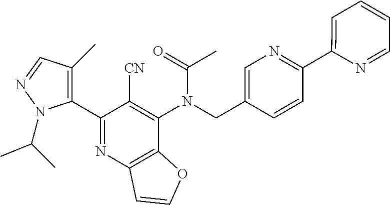 [SMILES: CC(=O)N(Cc1ccc(-c2ccccn2)nc1)c1c(C#N)c(-c2c(C)cnn2C(C)C)nc2ccoc12]

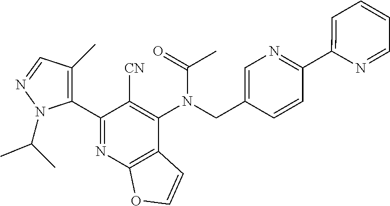 [SMILES: CC(=O)N(Cc1ccc(-c2ccccn2)nc1)c1c(C#N)c(-c2c(C)cnn2C(C)C)nc2occc12]

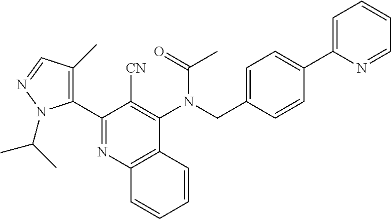 [SMILES: CC(=O)N(Cc1ccc(-c2ccccn2)cc1)c1c(C#N)c(-c2c(C)cnn2C(C)C)nc2ccccc12]

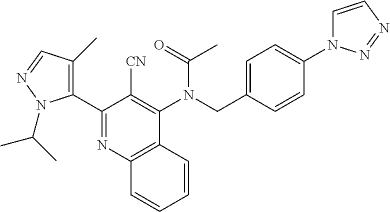 [SMILES: CC(=O)N(Cc1ccc(-n2ccnn2)cc1)c1c(C#N)c(-c2c(C)cnn2C(C)C)nc2ccccc12]

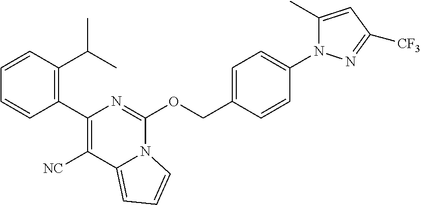 [SMILES: Cc1cc(C(F)(F)F)nn1-c1ccc(COc2nc(-c3ccccc3C(C)C)c(C#N)c3cccn23)cc1]

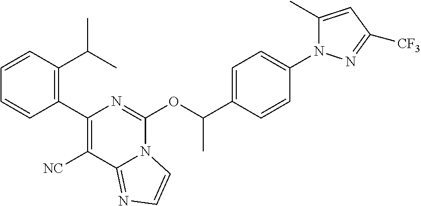 [SMILES: Cc1cc(C(F)(F)F)nn1-c1ccc(C(C)Oc2nc(-c3ccccc3C(C)C)c(C#N)c3nccn23)cc1]

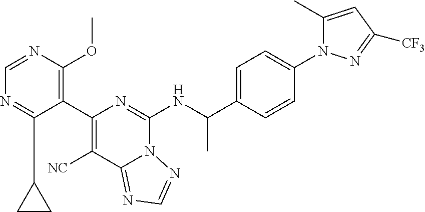 [SMILES: COc1ncnc(C2CC2)c1-c1nc(NC(C)c2ccc(-n3nc(C(F)(F)F)cc3C)cc2)n2ncnc2c1C#N]

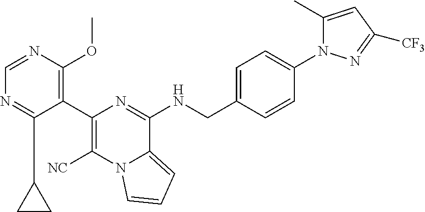 [SMILES: COc1ncnc(C2CC2)c1-c1nc(NCc2ccc(-n3nc(C(F)(F)F)cc3C)cc2)c2cccn2c1C#N]

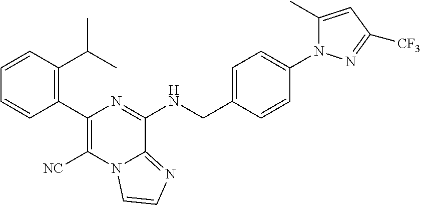 [SMILES: Cc1cc(C(F)(F)F)nn1-c1ccc(CNc2nc(-c3ccccc3C(C)C)c(C#N)n3ccnc23)cc1]

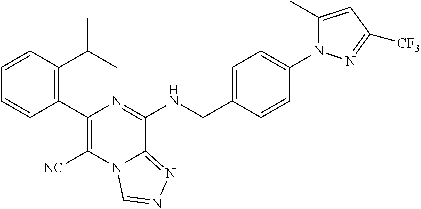 [SMILES: Cc1cc(C(F)(F)F)nn1-c1ccc(CNc2nc(-c3ccccc3C(C)C)c(C#N)n3cnnc23)cc1]

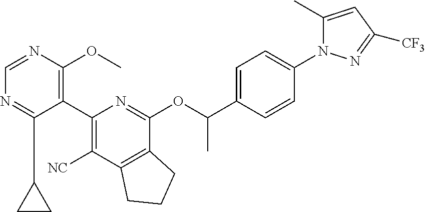 [SMILES: COc1ncnc(C2CC2)c1-c1nc(OC(C)c2ccc(-n3nc(C(F)(F)F)cc3C)cc2)c2c(c1C#N)CCC2]

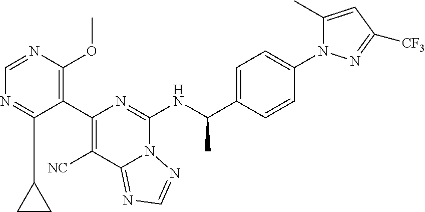 [SMILES: COc1ncnc(C2CC2)c1-c1nc(N[C@H](C)c2ccc(-n3nc(C(F)(F)F)cc3C)cc2)n2ncnc2c1C#N]

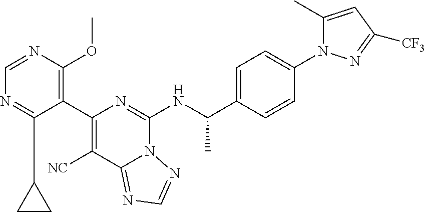 [SMILES: COc1ncnc(C2CC2)c1-c1nc(N[C@@H](C)c2ccc(-n3nc(C(F)(F)F)cc3C)cc2)n2ncnc2c1C#N]

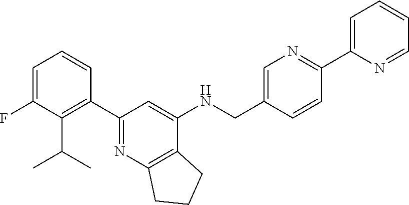 [SMILES: CC(C)c1c(F)cccc1-c1cc(NCc2ccc(-c3ccccn3)nc2)c2c(n1)CCC2]

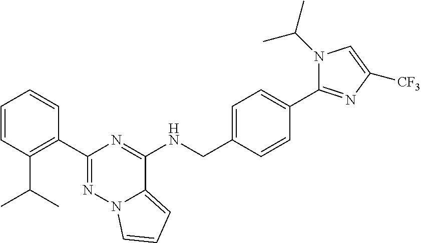 [SMILES: CC(C)c1ccccc1-c1nc(NCc2ccc(-c3nc(C(F)(F)F)cn3C(C)C)cc2)c2cccn2n1]

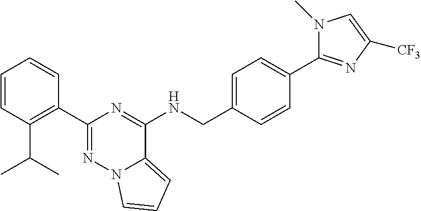 [SMILES: CC(C)c1ccccc1-c1nc(NCc2ccc(-c3nc(C(F)(F)F)cn3C)cc2)c2cccn2n1]

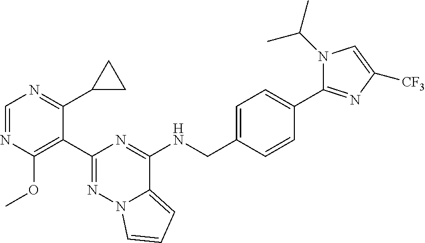 [SMILES: COc1ncnc(C2CC2)c1-c1nc(NCc2ccc(-c3nc(C(F)(F)F)cn3C(C)C)cc2)c2cccn2n1]

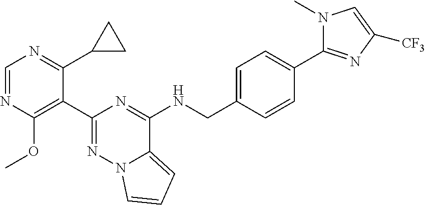 [SMILES: COc1ncnc(C2CC2)c1-c1nc(NCc2ccc(-c3nc(C(F)(F)F)cn3C)cc2)c2cccn2n1]

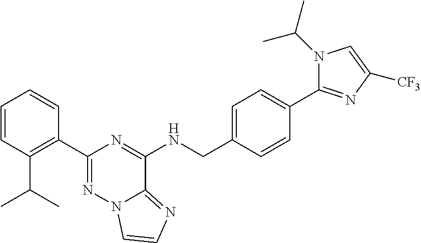 [SMILES: CC(C)c1ccccc1-c1nc(NCc2ccc(-c3nc(C(F)(F)F)cn3C(C)C)cc2)c2nccn2n1]

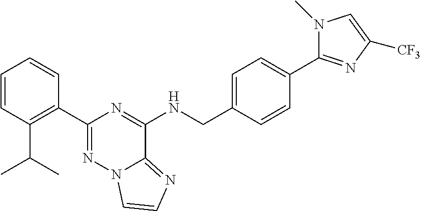 [SMILES: CC(C)c1ccccc1-c1nc(NCc2ccc(-c3nc(C(F)(F)F)cn3C)cc2)c2nccn2n1]

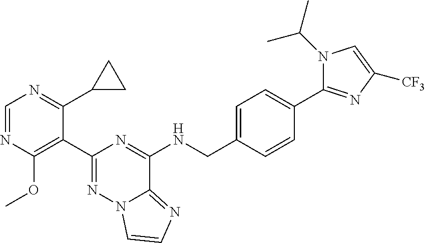 [SMILES: COc1ncnc(C2CC2)c1-c1nc(NCc2ccc(-c3nc(C(F)(F)F)cn3C(C)C)cc2)c2nccn2n1]

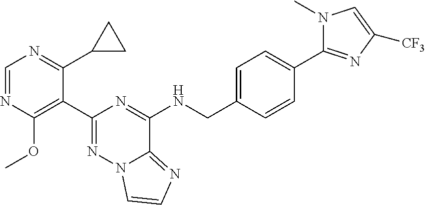 [SMILES: COc1ncnc(C2CC2)c1-c1nc(NCc2ccc(-c3nc(C(F)(F)F)cn3C)cc2)c2nccn2n1]

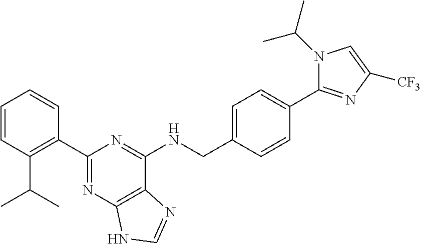 [SMILES: CC(C)c1ccccc1-c1nc(NCc2ccc(-c3nc(C(F)(F)F)cn3C(C)C)cc2)c2nc[nH]c2n1]

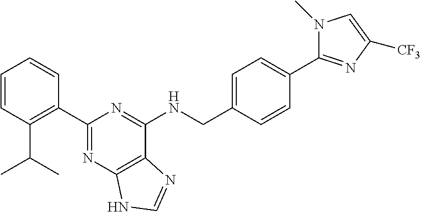 [SMILES: CC(C)c1ccccc1-c1nc(NCc2ccc(-c3nc(C(F)(F)F)cn3C)cc2)c2nc[nH]c2n1]

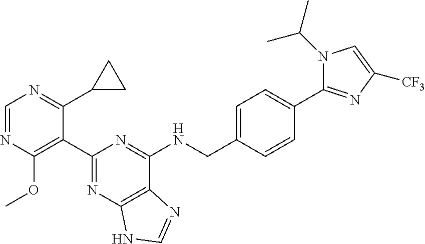 [SMILES: COc1ncnc(C2CC2)c1-c1nc(NCc2ccc(-c3nc(C(F)(F)F)cn3C(C)C)cc2)c2nc[nH]c2n1]

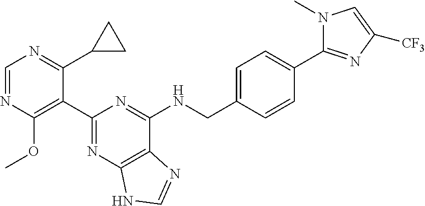 [SMILES: COc1ncnc(C2CC2)c1-c1nc(NCc2ccc(-c3nc(C(F)(F)F)cn3C)cc2)c2nc[nH]c2n1]

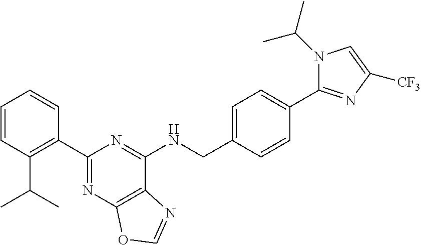 [SMILES: CC(C)c1ccccc1-c1nc(NCc2ccc(-c3nc(C(F)(F)F)cn3C(C)C)cc2)c2ncoc2n1]

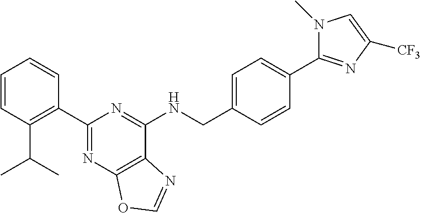 [SMILES: CC(C)c1ccccc1-c1nc(NCc2ccc(-c3nc(C(F)(F)F)cn3C)cc2)c2ncoc2n1]

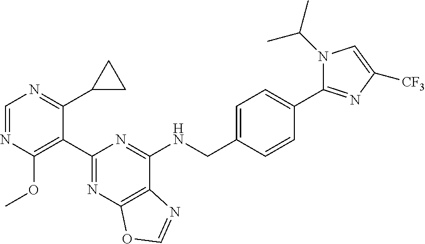 [SMILES: COc1ncnc(C2CC2)c1-c1nc(NCc2ccc(-c3nc(C(F)(F)F)cn3C(C)C)cc2)c2ncoc2n1]

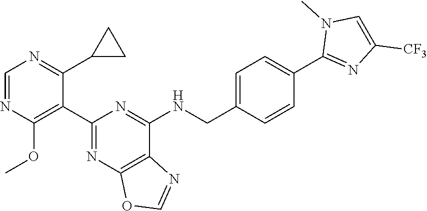 [SMILES: COc1ncnc(C2CC2)c1-c1nc(NCc2ccc(-c3nc(C(F)(F)F)cn3C)cc2)c2ncoc2n1]

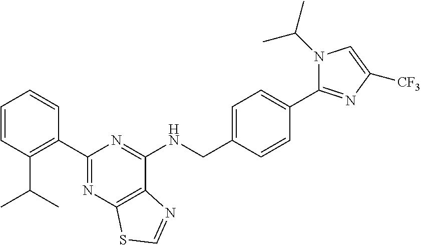 [SMILES: CC(C)c1ccccc1-c1nc(NCc2ccc(-c3nc(C(F)(F)F)cn3C(C)C)cc2)c2ncsc2n1]

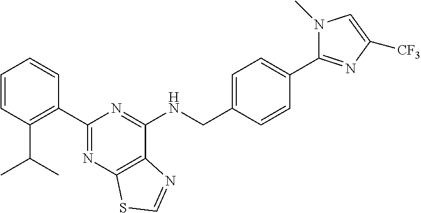 [SMILES: CC(C)c1ccccc1-c1nc(NCc2ccc(-c3nc(C(F)(F)F)cn3C)cc2)c2ncsc2n1]

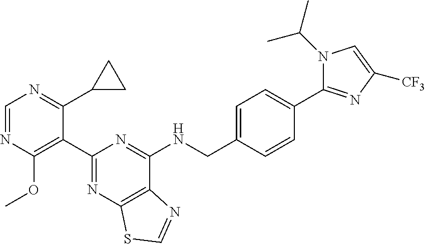 [SMILES: COc1ncnc(C2CC2)c1-c1nc(NCc2ccc(-c3nc(C(F)(F)F)cn3C(C)C)cc2)c2ncsc2n1]

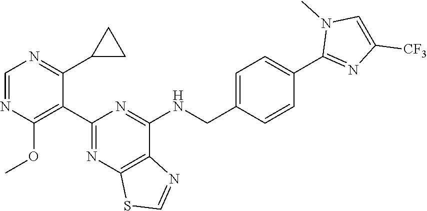 [SMILES: COc1ncnc(C2CC2)c1-c1nc(NCc2ccc(-c3nc(C(F)(F)F)cn3C)cc2)c2ncsc2n1]

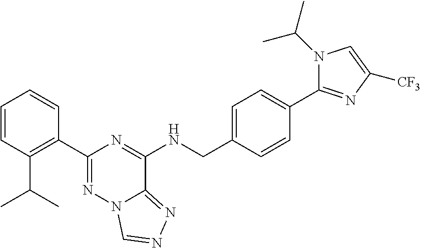 [SMILES: CC(C)c1ccccc1-c1nc(NCc2ccc(-c3nc(C(F)(F)F)cn3C(C)C)cc2)c2nncn2n1]

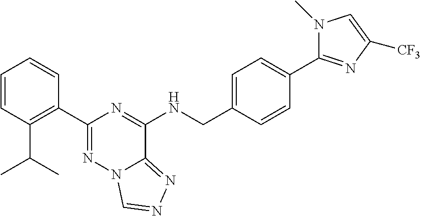 [SMILES: CC(C)c1ccccc1-c1nc(NCc2ccc(-c3nc(C(F)(F)F)cn3C)cc2)c2nncn2n1]

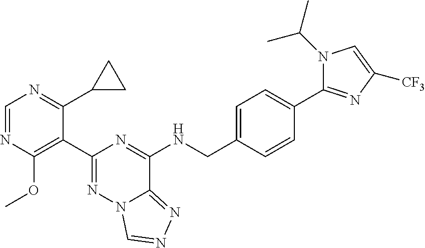 [SMILES: COc1ncnc(C2CC2)c1-c1nc(NCc2ccc(-c3nc(C(F)(F)F)cn3C(C)C)cc2)c2nncn2n1]

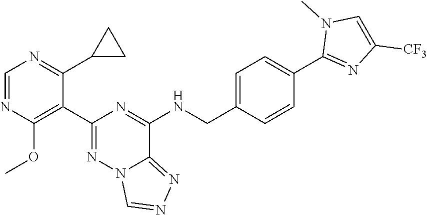 [SMILES: COc1ncnc(C2CC2)c1-c1nc(NCc2ccc(-c3nc(C(F)(F)F)cn3C)cc2)c2nncn2n1]

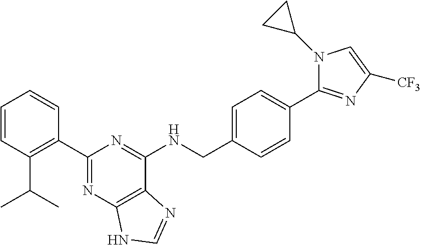 [SMILES: CC(C)c1ccccc1-c1nc(NCc2ccc(-c3nc(C(F)(F)F)cn3C3CC3)cc2)c2nc[nH]c2n1]

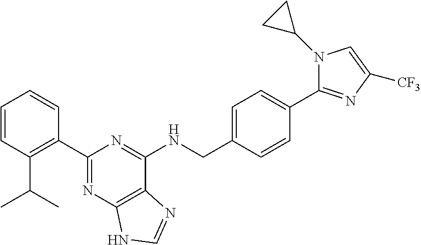 [SMILES: CC(C)c1ccccc1-c1nc(NCc2ccc(-c3nc(C(F)(F)F)cn3C3CC3)cc2)c2nc[nH]c2n1]

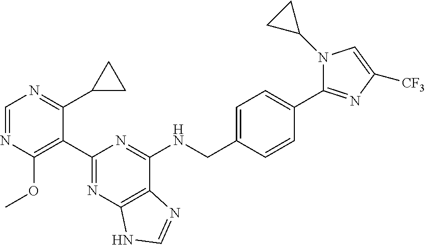 [SMILES: COc1ncnc(C2CC2)c1-c1nc(NCc2ccc(-c3nc(C(F)(F)F)cn3C3CC3)cc2)c2nc[nH]c2n1]

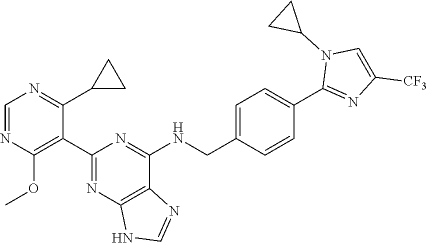 [SMILES: COc1ncnc(C2CC2)c1-c1nc(NCc2ccc(-c3nc(C(F)(F)F)cn3C3CC3)cc2)c2nc[nH]c2n1]

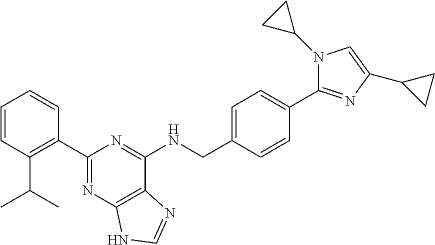 [SMILES: CC(C)c1ccccc1-c1nc(NCc2ccc(-c3nc(C4CC4)cn3C3CC3)cc2)c2nc[nH]c2n1]

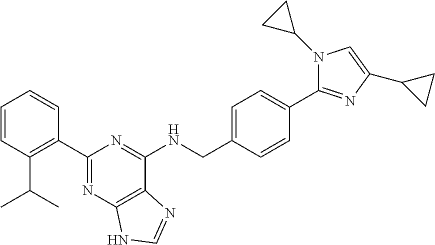 [SMILES: CC(C)c1ccccc1-c1nc(NCc2ccc(-c3nc(C4CC4)cn3C3CC3)cc2)c2nc[nH]c2n1]

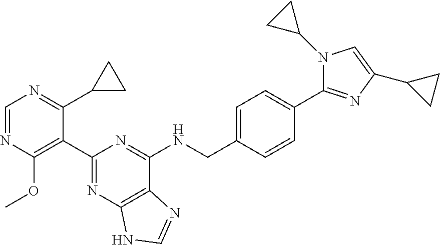 [SMILES: COc1ncnc(C2CC2)c1-c1nc(NCc2ccc(-c3nc(C4CC4)cn3C3CC3)cc2)c2nc[nH]c2n1]

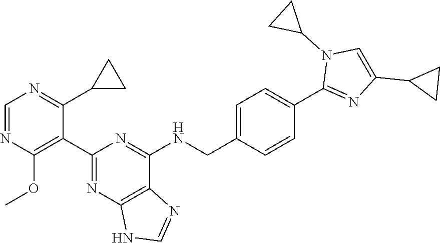 [SMILES: COc1ncnc(C2CC2)c1-c1nc(NCc2ccc(-c3nc(C4CC4)cn3C3CC3)cc2)c2nc[nH]c2n1]

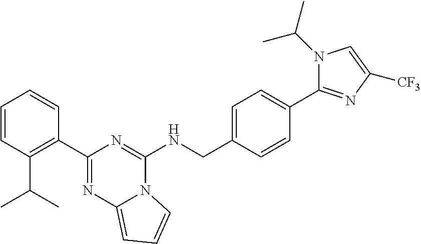 [SMILES: CC(C)c1ccccc1-c1nc(NCc2ccc(-c3nc(C(F)(F)F)cn3C(C)C)cc2)n2cccc2n1]

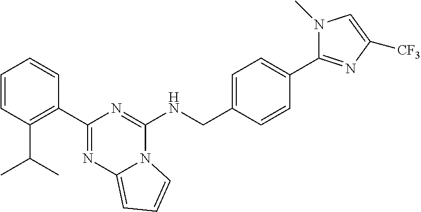 [SMILES: CC(C)c1ccccc1-c1nc(NCc2ccc(-c3nc(C(F)(F)F)cn3C)cc2)n2cccc2n1]

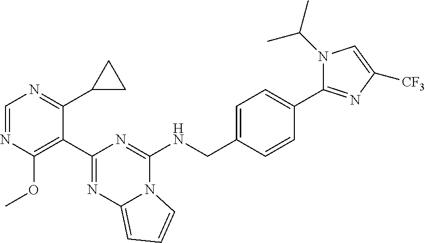 [SMILES: COc1ncnc(C2CC2)c1-c1nc(NCc2ccc(-c3nc(C(F)(F)F)cn3C(C)C)cc2)n2cccc2n1]

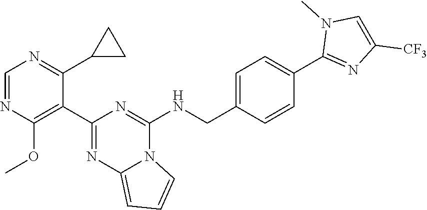 [SMILES: COc1ncnc(C2CC2)c1-c1nc(NCc2ccc(-c3nc(C(F)(F)F)cn3C)cc2)n2cccc2n1]

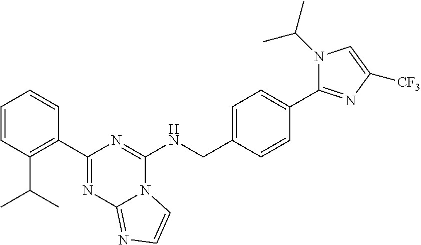 [SMILES: CC(C)c1ccccc1-c1nc(NCc2ccc(-c3nc(C(F)(F)F)cn3C(C)C)cc2)n2ccnc2n1]

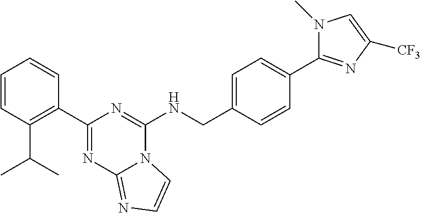 [SMILES: CC(C)c1ccccc1-c1nc(NCc2ccc(-c3nc(C(F)(F)F)cn3C)cc2)n2ccnc2n1]

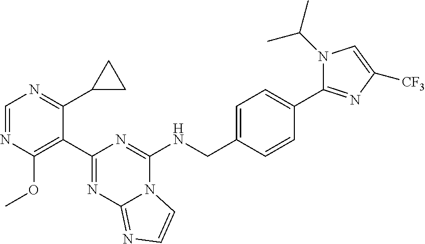 [SMILES: COc1ncnc(C2CC2)c1-c1nc(NCc2ccc(-c3nc(C(F)(F)F)cn3C(C)C)cc2)n2ccnc2n1]

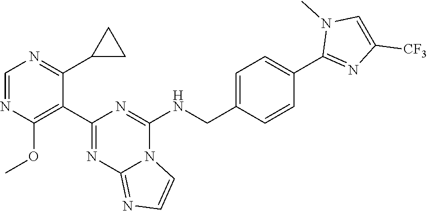 [SMILES: COc1ncnc(C2CC2)c1-c1nc(NCc2ccc(-c3nc(C(F)(F)F)cn3C)cc2)n2ccnc2n1]

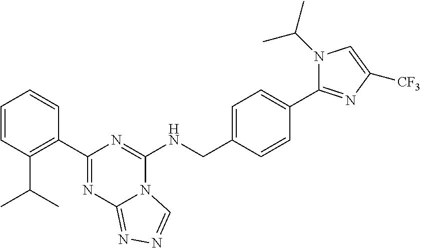 [SMILES: CC(C)c1ccccc1-c1nc(NCc2ccc(-c3nc(C(F)(F)F)cn3C(C)C)cc2)n2cnnc2n1]

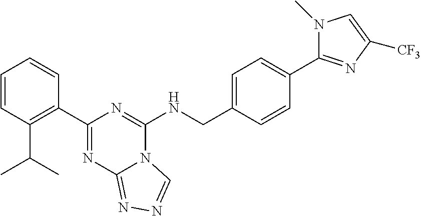 [SMILES: CC(C)c1ccccc1-c1nc(NCc2ccc(-c3nc(C(F)(F)F)cn3C)cc2)n2cnnc2n1]

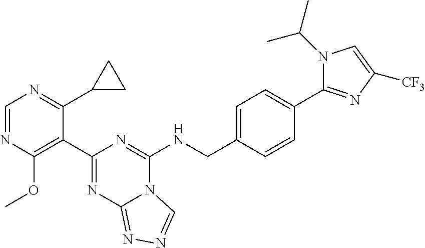 [SMILES: COc1ncnc(C2CC2)c1-c1nc(NCc2ccc(-c3nc(C(F)(F)F)cn3C(C)C)cc2)n2cnnc2n1]

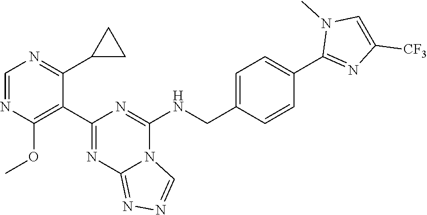 [SMILES: COc1ncnc(C2CC2)c1-c1nc(NCc2ccc(-c3nc(C(F)(F)F)cn3C)cc2)n2cnnc2n1]

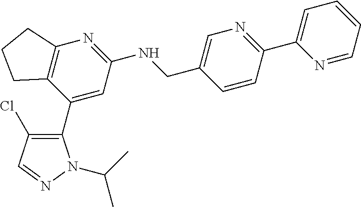 [SMILES: CC(C)n1ncc(Cl)c1-c1cc(NCc2ccc(-c3ccccn3)nc2)nc2c1CCC2]

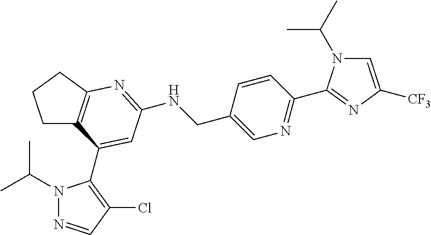

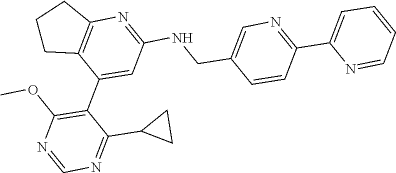 [SMILES: COc1ncnc(C2CC2)c1-c1cc(NCc2ccc(-c3ccccn3)nc2)nc2c1CCC2]

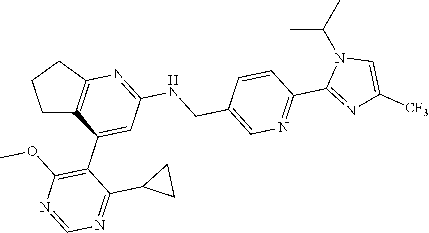

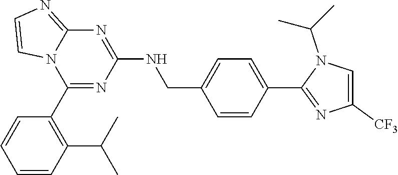 [SMILES: CC(C)c1ccccc1-c1nc(NCc2ccc(-c3nc(C(F)(F)F)cn3C(C)C)cc2)nc2nccn12]

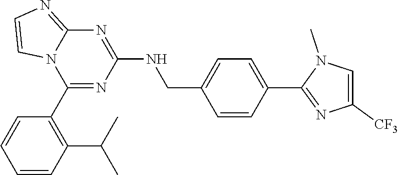 [SMILES: CC(C)c1ccccc1-c1nc(NCc2ccc(-c3nc(C(F)(F)F)cn3C)cc2)nc2nccn12]

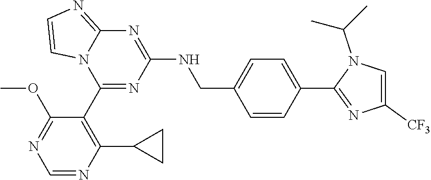 [SMILES: COc1ncnc(C2CC2)c1-c1nc(NCc2ccc(-c3nc(C(F)(F)F)cn3C(C)C)cc2)nc2nccn12]

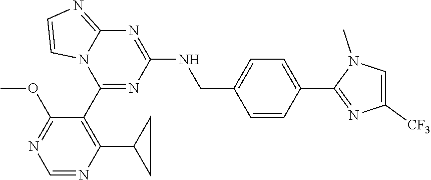 [SMILES: COc1ncnc(C2CC2)c1-c1nc(NCc2ccc(-c3nc(C(F)(F)F)cn3C)cc2)nc2nccn12]

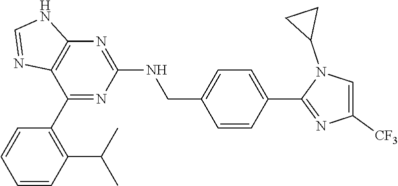 [SMILES: CC(C)c1ccccc1-c1nc(NCc2ccc(-c3nc(C(F)(F)F)cn3C3CC3)cc2)nc2[nH]cnc12]

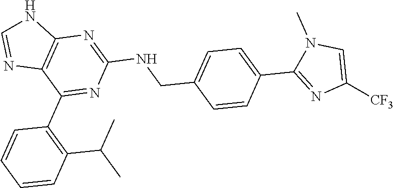 [SMILES: CC(C)c1ccccc1-c1nc(NCc2ccc(-c3nc(C(F)(F)F)cn3C)cc2)nc2[nH]cnc12]

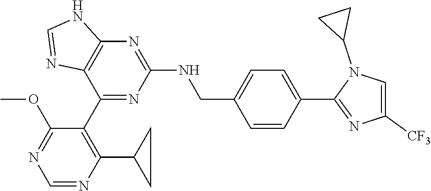 [SMILES: COc1ncnc(C2CC2)c1-c1nc(NCc2ccc(-c3nc(C(F)(F)F)cn3C3CC3)cc2)nc2[nH]cnc12]

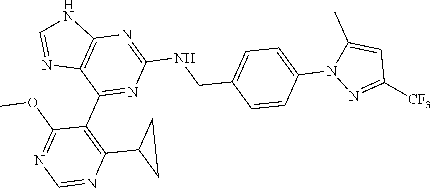 [SMILES: COc1ncnc(C2CC2)c1-c1nc(NCc2ccc(-n3nc(C(F)(F)F)cc3C)cc2)nc2[nH]cnc12]

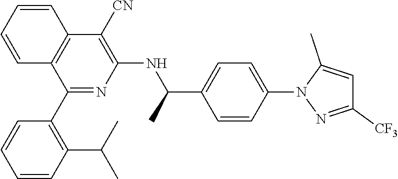 [SMILES: Cc1cc(C(F)(F)F)nn1-c1ccc([C@@H](C)Nc2nc(-c3ccccc3C(C)C)c3ccccc3c2C#N)cc1]

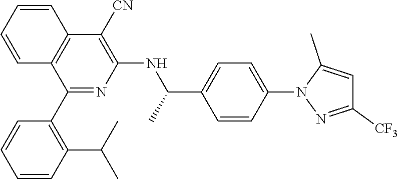 [SMILES: Cc1cc(C(F)(F)F)nn1-c1ccc([C@H](C)Nc2nc(-c3ccccc3C(C)C)c3ccccc3c2C#N)cc1]

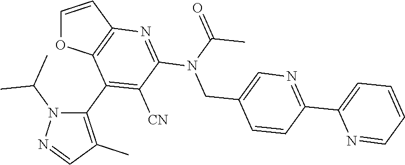 [SMILES: CC(=O)N(Cc1ccc(-c2ccccn2)nc1)c1nc2ccoc2c(-c2c(C)cnn2C(C)C)c1C#N]

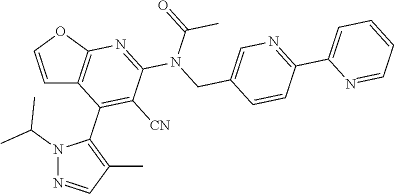 [SMILES: CC(=O)N(Cc1ccc(-c2ccccn2)nc1)c1nc2occc2c(-c2c(C)cnn2C(C)C)c1C#N]

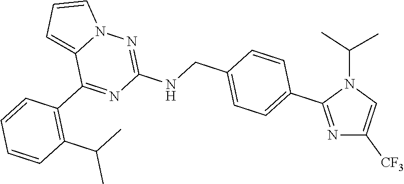 [SMILES: CC(C)c1ccccc1-c1nc(NCc2ccc(-c3nc(C(F)(F)F)cn3C(C)C)cc2)nn2cccc12]

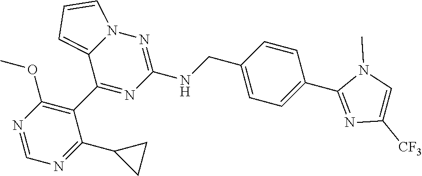 [SMILES: COc1ncnc(C2CC2)c1-c1nc(NCc2ccc(-c3nc(C(F)(F)F)cn3C)cc2)nn2cccc12]

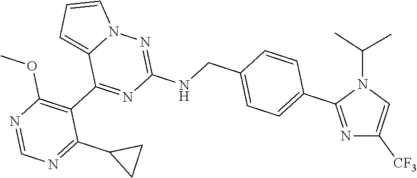 [SMILES: COc1ncnc(C2CC2)c1-c1nc(NCc2ccc(-c3nc(C(F)(F)F)cn3C(C)C)cc2)nn2cccc12]

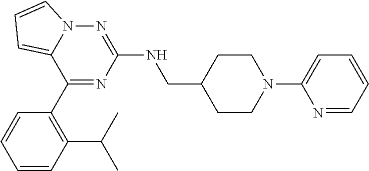 [SMILES: CC(C)c1ccccc1-c1nc(NCC2CCN(c3ccccn3)CC2)nn2cccc12]

Another embodiment of the invention is a composition comprising a compound of the invention, or a pharmaceutically acceptable salt thereof, and a pharmaceutically acceptable carrier.

Yet another embodiment of the invention is a method for treating cancer in a subject in need thereof, the method comprising administering to a subject in need thereof a therapeutically effective amount of a compound of the invention, or a pharmaceutically acceptable salt thereof, or a composition comprising a compound of the invention, or a pharmaceutically acceptable salt thereof.

Without being bound by a particular theory, provided herewith are the compounds that can modulate (e.g., inhibit) one or more members of the USP family. More specifically, and without being bound by a particular theory, it is believed that the compounds described herein can bind to one or more USP1.

As such, in another embodiment, the invention is a method of treating a USP1 mediated disorder in a subject in need thereof, in particular-USP1 mediated disorder comprising administering to the subject in need thereof a therapeutically effective amount of a compound of the invention, or a pharmaceutically acceptable salt thereof, or a pharmaceutical composition comprising a compound of the invention, or a pharmaceutically acceptable salt thereof.

Another embodiment of the invention is use of a compound of the invention for treating cancer or a USP-mediated disorder, in particular USP1, mediated disorder in a subject.

Another embodiment of the invention is use of a compound of the invention for treating cancer or a USP-mediated disorder, in particular USP1 mediated disorder in a subject.

Another embodiment of the invention is use of a compound of the invention for the manufacture of a medicament for treating cancer or a USP mediated disorder, in particular USP1 mediated disorder in a subject.

Another embodiment of the invention is use of a compound of the invention for the manufacture of a medicament for treating cancer or a USP mediated disorder, in particular USP1 mediated disorder in a subject.

Compounds of the present invention, and pharmaceutically acceptable salts and/or compositions thereof, are useful for treating a variety of cancers, such as solid cancer and, more specifically, USP1 associated solid cancers.

Compounds of the present invention, and pharmaceutically acceptable salts and/or compositions thereof, are useful for treating a variety of cancers, such as blood cancer and, more specifically, USP1 associated blood cancers.

### BRIEF DESCRIPTION OF THE DRAWING

FIG. 1: Line Graph depicting the antitumor effect of compounds of the invention in MDA-MB-436 Human Breast Cancer Xenograft.

### DETAILED DESCRIPTION

A description of example embodiments of the invention follows. Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by reference herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada.

Compounds of the present invention may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemates, racemic mixtures, and as individual diastereomers or enantiomers, with all possible isomers and mixtures thereof, including atrop and/or optical isomers, being included in the present invention.

As used herein the following definitions shall apply unless otherwise indicated. Further many of the groups defined herein can be optionally substituted. The listing of substituents in the definition is exemplary and is not to be construed to limit the substituents defined elsewhere in the specification.

The term “alkyl”, unless otherwise specified, refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (isopropyl), n-butyl, n-pentyl, and 1,1-dimethylethyl (t-butyl). The term “C₁₋₃alkyl” refers to an alkyl group as defined above having up to 3 carbon atoms. The term “C₁₋₆alkyl” refers to an alkyl group as defined above having up to 6 carbon atoms. In appropriate circumstances, the term “alkyl” refers to a hydrocarbon chain radical as mentioned above which is bivalent.

The term “alkenyl”, unless otherwise specified, refers to an aliphatic hydrocarbon group containing one or more carbon-carbon double bonds and which may be a straight or branched or branched chain having about 2 to about 10 carbon atoms, e.g., ethenyl, 1-propenyl, 2-propenyl (allyl), iso-propenyl, 2-methyl-1-propenyl, 1-butenyl, and 2-butenyl. The term “C₂₋₆alkenyl” refers to an alkenyl group as defined above having up to 6 carbon atoms. In appropriate circumstances, the term “alkenyl” refers to a hydrocarbon group as mentioned above which is bivalent.

The term “alkynyl”, unless otherwise specified, refers to a straight or branched chain hydrocarbyl radical having at least one carbon-carbon triple bond, and having in the range of 2 to up to 12 carbon atoms (with radicals having in the range of 2 to up to 10 carbon atoms presently being preferred) e.g., ethynyl, propynyl, and butnyl. The term “C₂₋₆ alkynyl” refers to an alkynyl group as defined above having up to 6 carbon atoms. In appropriate circumstances, the term “alkynyl” refers to a hydrocarbyl radical as mentioned above which is bivalent.

The term “alkoxy” unless otherwise specified, denotes an alkyl, cycloalkyl, or cycloalkylalkyl group as defined above attached via an oxygen linkage to the rest of the molecule. The term “substituted alkoxy” refers to an alkoxy group where the alkyl constituent is substituted (i.e., —O-(substituted alkyl). For example “alkoxy” refers to the group —O-alkyl, including from 1 to 8 carbon atoms of a straight, branched, cyclic configuration and combinations thereof attached to the parent structure through an oxygen atom. Examples include methoxy, ethoxy, propoxy, isopropoxy, cyclopropyloxy, and cyclohexyloxy. In appropriate circumstances, the term “alkoxy” refers to a group as mentioned above which is bivalent.

The term “cycloalkyl”, unless otherwise specified, denotes a non-aromatic mono or multicyclic ring system of about 3 to 12 carbon atoms such as cyclopropyl, cyclobutyl, cyclopentyl, and cyclohexyl. Examples of multicyclic cycloalkyl groups include perhydronaphthyl, adamantyl and norbornyl groups, bridged cyclic groups, and sprirobicyclic groups, e.g., sprio (4,4) non-2-yl. The term “C₃₋₆ cycloalkyl” refers to a cycloalkyl group as defined above having up to 6 carbon atoms.

The term “cycloalkylalkyl”, unless otherwise specified, refers to a cyclic ring-containing radical containing in the range of about 3 up to 8 carbon atoms directly attached to an alkyl group which is then attached to the main structure at any carbon from the alkyl group, such as cyclopropylmethyl, cyclobutylethyl, and cyclopentylethyl.

The term “cycloalkenyl”, unless otherwise specified, refers to cyclic ring-containing radicals containing in the range of about 3 up to 8 carbon atoms with at least one carbon-carbon double bond such as cyclopropenyl, cyclobutenyl, and cyclopentenyl. The term “cycloalkenylalkyl” refers to a cycloalkenyl group directly attached to an alkyl group which is then attached to the main structure at any carbon from the alkyl group.

The term “aryl”, unless otherwise specified, refers to aromatic radicals having in the range of 6 up to 20 carbon atoms such as phenyl, naphthyl, tetrahydronaphthyl, indanyl, and biphenyl.

The term “arylalkyl”, unless otherwise specified, refers to an aryl group as defined above directly bonded to an alkyl group as defined above, e.g., —CH₂C₆H₅ and —C₂H₅C₆H₅.

The term “heterocyclic ring”, unless otherwise specified, refers to a non-aromatic 3 to 15 member ring radical which consists of carbon atoms and at least one heteroatom selected from nitrogen, phosphorus, oxygen and sulfur. For purposes of this invention, the heterocyclic ring radical may be a mono-, bi-, tri- or tetracyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized. The heterocyclic ring radical may be attached to the main structure at any heteroatom or carbon atom.

The term “heterocyclyl”, unless otherwise specified, refers to a heterocylic ring radical as defined above. The heterocylcyl ring radical may be attached to the main structure at any heteroatom or carbon atom.

The term “heterocyclylalkyl”, unless otherwise specified, refers to a heterocylic ring radical as defined above directly bonded to an alkyl group. The heterocyclylalkyl radical may be attached to the main structure at any carbon atom in the alkyl group. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

The term “heteroaryl”, unless otherwise specified, refers to an optionally substituted 5 to 14 member aromatic ring having one or more heteroatoms selected from N, O, and S as ring atoms. The heteroaryl may be a mono-, bi- or tricyclic ring system. Examples of such “heterocyclic ring” or “heteroaryl” radicals include, but are not limited to, oxazolyl, thiazolyl, imidazolyl, pyrrolyl, furanyl, pyridinyl, pyrimidinyl, pyrazinyl, benzofuranyl, indolyl, benzothiazolyl, benzoxazolyl, carbazolyl, quinolyl, isoquinolyl, azetidinyl, acridinyl, benzodioxolyl, benzodioxanyl, benzofuranyl, carbazolyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, 4-piperidonyl, pyrrolidinyl, pyridazinyl, oxazolinyl, oxazolidinyl, triazolyl, indanyl, isoxazolyl, isoxazolidinyl, morpholinyl, thiazolinyl, thiazolidinyl, isothiazolyl, quinuclidinyl, isothiazolidinyl, isoindolyl, indolinyl, isoindolinyl, octahydroindolyl, octahydroisoindolyl, decahydroisoquinolyl, benzimidazolyl, thiadiazolyl, benzopyranyl, tetrahydrofuryl, tetrahydropyranyl, thienyl, benzothienyl, thiamorpholinyl, thiamorpholinyl sulfoxide, thiamorpholinyl sulfone, dioxaphospholanyl, oxadiazolyl, chromanyl, and isochromanyl. The heteroaryl ring radical may be attached to the main structure at any heteroatom or carbon atom. The term “substituted heteroaryl” also includes ring systems substituted with one or more oxide (—O—) substituents, such as pyridinyl N-oxides.

The term “heteroarylalkyl”, unless otherwise specified, refers to a heteroaryl ring radical as defined above directly bonded to an alkyl group. The heteroarylalkyl radical may be attached to the main structure at any carbon atom from alkyl group.

The term “cyclic ring” refers to a cyclic ring containing 3 to 10 carbon atoms.

The term “substituted” unless otherwise specified, refers to substitution with any one or any combination of the following substituents which may be the same or different and are independently selected from hydrogen, hydroxy, halogen, carboxyl, cyano, nitro, oxo (═O), thio (═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted heteroaryl, substituted or unsubstituted heteroarylalkyl, substituted or unsubstituted heterocyclic ring, substituted heterocyclylalkyl ring, substituted or unsubstituted guanidine, —COOR^{x}, —C(O)R^{x}, —C(S)R^{x}, —C(O)NR^{x}R^{y}, —C(O)ONR^{x}R^{y}, —NR^{y}R^{z}, —NR^{x}CONR^{y}R^{z}, —N(R^{x})SOR^{y}, —N(R^{x})SO₂R^{y}, —(═N—N(R^{x})R^{y}), —NR^{x}C(O)OR^{y}, —NR^{x}R^{y}, —NR^{x}C(O)R^{y}—, —NR^{x}C(S)R^{y}—, —NR^{x}C(S)NR^{y}R^{z}, —SONR^{x}R^{y}—, —SO₂NR^{x}R^{y}—, —OR^{x}, —OR^{x}C(O)NR^{y}R^{z}, —OR^{x}C(O)OR^{y}—, —OC(O)R^{x}, —OC(O)NR^{x}R^{y}, —R^{x}NR^{y}C(O)R^{z}, —R^{x}OR^{y}, —R^{x}C(O)OR^{y}, —R^{x}C(O)NR^{y}R^{z}, —R^{x}C(O)R^{x}, —R^{x}OC(O)R^{y}, —SR^{x}, —SOR^{x}, —SO₂R^{x}, and —ONO₂, wherein R^{x}, R^{y} and R^{z} in each of the above groups can be hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkylalkyl, substituted or unsubstituted cycloalkenyl, substituted or unsubstituted amino, substituted or unsubstituted heteroaryl, substituted or unsubstituted heteroarylalkyl, substituted or unsubstituted heterocyclic ring, or substituted heterocyclylalkyl ring, or any two of R^{x}, R^{y} and R^{z} may be joined to form a substituted or unsubstituted saturated or unsaturated 3-10 membered ring, which may optionally include heteroatoms which may be the same or different and are selected from O, NR^{x}(e.g., R^{x} can be hydrogen or C₁₋₆ alkyl) or S. Substitution or the combinations of substituents envisioned by this invention are preferably those that result in the formation of a stable or chemically feasible compound. The term stable as used herein refers to the compounds or the structure that are not substantially altered when subjected to conditions to allow for their production, detection and preferably their recovery, purification and incorporation into a pharmaceutical composition. The substituents in the aforementioned “substituted” groups cannot be further substituted. For example, when the substituent on “substituted alkyl” is “substituted aryl”, the substituent on “substituted aryl” cannot be “substituted alkenyl”.

The term “halo”, “halide”, or, alternatively, “halogen” means fluoro, chloro, bromo or iodo.

The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

The term “protecting group” or “PG” refers to a substituent that is employed to block or protect a particular functionality. Other functional groups on the compound may remain reactive. For example, an “amino-protecting group” is a substituent attached to an amino group that blocks or protects the amino functionality in the compound. Suitable amino-protecting groups include, but are not limited to, acetyl, trifluoroacetyl, tert-butoxycarbonyl (BOC), benzyloxycarbonyl (CBz) and 9-fluorenylmethylenoxycarbonyl (Fmoc). Similarly, a “hydroxy-protecting group” refers to a substituent of a hydroxy group that blocks or protects the hydroxy functionality. Suitable hydroxy-protecting groups include, but are not limited to, acetyl and silyl. A “carboxy-protecting group” refers to a substituent of the carboxy group that blocks or protects the carboxy functionality. Suitable carboxy-protecting groups include, but are not limited to, —CH₂CH₂SO₂Ph, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, and nitroethyl. For a general description of protecting groups and their use, see T. W. Greene, Protective Groups in Organic Synthesis, John Wiley & Sons, New York, 1991.

Certain of the compounds described herein contain one or more asymmetric centers and can thus give rise to enantiomers, diastereomers, and other stereoisomeric forms that can be defined, in terms of absolute stereochemistry, as (R)- or (S)-. The present chemical entities, pharmaceutical compositions and methods are meant to include all such possible isomers, including racemic mixtures, optically pure forms and intermediate mixtures. Non-limiting examples of intermediate mixtures include a mixture of isomers in a ratio of 10:90, 13:87, 17:83, 20:80, or 22:78. Optically active (R)- and (S)-isomers can be prepared using chiral synthons or chiral reagents or resolved using conventional techniques. When the compounds described herein contain olefinic double bonds or other centers of geometric asymmetry, and unless specified otherwise, it is intended that the compounds include both E and Z geometric isomers.

The term “tautomers” refers to compounds, which are characterized by relatively easy interconversion of isomeric forms in equilibrium. These isomers are intended to be covered by this invention. “Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g. in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4 (1H)-one tautomers.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ³C- or ¹⁴C-enriched carbon are within the scope of this invention. Such compounds are useful, for example, as analytical tools, as probes in biological assays, or as therapeutic agents in accordance with the present invention. For example, in the case of an variable say Z, the (C₁-C₄) alkyl, or —NZ—(C₁-C₄) alkyl the —O—(C₁-C₄) alkyl can be suitably deuterated (e.g., —CD₃, —NH—CD₃, —N—CD₃ or —OCD₃).

The compounds of the present invention may also contain unnatural proportions of atomic isotopes at one or more of atoms that constitute such compounds. For example, the compounds may be radiolabeled with radioactive isotopes, such as for example tritium (3H), iodine-125 (¹²⁵I) or carbon-14 (¹⁴C). All isotopic variations of the compounds of the present invention, whether radioactive or not, are encompassed within the scope of the present invention.

The term “stereoisomers” is a general term for all isomers of an individual molecule that differ only in the orientation of their atoms in space. It includes mirror image isomers (enantiomers), geometric (cis/trans) isomers and isomers of compounds with more than one chiral center that are not mirror images of one another (diastereomers).

“Electrophile” or “electrophilic moiety” is any moiety capable of reacting with a nucleophile (e.g., a moiety having a lone pair of electrons, a negative charge, a partial negative charge and/or an excess of electrons, for example a —SH group). Electrophiles typically are electron poor or comprise atoms which are electron poor. In certain embodiments an electrophile contains a positive charge or partial positive charge, has a resonance structure which contains a positive charge or partial positive charge or is a moiety in which delocalization or polarization of electrons results in one or more atom which contains a positive charge or partial positive charge. In some embodiments, the electrophiles comprise conjugated double bonds, for example an α, β-unsaturated carbonyl or α, β-unsaturated thiocarbonyl compound.

A “leaving group or atom” (Lg or lg) is any group or atom that will, under the reaction conditions, cleave from the starting material, thus promoting reaction at a specified site. Suitable examples of such groups unless otherwise specified are halogen atoms and mesyloxy, p-nitrobenzensulphonyloxy and tosyloxy groups.

“Prodrug” is meant to indicate a compound that may be converted under physiological conditions or by solvolysis to a biologically active compound described herein (e.g., compound of structure (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), or (IA-1B5). Thus, the term “prodrug” refers to a precursor of a biologically active compound that is pharmaceutically acceptable. In some aspects, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, Prodrug design is discussed generally in Hardma, et al. (Eds.), Goodman and Gilman's The Pharmacological Basis of Therapeutics, 9th ed., pp. 11-16 (1996) all of which are incorporated in full by reference herein. The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound and the like.

In some embodiments, prodrugs include compounds of structure (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), or (IA-1B5), having a phosphate, phosphoalkoxy, ester or boronic ester substituent. Without being bound by theory, it is believed that such substituents are converted to a hydroxyl group under physiological conditions. Accordingly, embodiments include any of the compounds disclosed herein, wherein a hydroxyl group has been replaced with a phosphate, phosphoalkoxy, ester or boronic ester group, for example a phosphate or phosphoalkoxy group. For example, in some embodiments a hydroxyl group on the R¹ moiety is replaced with a phosphate, phosphoalkoxy, ester or boronic ester group, for example a phosphate or alkoxy phosphate group.

The term “ester” refers to a compound, which is formed by reaction between an acid and an alcohol with elimination of water. An ester can be represented by the general formula RCOOR′.

These prodrugs and esters are intended to be covered within the scope of this invention.

Additionally, the instant invention also includes the compounds which differ only in the presence of one or more isotopically enriched atoms for example replacement of hydrogen with deuterium or tritium, or the replacement of a carbon by ^{13C}- or ^{14C}-enriched carbon.

When ranges are used herein for physical properties, such as molecular weight, or chemical properties, such as chemical formulae, all combinations and sub combinations of ranges and specific embodiments therein are intended to be included.

The term “about” when referring to a number or a numerical range means that the number or numerical range referred to is an approximation within experimental variability (or within statistical experimental error), and thus the number or numerical range may vary from, for example, between 1% and 15% of the stated number or numerical range.

The term “comprising” (and related terms such as “comprise” or “comprises” or “having” or “including”) includes those embodiments, for example, an embodiment of any composition of matter, composition, method, or process, or the like, that “consist of” or “consist essentially of” the described features.

The following abbreviations and terms have the indicated meanings throughout; Abbreviations used herein have their conventional meaning within the chemical and biological arts.

The term “cell proliferation” refers to a phenomenon by which the cell number has changed as a result of division. This term also encompasses cell growth by which the cell morphology has changed (e.g., increased in size) consistent with a proliferative signal.

The term “co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

The term “effective amount” or “therapeutically effective amount” refers to that amount of a compound described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g. reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried. In one embodiment, the amount of compound administered ranges from about 0.1 mg to 5 g, from about 1 mg to 2.0 g, from about 100 mg to 1.5 g, from about 200 mg to 1.5 g, from about 400 mg to 1.5 g, and from about 400 mg to 1.0 g.

As used herein, “treatment,” “treating,” or “ameliorating” refers to an approach for obtaining beneficial or desired results including but not limited to therapeutic benefit and/or a prophylactic benefit. By therapeutic benefit is meant eradication or amelioration of the underlying disorder being treated. In addition a therapeutic benefit is also achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. These terms are used interchangeably.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, an amount of a compound effective to treat a disorder, or a “therapeutically effective amount” refers to an amount of the compound which is effective, upon single or multiple dose administration to a subject or a cell, in curing, alleviating, relieving or improving one or more symptoms of a disorder.

As used herein, an amount of a compound effective to prevent a disorder, or a “prophylactically effective amount” of the compound refers to an amount effective, upon single- or multiple-dose administration to the subject, in preventing or delaying the onset or recurrence of a disorder or one or more symptoms of the disorder.

As used herein, the term “subject” or “patient” is intended to include human and non-human animals. Exemplary human subjects include a human patient having a disorder, e.g., a disorder described herein or a normal subject. The term “non-human animals” of the invention includes all vertebrates, e.g., non-mammals (such as chickens, amphibians, reptiles) and mammals, such as non-human primates, domesticated and/or agriculturally useful animals, e.g., sheep, cow, pig, etc., and companion animals (dog, cat, horse, etc.).

The methods described herein can be useful in both human therapeutics and veterinary applications (e.g., dogs, cats, cows, sheep, pigs, horses, goats, chickens, turkeys, ducks, and geese).

In some embodiments, the patient is a mammal, and in some embodiments, the patient is human.

“Radiation therapy” means exposing a patient, using routine methods and compositions known to the practitioner, to radiation emitters such as alpha-particle emitting radionuclides (e.g., actinium and thorium radionuclides), low linear energy transfer (LET) radiation emitters (i.e. beta emitters), conversion electron emitters (e.g. strontium-89 and samarium-153-EDTMP), or high-energy radiation, including without limitation x-rays, gamma rays, and neutrons.

The term “pharmaceutically acceptable carrier” or “pharmaceutically acceptable excipient” includes, but is not limited to, any and all, a non-toxic solvent, dispersant, excipient, adjuvant, fillers, salts, disintegrants, binders, lubricants, glidants, wetting agents, controlled release matrices, colorants/flavoring, carriers, buffers, stabilizers, solubilizers, or other material and combinations thereof which is mixed with the active ingredient in order to permit the formation of a pharmaceutical composition, i.e., a dosage form capable of being administered to a patient. One example of such a carrier is pharmaceutically acceptable oil typically used for parenteral administration. Pharmaceutically acceptable carriers are well known in the art.

It is understood that substituents and substitution patterns on the compounds of the invention can be selected by one of ordinary skill in the art to provide compounds that are chemically stable and that can be readily synthesized by techniques known in the art, as well as those methods set forth below. In general, the term “substituted,” whether preceded by the term “optionally” or not, means that one or more hydrogens of the designated moiety are replaced with a suitable substituent. Unless otherwise indicated, an “optionally substituted group” can have a suitable substituent at each substitutable position of the group and, when more than one position in any given structure may be substituted with more than one substituent selected from a specified group, the substituent can be either the same or different at every position. Alternatively, an “optionally substituted group” can be unsubstituted.

Combinations of substituents envisioned by this invention are preferably those that result in the formation of stable or chemically feasible compounds. If a substituent is itself substituted with more than one group, it is understood that these multiple groups can be on the same carbon atom or on different carbon atoms, as long as a stable structure results. The term “stable,” as used herein, refers to compounds that are not substantially altered when subjected to conditions to allow for their production, detection, and, in certain embodiments, their recovery, purification, and use for one or more of the purposes disclosed herein.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds of this invention include salts derived from suitable inorganic and organic acids and bases that are compatible with the treatment of patients.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable acid addition salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

In some embodiments, exemplary inorganic acids which form suitable salts include, but are not limited thereto, hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tricarboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicylic, 2 phenoxybenzoic, p-toluenesulfonic acid and other sulfonic acids such as methanesulfonic acid and 2-hydroxyethanesulfonic acid. Either the mono- or di-acid salts can be formed, or such salts can exist in either a hydrated, solvated or substantially anhydrous form. In general, the acid addition salts of these compounds are more soluble in water and various hydrophilic organic solvents, and generally demonstrate higher melting points in comparison to their free base forms.

In some embodiments, acid addition salts of the compounds of formula A are most suitably formed from pharmaceutically acceptable acids, and include, for example, those formed with inorganic acids, e.g., hydrochloric, sulfuric or phosphoric acids and organic acids e.g. succinic, maleic, acetic or fumaric acid.

Other non-pharmaceutically acceptable salts, e.g., oxalates can be used, for example, in the isolation of compounds of formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), or (IA-1B5), for laboratory use, or for subsequent conversion to a pharmaceutically acceptable acid addition salt. Also included within the scope of the invention are base addition salts (such as sodium, potassium and ammonium salts), solvates and hydrates of compounds of the invention. The conversion of a given compound salt to a desired compound salt is achieved by applying standard techniques, well known to one skilled in the art.

An “anti-cancer agent”, “anti-tumor agent” or “chemotherapeutic agent” refers to any agent useful in the treatment of a neoplastic condition. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository The term “cell proliferation” refers to a phenomenon by which the cell number has changed as a result of division. This term also encompasses cell growth by which the cell morphology has changed (e.g., increased in size) consistent with a proliferative signal.

The term “selective inhibition” or “selectively inhibit” refers to a biologically active agent refers to the agent's ability to preferentially reduce the target signalling activity as compared to off-target signalling activity, via direct or indirect interaction with the target.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients thereof.

When introducing elements disclosed herein, the articles “a,” “an,” “the,” and “said” are intended to mean that there are one or more of the elements. The terms “comprising,” “having” and “including” are intended to be open-ended and mean that there may be additional elements other than the listed elements.

For example, provided herein are methods of treating various cancers in mammals (including humans and non-humans), comprising administering to a patient in need thereof a compound of the invention, or a pharmaceutically acceptable salt thereof. Such cancers include hematologic malignancies (leukemias, lymphomas, myelomas, myelodysplastic and myeloproliferative syndromes) and solid tumors (carcinomas such as oral, gall bladder, prostate, breast, lung, colon, pancreatic, renal, ovarian as well as soft tissue and osteo sarcomas, and stromal tumors).

### Pharmaceutical Compositions

The invention provides a pharmaceutical composition comprising one or more compounds of the present invention. The pharmaceutical composition may include one or more additional active ingredients as described herein. The pharmaceutical composition may be administered for any of the disorders described herein.

The subject pharmaceutical compositions are typically formulated to provide a therapeutically effective amount of a compound of the present invention as the active ingredient. Where desired, the pharmaceutical compositions contain a compound of the present invention as the active ingredient and one or more pharmaceutically acceptable carriers or excipients, such as inert solid diluents and filers, diluents, including sterile aqueous solution and various organic solvents, permeation enhancers, solubilizers and adjuvants.

The pharmaceutical compositions can be administered alone or in combination with one or more other agents, which are also typically administered in the form of pharmaceutical compositions. Where desired, the subject compounds and other agent(s) may be mixed into a preparation or both components may be formulated into separate preparations to use them in combination separately or at the same time.

Methods include administration of a compound of the present invention by itself, or in combination as described herein, and in each case optionally including one or more suitable diluents, fillers, salts, disintegrants, binders, lubricants, glidants, wetting agents, controlled release matrices, colorants/flavouring, carriers, excipients, buffers, stabilizers, solubilizers, and combinations thereof.

Preparations of various pharmaceutical compositions are known in the art, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999), all of which are incorporated by reference herein in their entirety.

The compounds or pharmaceutical composition of the present invention can be administered by any route that enables delivery of the compounds to the site of action, such as oral routes, intraduodenal routes, parenteral injection (including intravenous, intraarterial, subcutaneous, intramuscular, intravascular, intraperitoneal or infusion), topical administration (e.g. transdermal application), rectal administration, via local delivery by catheter or stent or through inhalation. The compounds can also be administered intraadiposally or intrathecally.

The compositions can be administered in solid, semi-solid, liquid or gaseous form, or may be in dried powder, such as lyophilized form. The pharmaceutical compositions can be packaged in forms convenient for delivery, including, for example, solid dosage forms such as capsules, sachets, cachets, gelatins, papers, tablets, capsules, suppositories, pellets, pills, troches, and lozenges. The type of packaging will generally depend on the desired route of administration. Implantable sustained release formulations are also contemplated, as are transdermal formulations.

### Method of Treatment

In further aspects, the present invention provides a use of a compound of the invention, of a pharmaceutically acceptable salt thereof, for the manufacture of a medicament for the treatment of cancer. In some embodiments, the present invention provides a use of a compound of the invention in the manufacture of a medicament for the treatment of any of cancer and/or neoplastic disorders.

A compound or composition described herein can be used to treat a neoplastic disorder. A “neoplastic disorder” is a disease or disorder characterized by cells that have the capacity for autonomous growth or replication, e.g., an abnormal state or condition characterized by proliferative cell growth. Exemplary neoplastic disorders include but are not limited to: carcinoma, sarcoma, metastatic disorders, Solid tumor such as oral, gall bladder, prostate, breast, lung, colon, pancreatic, renal, ovarian as well as soft tissue and osteo sarcomas, and stromal tumors for e.g., tumors arising from prostate, brain, bone, colon, pancreas, lung, breast, ovarian, and liver origin, hematopoietic neoplastic disorders, e.g., leukemias, lymphomas, myelomas, myelodysplastic, myeloproliferative syndromes and other malignant plasma cell disorders, and metastatic tumors. Prevalent cancers include but not limited to: breast, prostate, colon, lung, liver, and pancreatic cancers. Treatment with the compound can be in an amount effective to ameliorate at least one symptom of the neoplastic disorder, e.g., reduced cell proliferation, reduced tumor mass, etc.

In another embodiment, a patient or population of patients to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a patient or population of patients to be treated with a pharmaceutical composition of the present disclosure have a hematological cancer. In some embodiments, the patient has a hematological cancer such as Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”). In some embodiments, a patient or population of patients to be treated having the cancer selected from the group consisting of ovarian cancer, lung cancer and melanoma.

Exemplary cancers including but not limited to tumors such as, the following: renal cancer, kidney cancer, glioblastoma multiforme, metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myclodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgen-dependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterer); renal carcinoma; Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas.

In another embodiment, the disclosure provides a method of treating cancer in a subject, comprising administering to the subject in need thereof an effective amount of a compound described herein, or a pharmaceutically acceptable salt or solvate thereof, or a pharmaceutical composition of described herein. In some embodiments, the cancer can comprise cancer cells with elevated levels of RAD 18 mRNA expression. In some embodiments, elevated levels of RAD 18 are elevated levels of RAD 18 protein. In some embodiments, RAD 18 levels can be detected using quantitative methods like microarray, RNA-Seq, or reverse transcriptase polymerase chain reaction (RT-PCR). In some embodiments, the levels of RAD 18 in a cancer cell can be detected prior to administration of the compounds described herein. In some embodiments, RAD 18 levels can be detected in a cancer sample obtained from a subject. In some embodiments, if a subject has elevated levels of RAD 18, the subject can be treated with the compounds described herein. In some embodiments, elevated levels of RAD 18 in cancer cells indicate that a subject administered the compounds or pharmaceutical compositions described herein is responsive to treatment using the compounds or pharmaceutical compositions described herein. In some embodiments, the compounds described herein are not administered to a subject with elevated levels of RAD 18.

In another embodiments, the cancer is a DNA damage repair pathway deficient cancer. In some embodiments, the cancer is a PARP inhibitor resistant or refractory BRCA1 or BRCA2-mutant cancer. In some embodiments, the cancer comprises cells with elevated levels of RAD 18, where the elevated levels of RAD 18 are at least as high as the RAD 18 mRNA and/or protein levels in ES2 cells or HEP3B217 cells.

In another embodiments, the cancer is a BRCA1 mutant cancer and/or a BRCA2 mutant cancer. In some embodiments, the cancer is a BRCA1 or BRCA2 wildtype cancer. In some embodiments, the cancer is a BRCA1-deficient cancer. In some embodiments, the cancer is a BRCA2-deficient cancer. In some embodiments, the cancer that comprises cancer cells with a mutation in a gene that encodes BRCA1 and/or BRCA2. In some embodiments, the cancer is a BRCA1 mutant cancer and BRCA2 deficient cancer. In some embodiments, the cancer is a BRCA1 deficient cancer and BRCA2 mutant cancer. In some embodiments, the cancer comprises cells with elevated levels of RAD 18, where the elevated levels of RAD 18 are at least as high as the RAD 18 mRNA and/or protein levels in ES2 cells or HEP3B217 cells.

Further preferred, the invention provides for methods for inhibiting USP activity in a cell, comprising contacting the cell in which inhibition of USP1 activity is desired with an effective amount of a compound of formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and (IA-1B5), pharmaceutically acceptable salts thereof or pharmaceutical compositions containing the compound or pharmaceutically acceptable salt thereof.

In one embodiment, a cell in which inhibition of USP1 activity is desired is contacted with an effective amount of a compound of formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and (IA-1B5), to negatively modulate the activity of USP1.

Further preferred, a therapeutically effective amount of pharmaceutically acceptable salt or pharmaceutical compositions containing a compound of formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and (IA-1B5), may be used.

By negatively modulating the activity of USP1, the methods described herein are designed to inhibit undesired cellular proliferation resulting from enhanced USP1 activity within the cell. The cells may be contacted in a single dose or multiple doses in accordance with a particular treatment regimen to effect the desired negative modulation of USP1.

Further preferred, methods of treating cancer in a patient in need thereof, comprising administering to said patient a therapeutically effective amount of a compound of formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and (IA-1B5), pharmaceutically acceptable salts thereof or pharmaceutical compositions comprising the compound or pharmaceutically acceptable salts thereof are provided.

The compositions and methods provided herein may be used for the treatment of a USP1 associated cancer in a patient in need thereof, comprising administering to said patient a therapeutically effective amount of a compound of formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and (IA-1B5), pharmaceutically acceptable salts thereof or pharmaceutical compositions comprising the compound or pharmaceutically acceptable salts thereof are provided. In one embodiment, the USP1-associated cancer is breast cancer or ovarian cancer.

Further preferred, the cancer is Breast cancer, ovarian cancer, prostate cancer, lung cancer, colorectal cancer or pancreatic cancer.

Further preferred, the cancer is prostate cancer or pancreatic cancer.

Further preferred, the cancer is lung cancer or colorectal cancer

Further preferred, the cancer is Breast cancer or ovarian cancer.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as in combination with other targeted agents or radiation or surgical intervention, either as an adjuvant prior to surgery or post-operatively.

The invention further provides herein a compound of formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and (IA-1B5), or a pharmaceutically acceptable salt or solvate thereof, or a pharmaceutical composition thereof as defined herein for use in therapy.

The invention further provides herein a compound of formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and (IA-1B5), or a pharmaceutically acceptable salt or solvate thereof or a pharmaceutical composition thereof as defined herein for use in the treatment of cancer.

The invention further provides herein is a compound of formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and (IA-1B5), or a pharmaceutically acceptable salt or solvate thereof for use in the inhibition of USP1.

The invention further provides herein is a compound of formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and (IA-1B5), or a pharmaceutically acceptable salt or solvate thereof or a pharmaceutical composition thereof as defined herein, for use in the treatment of a USP1 associated disease or disorder.

The invention further provides herein is the use of a compound of formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and (IA-1B5), or a pharmaceutically acceptable salt or solvate thereof, as defined herein in the manufacture of a medicament for the treatment of cancer.

The invention further provides herein is a use of a compound of formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and (IA-1B5), or a pharmaceutically acceptable salt or solvate thereof, as defined herein in the manufacture of a medicament for the inhibition of activity of USP1.

The invention further provides herein is the use of a compound of formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and (IA-1B5), or a pharmaceutically acceptable salt or solvate thereof, as defined herein, in the manufacture of a medicament for the treatment of a USP1 associated disease or disorder.

The invention further provides herein is a method for treating cancer in a patient in need thereof, the method comprising (a) determining that cancer is associated with a USP1 (e.g., a USP1-associated cancer) (e.g., as determined using a assay or kit); and (b) administering to the patient a therapeutically effective amount of a compound of formula (T), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and (IA-1B5), or a pharmaceutically acceptable salt thereof, or a pharmaceutical composition thereof.

Metastases of the aforementioned cancers can also be treated or prevented in accordance with the methods described herein.

### Combination Therapies

In some embodiments, a compound described herein is administered together with an additional “second” therapeutic agent or treatment. The choice of second therapeutic agent may be made from any agent that is typically used in a monotherapy to treat the indicated disease or condition. As used herein, the term “administered together” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of any of the formulas described herein, an additional therapeutic agent, and a pharmaceutically acceptable carrier, adjuvant, or vehicle.

The compounds, pharmaceutically acceptable salts thereof and pharmaceutical compositions comprising such compounds and salts also may be co-administered with other anti-neoplastic compounds, e.g., chemotherapy, or used in combination with other treatments, such as in combination with other targeted agents or radiation or surgical intervention, either as an adjuvant prior to surgery or post-operatively.

The compounds, pharmaceutically acceptable salts thereof and pharmaceutical compositions comprising such compounds comprising the step of administering simultaneously or sequentially to a subject in need thereof at least one other anti-cancer agent, anti-inflammatory agent, immunosuppressive agent, steroid, non-steroidal anti-inflammatory agent, antihistamine, analgesic, or a mixture thereof.

### Process for Preparation of the Compound of the Invention

The instant compounds of the present invention may be prepared by the following general process. The process provided herein can similarly be applied to synthesize all possible variation of the compound of the invention, and in particular compounds of formulas (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and (IA-1B5), as provided herein above with all intended modification or without any modification. Unless otherwise indicated, the variables such as X¹, X², X³, X⁴, R, R¹, R⁵, L, L¹, L², n and Cy¹, used here in various intermediates or compounds of formulas are to be constructed to be the variables defined herein above in relation to compound of the invention and in particular to the compound of formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and (IA-1B5).

Synthetic Scheme I: This schematic representation of scheme aims to provide a possible route for the preparation of a compound of formula (I), (IA), (IB), (IA-1), (IA-2), (IB-1), (IB-2), (IA-1A), (IA-1B), (IA-1A1), (IA-1A2), (IA-1A3), (IA-1A4), (IA-1A5) (IA-1B1), (IA-1B2), (IA-1B3), (IA-1B4), and (IA-1B5).

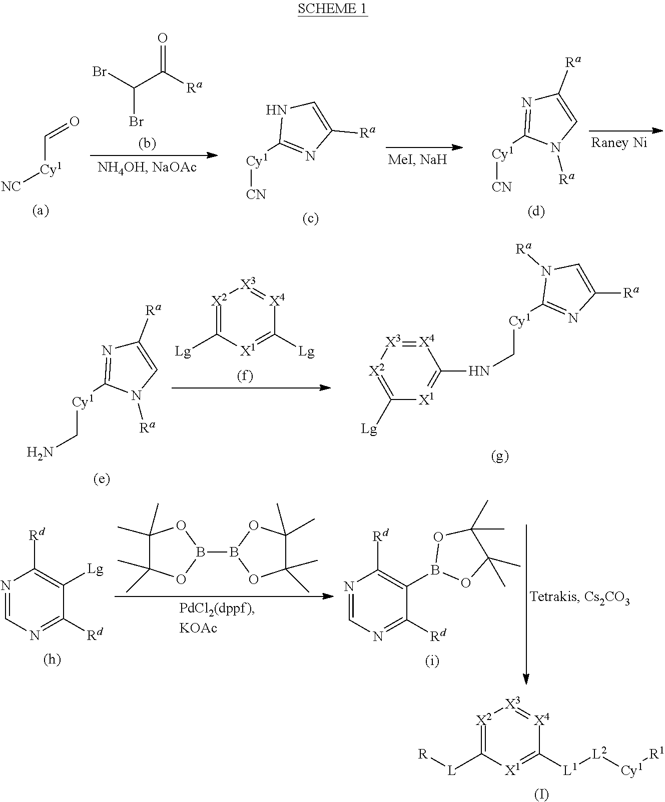

A compound of formula (a) is coupled with a compound of formula (b) to form a compound of formula (c). A compound of formula (c) can be converted as a compound of formula (d). A compound of formula (d) can be converted as a compound of formula (e). A compound of formula (e) can be coupled with a compound of formula (f) wherein Lg is a leaving group such as halogen to form a compound of formula (g).

A compound of formula (h) wherein Lg is a leaving group such as halogen can be coupled with Bis(pinacolato)diboron under suzuki reaction conditions in presence of palladium catalyst and a suitable base to form a compound of formula (i). A compound of formula (g) can be coupled with a compound of formula (i) under suzuki reaction conditions in presence of palladium catalyst and a suitable base to form desired compound of formula (I).

### Illustration-1:

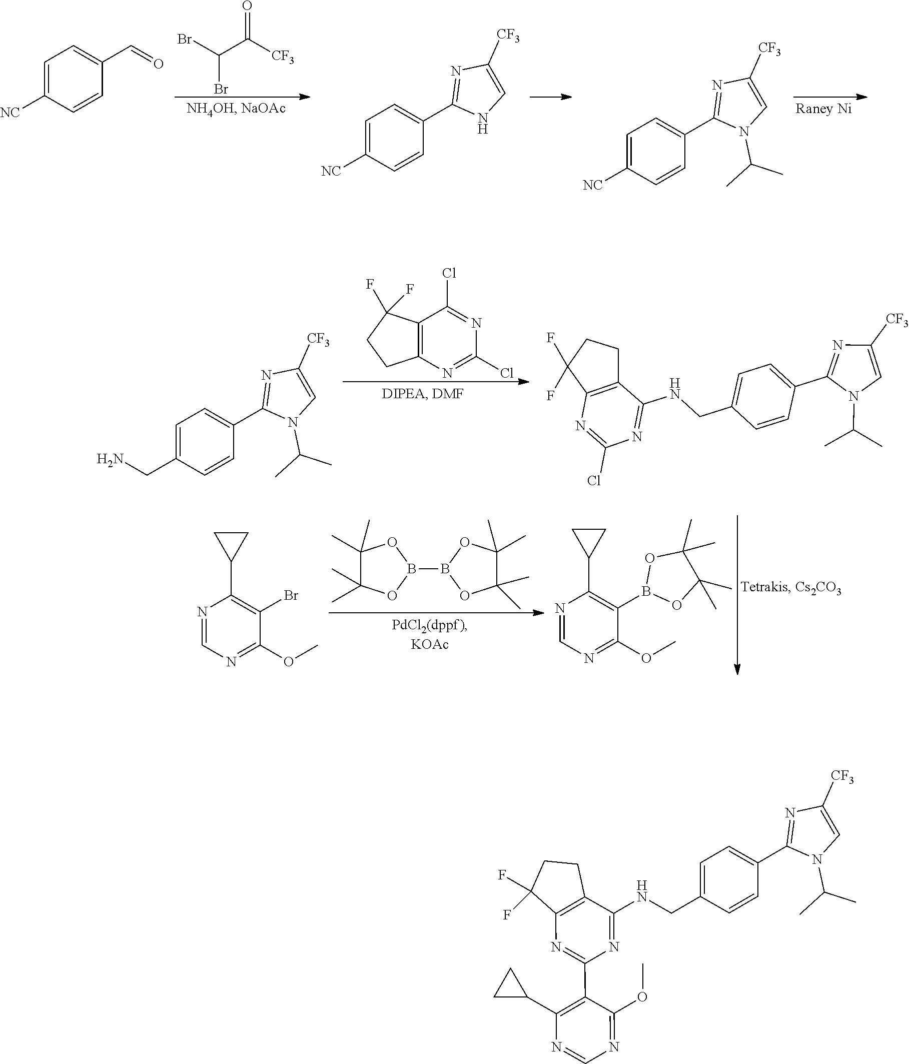

### Illustration-2:

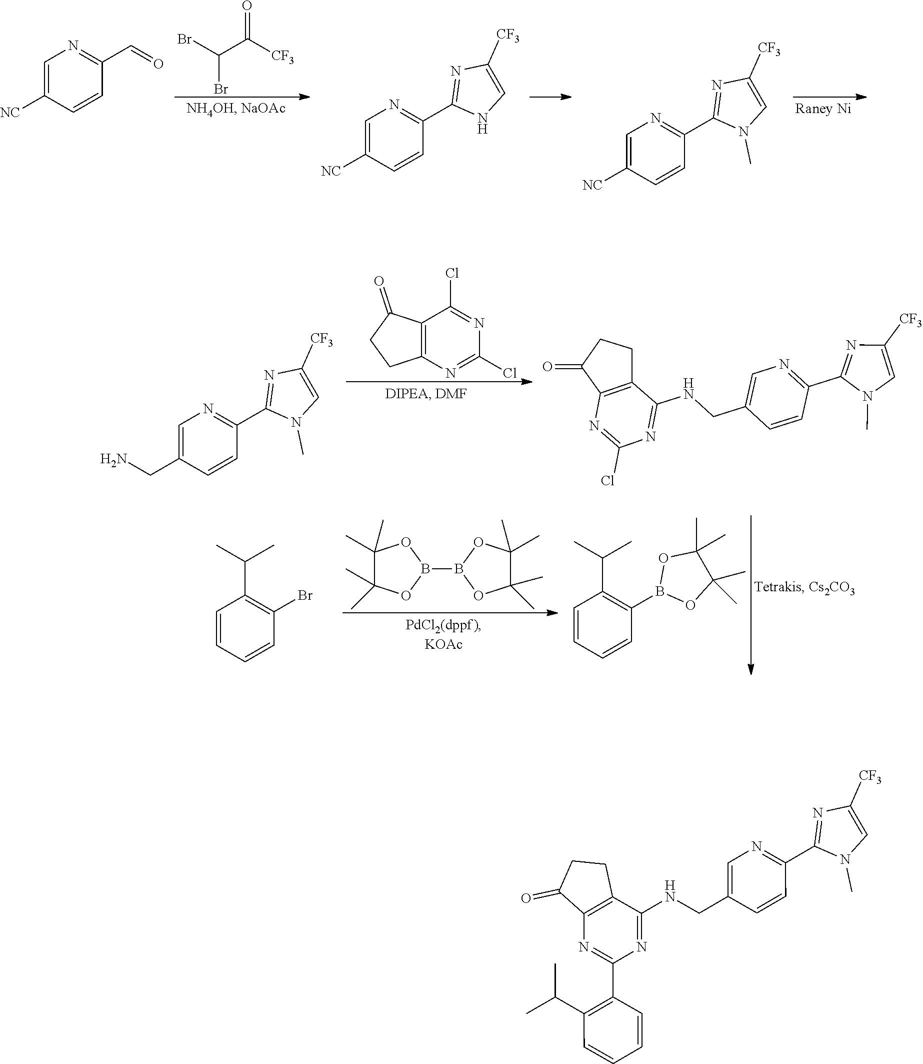

### Illustration-3:

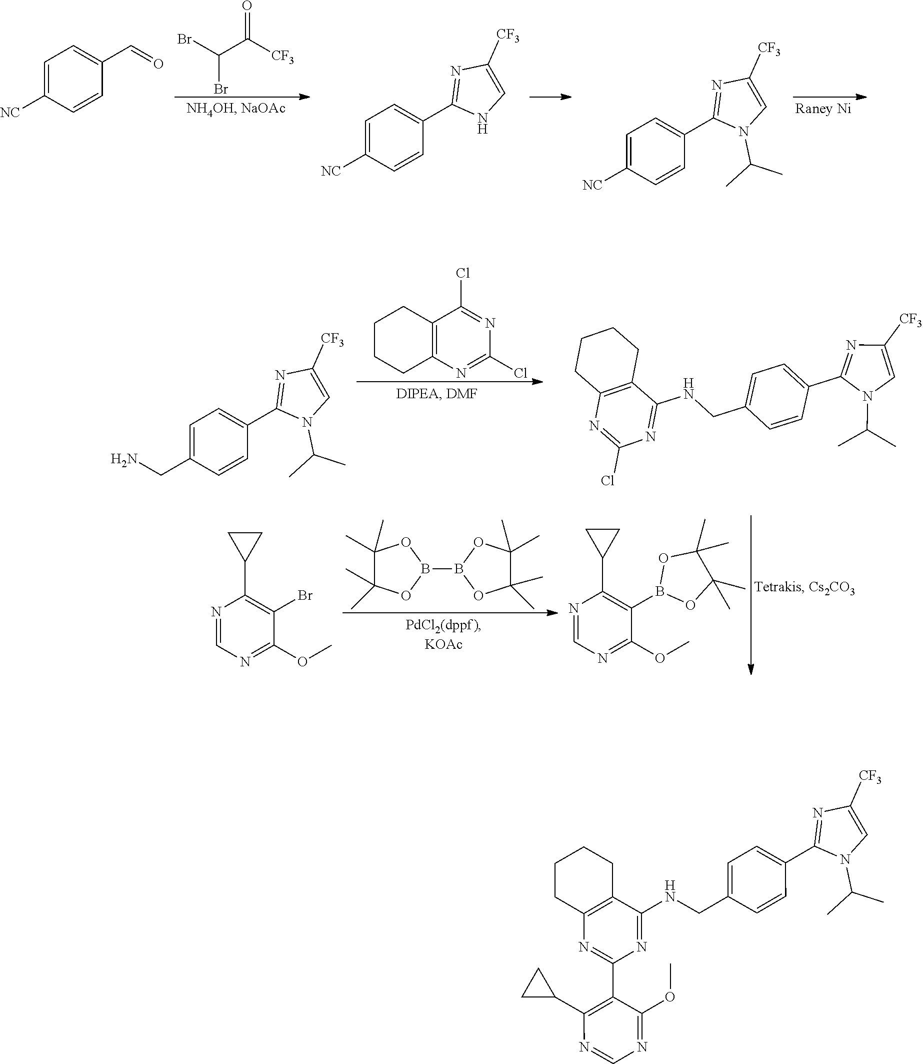

### Illustration-4:

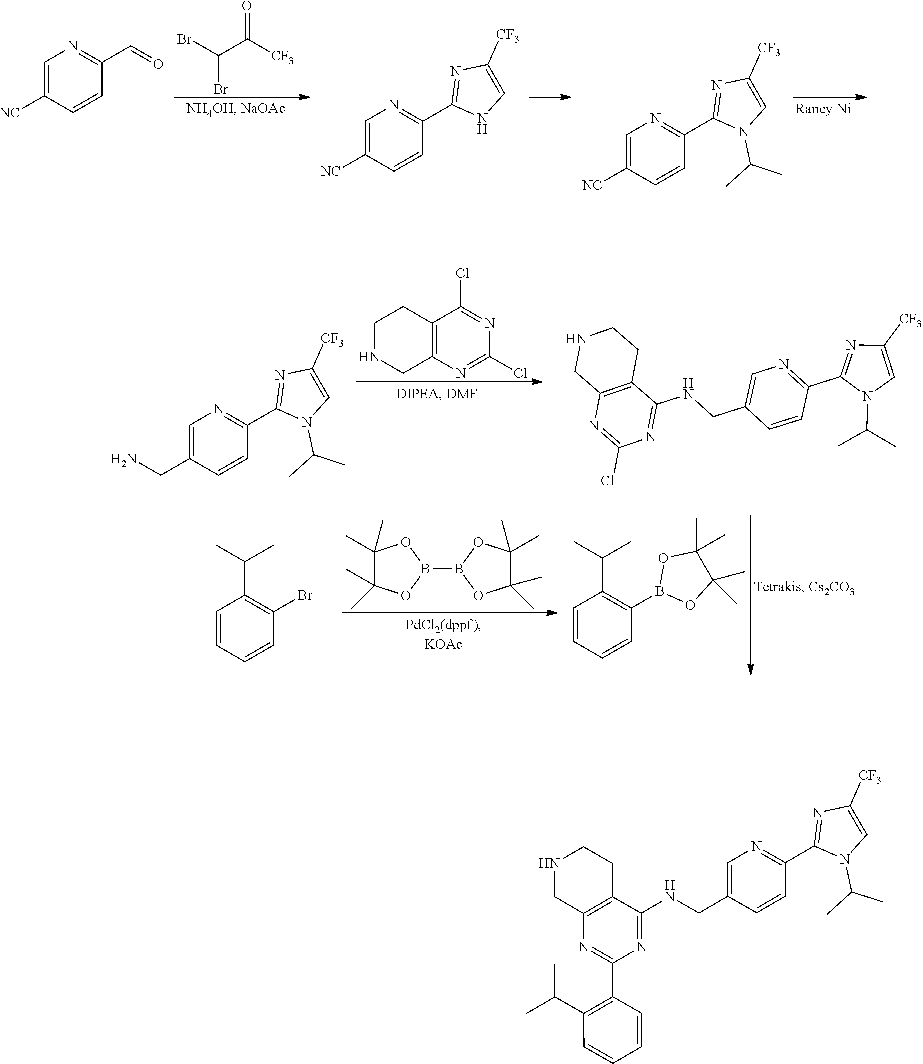

### Illustration-5:

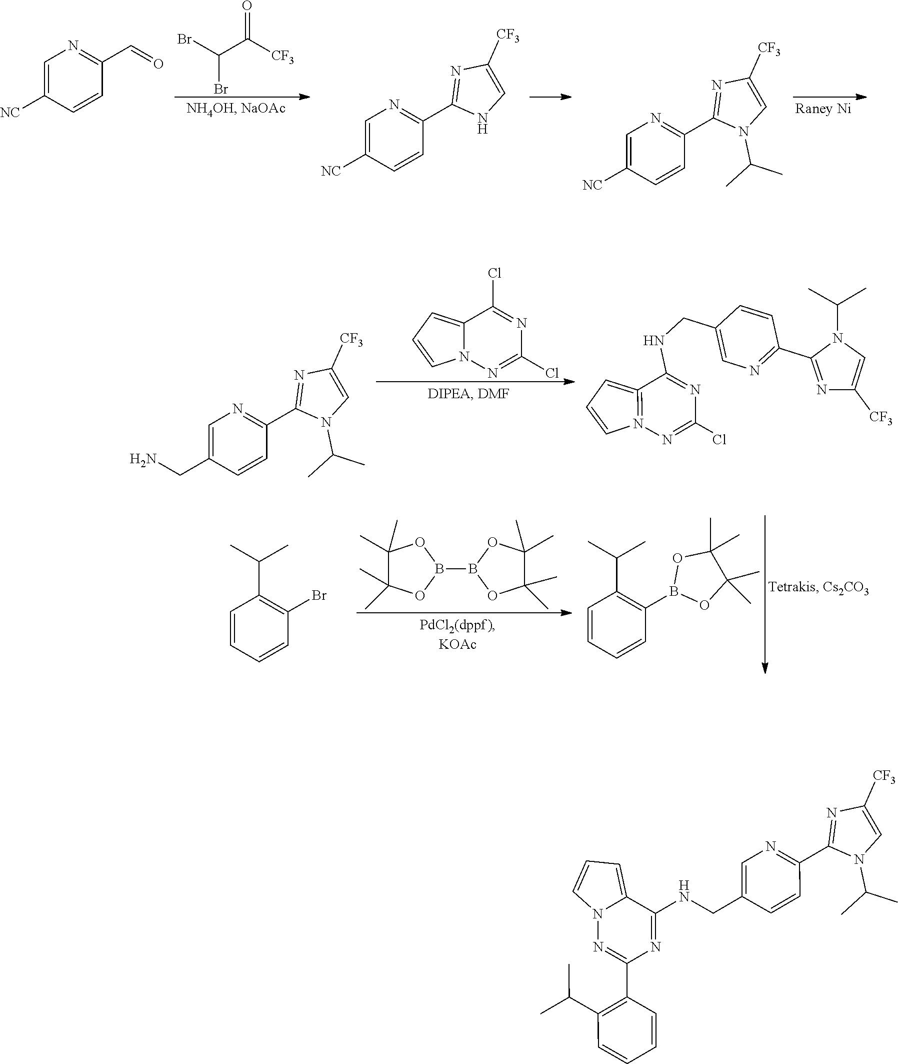

### Illustration-6:

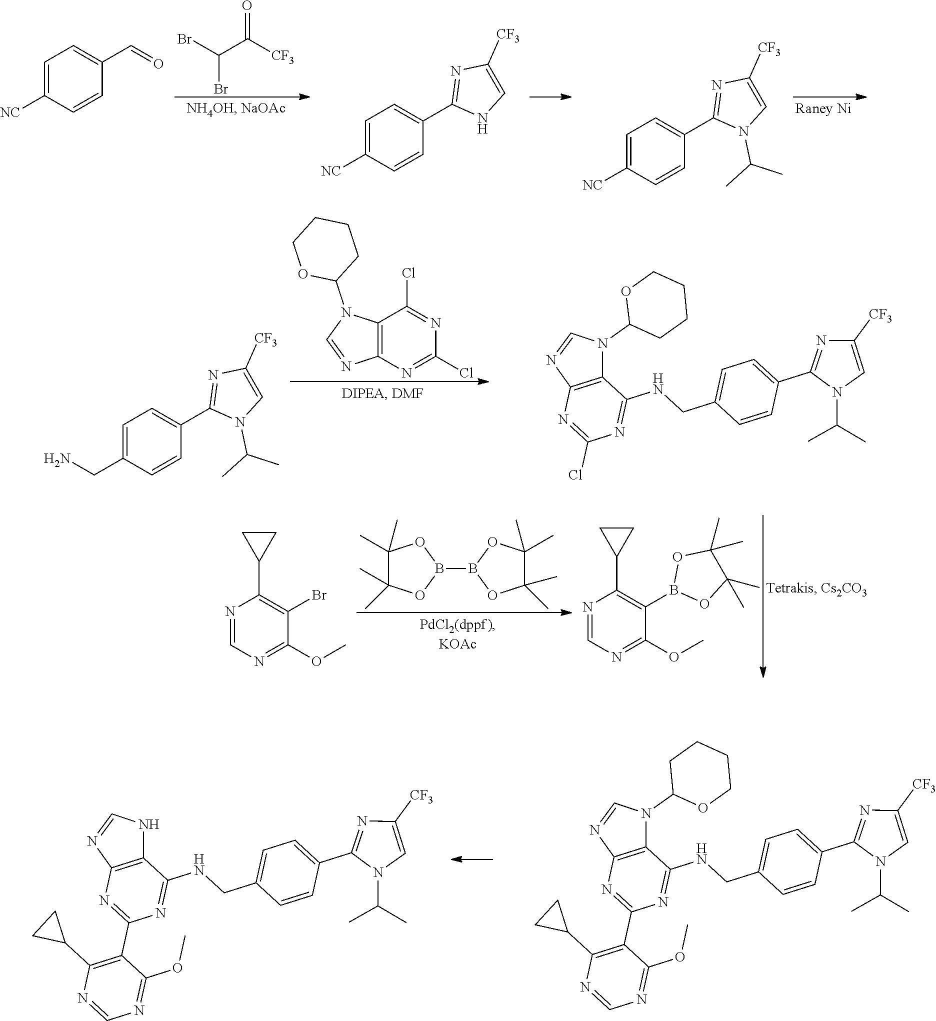

### Experimental Details

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

### General Procedure-1:

To a solution of Intermediate 15 (0.11 g) and a respective boronic acid (0.15 g) was dissolved in Dioxane. To it Cs₂CO₃ (3.0 eq) was added, N₂ gas purged for 15 mins. Tetrakis (0.05 eq) were added and purged again for 5 mins. Reaction mixture was heated at 110° C. for 16 to 20 hours. Reaction mixture was poured into ice-cold water (20 ml) and extracted with EtOAc (3×50 ml), combined organic layer and dried over Na₂SO₄. Purified by column chromatography compound eluted at 75-80% EtOAc-n-Hexane to Intermediate.

### General Procedure-2:

To a solution of respective intermediates selected from Intermediate 16 to 36 (1.0 eq) was dissolved in DCM (2 ml). To it TFA (2.0 ml) was added. Reaction mixture was Stirred at RT for 2 to 3 hours. Reaction mixture was evaporated under vacuum, and saturated NaHCO₃ solution (˜10 ml) was added and extracted with DCM (3×20 ml), combined organic layer was dried over Na₂SO₄ and evaporated under vacuum to give desired compounds with or without column purification.

### General Procedure-3:

To a solution of Intermediate 8 (0.10 g, 1.0 eq) and respective boronic acid (2.0 eq) was dissolved in Dioxane. To it Cs₂CO₃ (3.0 eq) was added, N₂ gas purged for 15 mins. Tetrakis (0.05 eq) were added and purged again for 5 mins. Reaction mixture was heated at 110° C. for 6 hours. Reaction mixture was poured into ice-cold water (20 ml) and extracted with EtOAc (3×50 ml), combined organic layer and dried over Na₂SO₄. Purified by column chromatography compound eluted at 75-80% EtOAc-n-Hexane to give desired compounds.

TABLE-3

| Int. | Structure |
|---|---|
| 1 | 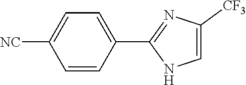 [SMILES: N#Cc1ccc(-c2nc(C(F)(F)F)c[nH]2)cc1] |
| 2 | 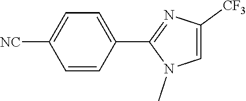 [SMILES: Cn1cc(C(F)(F)F)nc1-c1ccc(C#N)cc1] |
| 3 | 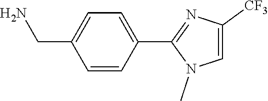 [SMILES: Cn1cc(C(F)(F)F)nc1-c1ccc(CN)cc1] |
| 4 | 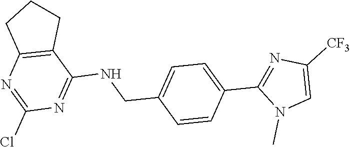 [SMILES: Cn1cc(C(F)(F)F)nc1-c1ccc(CNc2nc(Cl)nc3c2CCC3)cc1] |
| 5 | 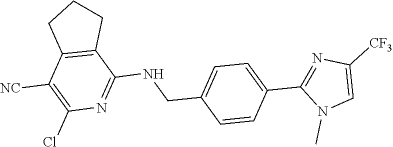 [SMILES: Cn1cc(C(F)(F)F)nc1-c1ccc(CNc2nc(Cl)c(C#N)c3c2CCC3)cc1] |
| 6 | 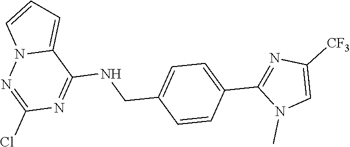 [SMILES: Cn1cc(C(F)(F)F)nc1-c1ccc(CNc2nc(Cl)nn3cccc23)cc1] |
| 7 | 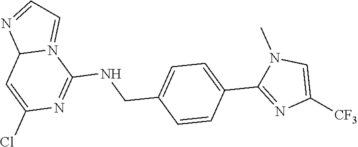 |
| 8 | 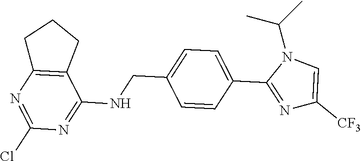 [SMILES: CC(C)n1cc(C(F)(F)F)nc1-c1ccc(CNc2nc(Cl)nc3c2CCC3)cc1] |
| 9 | 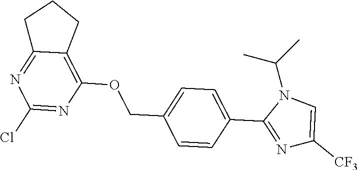 [SMILES: CC(C)n1cc(C(F)(F)F)nc1-c1ccc(COc2nc(Cl)nc3c2CCC3)cc1] |
| 10 | 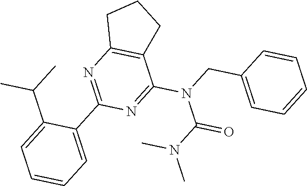 [SMILES: CC(C)c1ccccc1-c1nc2c(c(N(Cc3ccccc3)C(=O)N(C)C)n1)CCC2] |
| 11 | 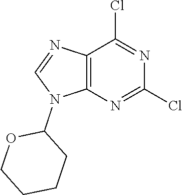 [SMILES: Clc1nc(Cl)c2ncn(C3CCCCO3)c2n1] |
| 12 | 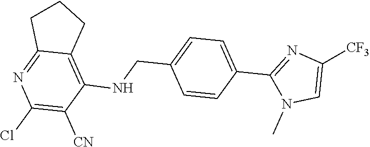 [SMILES: Cn1cc(C(F)(F)F)nc1-c1ccc(CNc2c(C#N)c(Cl)nc3c2CCC3)cc1] |
| 13 | 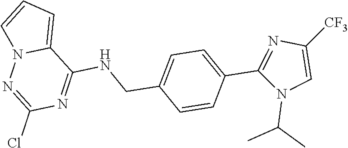 [SMILES: CC(C)n1cc(C(F)(F)F)nc1-c1ccc(CNc2nc(Cl)nn3cccc23)cc1] |
| 14 | 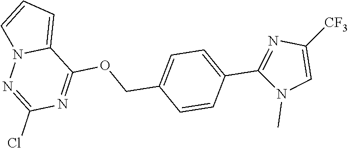 [SMILES: Cn1cc(C(F)(F)F)nc1-c1ccc(COc2nc(Cl)nn3cccc23)cc1] |
| 15 | 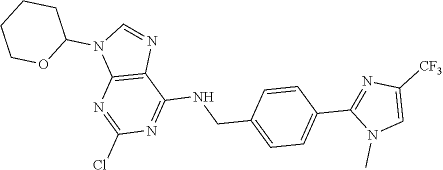 [SMILES: Cn1cc(C(F)(F)F)nc1-c1ccc(CNc2nc(Cl)nc3c2ncn3C2CCCCO2)cc1] |
| 16 | 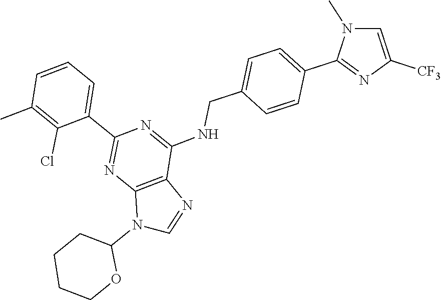 [SMILES: Cc1cccc(-c2nc(NCc3ccc(-c4nc(C(F)(F)F)cn4C)cc3)c3ncn(C4CCCCO4)c3n2)c1Cl] |
| 17 | 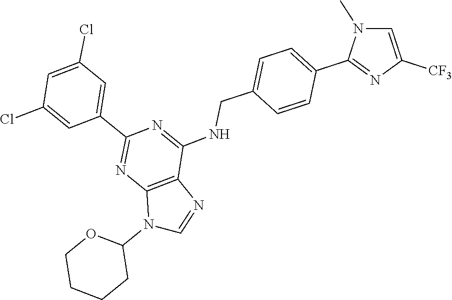 [SMILES: Cn1cc(C(F)(F)F)nc1-c1ccc(CNc2nc(-c3cc(Cl)cc(Cl)c3)nc3c2ncn3C2CCCCO2)cc1] |
| 18 | 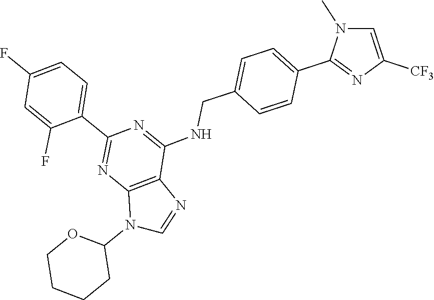 [SMILES: Cn1cc(C(F)(F)F)nc1-c1ccc(CNc2nc(-c3ccc(F)cc3F)nc3c2ncn3C2CCCCO2)cc1] |
| 19 | 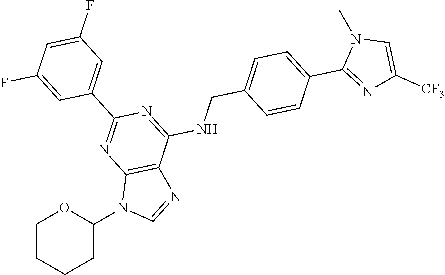 [SMILES: Cn1cc(C(F)(F)F)nc1-c1ccc(CNc2nc(-c3cc(F)cc(F)c3)nc3c2ncn3C2CCCCO2)cc1] |
| 20 | 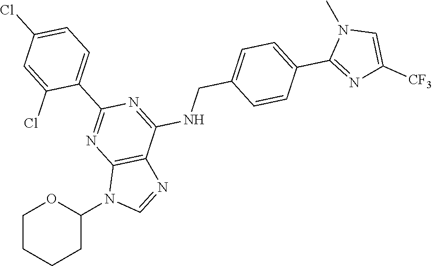 [SMILES: Cn1cc(C(F)(F)F)nc1-c1ccc(CNc2nc(-c3ccc(Cl)cc3Cl)nc3c2ncn3C2CCCCO2)cc1] |
| 21 | 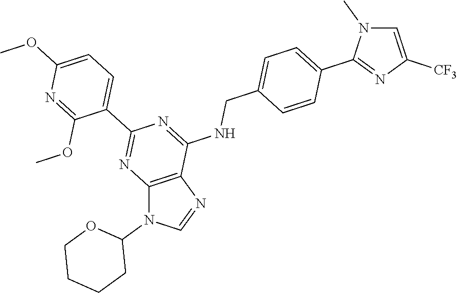 [SMILES: COc1ccc(-c2nc(NCc3ccc(-c4nc(C(F)(F)F)cn4C)cc3)c3ncn(C4CCCCO4)c3n2)c(OC)n1] |
| 22 | 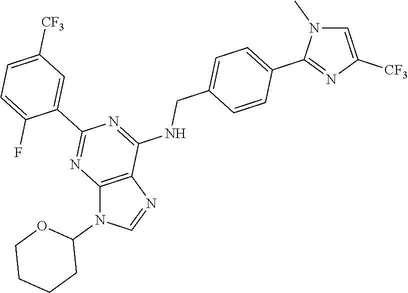 [SMILES: Cn1cc(C(F)(F)F)nc1-c1ccc(CNc2nc(-c3cc(C(F)(F)F)ccc3F)nc3c2ncn3C2CCCCO2)cc1] |
| 23 | 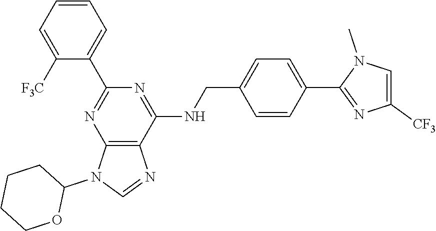 [SMILES: Cn1cc(C(F)(F)F)nc1-c1ccc(CNc2nc(-c3ccccc3C(F)(F)F)nc3c2ncn3C2CCCCO2)cc1] |
| 24 | 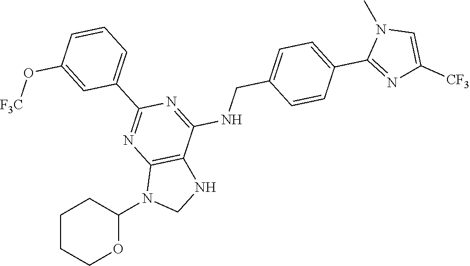 [SMILES: Cn1cc(C(F)(F)F)nc1-c1ccc(CNc2nc(-c3cccc(OC(F)(F)F)c3)nc3c2NCN3C2CCCCO2)cc1] |
| 25 | 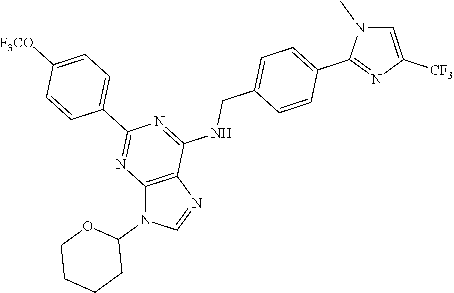 [SMILES: Cn1cc(C(F)(F)F)nc1-c1ccc(CNc2nc(-c3ccc(OC(F)(F)F)cc3)nc3c2ncn3C2CCCCO2)cc1] |
| 26 | 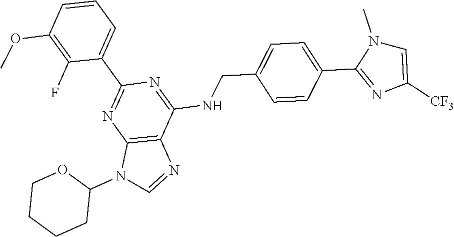 [SMILES: COc1cccc(-c2nc(NCc3ccc(-c4nc(C(F)(F)F)cn4C)cc3)c3ncn(C4CCCCO4)c3n2)c1F] |
| 27 | 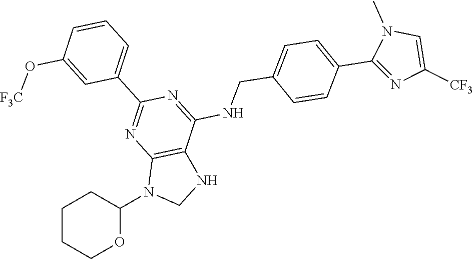 [SMILES: Cn1cc(C(F)(F)F)nc1-c1ccc(CNc2nc(-c3cccc(OC(F)(F)F)c3)nc3c2NCN3C2CCCCO2)cc1] |
| 28 | 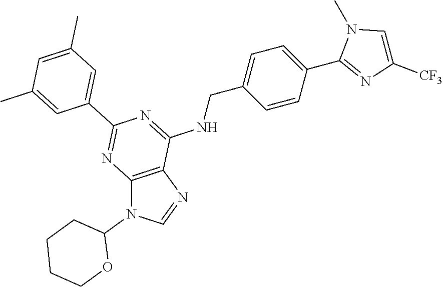 [SMILES: Cc1cc(C)cc(-c2nc(NCc3ccc(-c4nc(C(F)(F)F)cn4C)cc3)c3ncn(C4CCCCO4)c3n2)c1] |
| 29 | 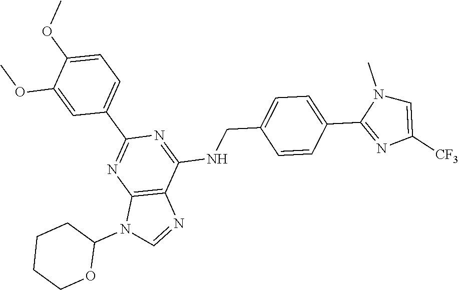 [SMILES: COc1ccc(-c2nc(NCc3ccc(-c4nc(C(F)(F)F)cn4C)cc3)c3ncn(C4CCCCO4)c3n2)cc1OC] |
| 30 | 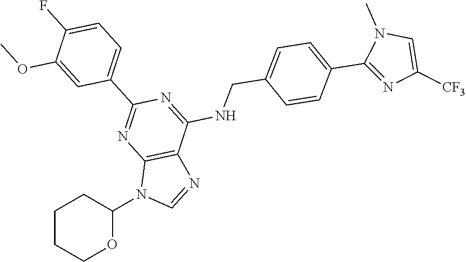 [SMILES: COc1cc(-c2nc(NCc3ccc(-c4nc(C(F)(F)F)cn4C)cc3)c3ncn(C4CCCCO4)c3n2)ccc1F] |
| 31 | 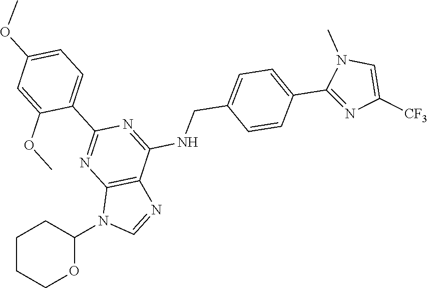 [SMILES: COc1ccc(-c2nc(NCc3ccc(-c4nc(C(F)(F)F)cn4C)cc3)c3ncn(C4CCCCO4)c3n2)c(OC)c1] |
| 32 | 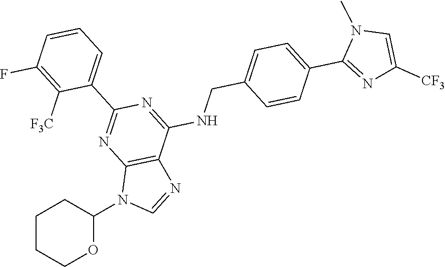 [SMILES: Cn1cc(C(F)(F)F)nc1-c1ccc(CNc2nc(-c3cccc(F)c3C(F)(F)F)nc3c2ncn3C2CCCCO2)cc1] |
| 33 | 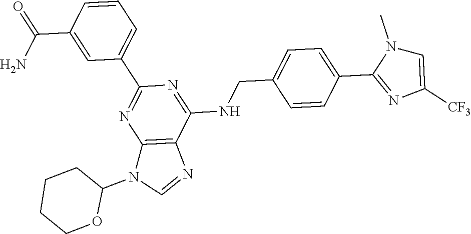 [SMILES: Cn1cc(C(F)(F)F)nc1-c1ccc(CNc2nc(-c3cccc(C(N)=O)c3)nc3c2ncn3C2CCCCO2)cc1] |
| 34 | 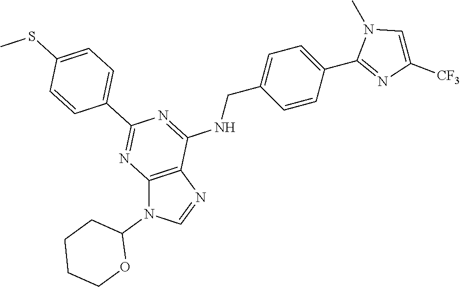 [SMILES: CSc1ccc(-c2nc(NCc3ccc(-c4nc(C(F)(F)F)cn4C)cc3)c3ncn(C4CCCCO4)c3n2)cc1] |
| 35 | 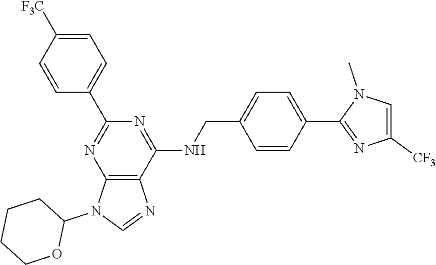 [SMILES: Cn1cc(C(F)(F)F)nc1-c1ccc(CNc2nc(-c3ccc(C(F)(F)F)cc3)nc3c2ncn3C2CCCCO2)cc1] |
| 36 | 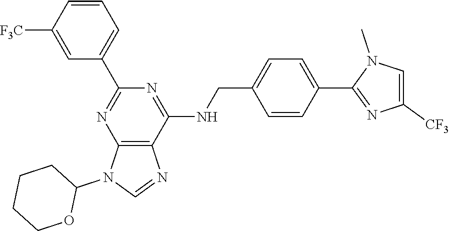 [SMILES: Cn1cc(C(F)(F)F)nc1-c1ccc(CNc2nc(-c3cccc(C(F)(F)F)c3)nc3c2ncn3C2CCCCO2)cc1] |
| 37 | 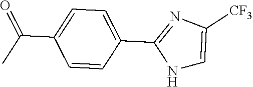 [SMILES: CC(=O)c1ccc(-c2nc(C(F)(F)F)c[nH]2)cc1] |
| 38 | 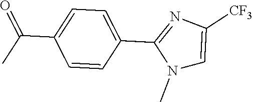 [SMILES: CC(=O)c1ccc(-c2nc(C(F)(F)F)cn2C)cc1] |
| 39 | 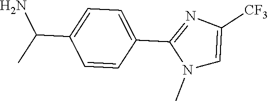 [SMILES: CC(N)c1ccc(-c2nc(C(F)(F)F)cn2C)cc1] |
| 40 | 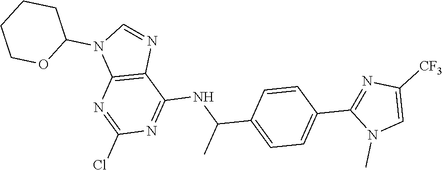 [SMILES: CC(Nc1nc(Cl)nc2c1ncn2C1CCCCO1)c1ccc(-c2nc(C(F)(F)F)cn2C)cc1] |
| 41 | 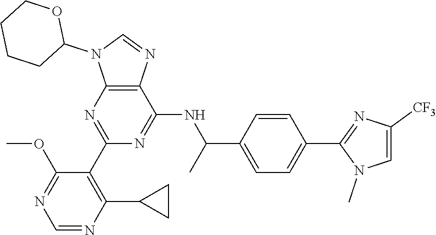 [SMILES: COc1ncnc(C2CC2)c1-c1nc(NC(C)c2ccc(-c3nc(C(F)(F)F)cn3C)cc2)c2ncn(C3CCCCO3)c2n1] |
| 42 | 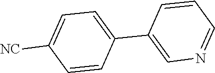 [SMILES: N#Cc1ccc(-c2cccnc2)cc1] |
| 43 | 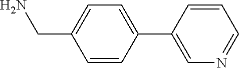 [SMILES: NCc1ccc(-c2cccnc2)cc1] |
| 44 | 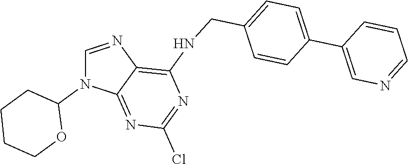 [SMILES: Clc1nc(NCc2ccc(-c3cccnc3)cc2)c2ncn(C3CCCCO3)c2n1] |
| 45 | 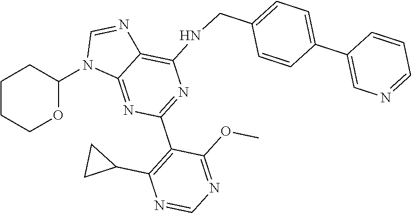 [SMILES: COc1ncnc(C2CC2)c1-c1nc(NCc2ccc(-c3cccnc3)cc2)c2ncn(C3CCCCO3)c2n1] |
| 46 | 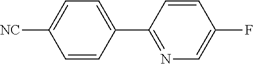 [SMILES: N#Cc1ccc(-c2ccc(F)cn2)cc1] |
| 47 | 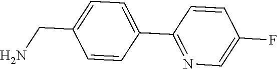 [SMILES: NCc1ccc(-c2ccc(F)cn2)cc1] |
| 48 | 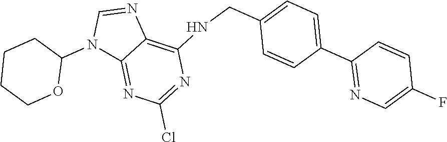 [SMILES: Fc1ccc(-c2ccc(CNc3nc(Cl)nc4c3ncn4C3CCCCO3)cc2)nc1] |
| 49 | 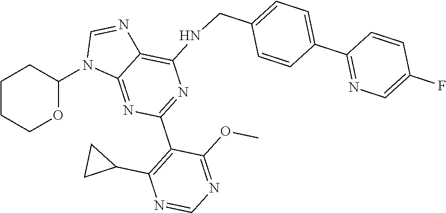 [SMILES: COc1ncnc(C2CC2)c1-c1nc(NCc2ccc(-c3ccc(F)cn3)cc2)c2ncn(C3CCCCO3)c2n1] |

Int: intermediate

### Intermediate 1: 4-(4-(trifluoromethyl)-1H-imidazol-2-yl)benzonitrile

To a solution of 3,3-dibromo-1,1,1-trifluoropropan-2-one (3.0 g, 1.0 eq) and NaOAc (0.93 gm, 1.0 eq) was dissolved in water (3 ml, 2 vol). Reaction mixture was heated at 100° C. for 1 hr. Cooled the reaction mixture at RT. 4-formylbenzonitrile (1.5 gm, 1 eq) was dissolved in MeOH (15 ml. 10 vol) added to reaction mixture followed by addition of NH₄OH (10 ml, 6 vol). Stirred the reaction mixture at RT for 2 hr. Reaction mixture was poured in water and extracted with (3×) EtOAc. Combined the organic layer and dried over Na₂SO₄, evaporated under vacuum. Crude was purified by column chromatography, eluted at 15-20% EtOAc-n-Hexane to get 4-(4-(trifluoromethyl)-1H-imidazol-2-yl)benzonitrile (0.5 gm). 1H NMR, CDCl3 (400 MHz): 10.1 (bs, 1H), 7.99-7.97 (m, 2H), 7.77-7.7- (m, 2H), 7.44 (s, 1H). Mass: 238.1[M+1].

### Intermediate 2: 4-(1-methyl-4-(trifluoromethyl)-1H-imidazol-2-yl)benzonitrile

To a solution of 4-(4-(trifluoromethyl)-1H-imidazol-2-yl)benzonitrile (0.3 gm, 1.0 eq) was dissolved in THF (3 ml, 10 vol). Reaction mixture was cooled at 0° C., to it NaH (0.04 g, 1.3 eq) was added portion wise at same temp. Iodomethane (0.23 g, 1.3 eq) was added and reaction mixture was stirred at RT for 3 hr. Reaction mixture was poured into water and extracted in EtOAc (3×), Combined the organic layer and dried over Na₂SO₄, evaporated under vacuum. Crude was purified by column chromatography, eluted at 10-25% EtOAc-n-Hexane to get 4-(1-methyl-4-(trifluoromethyl)-1H-imidazol-2-yl)benzonitrile (0.25 gm). 1H NMR, CDCl3 (400 MHz): 7.81-7.72 (m, 4H), 7.37 (s, 1H), 3.83 (s, 3H). Mass: 252.0 [M+1].

### Intermediate 3: (4-(1-methyl-4-(trifluoromethyl)-1H-imidazol-2-yl)phenyl)methanamine

To a solution of 4-(1-methyl-4-(trifluoromethyl)-1H-imidazol-2-yl) benzonitrile (0.25 gm, 1.0 eq) was EtOAc (3 ml, 10 vol). NH₄OH (4-5 drops) and Raney Ni (0.2 gm) were added. Reaction mixture was hydrogenated under H₂ balloon. Stirred Reaction mixture for 4 hr. Reaction was filtered through celite pad, filtrate was evaporated under vacuum to get (4-(1-methyl-4-(trifluoromethyl)-1H-imidazol-2-yl)phenyl)methanamine (0.15 gm). Mass: 256.7 [M+1].

### Intermediate 4: 2-chloro-N-(4-(1-methyl-4-(trifluoromethyl)-1H-imidazol-2-yl)benzyl)-6,7-dihydro-5H-cyclopenta[d]pyrimidin-4-amine

To a solution of 2,4-dichloro-6,7-dihydro-5H-cyclopenta[d]pyrimidine (0.18 g, 1 eq) and (4-(1-methyl-4-(trifluoromethyl)-1H-imidazol-2-yl)phenyl)methanamine (0.25 gm, 1 eq) was dissolved in DMF (2 ml). DIPEA (0.35 ml, 2 eq) was added to reaction mixture and heated at 100° C. for 12 h. Reaction mixture was cooled at RT and poured into ice cold water and extracted with (3×) EtOAc. Combined the organic layer and dried over Na₂SO₄, evaporated under vacuum. Crude was purified by column chromatography, eluted at 40-45% EtOAc-n-Hexane to get 2-chloro-N-(4-(1-methyl-4-(trifluoromethyl)-1H-imidazol-2-yl)benzyl)-6,7-dihydro-5H-cyclopenta[d]pyrimidin-4-amine (0.14 gm). Mass: 408.1 [M+1].

### Intermediate 5: 3-chloro-1-((4-(1-methyl-4-(trifluoromethyl)-1H-imidazol-2-yl)benzyl)amino)-6,7-dihydro-5H-cyclopenta[c]pyridine-4-carbonitrile

To a solution of 1,3-dichloro-6,7-dihydro-5H-cyclopenta[c]pyridine-4-carbonitrile (0.18 g, 1 eq) and (4-(1-methyl-4-(trifluoromethyl)-1H-imidazol-2-yl)phenyl)methanamine (0.25 gm, 1 eq) was dissolved in DMF (2 ml). DIPEA (0.35 ml, 2 eq) was added to reaction mixture and heated at 100° C. for 12 h. Reaction mixture was cooled at RT and poured into ice cold water and extracted with (3×) EtOAc. Combined the organic layer and dried over Na₂SO₄, evaporated under vacuum. Crude was purified by column chromatography, eluted at 40-45% EtOAc-n-Hexane to get 3-chloro-1-((4-(1-methyl-4-(trifluoromethyl)-1H-imidazol-2-yl)benzyl)amino)-6,7-dihydro-5H-cyclopenta[c]pyridine-4-carbonitrile (0.14 gm). Mass: 432.1 [M+1].

### Intermediate 6: 2-chloro-N-(4-(1-methyl-4-(trifluoromethyl)-1H-imidazol-2-yl)benzyl)pyrrolo[2,1-f][1,2,4]triazin-4-amine

To a solution of 2,4-dichloropyrrolo[2,1-f][1,2,4]triazine (0.5 g, 1 eq) and (4-(1-methyl-4-(trifluoromethyl)-1H-imidazol-2-yl)phenyl)methanamine (0. 910 gm, 1 eq) was dissolved in THE (2 ml). DIPEA (1.5 ml, 3 eq) was added to reaction mixture and stirred at RT for 12 h. Reaction mixture was cooled at RT and poured into ice cold water and extracted with (3×) EtOAc. Combined the organic layer and dried over Na₂SO₄, evaporated under vacuum. Crude was purified by column chromatography, eluted at 40-45% EtOAc-n-Hexane to get 2-chloro-N-(4-(1-methyl-4-(trifluoromethyl)-1H-imidazol-2-yl)benzyl)pyrrolo[2,1-f][1,2,4]triazin-4-amine (0.43 gm). Mass: 407.0 [M+1].

### Intermediate 7: 7-chloro-N-(4-(1-methyl-4-(trifluoromethyl)-1H-imidazol-2-yl)benzyl)imidazo[1,2-c]pyrimidin-5-amine

The titled compound was synthesized by using the procedure provided in Intermediate 6 by using 5,7-dichloroimidazo[1,2-c]pyrimidine and (4-(1-methyl-4-(trifluoromethyl)-1H-imidazol-2-yl)phenyl)methanamine.

### Intermediate 8: N-(4-(1-isopropyl-4-(trifluoromethyl)-1H-imidazol-2-yl)benzyl)-2-(2-isopropylphenyl)-6,7-dihydro-5H-cyclopenta[d]pyrimidin-4-amine

The titled compound was synthesized by using the procedure provided in Intermediate 6 by using 2,4-dichloro-6,7-dihydro-5H-cyclopenta[d]pyrimidine and (4-(1-isopropyl-4-(trifluoromethyl)-1H-imidazol-2-yl)phenyl)methanamine.

### Intermediate 9: 4-((4-(1-isopropyl-4-(trifluoromethyl)-1H-imidazol-2-yl)benzyl)oxy)-2-(2-isopropylphenyl)-6,7-dihydro-5H-cyclopenta[d]pyrimidine

The titled compound was synthesized by using the procedure provided in Intermediate 6 by using 2,4-dichloro-6,7-dihydro-5H-cyclopenta[d]pyrimidine and (4-(1-isopropyl-4-(trifluoromethyl)-1H-imidazol-2-yl)phenyl)methanol.

### Intermediate 10: 1-benzyl-1-(2-(2-isopropylphenyl)-6,7-dihydro-5H-cyclopenta[d]pyrimidin-4-yl)-3,3-dimethylurea

To a solution of N-benzyl-2-(2-isopropylphenyl)-6,7-dihydro-5H-cyclopenta[d]pyrimidin-4-amine (1.0 eq) was dissolved in THF, cooled at 0° C., to it DAMP (0.1 eq), and Et₃N (20.eq) was added. Triphosgene (5.0 eq) was added portion wise. Stirred Reaction mixture for 2 hr at RT. N—N dimethyl amine was added dropwise, stirred for 10 min. Reaction mixture was quenched by sat·NaHCO₃ solution and extracted with (3×) EtOAc. Combined the organic layer and dried over Na₂SO₄, evaporated under vacuum. Crude was purified by column chromatography, eluted at 65-80% EtOAc-n-Hexane to get 1-benzyl-1-(2-(2-isopropylphenyl)-6,7-dihydro-5H-cyclopenta[d]pyrimidin-4-yl)-3,3-dimethylurea.

### Intermediate 11: 2,6-dichloro-9-(tetrahydro-2H-pyran-2-yl)-9H-purine

To a solution of 2, 6-dichloro-9H-purine (1.0 g, 1.00 eq) was dissolved in DCM (10 ml). To it p-toluenesulfonic acid (0. 05 g, 0.05 eq) and 3, 4-dihydropyran (0.9 g, 2.00 eq) was added. Reaction mixture was stirred at RT for 12 h. Reaction mixture was cooled at RT and poured into ice cold water and extracted with (3×) DCM. Combined the organic layer and dried over Na₂SO₄, evaporated under vacuum. Crude was purified by column chromatography, eluted at 25-30% EtOAc-n-Hexane to get 2,6-dichloro-9-(tetrahydro-2H-pyran-2-yl)-9H-purine (1.1 gm). Mass: 274.2 [M+1].

### Intermediate 12: 2-chloro-4-((4-(1-methyl-4-(trifluoromethyl)-1H-imidazol-2-yl)benzyl)amino)-6,7-dihydro-5H-cyclopenta[b]pyridine-3-carbonitrile

The titled compound was synthesized by using the procedure provided in Intermediate 6 by using 2,4-dichloro-6,7-dihydro-5H-cyclopenta[b]pyridine-3-carbonitrile and (4-(1-methyl-4-(trifluoromethyl)-1H-imidazol-2-yl)phenyl)methanamine.

### Intermediate 13: 2-(4-cyclopropyl-6-methoxypyrimidin-5-yl)-N-(4-(1-isopropyl-4-(trifluoromethyl)-1H-imidazol-2-yl)benzyl)pyrrolo[2,1-f][1,2,4]triazin-4-amine

The titled compound was synthesized by using the procedure provided in Intermediate 6 by using 2,4-dichloropyrrolo[2,1-f][1,2,4]triazine and (4-(1-isopropyl-4-(trifluoromethyl)-1H-imidazol-2-yl)phenyl)methanamine.

### Intermediate 14: 2-(4-cyclopropyl-6-methoxypyrimidin-5-yl)-4-((4-(1-methyl-4-(trifluoromethyl)-1H-imidazol-2-yl)benzyl)oxy)pyrrolo[2,1-f][1,2,4]triazine

The titled compound was synthesized by using the procedure provided in Intermediate 6 by using 2,4-dichloropyrrolo[2,1-f][1,2,4]triazine and (4-(1-methyl-4-(trifluoromethyl)-1H-imidazol-2-yl)phenyl)methanol.

### Intermediate 15: 2-chloro-N-(4-(1-methyl-4-(trifluoromethyl)-1H-imidazol-2-yl)benzyl)-9-(tetrahydro-2H-pyran-2-yl)-9H-purin-6-amine

The titled compound was synthesized by using the procedure provided in Intermediate 4 by using Intermediate 11 and intermediate 3 to get 2-chloro-N-(4-(1-methyl-4-(trifluoromethyl)-1H-imidazol-2-yl)benzyl)-9-(tetrahydro-2H-pyran-2-yl)-9H-purin-6-amine.

### Intermediate 16: 2-(2-chloro-3-methylphenyl)-N-(4-(1-methyl-4-(trifluoromethyl)-1H-imidazol-2-yl)benzyl)-9-(tetrahydro-2H-pyran-2-yl)-9H-purin-6-amine

The titled compound (45 mg) was synthesized by using the general procedure-1 by using intermediate 15 and 2-Chloro-3-methylphenylboronic Acid. Mass: 583.03 [m+1].

### Intermediate 17: 2-(3,5-dichlorophenyl)-N-(4-(1-methyl-4-(trifluoromethyl)-1H-imidazol-2-yl)benzyl)-9-(tetrahydro-2H-pyran-2-yl)-9H-purin-6-amine

The titled compound (34 mg) was synthesized by using the general procedure-1 by using intermediate 15 and 3,5-Dichlorophenylboronic acid. Mass: 603.21 [m+1].

### Intermediate 18: 2-(2,4-difluorophenyl)-N-(4-(1-methyl-4-(trifluoromethyl)-1H-imidazol-2-yl)benzyl)-9-(tetrahydro-2H-pyran-2-yl)-9H-purin-6-amine

The titled compound (38 mg) was synthesized by using the general procedure-1 by using intermediate 15 and 2,4-Difluorophenylboronic acid. Mass: 570.34 [m+1].

### Intermediate 19: 2-(3,5-difluorophenyl)-N-(4-(1-methyl-4-(trifluoromethyl)-1H-imidazol-2-yl)benzyl)-9-(tetrahydro-2H-pyran-2-yl)-9H-purin-6-amine

The titled compound (39 mg) was synthesized by using the general procedure-1 by using intermediate 15 and 3,5-Difluorophenylboronic acid. Mass: 570.35 [m+1].

### Intermediate 20: 2-(2,4-dichlorophenyl)-N-(4-(1-methyl-4-(trifluoromethyl)-1H-imidazol-2-yl)benzyl)-9-(tetrahydro-2H-pyran-2-yl)-9H-purin-6-amine

The titled compound (28 mg) was synthesized by using the general procedure-1 by using intermediate 15 and 2,4-Dichlorophenylboronic acid. Mass: 603.22 [m+1].

### Intermediate 21: 2-fluoro-5-(6-((4-(1-methyl-4-(trifluoromethyl)-1H-imidazol-2-yl)benzyl)amino)-9-(tetrahydro-2H-pyran-2-yl)-9H-purin-2-yl)benzonitrile

The titled compound (25 mg) was synthesized by using the general procedure-1 by using intermediate 15 and (2,6-dimethoxypyridin-3-yl)boronic acid. Mass: 577.34 [m+1].

### Intermediate 22: 2-(2,6-dimethoxypyridin-3-yl)-N-(4-(1-methyl-4-(trifluoromethyl)-1H-imidazol-2-yl)benzyl)-9-(tetrahydro-2H-pyran-2-yl)-9H-purin-6-amine

The titled compound (51 mg) was synthesized by using the general procedure-1 by using intermediate 15 and 2-Fluoro-5-(trifluoromethyl)phenylboronic acid. Mass: 595.23 [m+1].

### Intermediate 23: N-(4-(1-methyl-4-(trifluoromethyl)-1H-imidazol-2-yl)benzyl)-9-(tetrahydro-2H-pyran-2-yl)-2-(2-(trifluoromethyl)phenyl)-9H-purin-6-amine

The titled compound (40 mg) was synthesized by using the general procedure-1 by using intermediate 15 and (2-Trifluoromethyl)phenylboronic acid. Mass: 602.34 [m+1].

### Intermediate 24: N-(4-(1-methyl-4-(trifluoromethyl)-1H-imidazol-2-yl)benzyl)-9-(tetrahydro-2H-pyran-2-yl)-2-(3-(trifluoromethoxy)phenyl)-8,9-dihydro-7H-purin-6-amine

The titled compound (39 mg) was synthesized by using the general procedure-1 by using intermediate 15 and (3-(Trifluoromethoxy)phenyl)boronic acid. Mass: 620.23 [m+1].

### Intermediate 25: N-(4-(1-methyl-4-(trifluoromethyl)-1H-imidazol-2-yl)benzyl)-9-(tetrahydro-2H-pyran-2-yl)-2-(4-(trifluoromethoxy)phenyl)-9H-purin-6-amine

The titled compound (42 mg) was synthesized by using the general procedure-1 by using intermediate 15 and (4-(Trifluoromethoxy)phenyl)boronic acid. Mass: 618.39 [m+1].

### Intermediate 26: 2-(2-fluoro-3-methoxyphenyl)-N-(4-(1-methyl-4-(trifluoromethyl)-1H-imidazol-2-yl)benzyl)-9-(tetrahydro-2H-pyran-2-yl)-9H-purin-6-amine

The titled compound (38 mg) was synthesized by using the general procedure-1 by using intermediate 15 and (2-Fluoro-3-methoxyphenyl)boronic acid. Mass: 582.12 [m+1].

### Intermediate 27: 2-fluoro-5-(6-((4-(1-methyl-4-(trifluoromethyl)-1H-imidazol-2-yl)benzyl)amino)-9-(tetrahydro-2H-pyran-2-yl)-9H-purin-2-yl)benzonitrile

The titled compound (45 mg) was synthesized by using the general procedure-1 by using intermediate 15 and 3-Cyano-4-fluorobenzeneboronic acid. Mass: 577.10 [m+1].

### Intermediate 28: 2-(3,5-dimethylphenyl)-N-(4-(1-methyl-4-(trifluoromethyl)-1H-imidazol-2-yl)benzyl)-9-(tetrahydro-2H-pyran-2-yl)-9H-purin-6-amine

The titled compound (24 mg) was synthesized by using the general procedure-1 by using intermediate 15 and 3,5-Dimethylphenylboronic acid. Mass: 562.31 [m+1].

### Intermediate 29: 2-(3,4-dimethoxyphenyl)-N-(4-(1-methyl-4-(trifluoromethyl)-1H-imidazol-2-yl)benzyl)-9-(tetrahydro-2H-pyran-2-yl)-9H-purin-6-amine

The titled compound (21 mg) was synthesized by using the general procedure-1 by using intermediate 15 and 3,4-Dimethoxyphenylboronic acid. Mass: 594.24 [m+1].

### Intermediate 30: 2-(4-fluoro-3-methoxyphenyl)-N-(4-(1-methyl-4-(trifluoromethyl)-1H-imidazol-2-yl)benzyl)-9-(tetrahydro-2H-pyran-2-yl)-9H-purin-6-amine

The titled compound (49 mg) was synthesized by using the general procedure-1 by using intermediate 15 and (4-Fluoro-3-methoxyphenyl)boronic acid. Mass: 582.13 [m+1].

### Intermediate 31: 2-(2,4-dimethoxyphenyl)-N-(4-(1-methyl-4-(trifluoromethyl)-1H-imidazol-2-yl)benzyl)-9-(tetrahydro-2H-pyran-2-yl)-9H-purin-6-amine

The titled compound (19 mg) was synthesized by using the general procedure-1 by using intermediate 15 and 2,4-Dimethoxyphenylboronic acid. Mass: 594.21 [m+1].

### Intermediate 32: 2-(3-fluoro-2-(trifluoromethyl)phenyl)-N-(4-(1-methyl-4-(trifluoromethyl)-1H-imidazol-2-yl)benzyl)-9-(tetrahydro-2H-pyran-2-yl)-9H-purin-6-amine

The titled compound (38 mg) was synthesized by using the general procedure-1 by using intermediate 15 and (3-fluoro-2-(trifluoromethyl)phenyl)boronic acid. Mass: 620.40 [m+1].

### Intermediate 33: 3-(6-((4-(1-methyl-4-(trifluoromethyl)-1H-imidazol-2-yl)benzyl)amino)-9-(tetrahydro-2H-pyran-2-yl)-9H-purin-2-yl)benzamide

The titled compound (24 mg) was synthesized by using the general procedure-1 by using intermediate 15 and (3-carbamoylphenyl)boronic acid. Mass: 577.28 [m+1].

### Intermediate 34: N-(4-(1-methyl-4-(trifluoromethyl)-1H-imidazol-2-yl)benzyl)-2-(4-(methylthio)phenyl)-9-(tetrahydro-2H-pyran-2-yl)-9H-purin-6-amine

The titled compound (26 mg) was synthesized by using the general procedure-1 by using intermediate 15 and (4-(methylthio)phenyl)boronic acid. Mass: 580.31 [m+1].

### Intermediate 35: N-(4-(1-methyl-4-(trifluoromethyl)-1H-imidazol-2-yl)benzyl)-9-(tetrahydro-2H-pyran-2-yl)-2-(4-(trifluoromethyl)phenyl)-9H-purin-6-amine

The titled compound (21 mg) was synthesized by using the general procedure-1 by using intermediate 15 and (4-(Trifluoromethyl)phenyl)boronic acid. Mass: 602.34 [m+1].

### Intermediate 36: N-(4-(1-methyl-4-(trifluoromethyl)-1H-imidazol-2-yl)benzyl)-9-(tetrahydro-2H-pyran-2-yl)-2-(3-(trifluoromethyl)phenyl)-9H-purin-6-amine

The titled compound (38 mg) was synthesized by using the general procedure-1 by using intermediate 15 and (3-(Trifluoromethyl)phenyl)boronic acid. Mass: 602.40 [m+1].

### Intermediate 37: 1-(4-(4-(trifluoromethyl)-1H-imidazol-2-yl)phenyl)ethan-1-one

The titled compound was synthesized by using the procedure provided in Intermediate 1 by using 4-acetylbenzaldehyde to get 1-(4-(4-(trifluoromethyl)-1H-imidazol-2-yl)phenyl)ethan-1-one. 255.12[M+1].

### Intermediate 38: 1-(4-(1-methyl-4-(trifluoromethyl)-1H-imidazol-2-yl)phenyl)ethan-1-one

The titled compound was synthesized by using the procedure provided in Intermediate 2 by using 1-(4-(4-(trifluoromethyl)-1H-imidazol-2-yl)phenyl)ethan-1-one to get 1-(4-(1-methyl-4-(trifluoromethyl)-1H-imidazol-2-yl)phenyl)ethan-1-one. Mass: 269.24 [M+1].

### Intermediate 39: 1-(4-(1-methyl-4-(trifluoromethyl)-1H-imidazol-2-yl)phenyl)ethan-1-amine

To a solution of 1-(4-(1-methyl-4-(trifluoromethyl)-1H-imidazol-2-yl)phenyl)ethan-1-one was MeOH. NH₄OAc (10 eq) and NaCNBH₃ (2.0 eq) were added. Reaction mixture was maintained at 60° C. for 16 h. Reaction was evaporated under vacuum and crude poured into water and extracted with (3×) EtOAc. Combined the organic layer and dried over Na₂SO₄, evaporated under vacuum. To get 1-(4-(1-methyl-4-(trifluoromethyl)-1H-imidazol-2-yl)phenyl)ethan-1-amine. Mass: 270.18 [M+1].

### Intermediate 40: 2-chloro-N-(1-(4-(1-methyl-4-(trifluoromethyl)-1H-imidazol-2-yl)phenyl)ethyl)-9-(tetrahydro-2H-pyran-2-yl)-9H-purin-6-amine

The titled compound was synthesized by using the procedure provided in Intermediate 3 by using Intermediate 15 and 1-(4-(1-methyl-4-(trifluoromethyl)-1H-imidazol-2-yl)phenyl)ethan-1-amine to get 2-chloro-N-(1-(4-(1-methyl-4-(trifluoromethyl)-1H-imidazol-2-yl)phenyl)ethyl)-9-(tetrahydro-2H-pyran-2-yl)-9H-purin-6-amine. Mass: 506.12 [M+1].

### Intermediate 41: 2-(4-cyclopropyl-6-methoxypyrimidin-5-yl)-N-(1-(4-(1-methyl-4-(trifluoromethyl)-1H-imidazol-2-yl)phenyl)ethyl)-9-(tetrahydro-2H-pyran-2-yl)-9H-purin-6-amine

The titled compound was synthesized by using the procedure provided in Example 1 by using 2-chloro-N-(1-(4-(1-methyl-4-(trifluoromethyl)-1H-imidazol-2-yl)phenyl)ethyl)-9-(tetrahydro-2H-pyran-2-yl)-9H-purin-6-amine and 4-cyclopropyl-6-methoxy-5-(4,4,5,5-tetramethyl-1,3,2-dioxaborolan-2-yl)pyrimidine under Suzuki reaction conditions. Mass: 620.21[M+1].

### Intermediate 42: 4-(pyridin-3-yl)benzonitrile

To a solution of 4-bromobenzonitrile (2.0 g) and pyridin-3-ylboronic acid (2.7 g) was dissolved in Dioxane. To it Cs₂CO₃ (10.83 g) was added, N₂ gas purged for 15 mins. Tetrakis (0.8 g) were added and purged again for 5 mins. Reaction mixture was heated at 110° C. for 16 h. Reaction mixture was poured into ice-cold water (20 ml) and extracted with EtOAc (3×50 ml), combined organic layer and dried over Na₂SO₄. Purified by column chromatography compound eluted at 20-25% EtOAc-n-Hexane to get 4-(pyridin-3-yl)benzonitrile (2.0 gm). Mass: 181.32 [M+1].

### Intermediate 43: (4-(pyridin-3-yl)phenyl)methanamine

The titled compound was synthesized by using the procedure provided in Intermediate 3 by using 4-(pyridin-3-yl)benzonitrile to get (4-(pyridin-3-yl)phenyl)methanamine. Mass: 185.26 [M+1].

### Intermediate 44: 2-chloro-N-(4-(pyridin-3-yl)benzyl)-9-(tetrahydro-2H-pyran-2-yl)-9H-purin-6-amine

The titled compound was synthesized by using the procedure provided in Intermediate 3 by using Intermediate 15 and (4-(pyridin-3-yl)phenyl)methanamine to get 2-chloro-N-(4-(pyridin-3-yl)benzyl)-9-(tetrahydro-2H-pyran-2-yl)-9H-purin-6-amine. Mass: 421.13 [M+1].

### Intermediate 45: 2-(4-cyclopropyl-6-methoxypyrimidin-5-yl)-N-(4-(pyridin-3-yl)benzyl)-9-(tetrahydro-2H-pyran-2-yl)-9H-purin-6-amine

The titled compound was synthesized by using the procedure provided in Example 1 by using 2-chloro-N-(4-(pyridin-3-yl)benzyl)-9-(tetrahydro-2H-pyran-2-yl)-9H-purin-6-amine and 4-cyclopropyl-6-methoxy-5-(4,4,5,5-tetramethyl-1,3,2-dioxaborolan-2-yl)pyrimidine under Suzuki reaction conditions. Mass: 536.15 [M+1]s.

### Intermediate 46: 4-(5-fluoropyridin-2-yl)benzonitrile

To a solution of 2-bromo-5-fluoropyridine (0.45 g) and (4-cyanophenyl)boronic acid (1.1 g) was dissolved in Dioxane. To it Cs₂CO₃ (2.5 g) was added, N₂ gas purged for 15 mins. Tetrakis (0.15 g) were added and purged again for 5 mins. Reaction mixture was heated at 110° C. for 16 h. Reaction mixture was poured into ice-cold water (20 ml) and extracted with EtOAc (3×50 ml), combined organic layer and dried over Na₂SO₄. Purified by column chromatography compound eluted at 20-25% EtOAc-n-Hexane to get 4-(5-fluoropyridin-2-yl)benzonitrile (0.45 gm). Mass: 200.12 [M+1].

### Intermediate 47: (4-(5-fluoropyridin-2-yl)phenyl)methanamine

To a solution of 4-(5-fluoropyridin-2-yl)benzonitrile was dissolved in THF, cooled at 0° C. To it LAH (1.5 M) was added dropwise at same temp. Reaction mixture was stirred at RT for 2 hr. DCM was added and stirred for 30 min. Reaction mixture was filtered off through celite pad. Filtrate was evaporated under vacuum to get. (4-(5-fluoropyridin-2-yl)phenyl)methanamine. Mass: 204.34 [M+1].

### Intermediate 48: 2-chloro-N-(4-(5-fluoropyridin-2-yl)benzyl)-9-(tetrahydro-2H-pyran-2-yl)-9H-purin-6-amine

The titled compound was synthesized by using the procedure provided in Intermediate 3 by using Intermediate 15 and (4-(5-fluoropyridin-2-yl)phenyl)methanamine to get 2-chloro-N-(4-(5-fluoropyridin-2-yl)benzyl)-9-(tetrahydro-2H-pyran-2-yl)-9H-purin-6-amine. Mass: 439.18 [M+1].

### Intermediate 49: 2-(4-cyclopropyl-6-methoxypyrimidin-5-yl)-N-(4-(5-fluoropyridin-2-yl)benzyl)-9-(tetrahydro-2H-pyran-2-yl)-9H-purin-6-amine

The titled compound was synthesized by using the procedure provided in Example 1 by using 2-chloro-N-(4-(5-fluoropyridin-2-yl)benzyl)-9-(tetrahydro-2H-pyran-2-yl)-9H-purin-6-amine and 4-cyclopropyl-6-methoxy-5-(4,4,5,5-tetramethyl-1,3,2-dioxaborolan-2-yl)pyrimidine under Suzuki reaction conditions. Mass: 553.20 [M+1].

### Example-1

### 2-(4-cyclopropyl-6-methoxypyrimidin-5-yl)-N-(4-(1-methyl-4-(trifluoromethyl)-1H-imidazol-2-yl)benzyl)-6,7-dihydro-5H-cyclopenta[d]pyrimidin-4-amine

To a solution of 2-chloro-N-(4-(1-methyl-4-(trifluoromethyl)-1H-imidazol-2-yl)benzyl)-6,7-dihydro-5H-cyclopenta[d]pyrimidin-4-amine (0.11 g) and 4-cyclopropyl-6-methoxy-5-(4,4,5,5-tetramethyl-1,3,2-dioxaborolan-2-yl)pyrimidine (0.15 g) was dissolved in Dioxane. To it K₂CO₃ was added, N₂ gas purged for 15 mins. Tetrakis were added and purged again for 5 mins. Reaction mixture was heated at 110° C. for 16 h. Reaction mixture was poured into ice-cold water (20 ml) and extracted with EtOAc (3×50 ml), combined organic layer and dried over Na₂SO₄. Purified by column chromatography compound eluted at 75-80% EtOAc-n-Hexane to give 2-(4-cyclopropyl-6-methoxypyrimidin-5-yl)-N-(4-(1-methyl-4-(trifluoromethyl)-1H-imidazol-2-yl)benzyl)-6,7-dihydro-5H-cyclopenta[d]pyrimidin-4-amine (C) (0.030 g). DMSO (400 MHz): 8.57 (s, 1H), 7.91 (bs, 1H), 7.67-7.62 (m, 3H), 7.42-7.40 (m, 2H), 4.64-4.63 (m, 2H), 3.80 (s, 3H), 3.74 (s, 3H), 2.82-2.74 (m, 4H), 2.08-2.04 (m, 2H), 1.65-1.64 (m, 1H), 0.93-0.92 (m, 2H), 0.76-0.75 (m, 2H). Mass: 522.1[m+1]. HPLC purity: 97.39%.

### Example-2

### 3-(4-cyclopropyl-6-methoxypyrimidin-5-yl)-1-((4-(1-methyl-4-(trifluoromethyl)-1H-imidazol-2-yl)benzyl)amino)-6,7-dihydro-5H-cyclopenta[c]pyridine-4-carbonitrile

To a solution of 3-chloro-1-((4-(1-methyl-4-(trifluoromethyl)-1H-imidazol-2-yl)benzyl)amino)-6,7-dihydro-5H-cyclopenta[c]pyridine-4-carbonitrile (0.12 g) and 4-cyclopropyl-6-methoxy-5-(4,4,5,5-tetramethyl-1,3,2-dioxaborolan-2-yl)pyrimidine (0.15 g) was dissolved in Dioxane. To it K₂CO₃ was added, N₂ gas purged for 15 mins. Tetrakis were added and purged again for 5 mins. Reaction mixture was heated at 110° C. for 16 h. Reaction mixture was poured into ice-cold water (20 ml) and extracted with EtOAc (3×50 ml), combined organic layer and dried over Na₂SO₄. Purified by column chromatography compound eluted at 75-80% EtOAc-n-Hexane to give 3-(4-cyclopropyl-6-methoxypyrimidin-5-yl)-1-((4-(1-methyl-4-(trifluoromethyl)-1H-imidazol-2-yl)benzyl)amino)-6,7-dihydro-5H-cyclopenta[c]pyridine-4-carbonitrile (0.035 g). DMSO (400 MHz): 8.64 (s, 1H), 7.91 (bs, 1H), 7.82-7.79 (m, 1H), 7.64-7.55 (m, 2H), 7.39-7.37 (m, 2H), 4.79-4.55 (m, 2H), 3.85 (s, 3H), 3.71 (s, 3H), 3.00-2.96 (m, 2H), 2.83-2.79 (m, 2H), 2.20-2.17 (m, 2H), 1.67-1.54 (m, 1H), 0.96-0.94 (m, 2H), 0.86-0.80 (m, 1H), 0.64-0.59 (m, 1H). Mass: 546.2[m+1]. HPLC purity: 97.47%.

### Example-3

### 2-(2-isopropylphenyl)-N-(4-(1-methyl-4-(trifluoromethyl)-1H-imidazol-2-yl)benzyl)-6,7-dihydro-5H-cyclopenta[d]pyrimidin-4-amine

To a solution of 2-chloro-N-(4-(1-methyl-4-(trifluoromethyl)-1H-imidazol-2-yl)benzyl)-6,7-dihydro-5H-cyclopenta[d]pyrimidin-4-amine (0.35 g) and (2-isopropylphenyl)boronic acid (0.211 g) was dissolved in Dioxane:H₂O. To it Cs₂CO₃ (0.84 g) was added, N₂ gas purged for 15 mins. Tetrakis (0.1 g) were added and purged again for 5 mins. Reaction mixture was heated at 110 0 C for 16 h. Reaction mixture was poured into ice-cold water (20 ml) and extracted with EtOAc (3×50 ml), combined organic layer and dried over Na₂SO₄. Purified by column chromatography compound eluted at 75-80% EtOAc-n-Hexane to give 2-(2-isopropylphenyl)-N-(4-(1-methyl-4-(trifluoromethyl)-1H-imidazol-2-yl)benzyl)-6,7-dihydro-5H-cyclopenta[d]pyrimidin-4-amine (0.24 g). 1H NMR, DMSO (400 MHz): 7.91 (s, 1H), 7.66-7.64 (m, 2H), 7.59-7.56 (m, 1H), 7.44-7.39 (m, 3H), 7.33-7.28 (m, 2H), 7.18-7.14 (m, 1H), 4.71-4.70 (m, 2H), 3.79 (s, 3H), 3.49-3.44 (m, 1H), 2.89-2.73 (m, 4H), 2.10-2.03 (m, 2H), 1.00-0.99 (m, 6H). Mass: 492.2[M+1]. HPLC purity: 98.41%.

### Example-4

### 3-(2-isopropylphenyl)-1-((4-(1-methyl-4-(trifluoromethyl)-1H-imidazol-2-yl)benzyl)amino)-6,7-dihydro-5H-cyclopenta[c]pyridine-4-carbonitrile

To a solution of 3-chloro-1-((4-(1-methyl-4-(trifluoromethyl)-1H-imidazol-2-yl)benzyl)amino)-6,7-dihydro-5H-cyclopenta[c]pyridine-4-carbonitrile (0.1 g) and (2-isopropylphenyl)boronic acid (0.061 g) was dissolved in Dioxane:H₂O. To it Cs₂CO₃ (0.24 g) was added, N₂ gas purged for 15 mins. Tetrakis (0.015 g) were added and purged again for 5 mins. Reaction mixture was heated at 110 0 C for 16 h. Reaction mixture was poured into ice-cold water (20 ml) and extracted with EtOAc (3×50 ml), combined organic layer and dried over Na₂SO₄. Purified by column chromatography compound eluted at 75-80% EtOAc-n-Hexane to give 3-(2-isopropylphenyl)-1-((4-(1-methyl-4-(trifluoromethyl)-1H-imidazol-2-yl)benzyl)amino)-6,7-dihydro-5H-cyclopenta[c]pyridine-4-carbonitrile (0.06 g). 1H NMR, DMSO (400 MHz): 7.91 (s, 1H), 7.74-7.71 (m, 1H), 7.62-7.60 (m, 1H), 7.38-7.35 (m, 4H), 7.24-7.20 (m, 1H), 7.16-7.14 (m, 1H), 4.68-4.67 (m, 2H), 3.70 (s, 3H), 3.00-2.98 (m, 2H), 2.84-2.76 (m, 3H), 2.20-2.14 (m, 2H), 1.09-0.94 (m, 6H). Mass: 516.0[M+1]. HPLC purity: 95.40%.

### Example-5

### 2-(2-isopropylphenyl)-N-(4-(1-methyl-4-(trifluoromethyl)-1H-imidazol-2-yl)benzyl)pyrrolo[2,1-f][1,2,4]triazin-4-amine

To a solution of 2-chloro-N-(4-(1-methyl-4-(trifluoromethyl)-1H-imidazol-2-yl)benzyl)pyrrolo[2,1-f][1,2,4]triazin-4-amine (0.1 g) and (2-isopropylphenyl)boronic acid (0.060 g) was dissolved in Dioxane:H₂O. To it Cs₂CO₃ (0.23 g) was added, N₂ gas purged for 15 mins. Tetrakis (0.015 g) were added and purged again for 5 mins. Reaction mixture was heated at 110 0 C for 16 h. Reaction mixture was poured into ice-cold water (20 ml) and extracted with EtOAc (3×50 ml), combined organic layer and dried over Na₂SO₄. Purified by column chromatography compound eluted at 75-80% EtOAc-n-Hexane to give 2-(2-isopropylphenyl)-N-(4-(1-methyl-4-(trifluoromethyl)-1H-imidazol-2-yl)benzyl)pyrrolo[2,1-f][1,2,4]triazin-4-amine (0.05 g). DMSO (400 MHz): 8.92-8.89 (m, 1H), 7.99-7.97 (bs, 1H), 7.69-7.67 (m, 3H), 7.46-7.40 (m, 3H), 7.38-7.37 (m, 2H), 7.23-7.19 (m, 1H), 6.99-6.98 (m, 1H), 6.69-6.67 (m, 1H), 4.84-4.82 (m, 2H), 3.74 (s, 3H), 3.39-3.36 (m, 1H), 1.07-1.06 (m, 6H). Mass: 491.1 [M+1]. HPLC purity: 89.97%.

### Example-6

### 2-(4-cyclopropyl-6-methoxypyrimidin-5-yl)-N-(4-(1-methyl-4-(trifluoromethyl)-1H-imidazol-2-yl)benzyl)pyrrolo[2,1-f][1,2,4]triazin-4-amine

The titled compound (N) was synthesized by using the procedure provided in Example 1 by using Intermediate 6 and 4-cyclopropyl-6-methoxy-5-(4,4,5,5-tetramethyl-1,3,2-dioxaborolan-2-yl)pyrimidine (0.15 g) under Suzuki reaction conditions. 1H NMR, DMSO (400 MHz): 9.01-8.98 (m, 1H), 8.60 (s, 1H), 7.98 (bs, 1H), 7.69 (s, 1H), 7.64-7.62 (s, 2H), 7.45-7.43 (m, 2H), 6.98-6.97 (m, 1H), 6.67-6.66 (m, 1H), 4.84-4.82 (m, 2H), 3.86 (s, 3H), 3.71 (s, 3H), 1.83-1.80 (m, 1H), 0.96-0.95 (m, 2H), 0.82-0.79 (m, 2H). Mass: 521.43 [m+1]. HPLC purity: 99.21%.

### Example-7

### 7-(2-isopropylphenyl)-N-(4-(1-methyl-4-(trifluoromethyl)-1H-imidazol-2-yl)benzyl)imidazo[1,2-c]pyrimidin-5-amine

The titled compound was synthesized by using the procedure provided in Example 3 by using Intermediate 7 and (2-isopropylphenyl)boronic acid (0.060 g) under Suzuki reaction conditions. 1H NMR, DMSO (400 MHz): 8.51-8.49 (m, 1H), 8.07 (bs, 1H), 7.91 (s, 1H), 7.68-7.66 (m, 2H), 7.62-7.61 (m, 1H), 7.57-7.55 (m, 2H), 7.38-7.36 (m, 2H), 7.32-7.27 (m, 1H), 7.22-7.18 (m, 1H), 6.85 (m, 1H) 4.84-4.82 (m, 2H), 3.75 (s, 3H), 3.33-3.25 (m, 1H), 1.03-1.01 (m, 6H). Mass: 491.2[m+1].

### Example-8

### 7-(4-cyclopropyl-6-methoxypyrimidin-5-yl)-N-(4-(1-methyl-4-(trifluoromethyl)-1H-imidazol-2-yl)benzyl)imidazo[1,2-c]pyrimidin-5-amine

The titled compound was synthesized by using the procedure provided in Example 1 by using Intermediate 7 and 4-cyclopropyl-6-methoxy-5-(4,4,5,5-tetramethyl-1,3,2-dioxaborolan-2-yl)pyrimidine (0.15 g) under Suzuki reaction conditions. 1H NMR, DMSO (400 MHz): 8.51-8.48 (m, 1H), 8.08 (bs, 1H), 7.91 (s, 1H), 7.62-7.61 (m, 1H), 7.57-7.55 (m, 2H), 7.38-7.36 (m, 2H), 7.32-7.27 (m, 1H), 7.22-7.18 (m, 1H), 4.84-4.82 (m, 2H), 3.86 (s, 3H), 3.71 (s, 3H), 1.83-1.80 (m, 1H), 0.96-0.95 (m, 2H), 0.82-0.79 (m, 2H). Mass: 521.26 [m+1].

### Example-9

### N-(4-(1-isopropyl-4-(trifluoromethyl)-1H-imidazol-2-yl)benzyl)-2-(2-isopropylphenyl)-6,7-dihydro-5H-cyclopenta[d]pyrimidin-4-amine

The titled compound was synthesized by using the procedure provided in Example 3 by using Intermediate 8 and (2-isopropylphenyl)boronic acid (0.060 g) under Suzuki reaction conditions. 1H NMR, DMSO (400 MHz): 8.15 (bs, 1H), 7.59-7.56 (m, 1H), 7.50-7.48 (m, 2H), 7.41-7.39 (m, 3H), 7.31-7.30 (m, 2H), 7.17-7.13 (m, 1H), 4.72-4.70 (m, 2H), 4.45-4.42 (m, 1H), 3.47-3.44 (m, 1H), 2.84-2.77 (m, 4H), 2.09-2.05 (m, 2H), 1.39-1.37 (m, 6H), 0.98-0.96 (m, 6H). Mass: 520.21[m+1].

### Example-10

### 2-(4-cyclopropyl-6-methoxypyrimidin-5-yl)-N-(4-(1-isopropyl-4-(trifluoromethyl)-1H-imidazol-2-yl)benzyl)-6,7-dihydro-5H-cyclopenta[d]pyrimidin-4-amine

The titled compound was synthesized by using the procedure provided in Example 1 by using Intermediate 8 and 4-cyclopropyl-6-methoxy-5-(4,4,5,5-tetramethyl-1,3,2-dioxaborolan-2-yl)pyrimidine (0.15 g) under Suzuki reaction conditions. 1H NMR, DMSO (400 MHz): 8.57 (s, 1H), 7.91 (bs, 1H), 7.67-7.61 (m, 3H), 7.45-7.41 (m, 2H), 4.64-4.63 (m, 2H), 4.45-4.39 (m, 1H), 3.80 (s, 3H), 2.83-2.74 (m, 4H), 2.10-2.02 (m, 2H), 1.66-1.63 (m, 1H), 1.39-1.38 (m, 6H), 0.94-0.91 (m, 2H), 0.76-0.71 (m, 2H). Mass: 550.3 [m+1]. HPLC purity: 97.39%.

### Example-11

### 2-(4-cyclopropyl-6-(difluoromethoxy)pyrimidin-5-yl)-N-(4-(1-isopropyl-4-(trifluoromethyl)-1H-imidazol-2-yl)benzyl)-6,7-dihydro-5H-cyclopenta[d]pyrimidin-4-amine

The titled compound was synthesized by using the procedure provided in Example 1 by using Intermediate 8 and 4-cyclopropyl-6-(difluoromethoxy)-5-(4,4,5,5-tetramethyl-1,3,2-dioxaborolan-2-yl)pyrimidine (0.15 g) under Suzuki reaction conditions. 1H NMR, DMSO (400 MHz): 8.75 (s, 1H), 8.16 (bs, 1H), 7.78-7.77 (m, 1H), 7.64-7.55 (m, 2H), 7.47-7.41 (m, 2H), 4.66-4.65 (m, 2H), 4.44-4.41 (m, 1H), 2.85-2.74 (m, 4H), 2.10-2.04 (m, 2H), 1.39-1.37 (m, 6H), 0.93-0.92 (m, 2H), 0.76-0.75 (m, 2H). Mass: 586.45 [m+1]. HPLC purity: 95.32%.

### Example-12

### 1-(2-(4-cyclopropyl-6-methoxypyrimidin-5-yl)-6,7-dihydro-5H-cyclopenta[d]pyrimidin-4-yl)-1-(4-(1-methyl-4-(trifluoromethyl)-1H-imidazol-2-yl)benzyl)urea

To a solution of 2-(4-cyclopropyl-6-methoxypyrimidin-5-yl)-N-(4-(1-methyl-4-(trifluoromethyl)-1H-imidazol-2-yl)benzyl)-6,7-dihydro-5H-cyclopenta[d]pyrimidin-4-amine (1.0 eq) was dissolved in THF, cooled at 0° C., to it DAMP (0.1 eq), and Et₃N (20.eq) was added. Triphosgene (5.0 eq) was added portion wise. Stirred Reaction mixture for 2 hr at RT. Nh₄OH (10 ml) was added dropwise, stirred for 2 hr. Reaction mixture was quenched by sat. NaHCO₃ solution and extracted with (3×) EtOAc. Combined the organic layer and dried over Na₂SO₄, evaporated under vacuum. Crude was purified by column chromatography, eluted at 65-80% EtOAc-n-Hexane to get 1-(2-(4-cyclopropyl-6-methoxypyrimidin-5-yl)-6,7-dihydro-5H-cyclopenta[d]pyrimidin-4-yl)-1-(4-(1-methyl-4-(trifluoromethyl)-1H-imidazol-2-yl)benzyl)urea 0.1H NMR, DMSO (400 MHz): 8.62-8.61 (m, 1H), 7.91-7.90 (m, 1H), 7.64-7.62 (m, 2H), 7.40-7.38 (m, 2H), 6.98 (bs, 2H), s3.80 (s, 3H), 3.71 (s, 3H), 2.82-2.74 (m, 4H), 2.08-2.04 (m, 2H), 1.65-1.64 (m, 1H), 0.93-0.92 (m, 2H), 0.76-0.75 (m, 2H). Mass: 565.4 [m+1].

### Example-13

### 4-((4-(1-isopropyl-4-(trifluoromethyl)-1H-imidazol-2-yl)benzyl)oxy)-2-(2-isopropylphenyl)-6,7-dihydro-5H-cyclopenta[d]pyrimidine

The titled compound was synthesized by using the procedure provided in Example 3 by using Intermediate 9 and (2-isopropylphenyl)boronic acid (0.060 g) under Suzuki reaction conditions. 1H NMR, DMSO (400 MHz): 8.19 (s, 1H), 7.60-7.57 (m, 4H), 7.52-7.50 (m, 1H), 7.44-7.38 (m, 2H), 7.26-7.22 (m, 1H), 5.59 (s, 2H), 4.49-4.46 (m, 1H), 3.50-3.45 (m, 1H), 2.99-2.90 (m, 4H), 2.16-2.11 (m, 2H), 1.41-1.39 (m, 6H), 1.11-1.10 (m, 6H). Mass: 521.46 [m+1].

### Example-14

### 1-(4-(1-isopropyl-4-(trifluoromethyl)-1H-imidazol-2-yl)benzyl)-1-(2-(2-isopropylphenyl)-6,7-dihydro-5H-cyclopenta[d]pyrimidin-4-yl)-3,3-dimethylurea

To a solution of N-(4-(1-isopropyl-4-(trifluoromethyl)-1H-imidazol-2-yl)benzyl)-2-(2-isopropylphenyl)-6,7-dihydro-5H-cyclopenta[d]pyrimidin-4-amine (1.0 eq) was dissolved in THF, cooled at 0° C., to it DAMP (0.1 eq), and Et₃N (20.eq) was added. Triphosgene (5.0 eq) was added portion wise. Stirred Reaction mixture for 2 hr at RT. N—N dimethyl amine was added dropwise, stirred for 10 min. Reaction mixture was quenched by sat·NaHCO₃ solution and extracted with (3×) EtOAc. Combined the organic layer and dried over Na₂SO₄, evaporated under vacuum. Crude was purified by column chromatography, eluted at 65-80% EtOAc-n-Hexane to get 1-(4-(1-isopropyl-4-(trifluoromethyl)-1H-imidazol-2-yl)benzyl)-1-(2-(2-isopropylphenyl)-6,7-dihydro-5H-cyclopenta[d]pyrimidin-4-yl)-3,3-dimethylurea. Example 9. 1H NMR, DMSO (400 MHz): 8.14 (s, 1H), 7.56-7.48 (m, 2H), 7.45-7.42 (m, 3H), 7.37-7.36 (m, 2H), 7.25-7.18 (m, 1H), 5.09 (s, 2H), 4.45-4.41 (m, 1H), 3.47-3.31 (m, 1H), 2.95-2.91 (m, 2H), 2.84 (s, 6H), 2.69-2.65 (m, 2H), 2.07-1.98 (m, 2H), 1.38-1.37 (m, 6H), 0.98-0.96 (m, 6H). Mass: 591.3 [m+1].

### Example-15

### 2-(2-isopropylphenyl)-N-(4-(1-methyl-4-(trifluoromethyl)-1H-imidazo-2-yl)benzyl)-7H-purin-6-amine

The titled compound (60 mg) was synthesized by using the general procedure-2 by using Example 18. 1H NMR, DMSO (400 MHz): 12.9 (s, 1H), 8.36 (bs, 1H), 8.176 (s, 1H), 7.91 (s, 1H), 7.65-7.63 (m, 2H), 7.36-7.34 (m, 2H), 7.32-7.30 (m, 2H), 7.20-7.17 (m, 1H), 4.84-4.82 (m, 2H), 3.74 (s, 3H), 3.57-3.53 (m, 1H), 1.03-1.01 (m, 6H). Mass: 492.3 [m+1].

### Example-16

### 2-(4-cyclopropyl-6-methoxypyrimidin-5-yl)-N-(4-(1-methyl-4-(trifluoromethyl)-1H-imidazol-2-yl)benzyl)-7H-purin-6-amine

The titled compound (A) (150 mg) was synthesized by using the general procedure-2 by using Example 19v. 1H NMR, DMSO (400 MHz): 13.0 (s, 1H), 8.60 (s, 1H), 8.40 (s, 1H), 7.91 (s, 1H), 7.64-7.62 (m, 1H), 7.57-7.55 (m, 2H), 7.47-7.45 (m, 2H), 4.7 (bs, 2H), 3.81 (s, 3H), 3.74 (s, 3H), 1.75-1.69 (m, 1H), 0.96-0.95 (m, 2H), 0.82-0.80 (m, 2H). Mass: 522.3[m+1].

### Example-17

### 2-chloro-N-(4-(1-methyl-4-(trifluoromethyl)-1H-imidazol-2-yl)benzyl)-9-(tetrahydro-2H-pyran-2-yl)-9H-purin-6-amine

To a solution of 2,6-dichloro-9-(tetrahydro-2H-pyran-2-yl)-9H-purine (0.5 g, 1.00 eq) and (4-(1-methyl-4-(trifluoromethyl)-1H-imidazol-2-yl)phenyl)methanamine (0. 46 gm, 1.00 eq) was dissolved in IPA (5 ml). DIPEA (0.8 ml, 2 eq) was added to reaction mixture and heated at 80° C. for 12 h. Reaction mixture was evaporated under vacuum, crude was poured into ice cold water and extracted with (3×) EtOAc. Combined the organic layer and dried over Na₂SO₄, evaporated under vacuum. Crude was purified by column chromatography, eluted at 90% EtOAc-n-Hexane to get 2-chloro-N-(4-(1-methyl-4-(trifluoromethyl)-1H-imidazol-2-yl)benzyl)-9-(tetrahydro-2H-pyran-2-yl)-9H-purin-6-amine (0.2 gm). Mass: 493.4 [M+1].

### Example-18

### 2-(2-isopropylphenyl)-N-(4-(1-methyl-4-(trifluoromethyl)-1H-imidazol-2-yl)benzyl)-9-(tetrahydro-2H-pyran-2-yl)-9H-purin-6-amine

The titled compound was synthesized by using the procedure provided in Example 3 by using Example 17 and (2-isopropylphenyl)boronic acid (0.060 g) under Suzuki reaction conditions. Mass: 576.1 [m+1].

### Example-19

### 2-(4-cyclopropyl-6-methoxypyrimidin-5-yl)-N-(4-(1-methyl-4-(trifluoromethyl)-1H-imidazol-2-yl)benzyl)-9-(tetrahydro-2H-pyran-2-yl)-9H-purin-6-amine

The titled compound was synthesized by using the procedure provided in Example 1 by using Example 17 and 4-cyclopropyl-6-methoxy-5-(4,4,5,5-tetramethyl-1,3,2-dioxaborolan-2-yl)pyrimidine (0.15 g) under Suzuki reaction conditions. Mass: 606.1[m+1].

### Example-20

### 2-(2-isopropylphenyl)-4-((4-(1-methyl-4-(trifluoromethyl)-1H-imidazol-2-yl)benzyl)amino)-6,7-dihydro-5H-cyclopenta[b]pyridine-3-carbonitrile

The titled compound was synthesized by using the procedure provided in Example 3 by using Intermediate 12 and (2-isopropylphenyl)boronic acid (0.060 g) under Suzuki reaction conditions. Mass: 516.3 [m+1].

### Example-21

### 2-(4-cyclopropyl-6-methoxypyrimidin-5-yl)-4-((4-(1-methyl-4-(trifluoromethyl)-1H-imidazol-2-yl)benzyl)amino)-6,7-dihydro-5H-cyclopenta[b]pyridine-3-carbonitriles

The titled compound was synthesized by using the procedure provided in Example 1 by using Intermediate 12 and 4-cyclopropyl-6-methoxy-5-(4,4,5,5-tetramethyl-1,3,2-dioxaborolan-2-yl)pyrimidine (0.15 g) under Suzuki reaction conditions. Mass: 546.2 [m+1].

### Example-22

### 2-(4-cyclopropyl-6-methoxypyrimidin-5-yl)-N-(4-(1-isopropyl-4-(trifluoromethyl)-1H-imidazol-2-yl)benzyl)pyrrolo[2,1-f][1,2,4]triazin-4-amine

The titled compound was synthesized by using the procedure provided in Example 1 by using Intermediate 13 and 4-cyclopropyl-6-methoxy-5-(4,4,5,5-tetramethyl-1,3,2-dioxaborolan-2-yl)pyrimidine (0.15 g) under Suzuki reaction conditions. 1H NMR, DMSO (400 MHz): 9.01-8.98 (m, 1H), 8.60 (s, 1H), 8.13 (s, 1H), 7.69 (s, 1H), 7.49-7.44 (m, 4H), 6.98-6.97 (m, 1H), 6.68-6.66 (m, 1H), 4.76-4.74 (m, 2H), 4.41-4.38 (m, 1H), 311 (s, 1H), 1.82-1.80 (m, 1H), 1.36-1.34 (m, 6H), 0.96-0.95 (m, 2H), 0.82-0.79 (m, 2H). Mass: 549.47 [m+1].

### Example-23

### 2-(4-cyclopropyl-6-methoxypyrimidin-5-yl)-4-((4-(1-methyl-4-(trifluoromethyl)-1H-imidazol-2-yl)benzyl)oxy)pyrrolo[2,1-f][1,2,4]triazine

The titled compound was synthesized by using the procedure provided in Example 1 by using Intermediate 14 and 4-cyclopropyl-6-methoxy-5-(4,4,5,5-tetramethyl-1,3,2-dioxaborolan-2-yl)pyrimidine (0.15 g) under Suzuki reaction conditions.

### Example-24

### 2-(4-cyclopropyl-6-methoxypyrimidin-5-yl)-4-((4-(1-methyl-4-(trifluoromethyl)-1H-imidazol-2-yl)benzyl)oxy)-6,7-dihydro-5H-cyclopenta[d]pyrimidine

The titled compound was synthesized by using the procedure provided in Example 3 by using Intermediate 9 and 4-cyclopropyl-6-methoxy-5-(4,4,5,5-tetramethyl-1,3,2-dioxaborolan-2-yl)pyrimidine (0.060 g) under Suzuki reaction conditions. 1H NMR, DMSO (400 MHz): 8.65 (s, 1H), 7.95 (bs, 1H), 7.73-7.71 (m, 2H), 7.64-7.56 (m, 2H), 5.56 (s, 2H), 3.88 (s, 3H), 3.75 (s, 3H), 2.98-2.83 (m, 4H), 2.10-2.04 (m, 2H), 1.66-1.63 (m, 1H), 1.13-1.11 (m, 2H), 0.86-0.84 (m, 2H). Mass: 523.21[M+1].

### Example 25

### 2-(4-cyclopropyl-6-methoxypyrimidin-5-yl)-6-((4-(1-methyl-4-(trifluoromethyl)-1H-imidazol-2-yl)benzyl)oxy)-9-(tetrahydro-2H-pyran-2-yl)-9H-purine

The titled compound was synthesized by using the procedure provided in Example 1 by using 2-chloro-6-((4-(1-methyl-4-(trifluoromethyl)-1H-imidazol-2-yl)benzyl)oxy)-9-(tetrahydro-2H-pyran-2-yl)-9H-purine and 4-cyclopropyl-6-methoxy-5-(4,4,5,5-tetramethyl-1,3,2-dioxaborolan-2-yl)pyrimidine (0.060 g) under Suzuki reaction conditions. Mass: 607.23[M+1].

### Example 26

### 2-(4-cyclopropyl-6-methoxypyrimidin-5-yl)-6-((4-(1-methyl-4-(trifluoromethyl)-1H-imidazol-2-yl)benzyl)oxy)-7H-purine

To a solution of 2-(4-cyclopropyl-6-methoxypyrimidin-5-yl)-6-((4-(1-methyl-4-(trifluoromethyl)-1H-imidazol-2-yl)benzyl)oxy)-9-(tetrahydro-2H-pyran-2-yl)-9H-purine (0.11 g, 1.0 eq) was dissolved in DCM (2 ml). To it TFA (2.0 ml, 100 eq) was added. Reaction mixture was Stirred at RT for 16 h. Reaction mixture was Evaporated under vacuum, crude was poured into Sat. NaHCO₃ solution (10 ml) and extracted with DCM (3×20 ml), combined organic layer and dried over Na₂SO₄ to give 2-(4-cyclopropyl-6-methoxypyrimidin-5-yl)-6-((4-(1-methyl-4-(trifluoromethyl)-1H-imidazol-2-yl)benzyl)oxy)-7H-purine (0.1 g). Mass: 523.1[M+1].

### Example 27

### 2-(2-fluoro-6-methoxyphenyl)-6-((4-(1-methyl-4-(trifluoromethyl)-1H-imidazol-2-yl)benzyl)oxy)-9-(tetrahydro-2H-pyran-2-yl)-9H-purine

The titled compound was synthesized by using the procedure provided in Example 1 by using 2-chloro-6-((4-(1-methyl-4-(trifluoromethyl)-1H-imidazol-2-yl)benzyl)oxy)-9-(tetrahydro-2H-pyran-2-yl)-9H-purine and (2-fluoro-6-methoxyphenyl)boronic acid (0.070 g) under Suzuki reaction conditions. Mass: 583.40[M+1].

### Example 28

### 2-(2-fluoro-6-methoxyphenyl)-6-((4-(1-methyl-4-(trifluoromethyl)-1H-imidazol-2-yl)benzyl)oxy)-7H-purine

To a solution of 2-chloro-6-((4-(1-methyl-4-(trifluoromethyl)-1H-imidazol-2-yl)benzyl)oxy)-9-(tetrahydro-2H-pyran-2-yl)-9H-purine (0.11 g, 1.0 eq) was dissolved in DCM (2 ml). To it TFA (2.0 ml, 100 eq) was added. Reaction mixture was Stirred at RT for 16 h. Reaction mixture was Evaporated under vacuum, crude was poured into Sat. NaHCO₃ solution (10 ml) and extracted with DCM (3×20 ml), combined organic layer and dried over Na₂SO₄ to give 2-(2-fluoro-6-methoxyphenyl)-6-((4-(1-methyl-4-(trifluoromethyl)-1H-imidazol-2-yl)benzyl)oxy)-7H-purine (0.1 g). Mass: 499.34[M+1].

### Example 29

### 2-(2-fluoro-6-methoxyphenyl)-N-(4-(1-methyl-4-(trifluoromethyl)-1H-imidazol-2-yl)benzyl)-9-(tetrahydro-2H-pyran-2-yl)-9H-purin-6-amine

The titled compound was synthesized by using the procedure provided in Example 1 by using 2-chloro-N-(4-(1-methyl-4-(trifluoromethyl)-1H-imidazol-2-yl)benzyl)-9-(tetrahydro-2H-pyran-2-yl)-9H-purin-6-amine and (2-fluoro-6-methoxyphenyl)boronic acid under Suzuki reaction conditions. Mass: 582.24[M+1].

### Example 30

### 2-(2-fluoro-6-methoxyphenyl)-N-(4-(1-methyl-4-(trifluoromethyl)-1H-imidazol-2-yl)benzyl)-7H-purin-6-amine

To a solution of 2-(2-methoxypyridin-3-yl)-N-(4-(1-methyl-4-(trifluoromethyl)-1H-imidazol-2-yl)benzyl)-9-(tetrahydro-2H-pyran-2-yl)-9H-purin-6-amine (0.11 g, 1.0 eq) was dissolved in DCM (2 ml). To it TFA (2.0 ml, 100 eq) was added. Reaction mixture was Stirred at RT for 16 h. Reaction mixture was Evaporated under vacuum, crude was poured into Sat. NaHCO₃ solution (10 ml) and extracted with DCM (3×20 ml), combined organic layer and dried over Na₂S04 to give 2-(2-fluoro-6-methoxyphenyl)-N-(4-(1-methyl-4-(trifluoromethyl)-1H-imidazol-2-yl)benzyl)-7H-purin-6-amine (0.1 g). Mass: 4981.35[M+1].

### Example 31

### 2-(2-methoxypyridin-3-yl)-N-(4-(1-methyl-4-(trifluoromethyl)-1H-imidazol-2-yl)benzyl)-9-(tetrahydro-2H-pyran-2-yl)-9H-purin-6-amine

The titled compound was synthesized by using the procedure provided in Example 1 by using 2-chloro-N-(4-(1-methyl-4-(trifluoromethyl)-1H-imidazol-2-yl)benzyl)-9-(tetrahydro-2H-pyran-2-yl)-9H-purin-6-amine and 2-methoxy-3-(4,4,5,5-tetramethyl-1,3,2-dioxaborolan-2-yl)pyridine (0.070 g) under Suzuki reaction conditions. Mass: 565.20[M+1].

### Example 32

### 2-(2-methoxypyridin-3-yl)-N-(4-(1-methyl-4-(trifluoromethyl)-1H-imidazol-2-yl)benzyl)-7H-purin-6-amine

To a solution of 2-(2-methoxypyridin-3-yl)-N-(4-(1-methyl-4-(trifluoromethyl)-1H-imidazol-2-yl)benzyl)-9-(tetrahydro-2H-pyran-2-yl)-9H-purin-6-amine (0.11 g, 1.0 eq) was dissolved in DCM (2 ml). To it TFA (2.0 ml, 100 eq) was added. Reaction mixture was Stirred at RT for 16 h. Reaction mixture was Evaporated under vacuum, crude was poured into Sat. NaHCO₃ solution (10 ml) and extracted with DCM (3×20 ml), combined organic layer and dried over Na₂SO₄ to give 2-(2-methoxypyridin-3-yl)-N-(4-(1-methyl-4-(trifluoromethyl)-1H-imidazol-2-yl)benzyl)-7H-purin-6-amine (0.1 g). Mass: 481.20[M+1].

### Example 33

### 2-(4-methoxypyridin-3-yl)-N-(4-(1-methyl-4-(trifluoromethyl)-1H-imidazol-2-yl)benzyl)-9-(tetrahydro-2H-pyran-2-yl)-9H-purin-6-amine

The titled compound was synthesized by using the procedure provided in Example 1 by using 2-chloro-N-(4-(1-methyl-4-(trifluoromethyl)-1H-imidazol-2-yl)benzyl)-9-(tetrahydro-2H-pyran-2-yl)-9H-purin-6-amine and 2-methoxy-3-(4,4,5,5-tetramethyl-1,3,2-dioxaborolan-2-yl)pyridine (0.070 g) under Suzuki reaction conditions. Mass: 565.20[M+1].

### Example 34

### 2-(4-methoxypyridin-3-yl)-N-(4-(1-methyl-4-(trifluoromethyl)-1H-imidazol-2-yl)benzyl)-7H-purin-6-amine

To a solution of 2-(4-methoxypyridin-3-yl)-N-(4-(1-methyl-4-(trifluoromethyl)-1H-imidazol-2-yl)benzyl)-9-(tetrahydro-2H-pyran-2-yl)-9H-purin-6-amine (0.11 g, 1.0 eq) was dissolved in DCM (2 ml). To it TFA (2.0 ml, 100 eq) was added. Reaction mixture was Stirred at RT for 16 h. Reaction mixture was Evaporated under vacuum, crude was poured into Sat. NaHCO₃ solution (10 ml) and extracted with DCM (3×20 ml), combined organic layer and dried over Na₂SO₄ to give 2-(4-methoxypyridin-3-yl)-N-(4-(1-methyl-4-(trifluoromethyl)-1H-imidazol-2-yl)benzyl)-7H-purin-6-amine (0.1 g). Mass: 481.20[M+1].

### Example 35

### N-(4-(1-methyl-4-(trifluoromethyl)-1H-imidazol-2-yl)benzyl)-2-morpholino-9-(tetrahydro-2H-pyran-2-yl)-9H-purin-6-amine

To a solution of 2-chloro-N-(4-(1-methyl-4-(trifluoromethyl)-1H-imidazol-2-yl)benzyl)-9-(tetrahydro-2H-pyran-2-yl)-9H-purin-6-amine (1.00 eq) and Morpholine (2.00 eq) was dissolved in ACN (5 ml). DIPEA (4 eq) was added to reaction mixture and heated at 80° C. for 12 h. Reaction mixture was poured into ice cold water and extracted with (3×) EtOAc. Combined the organic layer and dried over Na₂SO₄, evaporated under vacuum. Crude was purified by column chromatography, eluted at 45% EtOAc-n-Hexane to get N-(4-(1-methyl-4-(trifluoromethyl)-1H-imidazol-2-yl)benzyl)-2-morpholino-9-(tetrahydro-2H-pyran-2-yl)-9H-purin-6-amine (0.2 gm). Mass: 543.4 [M+1].

### Example 36

### N-(4-(1-methyl-4-(trifluoromethyl)-1H-imidazol-2-yl)benzyl)-2-morpholino-7H-purin-6-amine

To a solution of N-(4-(1-methyl-4-(trifluoromethyl)-1H-imidazol-2-yl)benzyl)-2-morpholino-9-(tetrahydro-2H-pyran-2-yl)-9H-purin-6-amine (0.11 g, 1.0 eq) was dissolved in DCM (2 ml). To it TFA (2.0 ml, 100 eq) was added. Reaction mixture was Stirred at RT for 16 h. Reaction mixture was Evaporated under vacuum, crude was poured into Sat. NaHCO₃ solution (10 ml) and extracted with DCM (3×20 ml), combined organic layer and dried over Na₂SO₄ to give N-(4-(1-methyl-4-(trifluoromethyl)-1H-imidazol-2-yl)benzyl)-2-morpholino-7H-purin-6-amine (0.1 g). Mass: 459.38[M+1].

### Example 37

### 2-(4-cyclopropyl-6-methoxypyrimidin-5-yl)-9-methyl-N-(4-(1-methyl-4-(trifluoromethyl)-1H-imidazol-2-yl)benzyl)-9H-purin-6-amine

To a solution of 2-(4-cyclopropyl-6-methoxypyrimidin-5-yl)-N-(4-(1-methyl-4-(trifluoromethyl)-1H-imidazol-2-yl)benzyl)-7H-purin-6-amine (1.0 eq) was dissolved in THF (10 vol). Reaction mixture was cooled at 0° C., to it NaH (1.3 eq) was added portion wise at same temp. Iodomethane (1.3 eq) was added and reaction mixture was stirred at RT for 3 hr. Reaction mixture was poured into water and extracted in EtOAc (3×), Combined the organic layer and dried over Na₂SO₄, evaporated under vacuum. Crude was purified by column chromatography, eluted at 35-45% EtOAc-n-Hexane to get 2-(4-cyclopropyl-6-methoxypyrimidin-5-yl)-9-methyl-N-(4-(1-methyl-4-(trifluoromethyl)-1H-imidazol-2-yl)benzyl)-9H-purin-6-amine. Mass: 536.43 [M+1].

### Example 38

### 2-(2-chloro-3-methylphenyl)-N-(4-(1-methyl-4-(trifluoromethyl)-1H-imidazol-2-yl)benzyl)-7H-purin-6-amine

The titled compound (25 mg) was synthesized by using the general procedure-2 by using intermediate 16. 1H NMR, DMSO (400 MHz): 13.0 (s, 1H), 8.28 (s, 1H), 8.17-8.15 (m, 1H), 7.95 (s, 2H), 7.88 (s, 1H), 7.65-7.63 (m, 2H), 7.56-7.54 (m, 2H), 7.38-7.36 (m, 1H), 4.90 (bs, 2H), 3.71 (s, 3H), 2.32 (s, 3H). Mass: 498.12 [M+1].

### Example 39

### 2-(3,5-dichlorophenyl)-N-(4-(1-methyl-4-(trifluoromethyl)-1H-imidazol-2-yl)benzyl)-7H-purin-6-amine

The titled compound (15 mg) was synthesized by using the general procedure-2 by using intermediate 17. 1H NMR, DMSO (400 MHz): 13.1 (s, 1H), 8.23 (s, 1H), 8.19-8.10 (m, 2H), 7.87 (s, 1H), 7.66-7.60 (m, 2H), 7.59-7.53 (m, 3H), 7.16-7.12 (m, 1H), 4.82 (s, 2H), 3.72 (s, 3H). Mass: 518.08 [M+1].

### Example 40

### 2-(2,4-difluorophenyl)-N-(4-(1-methyl-4-(trifluoromethyl)-1H-imidazol-2-yl)benzyl)-7H-purin-6-amine

The titled compound (20 mg) was synthesized by using the general procedure-2 by using intermediate 18. 1H NMR, DMSO (400 MHz): 13.0 (s, 1H), 8.11 (s, 1H), 8.05-7.99 (m, 1H), 7.90 (s, 1H), 7.66-7.61 (m, 2H), 7.59-7.53 (m, 3H), 7.29-7.24 (m, 1H), 7.16-7.12 (m, 1H), 4.85 (s, 2H), 3.71 (s, 3H). Mass: 486.43 [M+1].

### Example 41

### 2-(3,5-difluorophenyl)-N-(4-(1-methyl-4-(trifluoromethyl)-1H-imidazol-2-yl)benzyl)-7H-purin-6-amine

The titled compound (24 mg) was synthesized by using the general procedure-2 by using intermediate 19. 1H NMR, DMSO (400 MHz): 13.12 (s, 1H), 8.20 (s, 1H), 8.19-8.15 (m, 2H), 7.89 (s, 1H), 7.66-7.61 (m, 2H), 7.58-7.52 (m, 3H), 7.16-7.11 (m, 1H), 4.83 (s, 2H), 3.74 (s, 3H). Mass: 486.22 [M+1].

### Example 42

### 2-(2,4-dichlorophenyl)-N-(4-(1-methyl-4-(trifluoromethyl)-1H-imidazol-2-yl)benzyl)-7H-purin-6-amine

The titled compound (15 mg) was synthesized by using the general procedure-2 by using intermediate 20. 1H NMR, DMSO (400 MHz): 13.01 (s, 1H), 8.12 (s, 1H), 8.06-7.99 (m, 1H), 7.91 (s, 1H), 7.69-7.64 (m, 2H), 7.58-7.52 (m, 3H), 7.26-7.21 (m, 1H), 7.18-7.14 (m, 1H), 4.84 (s, 2H), 3.72 (s, 3H). Mass: 518.16 [M+1].

### Example 43

### 2-(2,6-dimethoxypyridin-3-yl)-N-(4-(1-methyl-4-(trifluoromethyl)-1H-imidazol-2-yl)benzyl)-7H-purin-6-amine

The titled compound (12 mg) was synthesized by using the general procedure-2 by using intermediate 21. DMSO (400 MHz): 12.99 (s, 1H), 8.27 (bs, 1H), 8.11 (s, 1H), 8.03-8.00 (m, 1H), 7.90 (s, 1H), 7.66-7.64 (m, 2H), 7.54-7.52 (m, 2H), 6.44-6.42 (m, 1H), 4.79 (s, 2H), 3.90 (s, 3H), 3.87 (s, 3H), 3.75 (s, 3H). Mass: 511.24 [M+1].

### Example 44

### 2-(2-fluoro-5-(trifluoromethyl)phenyl)-N-(4-(1-methyl-4-(trifluoromethyl)-1H-imidazol-2-yl)benzyl)-7H-purin-6-amine

The titled compound (33 mg) was synthesized by using the general procedure-2 by using intermediate 22. 1H NMR, DMSO (400 MHz): 13.18 (s, 1H), 8.61 (s, 1H), 8.33-8.31 (m, 1H), 8.22 (s, 1H), 7.90-7.84 (m, 3H), 7.66-7.59 (m, 2H), 7.52-7.50 (m, 2H), 4.96 (s, 2H), 3.74 (s, 3H). Mass: 534.33 [M+1].

### Example 45

### N-(4-(1-methyl-4-(trifluoromethyl)-1H-imidazol-2-yl)benzyl)-2-(2-(trifluoromethyl)phenyl)-7H-purin-6-amine

The titled compound (23 mg) was synthesized by using the general procedure-2 by using intermediate 23. 1H NMR, DMSO (400 MHz): 13.10 (s, 1H), 8.40 (s, 1H), 8.19 (s, 1H), 7.90 (s, 1H), 7.80-7.78 (m, 1H), 7.72-7.59 (m, 5H), 7.46-7.44 (m, 2H), 4.78 (s, 2H), 3.74 (s, 3H). Mass: 518.15 [M+1].

### Example 46

### N-(4-(1-methyl-4-(trifluoromethyl)-1H-imidazol-2-yl)benzyl)-2-(3-(trifluoromethoxy)phenyl)-7H-purin-6-amine

The titled compound (21 mg) was synthesized by using the general procedure-2 by using intermediate 24. 1H NMR, DMSO (400 MHz): 13.10 (s, 1H), 8.56 (s, 1H), 8.36-8.34 (m, 1H), 8.21-8.18 (m, 2H), 7.91 (s, 1H), 7.89-7.67 (m, 2H), 7.59-7.56 (m, 3H), 7.42-7.40 (m, 1H), 4.87 (bs, 2H), 3.74 (s, 3H). Mass: 534.13 [M+1].

### Example 47

### N-(4-(1-methyl-4-(trifluoromethyl)-1H-imidazol-2-yl)benzyl)-2-(4-(trifluoromethoxy)phenyl)-7H-purin-6-amine

The titled compound (29 mg) was synthesized by using the general procedure-2 by using intermediate 25. 1H NMR, DMSO (400 MHz): 13.06 (s, 1H), 8.49 (s, 1H), 8.47-8.44 (m, 2H), 8.16 (s, 1H), 7.89 (s, 1H), 7.68-7.66 (m, 2H), 7.58-7.56 (m, 2H), 7.44-7.42 (m, 2H), 4.87 (bs, 2H), 3.74 (s, 3H). Mass: 534.21 [M+1].

### Example 48

### 2-(2-fluoro-3-methoxyphenyl)-N-(4-(1-methyl-4-(trifluoromethyl)-1H-imidazol-2-yl)benzyl)-7H-purin-6-amine

The titled compound (24 mg) was synthesized by using the general procedure-2 by using intermediate 26. 1H NMR, DMSO (400 MHz): 12.96 (s, 1H), 8.13 (s, 1H), 8.12 (s, 1H), 8.00 (s, 1H), 7.67-7.55 (m, 5H), 7.25-7.23 (m, 2H), 4.87 (bs, 2H), 3.83 (s, 3H), 3.74 (s, 3H). Mass: 498.19 [M+1].

### Example 49

### 2-fluoro-5-(6-((4-(1-methyl-4-(trifluoromethyl)-1H-imidazol-2-yl)benzyl)amino)-7H-purin-2-yl)benzonitrile

The titled compound (28 mg) was synthesized by using the general procedure-2 by using intermediate 27. 1H NMR, DMSO (400 MHz): 13.11 (s, 1H), 8.67 (s, 1H), 8.19 (s, 1H), 7.68-7.66 (m, 1H), 7.64-7.56 (m, 7H), 4.87 (bs, 2H), 3.71 (s, 3H). Mass: 493.22 [M+1].

### Example 50

### 2-(3,5-dimethylphenyl)-N-(4-(1-methyl-4-(trifluoromethyl)-1H-imidazol-2-yl)benzyl)-7H-purin-6-amine

The titled compound (13 mg) was synthesized by using the general procedure-2 by using intermediate 28. 1H NMR, DMSO (400 MHz): 13.1 (s, 1H), 8.23 (s, 1H), 8.19-8.10 (m, 2H), 7.87 (s, 1H), 7.66-7.60 (m, 2H), 7.59-7.53 (m, 3H), 7.16-7.12 (m, 1H), 4.82 (s, 2H), 3.72 (s, 3H), 2.32 (s, 3H), 2.31 (s, 3H). Mass: 478.31 [M+1].

### Example 51

### 2-(3,4-dimethoxyphenyl)-N-(4-(1-methyl-4-(trifluoromethyl)-1H-imidazol-2-yl)benzyl)-7H-purin-6-amine

The titled compound (11 mg) was synthesized by using the general procedure-2 by using intermediate 29. 1H NMR, DMSO (400 MHz): 12.99 (s, 1H8.48 (s, 1H), 8.15 (s, 1H), 8.06-8.04 (m, 1H), 7.90 (m, 2H), 7.67-7.65 (m, 2H), 7.59-7.57 (m, 2H), 7.29-7.24 (m, 1H), 4.84 (3, 2H), 3.78 (s, 3H), 3.76 (s, 3H), 3.73 (s, 3H). Mass: 510.23 [M+1].

### Example 52

### 2-(4-fluoro-3-methoxyphenyl)-N-(4-(1-methyl-4-(trifluoromethyl)-1H-imidazol-2-yl)benzyl)-7H-purin-6-amine

The titled compound (35 mg) was synthesized by using the general procedure-2 by using intermediate 30. 1H NMR, DMSO (400 MHz): 13.03 (s, 1H), 8.48 (s, 1H), 8.15 (s, 1H), 8.06-8.04 (m, 1H), 7.90 (m, 2H), 7.67-7.65 (m, 2H), 7.59-7.57 (m, 2H), 7.29-7.24 (m, 1H), 4.85 (bs, 2H), 3.92 (s, 3H) 3.74 (s, 3H). Mass: 498.20 [M+1].

### Example 53

### 2-(2,4-dimethoxyphenyl)-N-(4-(1-methyl-4-(trifluoromethyl)-1H-imidazol-2-yl)benzyl)-7H-purin-6-amine

The titled compound (06 mg) was synthesized by using the general procedure-2 by using intermediate 31. 1H NMR, DMSO (400 MHz): 13.10 (s, 1H), 8.47 (s, 1H), 8.14 (s, 1H), 8.05-8.03 (m, 1H), 7.91 (m, 2H), 7.66-7.63 (m, 2H), 7.59-7.56 (m, 2H), 7.29-7.23 (m, 1H), 4.85-4.83 (m, 2H), 3.79 (s, 3H), 3.78 (s, 3H), 3.74 (s, 3H). Mass: 510.16 [M+1].

### Example 54

### 2-(3-fluoro-2-(trifluoromethyl)phenyl)-N-(4-(1-methyl-4-(trifluoromethyl)-1H-imidazol-2-yl)benzyl)-7H-purin-6-amine

The titled compound (23 mg) was synthesized by using the general procedure-2 by using intermediate 32. 1H NMR, DMSO (400 MHz): 13.1 (s, 1H), 8.63 (s, 1H), 8.62 (s, 1H), 8.20 (s, 1H), 7.97-7.77 (m, 3H), 7.67-7.65 (m, 2H), 7.62-7.57 (m, 2H), 4.85-4.83 (m, 2H), 3.79 (s, 3H), 3.75 (s, 3H), 3.70 (s, 3H). Mass: 536.18 [M+1].

### Example 55

### 3-(6-((4-(1-methyl-4-(trifluoromethyl)-1H-imidazol-2-yl)benzyl)amino)-7H-purin-2-yl)benzamide

The titled compound (10 mg) was synthesized by using the general procedure-2 by using intermediate 33. Mass: 493.20 [M+1].

### Example 56

### N-(4-(1-methyl-4-(trifluoromethyl)-1H-imidazol-2-yl)benzyl)-2-(4-(methylthio)phenyl)-7H-purin-6-amine

The titled compound (12 mg) was synthesized by using the general procedure-2 by using intermediate 34. Mass: 496.23 [M+1].

### Example 57

### N-(4-(1-methyl-4-(trifluoromethyl)-1H-imidazol-2-yl)benzyl)-2-(4-(trifluoromethyl)phenyl)-7H-purin-6-amine

The titled compound (10 mg) was synthesized by using the general procedure-2 by using intermediate 35. 1H NMR, DMSO (400 MHz): 13.11 (s, 1H), 8.61 (s, 1H), 8.49 (s, 1H), 8.13 (s, 1H), 7.95-7.89 (m, 2H), 7.72-7.70 (m, 2H), 7.67-7.65 (m, 2H), 7.62-7.57 (m, 2H), 4.84 (s, 2H), 3.73 (s, 3H). Mass: 518.16 [M+1].

### Example 58

### N-(4-(1-methyl-4-(trifluoromethyl)-1H-imidazol-2-yl)benzyl)-2-(3-(trifluoromethyl)phenyl)-7H-purin-6-amine

The titled compound (23 mg) was synthesized by using the general procedure-2 by using intermediate 36. 1H NMR, DMSO (400 MHz): 13.1 (s, 1H), 8.65 (s, 1H), 8.61 (s, 1H), 8.19 (s, 1H), 7.95-7.89 (m, 1H), 7.79-7.77 (m, 1H), 7.72-7.70 (m, 1H), 7.67-7.65 (m, 2H), 7.62-7.57 (m, 3H), 4.85-4.83 (m, 2H), 3.73 (s, 3H). Mass: 518.12 [M+1].

### Example 59

### N-(3-(4-((4-(1-methyl-4-(trifluoromethyl)-1H-imidazol-2-yl)benzyl)amino)-6,7-dihydro-5H-cyclopenta[d]pyrimidin-2-yl)phenyl)acetamide

The titled compound (10 mg) was synthesized by using the general procedure-3 by using intermediate 8 and boronic acid. Mass: 507.24 [M+1].

### Example 60

### 2-fluoro-5-(4-((4-(1-methyl-4-(trifluoromethyl)-1H-imidazol-2-yl)benzyl)amino)-6,7-dihydro-5H-cyclopenta[d]pyrimidin-2-yl)benzonitrile

The titled compound (09 mg) was synthesized by using the general procedure-3 by using intermediate 8 and boronic acid. 1H NMR, DMSO (400 MHz): 7.90 (bs, 1H), 7.87-7.85 (m, 1H), 7.82 (s, 1H), 7.67-7.65 (m, 2H), 7.60 (s, 1H), 7.55-7.52 (m, 2H), 6.98-6.96 (m, 1H), 4.75-4.74 (m, 2H), ss3.71 (s, 3H), 2.83-2.81 (m, 2H), 2.75-2.72 (m, 2H), 2.06-2.02 (m, 2H). Mass: 493.22 [M+1].

### Example 61

### 2-(5-chloro-2-fluorophenyl)-N-(4-(1-methyl-4-(trifluoromethyl)-1H-imidazol-2-yl)benzyl)-6,7-dihydro-5H-cyclopenta[d]pyrimidin-4-amine

The titled compound (35 mg) was synthesized by using the general procedure-3 by using intermediate 8 and boronic acid. DMSO (400 MHz): 7.91 (bs, 1H), 7.83-7.81 (m, 1H), 7.75 (s, 1H), 7.67-7.59 (m, 2H), 7.56-7.55 (m, 2H), 7.49-7.47 (m, 1H), 7.32-7.27ss (m, 1H), 4.71-4.70 (m, 2H), 3.72 (s, 3H), 2.84-2.80 (m, 2H), 2.78-2.74 (m, 2H), 2.07-2.04 (m, 2H). Mass: 502.19 [M+1].

### Example 62

### 2-(6-ethoxypyridin-3-yl)-N-(4-(1-methyl-4-(trifluoromethyl)-1H-imidazol-2-yl)benzyl)-6,7-dihydro-5H-cyclopenta[d]pyrimidin-4-amine

The titled compound (10 mg) was synthesized by using the general procedure-3 by using intermediate 8 and boronic acid. Mass: 495.18 [M+1].

### Example 63

### 4-(4-((4-(1-methyl-4-(trifluoromethyl)-1H-imidazol-2-yl)benzyl)amino)-6,7-dihydro-5H-cyclopenta[d]pyrimidin-2-yl)benzonitrile

The titled compound (42 mg) was synthesized by using the general procedure-3 by using intermediate 8 and boronic acid. 1H NMR, DMSO (400 MHz): 8.43 (bs, 1H), 8.41 (s, 1H), 7.91-7.87 (m, 3H), 7.80-7.77 (m, 1H), 7.68-7.66 (m, 2H), 7.53-7.51 (m, 2H), 4.79-4.78 (m, 2H), 3.71 (s, 3H), 2.87-2.83 (m, 2H), 2.78-64642.75 (m, 2H), 2.09-2.05 (m65, 2H). Mass: 475.21 [M+1].

### Example 64

### 2-([1,1′-biphenyl]-4-yl)-N-(4-(1-methyl-4-(trifluoromethyl)-1H-imidazol-2-yl)benzyl)-6,7-dihydro-5H-cyclopenta[d]pyrimidin-4-amine

The titled compound (32 mg) was synthesized by using the general procedure-3 by using intermediate 8 and boronic acid. 1H NMR, DMSO (400 MHz): 8.46 (bs, 1H), 8.26-8.24 (m, 1H), 7.89 (s, 1H), 7.72-7.62 (m, 6H), 7.56-7.52 (m, 2H), 7.50-7.46 (m, 2H), 7.38-7.35 (m, 1H), 4.78-4.76 (m, 2H), 3.63 (s, 3H), 2.87-2.83 (m, 2H), 2.79-2.76 (m, 2H), 2.09-2.05 (m, 2H). Mass: 526.26 [M+1].

### Example 65

### 2-(2,6-dimethoxypyridin-3-yl)-N-(4-(1-methyl-4-(trifluoromethyl)-1H-imidazol-2-yl)benzyl)-6,7-dihydro-5H-cyclopenta[d]pyrimidin-4-amine

The titled compound (15 mg) was synthesized by using the general procedure-3 by using intermediate 8 and boronic acid. 1H NMR, DMSO (400 MHz): 8.27 (bs, 1H), 8.11 (s, 1H), 8.05-8.00 (m, 2H), 7.66-7.64 (m, 2H), 7.54-7.52 (m, 2H), 4.79-4.76 (m, 2H), 3.90 (s, 3H), 3.87 (s, 3H), 3.63 (s, 3H), 2.87-2.83 (m, 2H), 2.79-2.76 (m, 2H), 2.09-2.05 (m, 2H). Mass: 511.25 [M+1].

### Example 66

### 2-(2,5-dimethoxyphenyl)-N-(4-(1-methyl-4-(trifluoromethyl)-1H-imidazol-2-yl)benzyl)-6,7-dihydro-5H-cyclopenta[d]pyrimidin-4-amine

The titled compound (29 mg) was synthesized by using the general procedure-3 by using intermediate 8 and boronic acid. 1H NMR, DMSO (400 MHz): 7.90 (bs, 1H), 7.87-7.85 (m, 1H), 7.84 (s, 1H), 7.69-7.66 (m, 2H), 7.65 (s, 1H), 7.55-7.53 (m, 2H), 7.03-6.97 (m, 1H), 4.79-4.75 (m, 2H), 3.83-3.82 (m, 6H), 3.70 (s, 3H), 2.90-2.87 (m, 2H), 2.79-2.76 (m, 2H), 2.06-2.02 (m, 2H). Mass: 510.24 [M+1].

### Example 67

### 2-(3-chloro-2-fluorophenyl)-N-(4-(1-methyl-4-(trifluoromethyl)-1H-imidazol-2-yl)benzyl)-6,7-dihydro-5H-cyclopenta[d]pyrimidin-4-amine

The titled compound (30 mg) was synthesized by using the general procedure-3 by using intermediate 8 and boronic acid. 1H NMR, DMSO (400 MHz): 7.91 (bs, 1H), 7.82-7.78 (m, 1H), 7.73-7.71 (m, 1H), 7.70-7.59 (m, 3H), 7.48-7.64 (m, 2H), 7.27-7.23 (m, 1H), 4.73-4.71 (m, 2H), 3.72 (s, 3H), 2.84-2.80 (m, 2H), 2.77-2.74 (m, 2H), 2.10-2.02 (m, 2H). Mass: 502.18 [M+1].

### Example 68

### 2-(2,4-dimethoxyphenyl)-N-(4-(1-methyl-4-(trifluoromethyl)-1H-imidazol-2-yl)benzyl)-6,7-dihydro-5H-cyclopenta[d]pyrimidin-4-amine

The titled compound (23 mg) was synthesized by using the general procedure-3 by using intermediate 8 and boronic acid. 1H NMR, DMSO (400 MHz): 7.91 (bs, 1H), 7.86-7.84 (m, 1H), 7.83 (s, 1H), 7.68-7.65 (m, 2H), 7.62 (s, 1H), 7.54-7.52 (m, 2H), 6.99-6.97 (m, 1H), 4.78-4.73 (m, 2H), 3.85-3.84 (m, 6H), 3.71 (s, 3H), 2.92-2.89 (m, 2H), 2.78-2.75 (m, 2H), 2.10-2.06 (m, 2H). Mass: 510.24 [M+1].

### Example 69

### 2-(3,4-dimethoxyphenyl)-N-(4-(1-methyl-4-(trifluoromethyl)-1H-imidazol-2-yl)benzyl)-6,7-dihydro-5H-cyclopenta[d]pyrimidin-4-amine

The titled compound (29 mg) was synthesized by using the general procedure-3 by using intermediate 8 and boronic acid. 1H NMR, DMSO (400 MHz): 7.90 (bs, 1H), 7.87-7.85 (m, 1H), 7.82 (s, 1H), 7.67-7.65 (m, 2H), 7.60 (s, 1H), 7.55-7.52 (m, 2H), 6.98-6.96 (m, 1H), 4.75-4.74 (m, 2H), 3.77 (s, 6H), 3.71 (s, 3H), 2.83-2.81 (m, 2H), 2.75-2.72 (m, 2H), 2.06-2.02 (m, 2H). Mass: 510.23 [M+1].

### Example 70

### N-(4-(1-methyl-4-(trifluoromethyl)-1H-imidazol-2-yl)benzyl)-2-(2-(trifluoromethyl)phenyl)-6,7-dihydro-5H-cyclopenta[d]pyrimidin-4-amine

The titled compound (41 mg) was synthesized by using the general procedure-3 by using intermediate 8 and boronic acid. 1H NMR, DMSO (400 MHz): 7.91 (bs, 1H), 7.77-7.75 (m, 1H), 7.68-7.55 (m, 6H), 4.70-4.69 (m, 2H), 3.71 (s, 3H), 2.83-2.78 (m, 2H), 2.76-2.75 (m, 2H), 2.08-2.05 (m, 2H). Mass: 518.20 [M+1].

### Example 71

### N-(4-(1-methyl-4-(trifluoromethyl)-1H-imidazol-2-yl)benzyl)-2-(4-(trifluoromethyl)phenyl)-6,7-dihydro-5H-cyclopenta[d]pyrimidin-4-amine

The titled compound (36 mg) was synthesized by using the general procedure-3 by using intermediate 8 and boronic acid. 1H NMR, DMSO (400 MHz): 8.43-8.41 (m, 2H), 7.91 (bs, 1H), 7.89-7.87 (m, 2H), 7.80-7.78 (m, 1H), 7.68-7.66 (m, 2H), 7.53-7.51 (m, 2H), 4.80-4.78 (m, 2H), 3.71 (s, 3H), 2.87-2.83 (m, 2H), 2.77-2.75 (m, 2H), 2.09-2.05 (m, 2H). Mass: 518.17 [M+1].

### Example 72

### 3-(4-((4-(1-methyl-4-(trifluoromethyl)-1H-imidazol-2-yl)benzyl)amino)-6,7-dihydro-5H-cyclopenta[d]pyrimidin-2-yl)benzamide

The titled compound (7 mg) was synthesized by using the general procedure-3 by using intermediate 8 and boronic acid. Mass: 493.29 [M+1].

### Example 73

### 2-(3,5-dichlorophenyl)-N-(4-(1-methyl-4-(trifluoromethyl)-1H-imidazol-2-yl)benzyl)-6,7-dihydro-5H-cyclopenta[d]pyrimidin-4-amine

The titled compound (20 mg) was synthesized by using the general procedure-3 by using intermediate 8 and boronic acid. 1H NMR, DMSO (400 MHz): 8.15-8.14 (m, 2H), 7.90 (bs, 1H), 7.84-7.81 (m, 1H), 7.68-7.65 (m, 3H), 7.53-7.51 (m, 2H), 4.76-4.74 (m, 2H), 3.71 (s, 3H), 2.85-2.82 (m, 2H), 2.78-2.75 (m, 2H), 2.08-2.05 (m, 2H). Mass: 518.14 [M+1].

### Example 74

### N-(4-(1-methyl-4-(trifluoromethyl)-1H-imidazol-2-yl)benzyl)-2-(2,3,5-trifluorophenyl)-6,7-dihydro-5H-cyclopenta[d]pyrimidin-4-amine

The titled compound (15 mg) was synthesized by using the general procedure-3 by using intermediate 8 and boronic acid. 1H NMR, DMSO (400 MHz): 7.91 (bs, 1H), 7.81-7.78 (m, 1H), 7.67-7.65 (m, 2H), 7.60-7.58 (m, 1H), 7.48-7.46 (m, 3H), 4.73-4.71 (m, 2H), 3.72 (s, 3H), 2.85-2.81 (m, 2H), 2.78-2.75 (m, 2H), 2.10-2.03 (m, 2H). Mass: 504.21 [M+1].

### Example 75

### 2-(4-cyclopropyl-6-methoxypyrimidin-5-yl)-N-(1-(4-(1-methyl-4-(trifluoromethyl)-1H-imidazol-2-yl)phenyl)ethyl)-7H-purin-6-amine

To a solution of 2-(4-cyclopropyl-6-methoxypyrimidin-5-yl)-N-(1-(4-(1-methyl-4-(trifluoromethyl)-1H-imidazol-2-yl)phenyl)ethyl)-9-(tetrahydro-2H-pyran-2-yl)-9H-purin-6-amine (1.0 eq) was dissolved in DCM (2 ml). To it TFA (30 eq) was added. Reaction mixture was Stirred at RT for 16 h. Reaction mixture was Evaporated under vacuum, crude was poured into Sat. NaHCO₃ solution (10 ml) and extracted with DCM (3×20 ml), combined organic layer and dried over Na₂SO₄ to give 2-(4-cyclopropyl-6-methoxypyrimidin-5-yl)-N-(1-(4-(1-methyl-4-(trifluoromethyl)-1H-imidazol-2-yl)phenyl)ethyl)-7H-purin-6-amine (0.06 g). Mass: 535.26 [M+1]. 1H NMR, DMSO (400 MHz): 13.0 (s, 1H), 8.60 (s, 1H), 8.35 (s, 1H), 8.19 (s, 1H), 7.90 (s, 1H), 7.64-7.65 (m, 4H), 4.7 (bs, 1H), 3.78 (s, 3H), 3.75 (s, 3H), 1.66-1.54 (m, 1H), 1.58-1.56 (m, 3H), 0.94-0.92 (m, 2H), 0.87-0.85 (m, 2H).

### Example 76

### 2-(4-cyclopropyl-6-methoxypyrimidin-5-yl)-N-(4-(pyridin-3-yl)benzyl)-7H-purin-6-amine

To a solution of 2-(4-cyclopropyl-6-methoxypyrimidin-5-yl)-N-(4-(pyridin-3-yl)benzyl)-9-(tetrahydro-2H-pyran-2-yl)-9H-purin-6-amine (1.0 eq) was dissolved in DCM (2 ml). To it TFA (30 eq) was added. Reaction mixture was Stirred at RT for 16 h. Reaction mixture was Evaporated under vacuum, crude was poured into Sat. NaHCO₃ solution (10 ml) and extracted with DCM (3×20 ml), combined organic layer and dried over Na₂SO₄ to give 2-(4-cyclopropyl-6-methoxypyrimidin-5-yl)-N-(4-(pyridin-3-yl)benzyl)-7H-purin-6-amine (0.05 g). Mass: 451.30 [M+1]. 1H NMR, DMSO (400 MHz): 13.0 (s, 1H), 8.90 (s, 1H), 8.60-8.58 (m, 2H), 8.45 (s, 1H), 8.21 (s, 1H), 8.15-8.13 (m, 1H), 7.68-7.66 (m, 2H), 7.57-7.54 (m, 1H), 7.47-7.45 (m, 2H) 4.72 (bs, 2H), 3.81 (s, 3H), 1.72-1.70 (m, 1H), 0.95-0.93 (m, 2H), 0.86-0.84 (m, 2H).

### Example 77

### 2-(4-cyclopropyl-6-methoxypyrimidin-5-yl)-N-(4-(5-fluoropyridin-2-yl)benzyl)-7H-purin-6-amine

To a solution of 2-(4-cyclopropyl-6-methoxypyrimidin-5-yl)-N-(4-(5-fluoropyridin-2-yl)benzyl)-9-(tetrahydro-2H-pyran-2-yl)-9H-purin-6-amine (1.0 eq) was dissolved in DCM (2 ml). To it TFA (30 eq) was added. Reaction mixture was Stirred at RT for 16 h. Reaction mixture was Evaporated under vacuum, crude was poured into Sat. NaHCO₃ solution (10 ml) and extracted with DCM (3×20 ml), combined organic layer and dried over Na₂SO₄ to give 2-(4-cyclopropyl-6-methoxypyrimidin-5-yl)-N-(4-(5-fluoropyridin-2-yl)benzyl)-7H-purin-6-amine (0.06 g). Mass: 469.23 [M+1]. 1H NMR, DMSO (400 MHz): 13.01 (s, 1H), 8.63-8.62 (m, 1H), 8.58 (s, 1H), 8.11 (bs, 1H), 8.00 (s, 1H), 7.99-7.94 (m, 3H), 7.79-7.78 (m, 1H), 7.45-7.43 (m, 2H), 4.73 (bs, 2H), 3.80 (s, 3H), 1.73-1.71 (m, 1H), 0.95-0.94 (m, 2H), 0.76-0.74 (m, 2H).

### PART C: Biological Activity

The biological and/or pharmacological properties of the compounds of this invention may be confirmed by variety of assays. The biological and/or pharmacological assays which can be carried out with the compounds according to the invention and/or their pharmaceutically acceptable salts is exemplified below.

### Test-1: USP1/UAF 1 Assay:

Deubiquitinase activity of USP1/UAF1 was monitored in a fluorometric assay using ubiquitin-rhodamine110 as substrate. Briefly, enzymatic reactions were conducted in an assay buffer composed of 50 mM HEPES, pH 7.8, 0.5 mM EDTA, 100 mM NaCl, 1 mM TCEP, 0.1 mg/mL BSA, and 0.01% Tween-20 that contained 1 nM USP1/UAF1. Compounds were tested at desired concentrations. The plates were incubated for 15 min to attain equilibrium, and then the enzymatic reaction was initiated by dispensing 150 nM Ub-Rho solution. Fluorescence measurement was performed in a high throughput CCD imager, where kinetic measurements of rhodamine fluorescence were acquired using a 480 nm excitation/540 nm emission filter set. The change in fluorescence intensity over a 5-minute reaction period (typically associated with less than 10% substrate conversion) was normalized against no-inhibitor and no-enzyme controls and the resulting percent inhibition data were fitted to a sigmoidal dose response using a four-parameter Hill equation. Data was be analysed and percent inhibition and/or IC50 for each test compound is calculated accordingly.

TABLE 4

| Example No. | Biochemical assay |
|---|---|
| 1 | +++ |
| 2 | ++ |
| 3 | ++++ |
| 4 | +++ |
| 5 | ++++ |
| 6 | ++++ |
| 7 | ++ |
| 8 | − |
| 9 | ++++ |
| 10 | ++++ |
| 11 | ++++ |
| 12 | A+ |
| 13 | B |
| 14 | B |
| 15 | ++++ |
| 16 | +++ |
| 17 | A+ |
| 18 | B+++ |
| 19 | A |
| 20 | ++ |
| 21 | ++ |
| 22 | ++++ |
| 26 | A^{#} |
| 30 | A^{#} |
| 32 | A^{#} |
| 34 | A^{#} |
| 36 | B^{#} |
| 37 | A^{#} |
| 38 | A |
| 39 | A |
| 40 | A |
| 41 | A |
| 42 | A |
| 43 | A |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 55 | A |
| 56 | A |
| 57 | A |
| 58 | A |
| 59 | A |
| 60 | A |
| 61 | A |
| 62 | A |
| 63 | A |
| 64 | B |
| 65 | A |
| 66 | A |
| 67 | A |
| 68 | A |
| 69 | A |
| 70 | A+ |
| 71 | A |
| 72 | A |
| 73 | A |
| 74 | A |

IC50: ++++ represents <50 nM; +++ represents >50 nM to ≤100 nM; ++ represents >100 nM to ≤1000 nM and D represents ≥1000 nM;
% inhibition @1 μM: A+ represents >75%; A represent >50% to ≤75%; B represents >25% to ≤50% and C represents <25%; % inhibition @10 μM: A^{#} represents >75%; B^{#} represent >50% to ≤75%; C^{#} represents >25% to ≤50% and D^{#} represents <25%

Test-2: Cell Viability assay (MTT assay):

Cell viability assay was carried out using 10% FBS supplemented media. Cells were seeded at desired density in 96 well plate and incubated in a humidified CO₂ incubator. After 24 hours of plating, test compounds at desired concentration range were added to the plates and incubated in the CO₂ incubator for 72 hours. Post 72 hrs treatment, 12.5 μL of MTT was added to all wells including DMSO control. MTT treated plates were incubated further for 3 hours and MTT crystals were dissolved using 125 μL of DMSO. Absorbance was read on a BIO-RAD iMark Microplate plate reader at a wavelength of 570 nm. Data was analysed and percent inhibition (IC50) due to the test compound compared to the control was calculated using GraphPad Prism.

### Test-3: Cell Viability Assay (MTT Assay):

Cell viability assay was carried out using 10% FBS supplemented media. Cells were seeded at desired density in 96 well plate and incubated in a humidified CO₂ incubator. After 24 hours of plating, test compounds at desired concentration range were added to the plates and incubated in the CO₂ incubator for 168 hours. Post 168 hrs treatment, 12.5 μL of MTT was added to all wells including DMSO control. MTT treated plates were incubated further for 3 hours and MTT crystals were dissolved using 125 μL of DMSO. Absorbance was read on a BIO-RAD iMark Microplate plate reader at a wavelength of 570 nm. Data was analysed and percent inhibition (IC50) due to the test compound compared to the control was calculated using GraphPad Prism.

TABLE 5
Cell Line Used for MTT Assay: NCI-H1792

| Examples | % Inhibition |
|---|---|
| 1 | C |
| 6 | B |
| 31 | C |
| 32 | B |
| 33 | B |
| 76 | C |
| 77 | B |

A represents >75% to 100%; B represents >50% to ≤75%; C represents >25% to ≤50% and D represents ≤25%, Tested at 10 uM

### Test-4: Cell Viability Assay (MTT Assay):

Cell viability assay was carried out using 10% FBS supplemented media. Cells were seeded at desired density in 96 well plate and incubated in a humidified CO₂ incubator. After 24 hours of plating, test compounds at desired concentration range were added to the plates and incubated in the CO₂ incubator for 336 hours. Post 168 hrs treatment, 12.5 μL of MTT was added to all wells including DMSO control. MTT treated plates were incubated further for 3 hours and MTT crystals were dissolved using 125 μL of DMSO. Absorbance was read on a BIO-RAD iMark Microplate plate reader at a wavelength of 570 nm. Data was analysed and percent inhibition (IC50) due to the test compound compared to the control was calculated using raphPad Prism.

Results: Representative compounds of the invention demonstrated an IC50 value of K<100 nM wherein some compounds have an IC50 of 50 nM with some compounds showing an IC50 of <30 nM.

### Test 5: Cell Viability Assay (CTG Assay):

Growth inhibition assays was carried out using 1000 FB S supplemented media. Cells were seeded at a desired concentration of 1000-6,000 cells/well in a well plate with desired number of wells. Test compounds at a desired concentration range were added after 24 hours. Growth was assessed using the Cell Titer-Glo (CTG, Cell Signaling) for measuring reduction at 7th day after the addition of test compound chemiluminescence values were read by a plate reader. Data was be analysed and percent inhibition and/or IC50 for each test compound is calculated accordingly.

TABLE 6

| Example No. | MD-MBA-436 (IC50) |
|---|---|
| 1 | A |
| 2 | B* |
| 3 | A |
| 4 | D |
| 5 | D |
| 6 | A |
| 10 | A |
| 11 | A# |
| 12 | B* |
| 16 | B* |
| 19 | A* |
| 21 | B* |
| 22 | B |
| 24 | A* |

IC50: A represents <50 nM; B represents >50 nM to ≤100 nM; C represents >100 nM to ≤1000 nM and D represents ≥1000 nM; % inhibition@10 μM: A* represents >75% to 100%; B* represents >50% to ≤75%; % inhibition@10 μM: A* represents >75% to 100%;

### Test-6: Colony Formation Assay:

For the colony formation assay, cells were seeded at low density in 12 or 6-well plates and grown overnight in humidified incubator. The medium was replaced with growth medium containing different concentrations of the test compound. After 48 hrs, media containing fresh compound was replenished and the plates were incubated for an additional 7-8 days to allow for colony formation. Cells were then fixed with methanol and stained with 0.1% crystal. The number of colonies consisting of 50 or more cells was scored. Cells treated with equal volume of DMSO, and saline were used as a control and designated as 100%. Dose-response curves were generated by using GraphPad Prism.

### Test 7: Anti-Tumor Effect of Compounds of the Invention in Female Balb/c Nude Mice Bearing MDA-MB-436 Human Breast Cancer Xenograft

MDA-MB-436 Xenograft was used to test representative compound of the invention where in cells were inoculated in a mice model representing Breast Cancer in different cohorts/group comprising of Control (Vehicle treated, V1), Control (Vehicle treated, V2) and four treatment cohorts/group namely Olaparib (0, 50 mg/kg BID), Compound (A, 15 mg/kg BID), Compound (C, 30 mg/kg BID) and Compound (N, 15 mg/kg BID), with each cohorts/group. Treatment was initiated when maximum tumor growth reached >170 mm³.

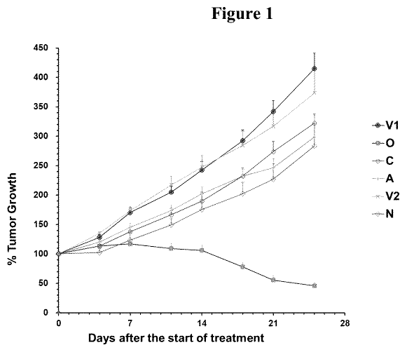

Results: Test compound showed a desired Tumor Growth inhibition (TGI) and was statistically significant (P<0.01\hat{ }, n=4) (see FIG. 1). No significant effect was seen on overall body weight over the duration of study period.

### Test-8: Metabolic Stability

Metabolic stability studies were conducted using mouse, rat, and human liver microsomes. The protocol for the studies with mouse and human liver microsomes (Gibco-Thermo) is provided below.

In brief, 0.25 mg protein was preincubated in phosphate buffer (pH˜7.4) for 5 min at 37° C. and then added with 1 μM test item followed by addition of 1 mM NADPH (cof actor) and incubated further over a period 60 minutes in duplicate. The reaction mixture was terminated with acetonitrile containing an internal standard and centrifuged further to analyse the test item remaining in the supernatant by LC-MS/MS at different intervals. The percent parent compound remaining was calculated in comparison with similar samples terminated at 0 minutes. The results as Ti/2 in minutes are provided in the tables below

TABLE 7
Mouse liver microsomes

| Compound | T_{1/2} |
|---|---|
| ML323 | 7.58 |
| Example 1 | 89.62 |
| Example 3 | 14.42 |
| Example 4 | 7.71 |
| Example 6 | ≥30 |
| Example 10 | 47 |
| Example 11 | 45.5 |
| Example 12 | 40.24 |
| Example 13 | 9.76 |
| Example 16 | ≥230 |
| Example 24 | 22.65 |
| Example 26 | 284.16 |
| Example 27 | 121.59 |
| Example 76 | 10.06 |
| ML52 | 10.95 |

The data shown above in table 7 surprisingly show that the compound of present invention demonstrates significantly greater metabolic stability in mouse liver microsomes as compared to that of reference compound namely ML323 and MHL52 (compound 52 at table 3 in page no. 37 of WO2014/105952).

TABLE 8
Human liver microsomes

| Compound | T_{1/2} |
|---|---|
| ML323 | 30.31 |
| Example 1 | 108.39 |
| Example 2 | 20.43 |
| Example 3 | 14 |
| Example 4 | 7.13 |
| Example 6 | ≥300 |
| Example 9 | 10.50 |
| Example 10 | 134 |
| Example 12 | 9 |
| Example 14 | 3.21 |
| Example 16 | ≥700 |
| Example 21 | 52.67 |
| Example 24 | 227.27 |
| Example 26 | 683.30 |

The data shown in table 8 above surprisingly show that compounds of the present invention demonstrate significantly greater metabolic stability in human liver microsomes over the reference compound ML323.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

1. A compound of Formula (I): 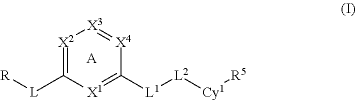
or a tautomer thereof, prodrug thereof, N-oxide thereof, a pharmaceutically acceptable ester thereof, a pharmaceutically acceptable salt thereof, or a stereoisomer thereof,
wherein
X¹ is CR¹ or N;
X² is CR², NR² or N;
X³ is CR³, NR³ or N;
X⁴ is CR⁴, NR⁴ or N;
each occurrence of R¹ is independently selected from hydrogen, hydroxy, halogen, CN (Cyano), substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino;
each occurrence of R², R³ and R⁴ are independently selected from hydrogen, hydroxy, halogen, CN (Cyano), substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino; any two variables of R² and R³ or R³ and R⁴ together with the ring they attached can form a 5 to 8 membered optionally substituted ring, which may optionally include one or more heteroatoms which may be the same or different and are selected from O, NR^{x} or S;
each occurrence of L, L¹ and L² are independently absent or independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₙ—, O, S, —S(═O)ₚ—, —C(═O)—, —NR^{x}—, —S(═O)ₚ—NH—, —NH—S(═O)ₚ—, —CO—NR^{x}— and —NR^{x}—CO—;
Cy¹ is selected from a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl;
each occurrence of R and R⁵ are independently selected from hydrogen, hydroxy, halogen, CN (Cyano), substituted or unsubstituted alkyl, substituted or unsubstituted alkoxy, substituted or unsubstituted haloalkyl, substituted or unsubstituted haloalkenyl, substituted or unsubstituted haloalkynyl, substituted or unsubstituted haloalkoxy, COOH, CONH₂, SO₃H, C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b}, —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring or substituted heterocyclylalkyl ring;
each occurrence of R^{a} is independently selected from hydrogen, hydroxy, halogen, cyano, carboxyl, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted haloalkyl, substituted or unsubstituted haloalkenyl, substituted or unsubstituted haloalkynyl, substituted or unsubstituted haloalkoxy, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{c} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio (═S) or imino (═NR^{x}) group;
each occurrence of R^{x} and R^{z} are independently selected from hydrogen, hydroxy, cyano, halogen, —OR^{a}, —COOR^{a}, —S(═O)q-R^{a}, —NR^{a}R^{b}, —C(═Z′)—R^{a}, substituted or unsubstituted alkyl group, substituted or unsubstituted alkenyl, substituted or unsubstituted alkynyl, and substituted or unsubstituted Cycloalkyl;
each occurrence of
n is 0, 1, 2, 3, 4, 5, 6, 7 or 8; and
p and q are independently 0, 1 or 2;
with the proviso that
(a) when L is absent, and L¹ is —NH— or —O—, then ring A in the compound of formula (I) is not furo[3,2-d]pyrimidine;
(b) when L is absent, L¹ is —NH—, L² is CH₂, Cy¹ is unsubstituted phenyl, X¹═X²═N, X³ ═CR³, and X⁴═CR⁴, wherein R³ and R⁴ together form an unsubstituted phenyl, then
(i) R in the compound of formula (I) is not 2-trifluoromethyl phenyl, and
(ii) R⁵ in the compound of formula (I) is not unsubstituted pyridine-3-yl;
(c) when X¹═X²═N, X³═CR³, X⁴═CR⁴, L is absent, L¹ is —NH—, L² is CH₂, Cy¹ is unsubstituted phenyl, and R⁵ is unsubstituted pyridin-3-yl then R in the compound of formula (I) is not 2-isopropylphenyl; and
(d) the compound of the formula (I) is not
(i) 2-(2-isopropylphenyl)-N-(4-(1-methyl-4-(trifluoromethyl)-1H-imidazol-2-yl)benzyl)pyrido[3,2-d]pyrimidin-4-amine;
(ii) 2-(4-cyclopropyl-6-methoxypyrimidin-5-yl)-4-((4-(1-methyl-4-(trifluoromethyl)-1H-imidazol-2-yl)benzyl)oxy)pyrido[3,2-d]pyrimidine; and
(iii) 2-(2-isopropylphenyl)-N-(4-(pyridin-2-yl)benzyl)-5H-pyrrolo[3,2-d]pyrimidin-4-amine.

2. A compound of Formula (IA) or (IB): 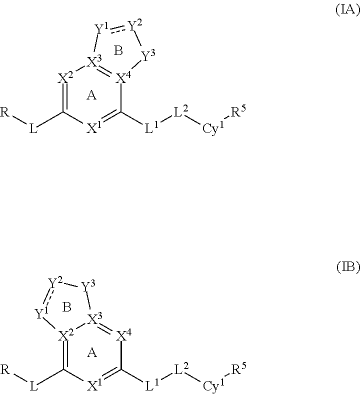
or a tautomer thereof, prodrug thereof, N-oxide thereof, a pharmaceutically acceptable ester thereof, a pharmaceutically acceptable salt thereof, or a stereoisomer thereof,
wherein
Y¹ is —CR^{d}, —NR^{d}, —CR^{d}R^{e}—CR^{d}R^{e}—, —CR^{d}R^{e}—N—, —N—CR^{d}R^{e}—, —NR^{d}—NR^{d}—, —CR^{d}═CR^{d}—, —CR^{d}═N—, —N═CR^{d}—, —N═N—, —O—, —S— or —N—;
Y² is —CR^{d}, —NR^{d}, —CR^{d}R^{e}—CR^{d}R^{e}—, —CR^{d}R^{e}—N—, —N—CR^{d}R^{e}—, —NR^{d}—NR^{e}—, —CR^{d}═CR^{d}—, —CR^{a}═N—, —N═CR^{a}—, —N═N—, —O—, —S— or —N—;
Y³ is —CR^{d}, —NR^{d}, —CR^{d}R^{e}—CR^{d}R^{e}—, —CR^{d}R^{e}—N—, —N—CR^{d}R^{e}—, —NR^{d}—NR^{d}—, —CR^{d}═CR^{d}—, —CR^{d}═N—, —N═CR^{d}—, —N═N—, —O—, —S— or —N—;
each occurrence of R^{d} or R^{e} are independently selected from hydrogen, hydroxy, halogen, oxo, CN (Cyano), substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino; any two variables of R^{d} and R^{e} together with the ring they attached can form an oxo (═O) or a 3 to 8 membered optionally substituted ring, which may optionally include one or more heteroatoms which may be the same or different and are selected from O, NR^{x} or S;
and all the other variables (X¹, X², X³, X⁴, L, L¹, L², R, R⁵, and Cy¹) are independently as defined in claim 1;
with the proviso that
(a) when L is absent, L¹ is —NH— or —O—, then ring A in the compound of formula (IA) is not furo[3,2-d]pyrimidine;
(b) when L is absent, L¹ is —NH—, L² is CH₂, Cy¹ is unsubstituted phenyl, X¹═X²═N, X³ ═CR³, X⁴═CR⁴, wherein R³ and R⁴ together form an unsubstituted phenyl, then
(i) R in the compound of formula (IA) is not 2-trifluoromethyl phenyl, and
(ii) R⁵ in the compound of formula (IA) is not unsubstituted pyridine-3-yl;
(c) when X¹═X²═N, X³═CR³, X⁴═CR⁴, L is absent, L¹ is —NH—, L² is CH₂, Cy¹ is unsubstituted phenyl, R⁵ is unsubstituted pyridin-3-yl then R in the compound of formula (IA) is not 2-isopropylphenyl; and
(d) the compound of the formula (IA) is not
(iv) 2-(2-isopropylphenyl)-N-(4-(1-methyl-4-(trifluoromethyl)-1H-imidazol-2-yl)benzyl)pyrido[3,2-d]pyrimidin-4-amine;
(v) 2-(4-cyclopropyl-6-methoxypyrimidin-5-yl)-4-((4-(1-methyl-4-(trifluoromethyl)-1H-imidazol-2-yl)benzyl)oxy)pyrido[3,2-d]pyrimidine; and
(vi) 2-(2-isopropylphenyl)-N-(4-(pyridin-2-yl)benzyl)-5H-pyrrolo[3,2-d]pyrimidin-4-amine.

3. The compound of claim 2 is selected from a compound of Formula (IA-1), (IA-2), (IB-1) or (IB-2): 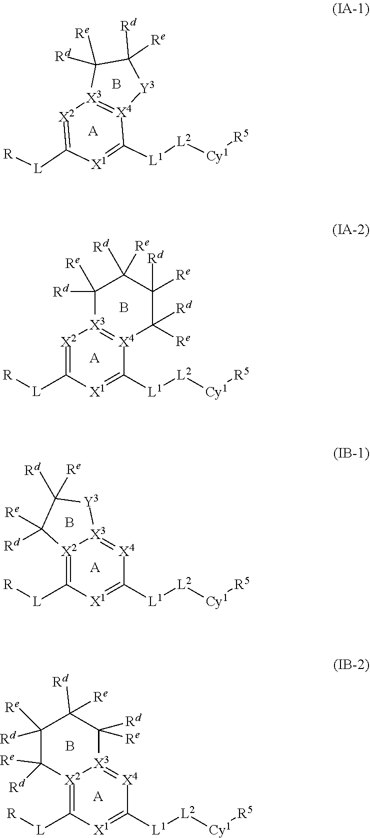
or a tautomer thereof, prodrug thereof, N-oxide thereof, a pharmaceutically acceptable ester thereof, a pharmaceutically acceptable salt thereof, or a stereoisomer thereof,
wherein
all the variables X¹, X², X³, X⁴, Y³, L, L¹, L², R, R⁵, R^{d}, R^{e} and Cy¹ independently are as defined in claim 2.

4. The compound of claim 1, wherein
(i) X¹ is CR¹ or N; X² is CR² or N; X³ is CR³ or N; or X⁴ is CR⁴ or N;
(ii) X¹ is N or CR¹, wherein R¹ is H or CN; X² is N or CR², wherein R² is H or CN; X³ is N or CR³, wherein R³ is H or CN; or X⁴ is N or CR⁴, wherein R⁴ is H or CN; or
(iii) X¹ is N; X² is N; X³ is CH or N; or X⁴ is CH or N.

5. The compound of claim 1, wherein
Y¹ is selected from —CR^{d}, —NR^{d}, —CR^{d}R^{e}—CR^{d}R^{e}—, —CR^{d}R^{e}—N—, —N—CR^{d}R^{e}—, —O—, —S— or —N—;
Y² is selected from —CR^{d}, —NR^{d}, —CR^{d}R^{e}—CR^{d}R^{e}—, CR^{d}R^{e}—N—, —N—CR^{d}R^{e}—, —O—, —S— or —N—;
Y³ is selected from —CR^{d}, —NR^{d}, —CR^{d}R^{e}—CR^{d}R^{e}—, —CR^{d}R^{e}—N—, —N—CR^{d}R^{e}—, —O—, —S— or —N—;
R^{d} is selected from hydrogen, hydroxy, halogen, oxo, CN (Cyano) or substituted or unsubstituted alkyl; and
R^{e} is selected from hydrogen, hydroxy, halogen, oxo, CN (Cyano) or substituted or unsubstituted alkyl.

6. The compound of claim 1, wherein L is
(i) absent or selected from substituted or unsubstituted C₁₋₄ alkyl, O, S, —S(═O)ₚ or NR^{x};
(ii) absent or selected from substituted or unsubstituted C₁₋₄ alkyl or
(iii) absent.

7. The compound of claim 1, wherein ring A including variables X¹, X², X³ and X⁴ is independently selected from

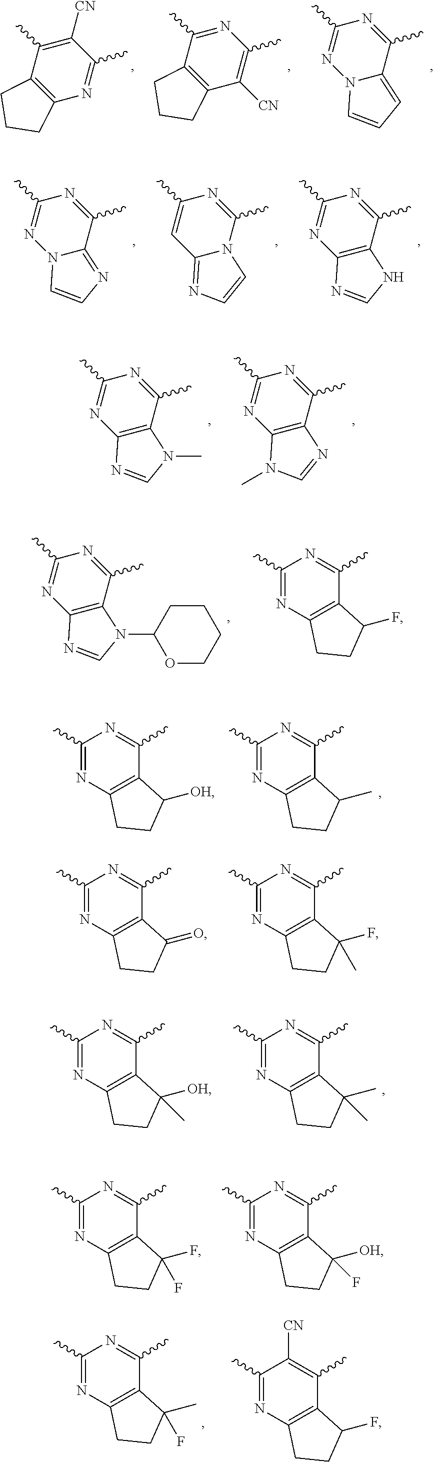

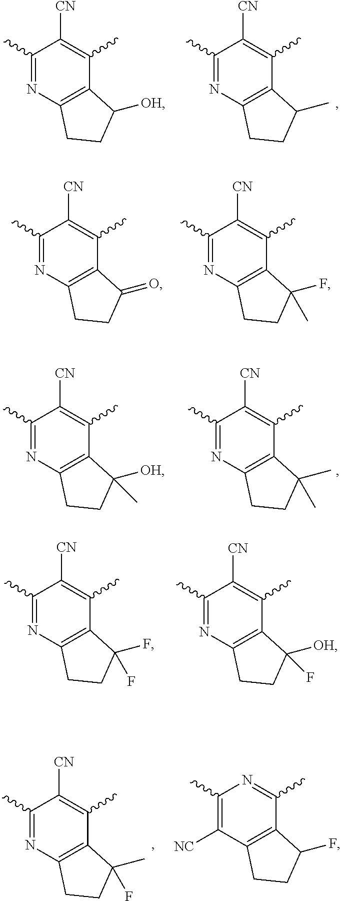

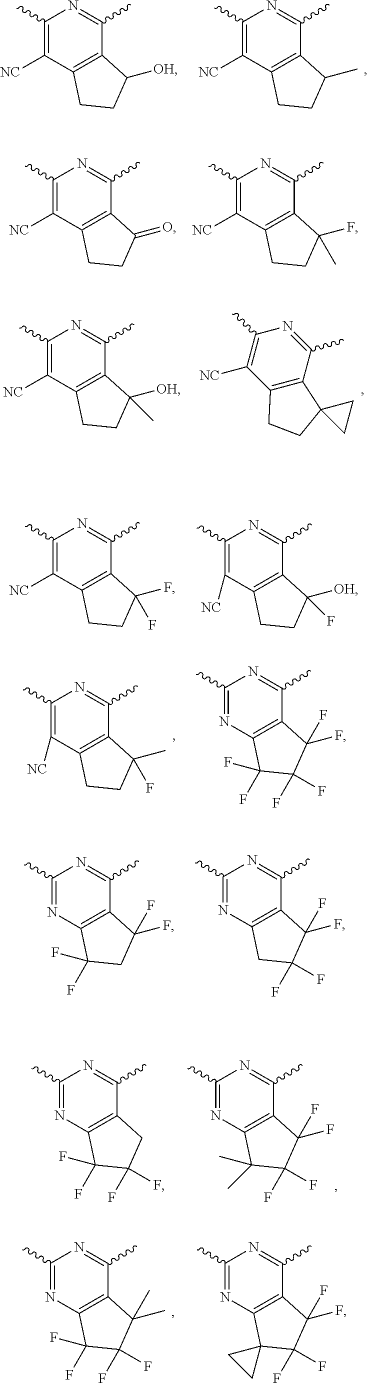

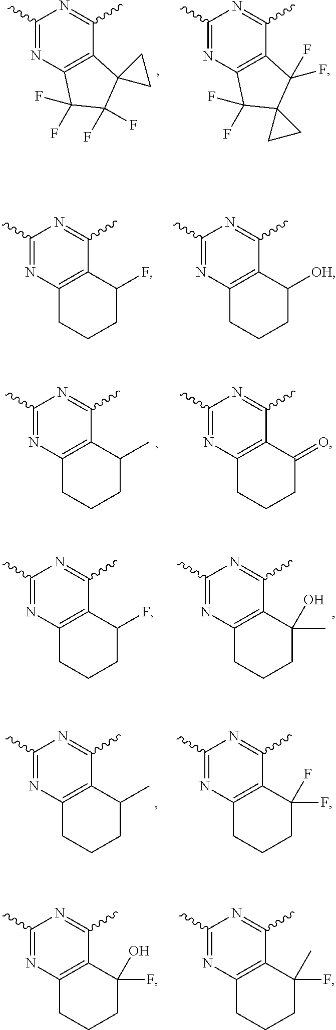

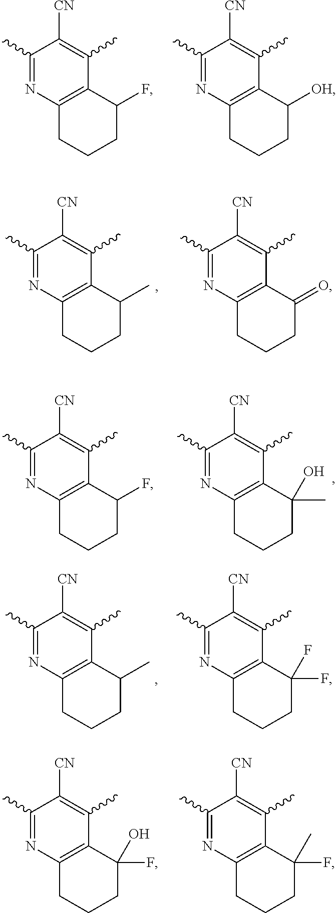

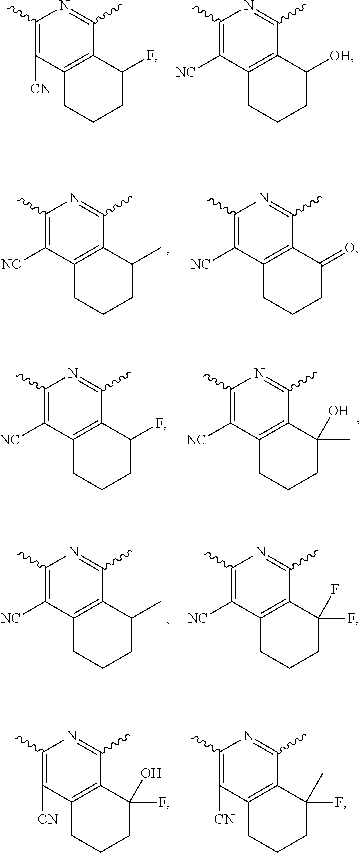

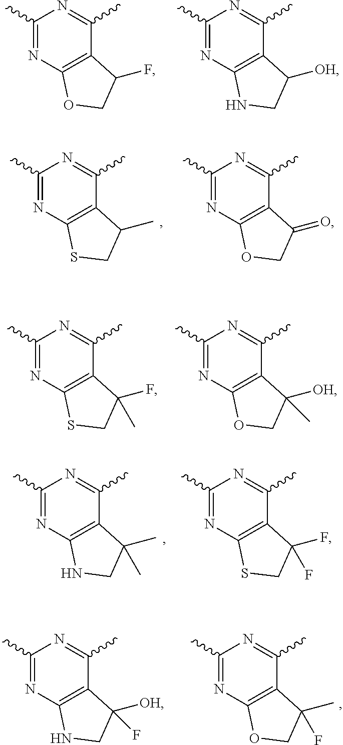

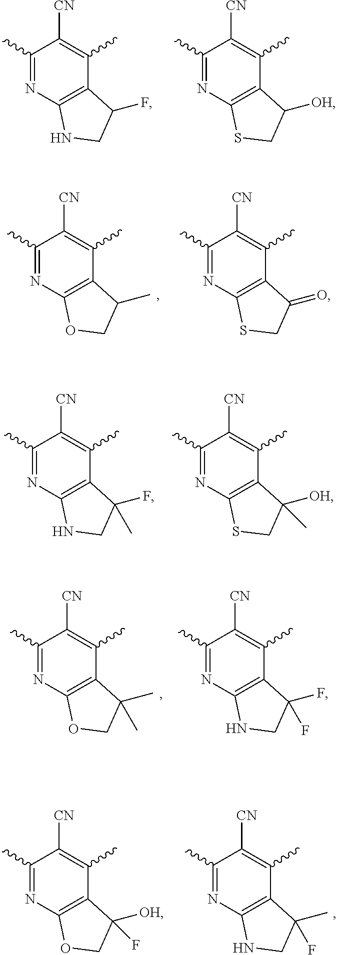

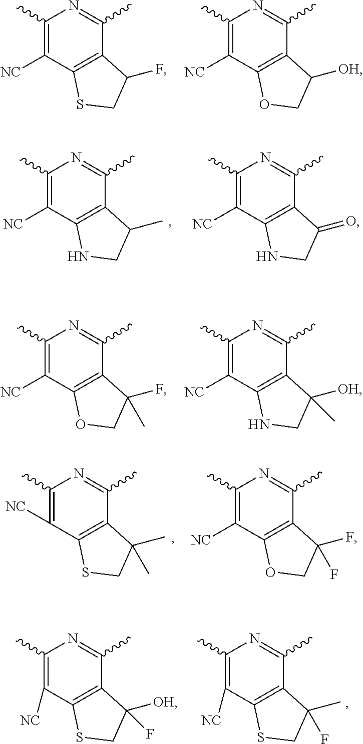

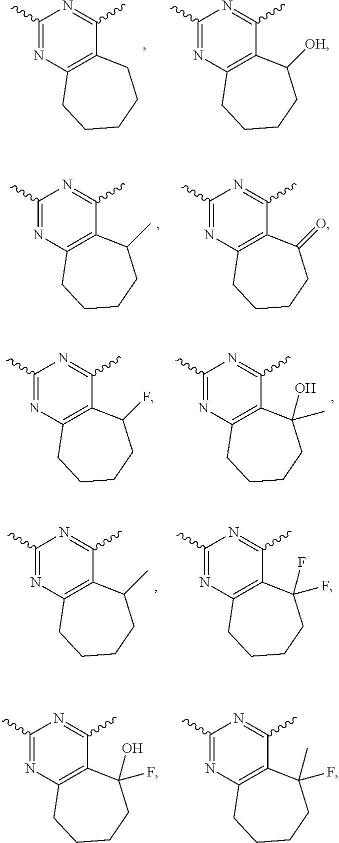

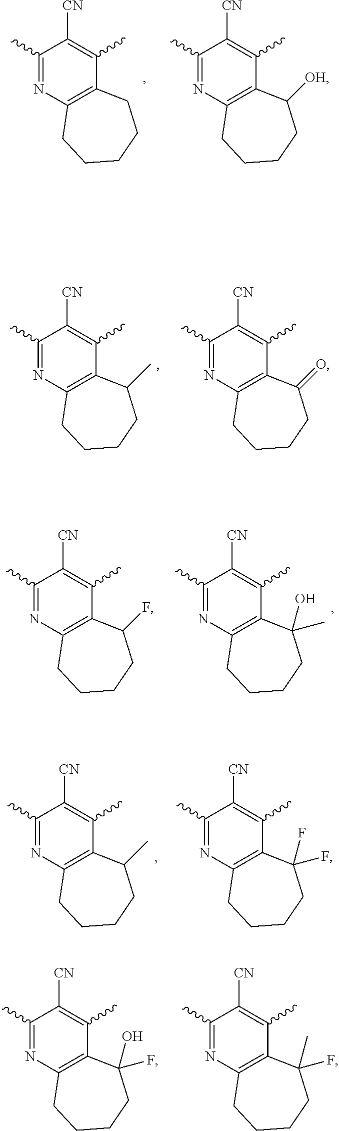

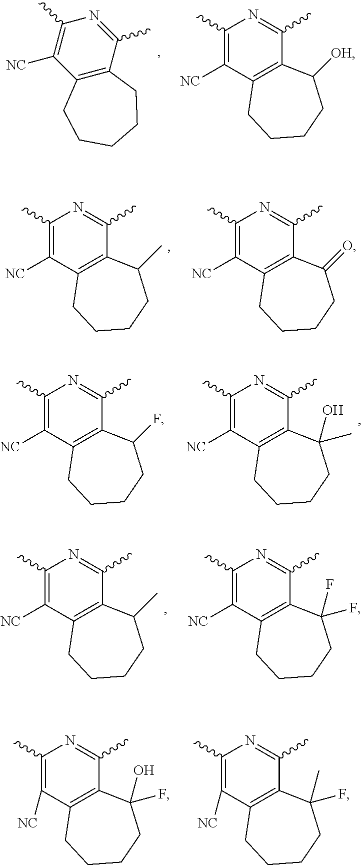

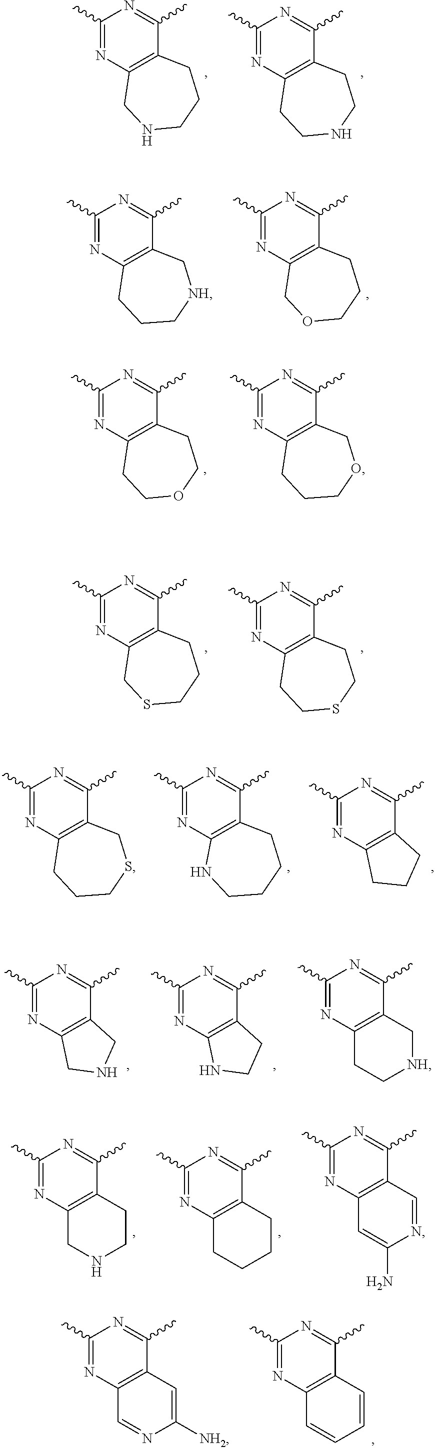

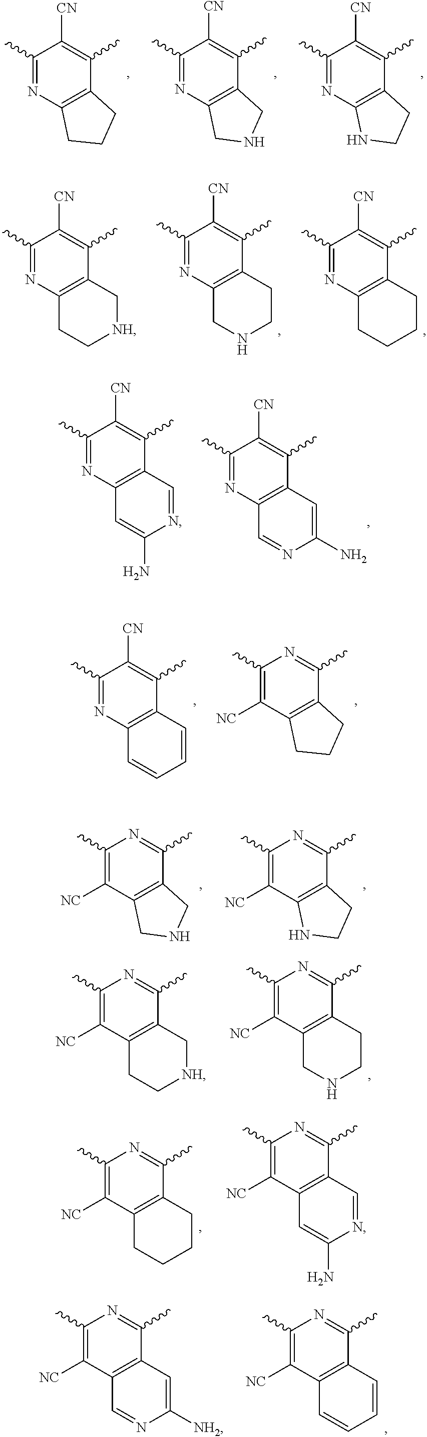

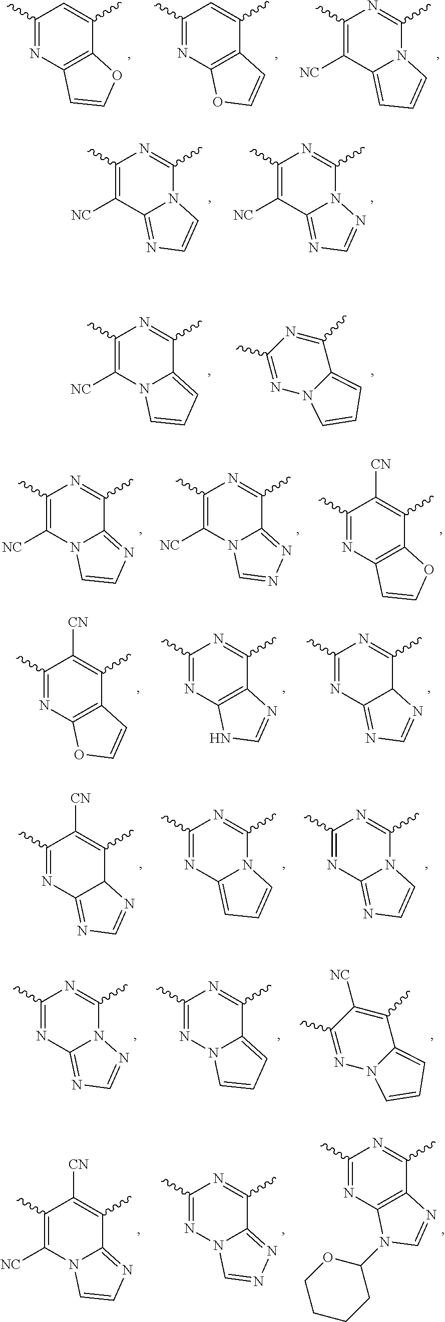

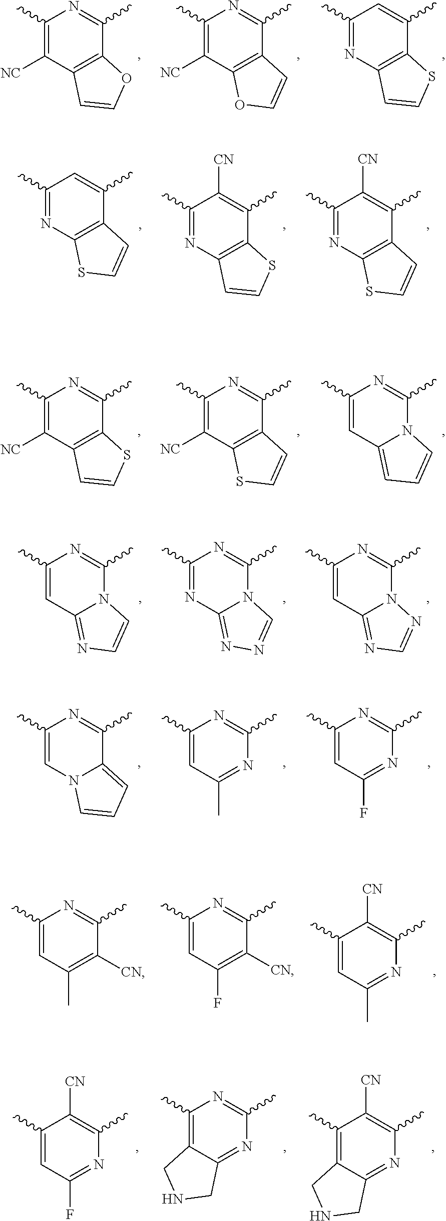

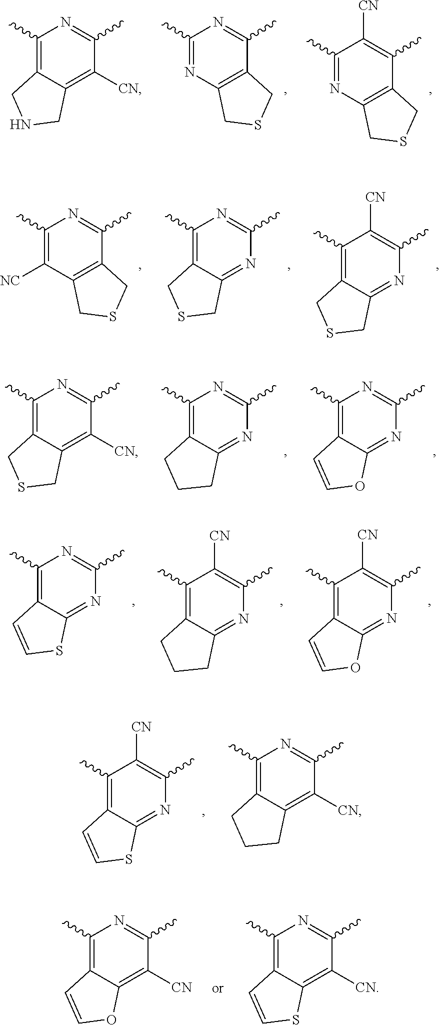

8. The compound of claim 1, wherein ring A including variables X¹, X², X³ and X⁴ is selected from

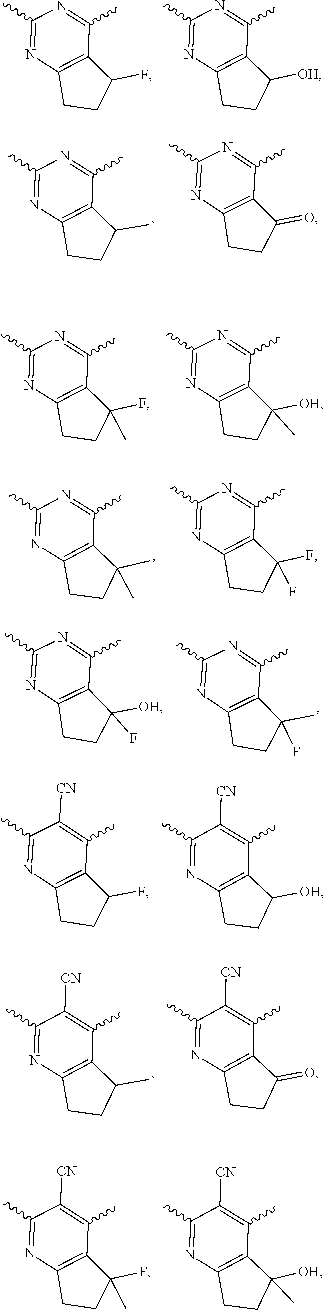

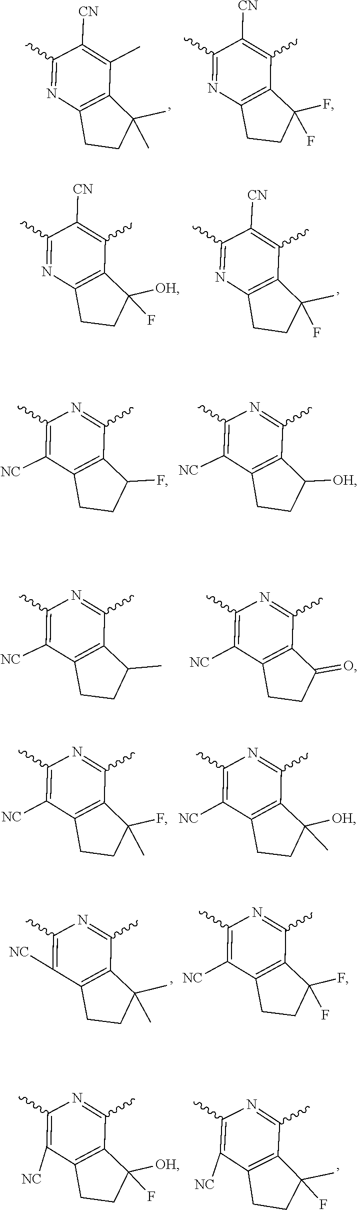

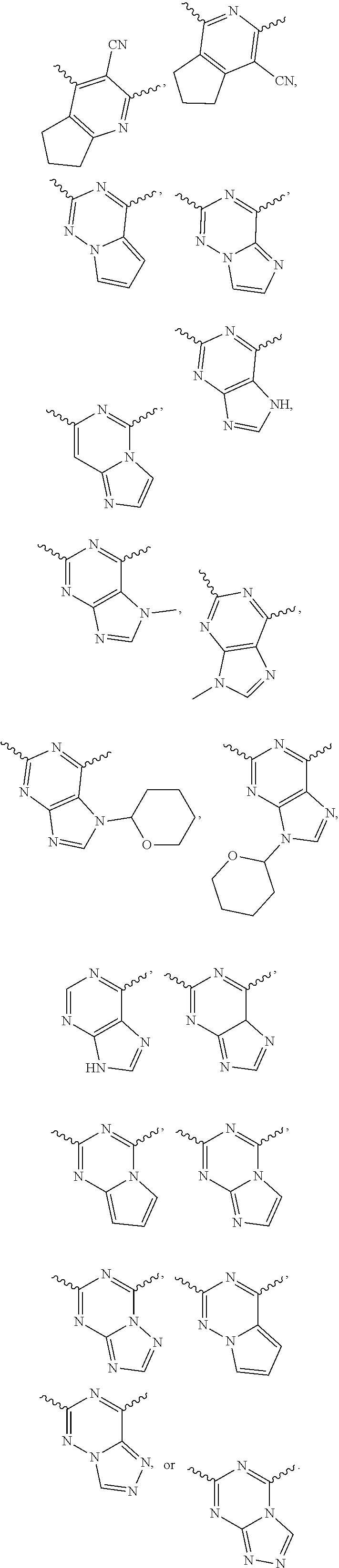

9. The compound of claim 1, wherein ring A including variables X¹, X², X³ and X⁴ is independently selected from

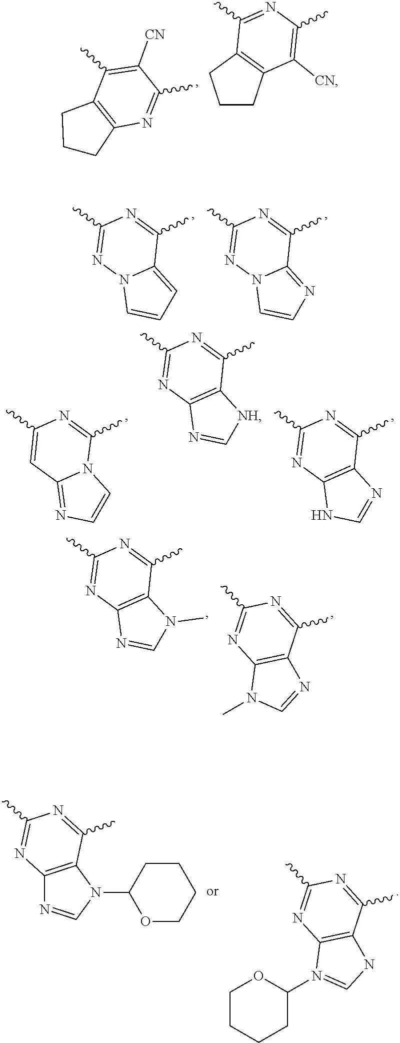

10. The compound of claim 1, wherein R is selected from selected from substituted or unsubstituted aryl, substituted or unsubstituted heteroaryl, substituted or unsubstituted cycloalkyl or substituted or unsubstituted heterocyclic ring.

11. The compound of claim 1, wherein R is selected from
hydrogen, halogen, 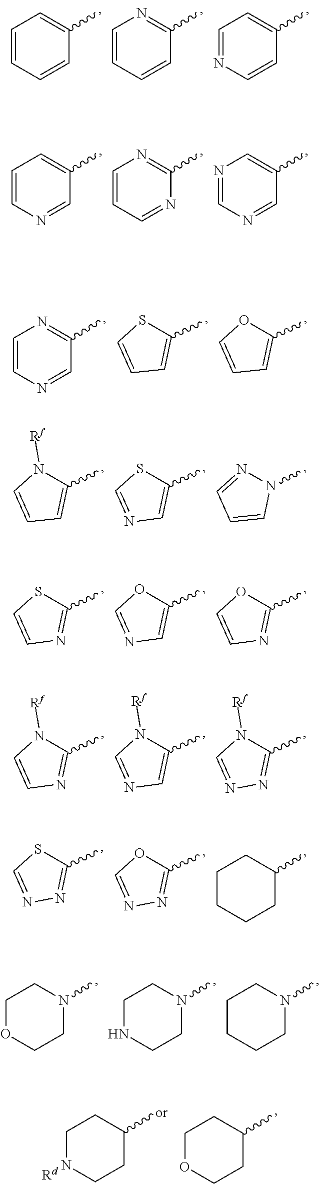
wherein R is optionally substituted with one or more R^{f}; and
each occurrence of R^{f} is independently selected from hydrogen, hydroxy, halogen, CN (Cyano), substituted or unsubstituted alkyl, substituted or unsubstituted alkoxy, substituted or unsubstituted haloalkyl, substituted or unsubstituted haloalkenyl, substituted or unsubstituted haloalkynyl, substituted or unsubstituted haloalkoxy, COOH, CONH₂, SO₃H, C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b}, —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring or substituted heterocyclylalkyl ring.

12. The compound of claim 1, wherein R is selected from
hydrogen, halogen,

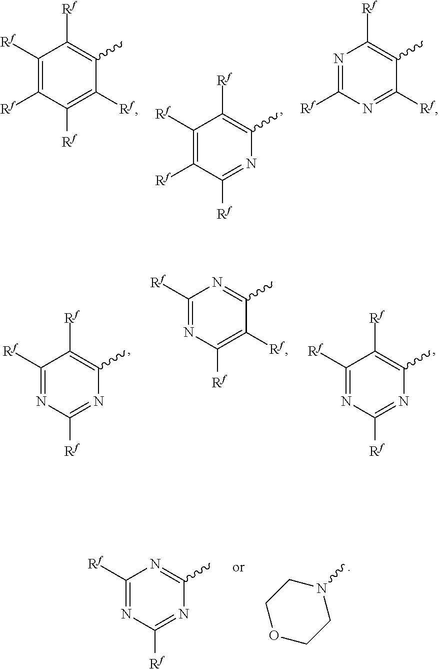

13. The compound of claim 1, wherein R is selected from
hydrogen, fluorine, chlorine,

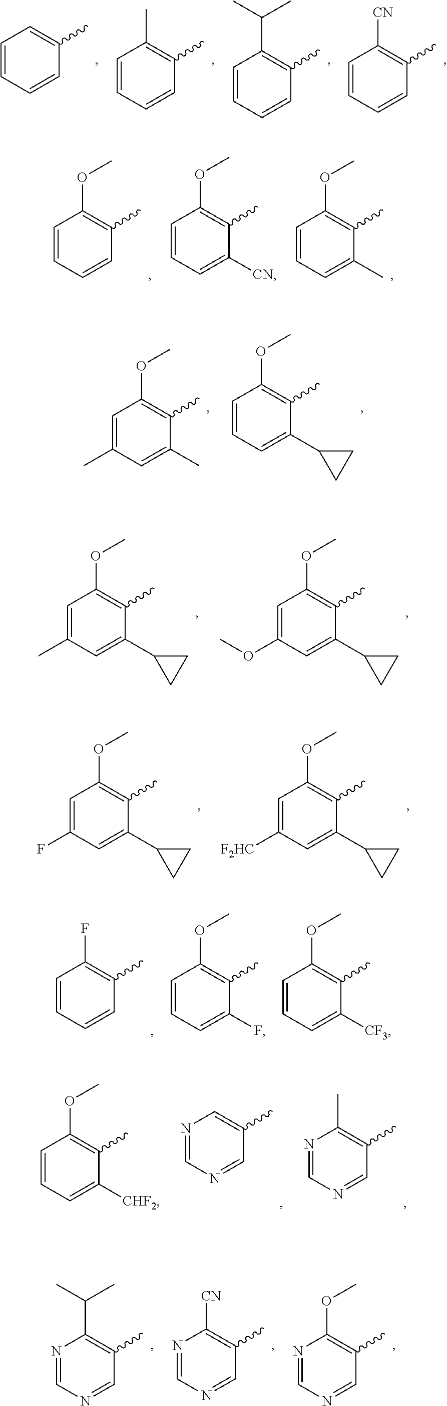

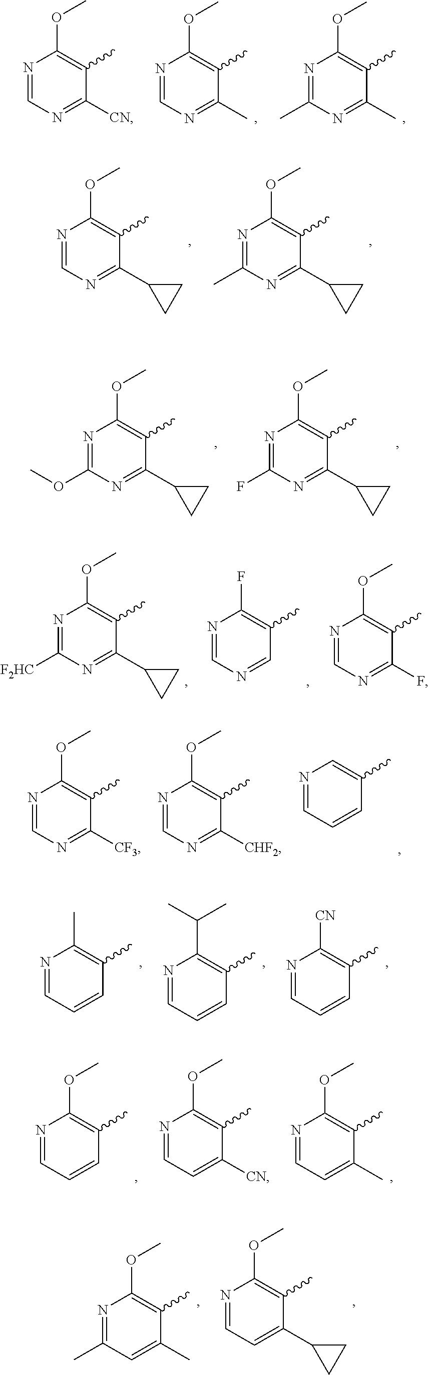

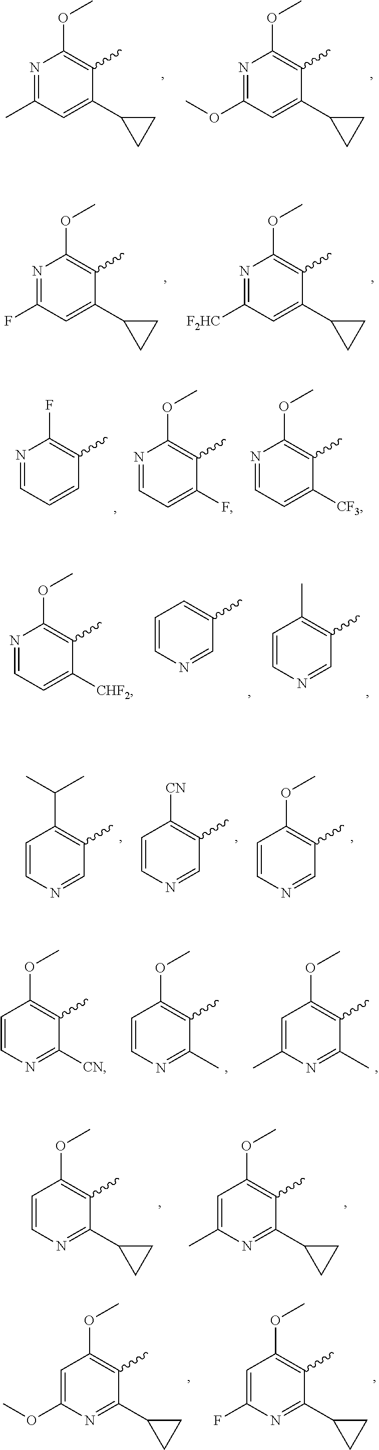

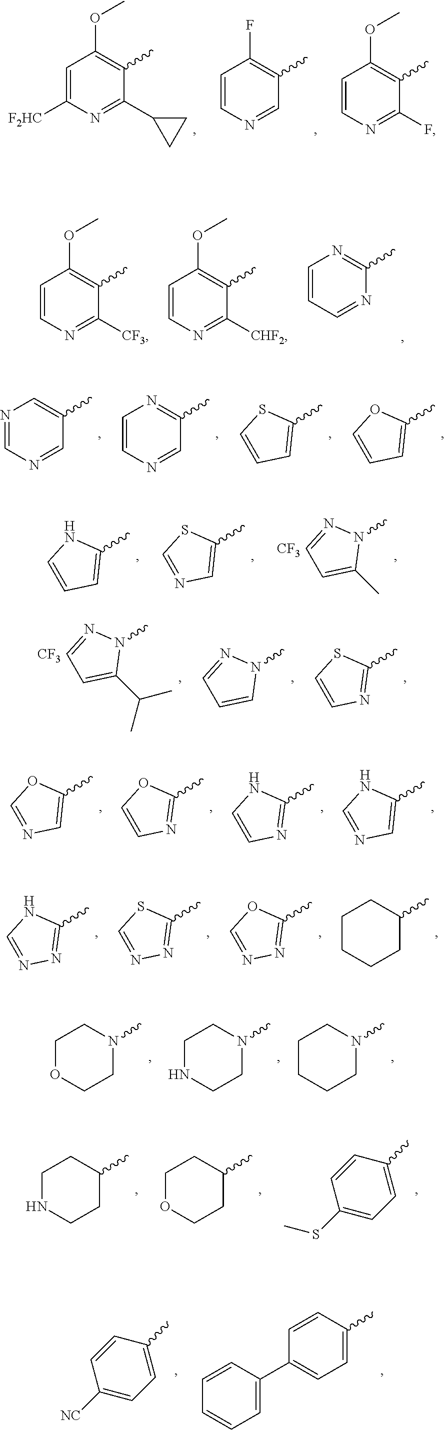

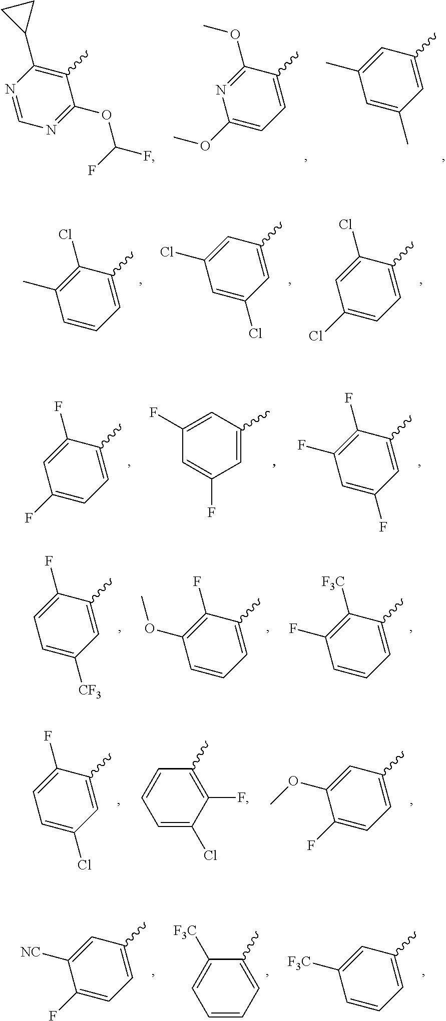

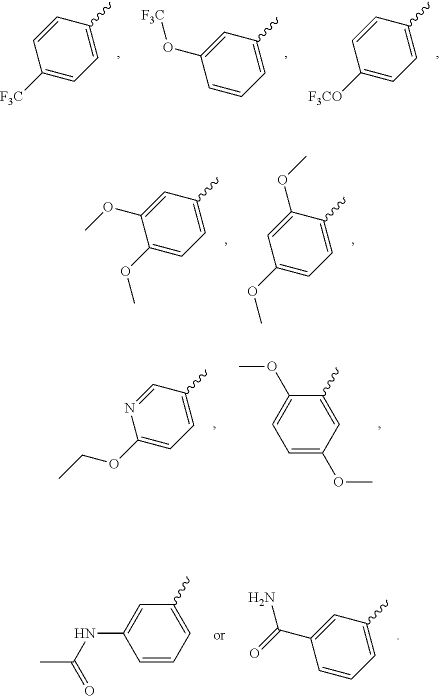

14. The compound of claim 1, wherein R^{f} is selected from hydrogen, hydroxy, halogen, CN (Cyano), substituted or unsubstituted alkyl, substituted or unsubstituted alkoxy, substituted or unsubstituted haloalkyl, substituted or unsubstituted cycloalkyl, COOH, CONH₂, —OR^{b}, —SR^{b}, —SOR^{c} or —SO₂R^{b}.

15. The compound of claim 1, wherein Cy¹ is selected from substituted or unsubstituted aryl, substituted or unsubstituted heteroaryl, substituted or unsubstituted cycloalkyl or substituted or unsubstituted heterocyclic ring.

16. A compound of Formula (IA-1A), or (IA-1B): 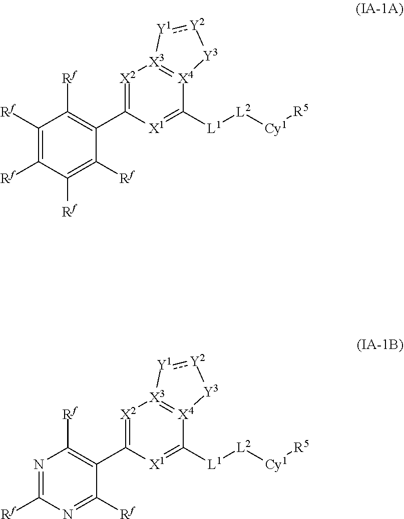
or a tautomer thereof, prodrug thereof, N-oxide thereof, a pharmaceutically acceptable ester thereof, a pharmaceutically acceptable salt thereof, or a stereoisomer thereof,
wherein
Cy¹ is selected from 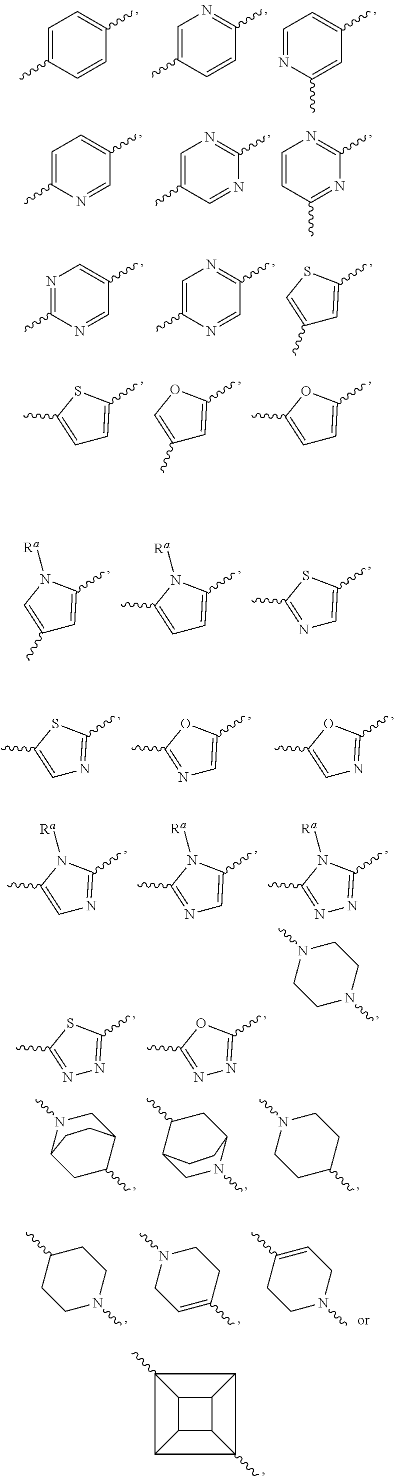
wherein Cy¹ is optionally substituted with one or more R^{a};
X¹ is CR¹ or N;
X² is CR², NR² or N;
X³ is CR³, NR³ or N;
X⁴ is CR⁴, NR⁴ or N;
Y¹ is —CR^{d}, —NR^{d}, —CR^{d}R^{e}—CR^{d}R^{e}—, —CR^{d}R^{e}—N—, —N—CR^{d}R^{e}, —NR^{d}—NR^{d}—, CR^{d}═CR^{d}—, —CR^{d}═N—, —N═CR^{d}—, —N═N—, —O—, —S— or —N—;
Y² is —CR^{d}, —NR^{d}—, —CR^{d}R^{e}—CR^{d}R^{e}—, —CR^{d}R^{e}—N—, —N—CR^{d}R^{e}—, —NR^{d}—NR^{e}—, —CR^{d}═CR^{d}—, —CR^{a}═N—, —N═CR^{a}—, —N═N—, —O—, —S— or —N—;
Y³ is —CR^{d}, —NR^{d}, —CR^{d}R^{e}—CR^{d}R^{e}—, —CR^{d}R^{e}—N—, —N—CR^{d}R^{e}—, —NR^{d}—NR^{d}—, —CR^{d}═CR^{d}—, —CR^{d}═N—, —N═CR^{d}—, —N═N—, —O—, —S— or —N—;
each occurrence of R¹ is independently selected from hydrogen, hydroxy, halogen, CN (Cyano), substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino;
each occurrence of R², R³ and R⁴ are independently selected from hydrogen, hydroxy, halogen, CN (Cyano), substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino; any two variables of R² and R³ or R³ and R⁴ together with the ring they attached can form a 5 to 8 membered optionally substituted ring, which may optionally include one or more heteroatoms which may be the same or different and are selected from O, NR^{x} or S;
each occurrence of L¹ and L² are independently absent or independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₙ—, O, S, —S(═O)—, —C(═O)—, —NR^{x}—, —S(═O)ₚ—NH—, —NH—S(═O)ₚ—, —CO—NR^{x}— and —NR^{x}—CO—;
Cy¹ is selected from a cyclic group selected from substituted or unsubstituted cycloalkyl, substituted or unsubstituted cycloalkenyl, substituted or unsubstituted cycloalkylalkyl, substituted or unsubstituted cycloalkenylalkyl, substituted or unsubstituted heterocyclyl, substituted or unsubstituted heterocyclylalkyl, substituted or unsubstituted aryl, substituted or unsubstituted arylalkyl, substituted or unsubstituted heteroaryl, substituted or unsubstituted heteroarylalkyl;
R⁵ is selected from hydrogen, hydroxy, halogen, CN (cyano), substituted or unsubstituted alkyl, substituted or unsubstituted alkoxy, substituted or unsubstituted haloalkyl, substituted or unsubstituted haloalkenyl, substituted or unsubstituted haloalkynyl, substituted or unsubstituted haloalkoxy, COOH, CONH₂, SO₃H, C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b}, —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring or substituted heterocyclylalkyl ring;
each occurrence of R^{a} is independently selected from hydrogen, hydroxy, halogen, cyano, carboxyl, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted haloalkyl, substituted or unsubstituted haloalkenyl, substituted or unsubstituted haloalkynyl, substituted or unsubstituted haloalkoxy, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{c} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio (═S) or imino (═NR^{x}) group;
each occurrence of R^{d} or R^{c} are independently selected from hydrogen, hydroxy, halogen, oxo, CN (cyano), substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino; any two variables of R^{d} and R^{c} together with the ring they attached can form an oxo (═O) or a 3 to 8 membered optionally substituted ring, which may optionally include one or more heteroatoms which may be the same or different and are selected from O, NR^{x} or S;
each occurrence of R^{f} is independently selected from hydrogen, hydroxy, halogen, CN (cyano), substituted or unsubstituted alkyl, substituted or unsubstituted alkoxy, substituted or unsubstituted haloalkyl, substituted or unsubstituted haloalkenyl, substituted or unsubstituted haloalkynyl, substituted or unsubstituted haloalkoxy, COOH, CONH₂, SO₃H, C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b}, —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring or substituted heterocyclylalkyl ring;
each occurrence of R^{x} and R^{z} are independently selected from hydrogen, hydroxy, cyano, halogen, —OR^{a}, —COOR^{a}, —S(═O)_{q}—R^{a}, —NR^{a}R^{b}, —C(═Z′)—R^{a}, substituted or unsubstituted alkyl group, substituted or unsubstituted alkenyl, substituted or unsubstituted alkynyl, and substituted or unsubstituted cycloalkyl;
each occurrence of
n is 0, 1, 2, 3, 4, 5, 6, 7 or 8; and
p and q are independently 0, 1 or 2:
with the proviso that
(a) when L¹ is —NH— or —O—, then bicyclic ring including X¹, X², X³, X⁴, Y¹, Y², and Y³ in the compound of formula (IA-1A) or (IA-1B) is not furo[3,2-d]pyrimidine;
(b) when L¹ is —NH—, L² is CH₂, Cy¹ is unsubstituted phenyl, X¹═X²═N, X³═CR³, and X⁴═CR⁴, wherein R³ and R⁴ together form an unsubstituted phenyl, then R⁵ is not unsubstituted pyridine-3-yl; and
(c) when X¹═X²═N, X³═CR³, X⁴═CR⁴, L¹ is —NH—, L² is CH₂, Cy¹ is unsubstituted phenyl, and R⁵ is unsubstituted pyridin-3-yl then the R^{f}-substituted phenyl group of the compound of formula (IA-1A) is not 2-isopropylphenyl.

17. A compound of Formula (IA-1A1), (IA-1A2), (IA-1A3), (IA-1A4), (IA-1A4), (IA-1B1), (IA-1B2), (IA-1B3), (IA-1B4) or (IA-1B5): 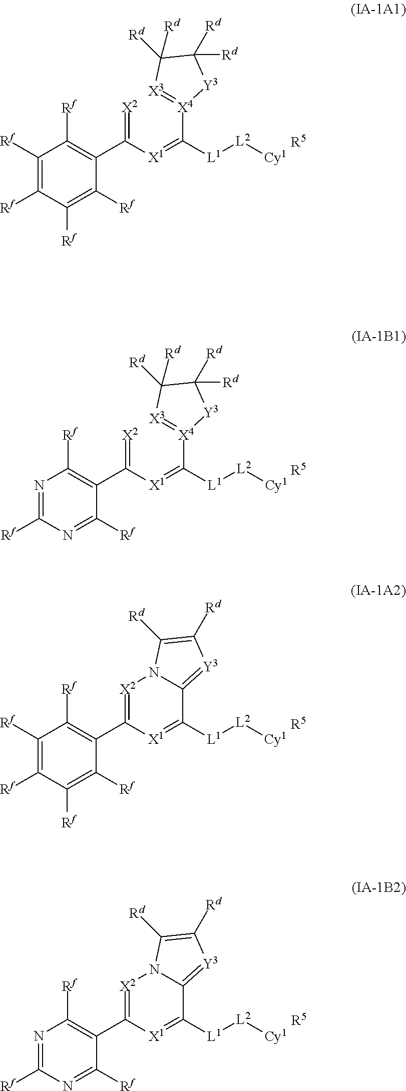 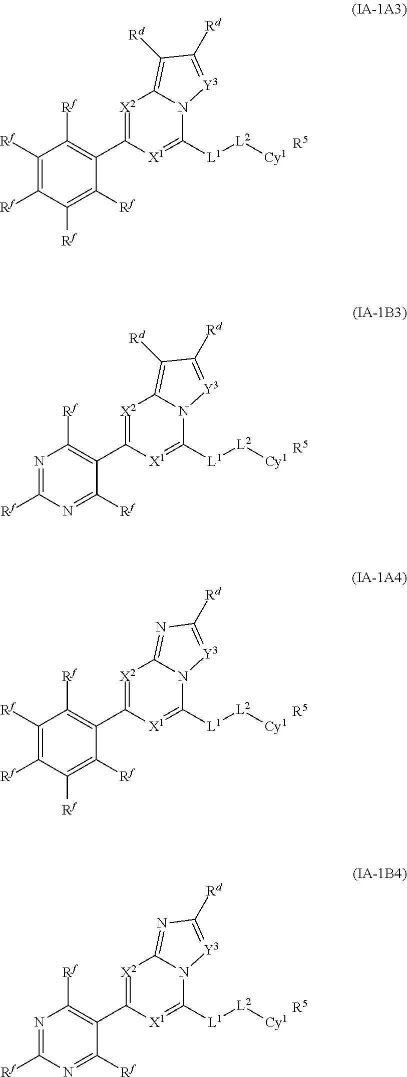 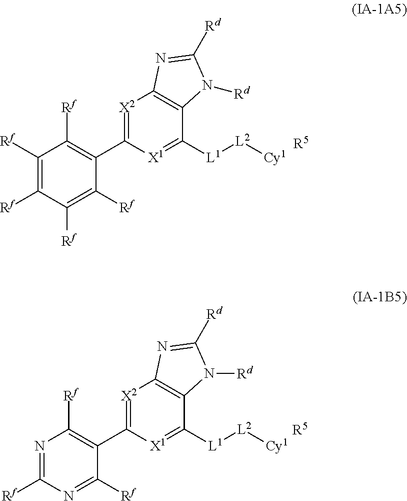
or a tautomer thereof, prodrug thereof, N-oxide thereof, a pharmaceutically acceptable ester thereof, a pharmaceutically acceptable salt thereof, or a stereoisomer thereof,
wherein
Cy¹ is selected from 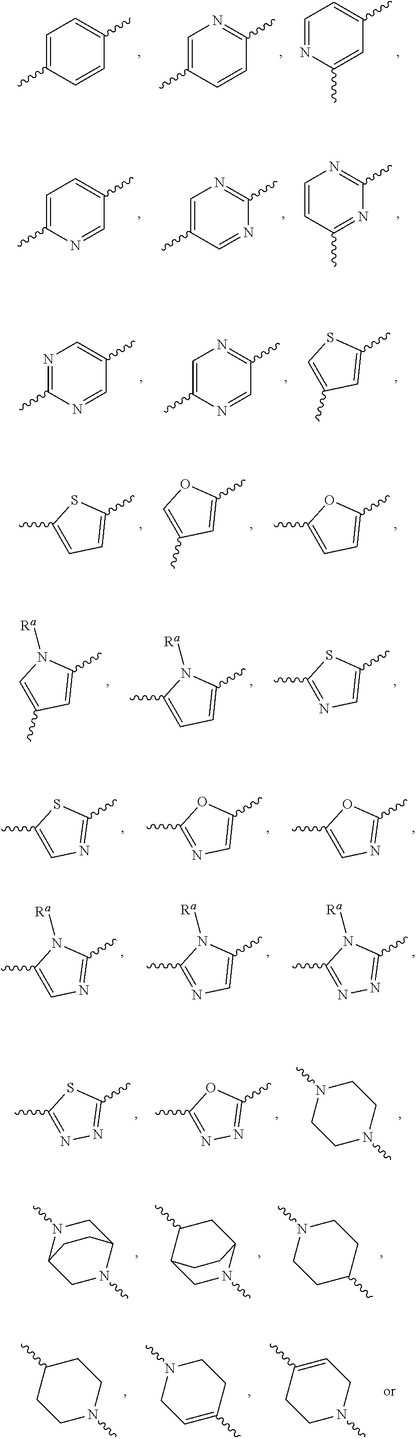 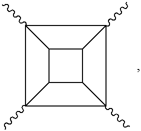
wherein Cy¹ is optionally substituted with one or more R^{a}.
X¹ is CR¹ or N;
X² is CR², NR² or N;
X³ is CR³, NR³ or N;
X⁴ is CR⁴, NR⁴ or N;
Y³ is —CR^{d}, —NR^{d}, —CR^{d}R^{e}—CR^{d}R^{e}—, —CR^{d}R^{e}—N—, —N—CR^{d}R^{e}, —NR^{d}—NR^{d}—, —CR^{d}═CR^{d}═N—, —N═CR^{d}—, —N═N—, —O—, —S— or —N—;
each occurrence of R¹ is independently selected from hydrogen, hydroxy, halogen, CN (Cyano), substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino;
each occurrence of R², R³ and R⁴ are independently selected from hydrogen, hydroxy, halogen, CN (Cyano), substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino; any two variables of R² and R³ or R³ and R⁴ together with the ring they attached can form a 5 to 8 membered optionally substituted ring, which may optionally include one or more heteroatoms which may be the same or different and are selected from O, NR^{x} or S;
each occurrence of L¹ and L² are independently absent or independently selected from substituted or unsubstituted C₁₋₄ alkyl, substituted or unsubstituted C₂₋₄ alkenyl, substituted or unsubstituted C₂₋₄ alkynyl, substituted or unsubstituted C₃₋₁₀ cycloalkyl, substituted or unsubstituted C₃₋₁₀ heterocycloalkyl, —(CR^{b}R^{c})ₙ—, O, S, —S(═O)—, —C(═O)—, —NR^{x}—, —S(═O)ₚ—NH—, —NH—S(═O)ₚ—, —CO—NR^{x}— and —NR^{x}—CO—;
R⁵ is selected from hydrogen, hydroxy, halogen, CN (cyano), substituted or unsubstituted alkyl, substituted or unsubstituted alkoxy, substituted or unsubstituted haloalkyl, substituted or unsubstituted haloalkenyl, substituted or unsubstituted haloalkynyl, substituted or unsubstituted haloalkoxy, COOH, CONH₂, SO₃H, C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(O)OR^{c}, —NR^{b}C(═O)R^{c}, —NR^{b}C(═S)R^{c}, —WC(═S)NR^{b}R^{c}, —SONR^{b}R^{c}, —SO₂NR^{b}R^{c}, —OR, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b}, —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring or substituted heterocyclylalkyl ring;
each occurrence of R^{a} is independently selected from hydrogen, hydroxy, halogen, cyano, carboxyl, substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z};
each occurrence of R^{b} and R^{c} is independently selected from hydrogen, substituted or unsubstituted alkyl, substituted or unsubstituted alkoxy, substituted or unsubstituted alkenyl, substituted or unsubstituted alkynyl, substituted or unsubstituted haloalkyl, substituted or unsubstituted haloalkenyl, substituted or unsubstituted haloalkynyl, substituted or unsubstituted haloalkoxy, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino, or any two of R^{b} and R^{c} when bound to a common atom may be joined to form (i) a substituted or unsubstituted saturated or unsaturated 3-14 membered ring, which may optionally include one or more heteroatoms which may be the same or different and are selected from O, NR^{x} or S, or (ii) an oxo (═O), thio (═S) or imino (═NR^{x}) group;
each occurrence of R^{d} or R^{c} are independently selected from hydrogen, hydroxy, halogen, oxo, CN (cyano), substituted or unsubstituted alkyl, substituted or unsubstituted alkoxy, —C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}—OR^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b} or —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring, substituted heterocyclylalkyl ring, or substituted or unsubstituted amino; any two variables of R^{d} and R^{c} together with the ring they attached can form an oxo (═O) or a 3 to 8 membered optionally substituted ring, which may optionally include one or more heteroatoms which may be the same or different and are selected from O, NR^{x} or S;
each occurrence of R^{f} is independently selected from hydrogen, hydroxy, halogen, CN (cyano), substituted or unsubstituted alkyl, substituted or unsubstituted alkoxy, substituted or unsubstituted haloalkyl, substituted or unsubstituted haloalkenyl, substituted or unsubstituted haloalkynyl, substituted or unsubstituted haloalkoxy, COOH, CONH₂, SO₃H, C(═O)OR^{b}, —C(═O)R^{b}, —C(═S)R^{b}, —C(═O)NR^{b}R^{c}, —C(═O)ONR^{b}R^{c}, —NR^{b}R^{c}, —NR^{b}C(═O)NR^{b}R^{c}, —NR^{b}S(═O)R^{c}, —NR^{b}S(═O)₂R^{c}, —NR^{b}—OR^{c}, ═N—NR^{b}R^{c}, —NR^{b}C(═O)OR^{c}, —NR^{b}C(═O)R^{c}, —NR^{b}C(═S)R^{c}, —NR^{b}C(═S)NR^{b}R^{c}, —SONR^{b}R^{c}, —SO₂NR^{b}R^{c}, —OR^{b}, —OR^{b}C(═O)NR^{b}R^{c}, —OR^{b}C(═O)OR^{c}, —OC(═O)R^{b}, —OC(═O)NR^{b}R^{c}, —R^{b}NR^{c}C(═O)R^{b}, —R^{b}OR^{c}, —R^{b}C(═O)OR^{c}, —R^{b}C(═O)NR^{b}R^{c}, —R^{b}C(═O)R^{c}, —R^{b}OC(═O)R^{c}, —SR^{b}, —SOR^{c}, —SO₂R^{b}, —CR^{b}R^{c}C(═O)R^{b}, —CR^{b}R^{c}C(═S)R^{z}, substituted or unsubstituted aryl, substituted or unsubstituted arylalkyl, substituted or unsubstituted heteroaryl, substituted or unsubstituted heteroarylalkyl, substituted or unsubstituted cycloalkyl, substituted or unsubstituted cycloalkylalkyl, substituted or unsubstituted cycloalkenyl, substituted or unsubstituted heterocyclic ring or substituted heterocyclylalkyl ring;
each occurrence of R^{x} and R^{z} are independently selected from hydrogen, hydroxy, cyano, halogen, —OR^{a}, —COOR^{a}, —S(═O)_{q}—R^{a}, —NR^{a}R^{b}, —C(═Z′)—R^{a}, substituted or unsubstituted alkyl group, substituted or unsubstituted alkenyl, substituted or unsubstituted alkynyl, and substituted or unsubstituted cycloalkyl;
each occurrence of
n is 0, 1, 2, 3, 4, 5, 6, 7 or 8; and
p and q are independently 0, 1 or 2:
with the proviso that when X¹═X²═N, L¹ is —NH—, L² is CH₂, Cy¹ is unsubstituted phenyl, and R⁵ is unsubstituted pyridin-3-yl then the R^{f}-substituted phenyl group of the compound of formula (IA-1A5) or (IA-1B5) is not 2-isopropylphenyl.

18. The compound of claim 1, wherein L¹ is
(i) absent or selected from substituted or unsubstituted C₁₋₄ alkyl, O, S, —S(═O)ₚ or NR^{x};
(ii) —NH— or —O— or
(iii) absent.

19. The compound of claim 1, wherein L² is
(i) selected from substituted or unsubstituted C₁₋₄ alkyl, O, S, —S(═O)ₚ or NR^{x};
(ii) substituted or unsubstituted C₁₋₄ alkyl;
(iii) —(CR^{b}R^{c})n-; wherein each of R^{b} and R^{c} are independently selected form hydrogen, halogen or substituted or unsubstituted alkyl and n is 1-2;
(iv) —(CR^{b}R^{c})ₙ—; wherein each of R^{b}═R^{c}═H, R^{b}═H and R^{c}=methyl and n is 1; or
(v) —CH₂—, —CH(Me)-.

20. The compound of claim 1, wherein R⁵ is selected from substituted or unsubstituted aryl or substituted or unsubstituted heteroaryl.

21. The compound of claim 1, wherein R⁵ is selected from 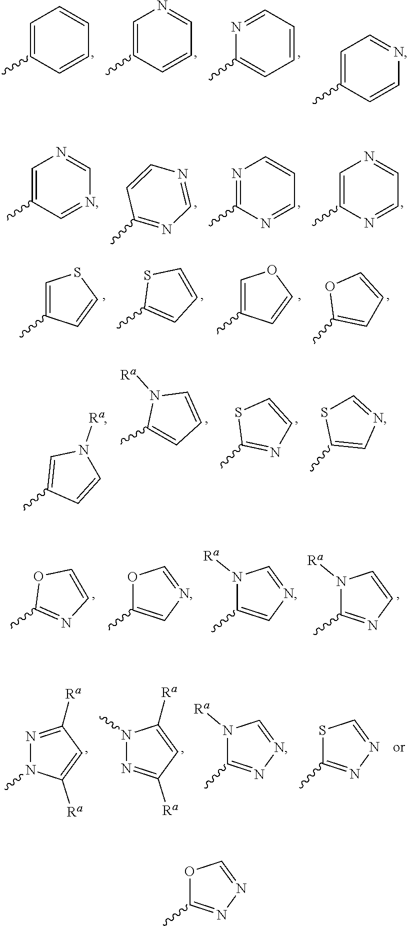
wherein R⁵ is optionally substituted with one or more R^{a}.

22. The compound of claim 1, wherein R⁵ is selected from

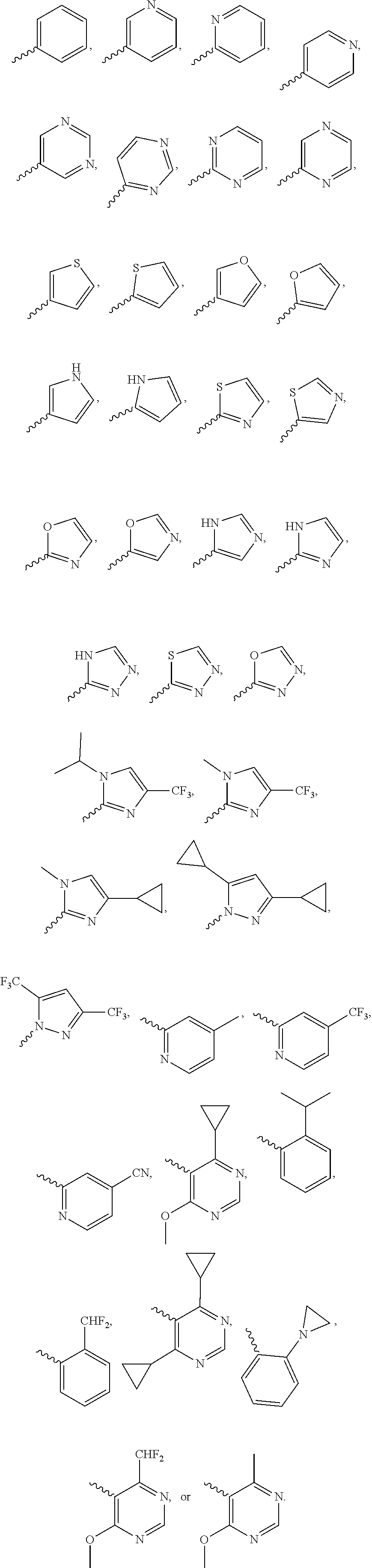

23. A compound selected from
2-(4-cyclopropyl-6-methoxypyrimidin-5-yl)-N-(4-(1-methyl-4-(trifluoromethyl)-1H-imidazol-2-yl)benzyl)-6,7-dihydro-5H-cyclopenta[d]pyrimidin-4-amine;
3-(4-cyclopropyl-6-methoxypyrimidin-5-yl)-1-((4-(1-methyl-4-(trifluoromethyl)-1H-imidazol-2-yl)benzyl)amino)-6,7-dihydro-5H-cyclopenta[c]pyridine-4-carbonitrile;
2-(2-isopropylphenyl)-N-(4-(1-methyl-4-(trifluoromethyl)-1H-imidazol-2-yl)benzyl)-6,7-dihydro-5H-cyclopenta[d]pyrimidin-4-amine;
3-(2-isopropylphenyl)-1-((4-(1-methyl-4-(trifluoromethyl)-1H-imidazol-2-yl)benzyl)amino)-6,7-dihydro-5H-cyclopenta[c]pyridine-4-carbonitrile;
2-(2-isopropylphenyl)-N-(4-(1-methyl-4-(trifluoromethyl)-1H-imidazol-2-yl)benzyl)pyrrolo[2,1-f][1,2,4]triazin-4-amine;
2-(4-cyclopropyl-6-methoxypyrimidin-5-yl)-N-(4-(1-methyl-4-(trifluoromethyl)-1H-imidazol-2-yl)benzyl)pyrrolo[2,1-f][1,2,4]triazin-4-amine;
7-(2-isopropylphenyl)-N-(4-(1-methyl-4-(trifluoromethyl)-1H-imidazol-2-yl)benzyl)imidazo[1,2-c]pyrimidin-5-amine;
7-(4-cyclopropyl-6-methoxypyrimidin-5-yl)-N-(4-(1-methyl-4-(trifluoromethyl)-1H-imidazol-2-yl)benzyl)imidazo[1,2-c]pyrimidin-5-amine;
N-(4-(1-isopropyl-4-(trifluoromethyl)-1H-imidazol-2-yl)benzyl)-2-(2-isopropylphenyl)-6,7-dihydro-5H-cyclopenta[d]pyrimidin-4-amine;
2-(4-cyclopropyl-6-methoxypyrimidin-5-yl)-N-(4-(1-isopropyl-4-(trifluoromethyl)-1H-imidazol-2-yl)benzyl)-6,7-dihydro-5H-cyclopenta[d]pyrimidin-4-amine;
2-(4-cyclopropyl-6-(difluoromethoxy)pyrimidin-5-yl)-N-(4-(1-isopropyl-4-(trifluoromethyl)-1H-imidazol-2-yl)benzyl)-6,7-dihydro-5H-cyclopenta[d]pyrimidin-4-amine;
1-(2-(4-cyclopropyl-6-methoxypyrimidin-5-yl)-6,7-dihydro-5H-cyclopenta[d]pyrimidin-4-yl)-1-(4-(1-methyl-4-(trifluoromethyl)-1H-imidazol-2-yl)benzyl)urea;
4-((4-(1-isopropyl-4-(trifluoromethyl)-1H-imidazol-2-yl)benzyl)oxy)-2-(2-isopropylphenyl)-6,7-dihydro-5H-cyclopenta[d]pyrimidine;
1-(4-(1-isopropyl-4-(trifluoromethyl)-1H-imidazol-2-yl)benzyl)-1-(2-(2-isopropylphenyl)-6,7-dihydro-5H-cyclopenta[d]pyrimidin-4-yl)-3,3-dimethylurea;
2-(2-isopropylphenyl)-N-(4-(1-methyl-4-(trifluoromethyl)-1H-imidazol-2-yl)benzyl)-7H-purin-6-amine;
2-(4-cyclopropyl-6-methoxypyrimidin-5-yl)-N-(4-(1-methyl-4-(trifluoromethyl)-1H-imidazol-2-yl)benzyl)-7H-purin-6-amine;
2-chloro-N-(4-(1-methyl-4-(trifluoromethyl)-1H-imidazol-2-yl)benzyl)-9-(tetrahydro-2H-pyran-2-yl)-9H-purin-6-amine;
2-(2-isopropylphenyl)-N-(4-(1-methyl-4-(trifluoromethyl)-1H-imidazol-2-yl)benzyl)-9-(tetrahydro-2H-pyran-2-yl)-9H-purin-6-amine;
2-(4-cyclopropyl-6-methoxypyrimidin-5-yl)-N-(4-(1-methyl-4-(trifluoromethyl)-1H-imidazol-2-yl)benzyl)-9-(tetrahydro-2H-pyran-2-yl)-9H-purin-6-amine;
2-(2-isopropylphenyl)-4-((4-(1-methyl-4-(trifluoromethyl)-1H-imidazol-2-yl)benzyl)amino)-6,7-dihydro-5H-cyclopenta[b]pyridine-3-carbonitrile;
2-(4-cyclopropyl-6-methoxypyrimidin-5-yl)-4-((4-(1-methyl-4-(trifluoromethyl)-1H-imidazol-2-yl)benzyl)amino)-6,7-dihydro-5H-cyclopenta[b]pyridine-3-carbonitrile;
2-(4-cyclopropyl-6-methoxypyrimidin-5-yl)-N-(4-(1-isopropyl-4-(trifluoromethyl)-1H-imidazol-2-yl)benzyl)pyrrolo[2,1-f][1,2,4]triazin-4-amine;
2-(4-cyclopropyl-6-methoxypyrimidin-5-yl)-4-((4-(1-methyl-4-(trifluoromethyl)-1H-imidazol-2-yl)benzyl)oxy)pyrrolo[2,1-f][1,2,4]triazine;
2-(4-cyclopropyl-6-methoxypyrimidin-5-yl)-4-((4-(1-methyl-4-(trifluoromethyl)-1H-imidazol-2-yl)benzyl)oxy)-6,7-dihydro-5H-cyclopenta[d]pyrimidine;
2-(4-cyclopropyl-6-methoxypyrimidin-5-yl)-6-((4-(1-methyl-4-(trifluoromethyl)-1H-imidazol-2-yl)benzyl)oxy)-9-(tetrahydro-2H-pyran-2-yl)-9H-purine;
2-(4-cyclopropyl-6-methoxypyrimidin-5-yl)-6-((4-(1-methyl-4-(trifluoromethyl)-1H-imidazol-2-yl)benzyl)oxy)-7H-purine;
2-(2-fluoro-6-methoxyphenyl)-6-((4-(1-methyl-4-(trifluoromethyl)-1H-imidazol-2-yl)benzyl)oxy)-9-(tetrahydro-2H-pyran-2-yl)-9H-purine;
2-(2-fluoro-6-methoxyphenyl)-6-((4-(1-methyl-4-(trifluoromethyl)-1H-imidazol-2-yl)benzyl)oxy)-7H-purine;
2-(2-fluoro-6-methoxyphenyl)-N-(4-(1-methyl-4-(trifluoromethyl)-1H-imidazol-2-yl)benzyl)-9-(tetrahydro-2H-pyran-2-yl)-9H-purin-6-amine;
2-(2-fluoro-6-methoxyphenyl)-N-(4-(1-methyl-4-(trifluoromethyl)-1H-imidazol-2-yl)benzyl)-7H-purin-6-amine;
2-(2-methoxypyridin-3-yl)-N-(4-(1-methyl-4-(trifluoromethyl)-1H-imidazol-2-yl)benzyl)-9-(tetrahydro-2H-pyran-2-yl)-9H-purin-6-amine;
2-(2-methoxypyridin-3-yl)-N-(4-(1-methyl-4-(trifluoromethyl)-1H-imidazol-2-yl)benzyl)-7H-purin-6-amine;
2-(4-methoxypyridin-3-yl)-N-(4-(1-methyl-4-(trifluoromethyl)-1H-imidazol-2-yl)benzyl)-9-(tetrahydro-2H-pyran-2-yl)-9H-purin-6-amine;
2-(4-methoxypyridin-3-yl)-N-(4-(1-methyl-4-(trifluoromethyl)-1H-imidazol-2-yl)benzyl)-7H-purin-6-amine;
N-(4-(1-methyl-4-(trifluoromethyl)-1H-imidazol-2-yl)benzyl)-2-morpholino-9-(tetrahydro-2H-pyran-2-yl)-9H-purin-6-amine;
N-(4-(1-methyl-4-(trifluoromethyl)-1H-imidazol-2-yl)benzyl)-2-morpholino-7H-purin-6-amine;
2-(4-cyclopropyl-6-methoxypyrimidin-5-yl)-9-methyl-N-(4-(1-methyl-4-(trifluoromethyl)-1H-imidazol-2-yl)benzyl)-9H-purin-6-amine;
2-(2-chloro-3-methylphenyl)-N-(4-(1-methyl-4-(trifluoromethyl)-1H-imidazol-2-yl)benzyl)-7H-purin-6-amine;
2-(3,5-dichlorophenyl)-N-(4-(1-methyl-4-(trifluoromethyl)-1H-imidazol-2-yl)benzyl)-7H-purin-6-amine;
2-(2,4-difluorophenyl)-N-(4-(1-methyl-4-(trifluoromethyl)-1H-imidazol-2-yl)benzyl)-7H-purin-6-amine;
2-(3,5-difluorophenyl)-N-(4-(1-methyl-4-(trifluoromethyl)-1H-imidazol-2-yl)benzyl)-7H-purin-6-amine;
2-(2,4-dichlorophenyl)-N-(4-(1-methyl-4-(trifluoromethyl)-1H-imidazol-2-yl)benzyl)-7H-purin-6-amine;
2-(2,6-dimethoxypyridin-3-yl)-N-(4-(1-methyl-4-(trifluoromethyl)-1H-imidazol-2-yl)benzyl)-7H-purin-6-amine;
2-(2-fluoro-5-(trifluoromethyl)phenyl)-N-(4-(1-methyl-4-(trifluoromethyl)-1H-imidazol-2-yl)benzyl)-7H-purin-6-amine;
N-(4-(1-methyl-4-(trifluoromethyl)-1H-imidazol-2-yl)benzyl)-2-(2-(trifluoromethyl)phenyl)-7H-purin-6-amine;
N-(4-(1-methyl-4-(trifluoromethyl)-1H-imidazol-2-yl)benzyl)-2-(3-(trifluoromethoxy)phenyl)-7H-purin-6-amine;
N-(4-(1-methyl-4-(trifluoromethyl)-1H-imidazol-2-yl)benzyl)-2-(4-(trifluoromethoxy)phenyl)-7H-purin-6-amine;
2-(2-fluoro-3-methoxyphenyl)-N-(4-(1-methyl-4-(trifluoromethyl)-1H-imidazol-2-yl)benzyl)-7H-purin-6-amine;
2-fluoro-5-(6-((4-(1-methyl-4-(trifluoromethyl)-1H-imidazol-2-yl)benzyl)amino)-7H-purin-2-yl)benzonitrile;
2-(3,5-dimethylphenyl)-N-(4-(1-methyl-4-(trifluoromethyl)-1H-imidazol-2-yl)benzyl)-7H-purin-6-amine;
2-(3,4-dimethoxyphenyl)-N-(4-(1-methyl-4-(trifluoromethyl)-1H-imidazol-2-yl)benzyl)-7H-purin-6-amine;
2-(4-fluoro-3-methoxyphenyl)-N-(4-(1-methyl-4-(trifluoromethyl)-1H-imidazol-2-yl)benzyl)-7H-purin-6-amine;
2-(2,4-dimethoxyphenyl)-N-(4-(1-methyl-4-(trifluoromethyl)-1H-imidazol-2-yl)benzyl)-7H-purin-6-amine;
2-(3-fluoro-2-(trifluoromethyl)phenyl)-N-(4-(1-methyl-4-(trifluoromethyl)-1H-imidazol-2-yl)benzyl)-7H-purin-6-amine;
3-(6-((4-(1-methyl-4-(trifluoromethyl)-1H-imidazol-2-yl)benzyl)amino)-7H-purin-2-yl)benzamide;
N-(4-(1-methyl-4-(trifluoromethyl)-1H-imidazol-2-yl)benzyl)-2-(4-(methylthio)phenyl)-7H-purin-6-amine;
N-(4-(1-methyl-4-(trifluoromethyl)-1H-imidazol-2-yl)benzyl)-2-(4-(trifluoromethyl)phenyl)-7H-purin-6-amine;
N-(4-(1-methyl-4-(trifluoromethyl)-1H-imidazol-2-yl)benzyl)-2-(3-(trifluoromethyl)phenyl)-7H-purin-6-amine;
N-(3-(4-((4-(1-methyl-4-(trifluoromethyl)-1H-imidazol-2-yl)benzyl)amino)-6,7-dihydro-5H-cyclopenta[d]pyrimidin-2-yl)phenyl)acetamide;
2-fluoro-5-(4-((4-(1-methyl-4-(trifluoromethyl)-1H-imidazol-2-yl)benzyl)amino)-6,7-dihydro-5H-cyclopenta[d]pyrimidin-2-yl)benzonitrile;
2-(5-chloro-2-fluorophenyl)-N-(4-(1-methyl-4-(trifluoromethyl)-1H-imidazol-2-yl)benzyl)-6,7-dihydro-5H-cyclopenta[d]pyrimidin-4-amine;
2-(6-ethoxypyridin-3-yl)-N-(4-(1-methyl-4-(trifluoromethyl)-1H-imidazol-2-yl)benzyl)-6,7-dihydro-5H-cyclopenta[d]pyrimidin-4-amine;
4-(4-((4-(1-methyl-4-(trifluoromethyl)-1H-imidazol-2-yl)benzyl)amino)-6,7-dihydro-5H-cyclopenta[d]pyrimidin-2-yl)benzonitrile;
2-([1,1′-biphenyl]-4-yl)-N-(4-(1-methyl-4-(trifluoromethyl)-1H-imidazol-2-yl)benzyl)-6,7-dihydro-5H-cyclopenta[d]pyrimidin-4-amine;
2-(2,6-dimethoxypyridin-3-yl)-N-(4-(1-methyl-4-(trifluoromethyl)-1H-imidazol-2-yl)benzyl)-6,7-dihydro-5H-cyclopenta[d]pyrimidin-4-amine;
2-(2,5-dimethoxyphenyl)-N-(4-(1-methyl-4-(trifluoromethyl)-1H-imidazol-2-yl)benzyl)-6,7-dihydro-5H-cyclopenta[d]pyrimidin-4-amine;
2-(3-chloro-2-fluorophenyl)-N-(4-(1-methyl-4-(trifluoromethyl)-1H-imidazol-2-yl)benzyl)-6,7-dihydro-5H-cyclopenta[d]pyrimidin-4-amine;
2-(2,4-dimethoxyphenyl)-N-(4-(1-methyl-4-(trifluoromethyl)-1H-imidazol-2-yl)benzyl)-6,7-dihydro-5H-cyclopenta[d]pyrimidin-4-amine;
2-(3,4-dimethoxyphenyl)-N-(4-(1-methyl-4-(trifluoromethyl)-1H-imidazol-2-yl)benzyl)-6,7-dihydro-5H-cyclopenta[d]pyrimidin-4-amine;
N-(4-(1-methyl-4-(trifluoromethyl)-1H-imidazol-2-yl)benzyl)-2-(2-(trifluoromethyl)phenyl)-6,7-dihydro-5H-cyclopenta[d]pyrimidin-4-amine;
N-(4-(1-methyl-4-(trifluoromethyl)-1H-imidazol-2-yl)benzyl)-2-(4-(trifluoromethyl)phenyl)-6,7-dihydro-5H-cyclopenta[d]pyrimidin-4-amine;
3-(4-((4-(1-methyl-4-(trifluoromethyl)-1H-imidazol-2-yl)benzyl)amino)-6,7-dihydro-5H-cyclopenta[d]pyrimidin-2-yl)benzamide;
2-(3,5-dichlorophenyl)-N-(4-(1-methyl-4-(trifluoromethyl)-1H-imidazol-2-yl)benzyl)-6,7-dihydro-5H-cyclopenta[d]pyrimidin-4-amine;
N-(4-(1-methyl-4-(trifluoromethyl)-1H-imidazol-2-yl)benzyl)-2-(2,3,5-trifluorophenyl)-6,7-dihydro-5H-cyclopenta[d]pyrimidin-4-amine;
2-(4-cyclopropyl-6-methoxypyrimidin-5-yl)-N-(1-(4-(1-methyl-4-(trifluoromethyl)-1H-imidazol-2-yl)phenyl)ethyl)-7H-purin-6-amine;
2-(4-cyclopropyl-6-methoxypyrimidin-5-yl)-N-(4-(pyridin-3-yl)benzyl)-7H-purin-6-amine; and
2-(4-cyclopropyl-6-methoxypyrimidin-5-yl)-N-(4-(5-fluoropyridin-2-yl)benzyl)-7H-purin-6-amine and pharmaceutically acceptable salts thereof.

24. A compound selected from
2-(4-cyclopropyl-6-methoxypyrimidin-5-yl)-N-(4-(1-methyl-4-(trifluoromethyl)-1H-imidazol-2-yl)benzyl)-6,7-dihydro-5H-cyclopenta[d]pyrimidin-4-amine;
2-(4-cyclopropyl-6-methoxypyrimidin-5-yl)-N-(4-(1-methyl-4-(trifluoromethyl)-1H-imidazol-2-yl)benzyl)pyrrolo[2,1-f][1,2,4]triazin-4-amine;
2-(4-cyclopropyl-6-methoxypyrimidin-5-yl)-N-(4-(1-isopropyl-4-(trifluoromethyl)-1H-imidazol-2-yl)benzyl)-6,7-dihydro-5H-cyclopenta[d]pyrimidin-4-amine;
2-(4-cyclopropyl-6-methoxypyrimidin-5-yl)-N-(4-(1-methyl-4-(trifluoromethyl)-1H-imidazol-2-yl)benzyl)-7H-purin-6-amine;
2-(4-cyclopropyl-6-methoxypyrimidin-5-yl)-N-(4-(1-isopropyl-4-(trifluoromethyl)-1H-imidazol-2-yl)benzyl)pyrrolo[2,1-f][1,2,4]triazin-4-amine;
2-(2-methoxypyridin-3-yl)-N-(4-(1-methyl-4-(trifluoromethyl)-1H-imidazol-2-yl)benzyl)-7H-purin-6-amine;
2-(4-methoxypyridin-3-yl)-N-(4-(1-methyl-4-(trifluoromethyl)-1H-imidazol-2-yl)benzyl)-7H-purin-6-amine; and pharmaceutically acceptable salts thereof.

25. The compound of claim 23 is selected from
2-(4-cyclopropyl-6-methoxypyrimidin-5-yl)-N-(4-(1-methyl-4-(trifluoromethyl)-1H-imidazol-2-yl)benzyl)-6,7-dihydro-5H-cyclopenta[d]pyrimidin-4-amine;
3-(4-cyclopropyl-6-methoxypyrimidin-5-yl)-1-((4-(1-methyl-4-(trifluoromethyl)-1H-imidazol-2-yl)benzyl)amino)-6,7-dihydro-5H-cyclopenta[c]pyridine-4-carbonitrile;
2-(2-isopropylphenyl)-N-(4-(1-methyl-4-(trifluoromethyl)-1H-imidazol-2-yl)benzyl)-6,7-dihydro-5H-cyclopenta[d]pyrimidin-4-amine;
3-(2-isopropylphenyl)-1-((4-(1-methyl-4-(trifluoromethyl)-1H-imidazol-2-yl)benzyl)amino)-6,7-dihydro-5H-cyclopenta[c]pyridine-4-carbonitrile;
N-(4-(1-isopropyl-4-(trifluoromethyl)-1H-imidazol-2-yl)benzyl)-2-(2-isopropylphenyl)-6,7-dihydro-5H-cyclopenta[d]pyrimidin-4-amine;
2-(4-cyclopropyl-6-methoxypyrimidin-5-yl)-N-(4-(1-isopropyl-4-(trifluoromethyl)-1H-imidazol-2-yl)benzyl)-6,7-dihydro-5H-cyclopenta[d]pyrimidin-4-amine;
2-(4-cyclopropyl-6-(difluoromethoxy)pyrimidin-5-yl)-N-(4-(1-isopropyl-4-(trifluoromethyl)-1H-imidazol-2-yl)benzyl)-6,7-dihydro-5H-cyclopenta[d]pyrimidin-4-amine;
1-(2-(4-cyclopropyl-6-methoxypyrimidin-5-yl)-6,7-dihydro-5H-cyclopenta[d]pyrimidin-4-yl)-1-(4-(1-methyl-4-(trifluoromethyl)-1H-imidazol-2-yl)benzyl)urea;
4-((4-(1-isopropyl-4-(trifluoromethyl)-1H-imidazol-2-yl)benzyl)oxy)-2-(2-isopropylphenyl)-6,7-dihydro-5H-cyclopenta[d]pyrimidine;
1-(4-(1-isopropyl-4-(trifluoromethyl)-1H-imidazol-2-yl)benzyl)-1-(2-(2-isopropylphenyl)-6,7-dihydro-5H-cyclopenta[d]pyrimidin-4-yl)-3,3-dimethylurea;
2-(2-isopropylphenyl)-4-((4-(1-methyl-4-(trifluoromethyl)-1H-imidazol-2-yl)benzyl)amino)-6,7-dihydro-5H-cyclopenta[b]pyridine-3-carbonitrile;
2-(4-cyclopropyl-6-methoxypyrimidin-5-yl)-4-((4-(1-methyl-4-(trifluoromethyl)-1H-imidazol-2-yl)benzyl)amino)-6,7-dihydro-5H-cyclopenta[b]pyridine-3-carbonitrile;
2-(4-cyclopropyl-6-methoxypyrimidin-5-yl)-4-((4-(1-methyl-4-(trifluoromethyl)-1H-imidazol-2-yl)benzyl)oxy)-6,7-dihydro-5H-cyclopenta[d]pyrimidine;
N-(3-(4-((4-(1-methyl-4-(trifluoromethyl)-1H-imidazol-2-yl)benzyl)amino)-6,7-dihydro-5H-cyclopenta[d]pyrimidin-2-yl)phenyl)acetamide;
2-fluoro-5-(4-((4-(1-methyl-4-(trifluoromethyl)-1H-imidazol-2-yl)benzyl)amino)-6,7-dihydro-5H-cyclopenta[d]pyrimidin-2-yl)benzonitrile;
2-(5-chloro-2-fluorophenyl)-N-(4-(1-methyl-4-(trifluoromethyl)-1H-imidazol-2-yl)benzyl)-6,7-dihydro-5H-cyclopenta[d]pyrimidin-4-amine;
2-(6-ethoxypyridin-3-yl)-N-(4-(1-methyl-4-(trifluoromethyl)-1H-imidazol-2-yl)benzyl)-6,7-dihydro-5H-cyclopenta[d]pyrimidin-4-amine;
4-(4-((4-(1-methyl-4-(trifluoromethyl)-1H-imidazol-2-yl)benzyl)amino)-6,7-dihydro-5H-cyclopenta[d]pyrimidin-2-yl)benzonitrile;
2-([1,1′-biphenyl]-4-yl)-N-(4-(1-methyl-4-(trifluoromethyl)-1H-imidazol-2-yl)benzyl)-6,7-dihydro-5H-cyclopenta[d]pyrimidin-4-amine;
2-(2,6-dimethoxypyridin-3-yl)-N-(4-(1-methyl-4-(trifluoromethyl)-1H-imidazol-2-yl)benzyl)-6,7-dihydro-5H-cyclopenta[d]pyrimidin-4-amine;
2-(2,5-dimethoxyphenyl)-N-(4-(1-methyl-4-(trifluoromethyl)-1H-imidazol-2-yl)benzyl)-6,7-dihydro-5H-cyclopenta[d]pyrimidin-4-amine;
2-(3-chloro-2-fluorophenyl)-N-(4-(1-methyl-4-(trifluoromethyl)-1H-imidazol-2-yl)benzyl)-6,7-dihydro-5H-cyclopenta[d]pyrimidin-4-amine;
2-(2,4-dimethoxyphenyl)-N-(4-(1-methyl-4-(trifluoromethyl)-1H-imidazol-2-yl)benzyl)-6,7-dihydro-5H-cyclopenta[d]pyrimidin-4-amine;
2-(3,4-dimethoxyphenyl)-N-(4-(1-methyl-4-(trifluoromethyl)-1H-imidazol-2-yl)benzyl)-6,7-dihydro-5H-cyclopenta[d]pyrimidin-4-amine;
N-(4-(1-methyl-4-(trifluoromethyl)-1H-imidazol-2-yl)benzyl)-2-(2-(trifluoromethyl)phenyl)-6,7-dihydro-5H-cyclopenta[d]pyrimidin-4-amine;
N-(4-(1-methyl-4-(trifluoromethyl)-1H-imidazol-2-yl)benzyl)-2-(4-(trifluoromethyl)phenyl)-6,7-dihydro-5H-cyclopenta[d]pyrimidin-4-amine;
3-(4-((4-(1-methyl-4-(trifluoromethyl)-1H-imidazol-2-yl)benzyl)amino)-6,7-dihydro-5H-cyclopenta[d]pyrimidin-2-yl)benzamide;
2-(3,5-dichlorophenyl)-N-(4-(1-methyl-4-(trifluoromethyl)-1H-imidazol-2-yl)benzyl)-6,7-dihydro-5H-cyclopenta[d]pyrimidin-4-amine;
N-(4-(1-methyl-4-(trifluoromethyl)-1H-imidazol-2-yl)benzyl)-2-(2,3,5-trifluorophenyl)-6,7-dihydro-5H-cyclopenta[d]pyrimidin-4-amine; and pharmaceutically acceptable salts thereof.

26. The compound of claim 23 is selected from
2-(2-isopropylphenyl)-N-(4-(1-methyl-4-(trifluoromethyl)-1H-imidazol-2-yl)benzyl)pyrrolo[2,1-f][1,2,4]triazin-4-amine;
2-(4-cyclopropyl-6-methoxypyrimidin-5-yl)-N-(4-(1-methyl-4-(trifluoromethyl)-1H-imidazol-2-yl)benzyl)pyrrolo[2,1-f][1,2,4]triazin-4-amine;
7-(2-isopropylphenyl)-N-(4-(1-methyl-4-(trifluoromethyl)-1H-imidazol-2-yl)benzyl)imidazo[1,2-c]pyrimidin-5-amine;
7-(4-cyclopropyl-6-methoxypyrimidin-5-yl)-N-(4-(1-methyl-4-(trifluoromethyl)-1H-imidazol-2-yl)benzyl)imidazo[1,2-c]pyrimidin-5-amine;
2-(4-cyclopropyl-6-methoxypyrimidin-5-yl)-N-(4-(1-isopropyl-4-(trifluoromethyl)-1H-imidazol-2-yl)benzyl)pyrrolo[2,1-f][1,2,4]triazin-4-amine;
2-(4-cyclopropyl-6-methoxypyrimidin-5-yl)-4-((4-(1-methyl-4-(trifluoromethyl)-1H-imidazol-2-yl)benzyl)oxy)pyrrolo[2,1-f][1,2,4]triazine; and pharmaceutically acceptable salts thereof.

27. The compound of claim 23 is selected from
2-(2-isopropylphenyl)-N-(4-(1-methyl-4-(trifluoromethyl)-1H-imidazol-2-yl)benzyl)-7H-purin-6-amine;
2-(4-cyclopropyl-6-methoxypyrimidin-5-yl)-N-(4-(1-methyl-4-(trifluoromethyl)-1H-imidazol-2-yl)benzyl)-7H-purin-6-amine;
2-chloro-N-(4-(1-methyl-4-(trifluoromethyl)-1H-imidazol-2-yl)benzyl)-9-(tetrahydro-2H-pyran-2-yl)-9H-purin-6-amine;
2-(2-isopropylphenyl)-N-(4-(1-methyl-4-(trifluoromethyl)-1H-imidazol-2-yl)benzyl)-9-(tetrahydro-2H-pyran-2-yl)-9H-purin-6-amine;
2-(4-cyclopropyl-6-methoxypyrimidin-5-yl)-N-(4-(1-methyl-4-(trifluoromethyl)-1H-imidazol-2-yl)benzyl)-9-(tetrahydro-2H-pyran-2-yl)-9H-purin-6-amine;
2-(4-cyclopropyl-6-methoxypyrimidin-5-yl)-6-((4-(1-methyl-4-(trifluoromethyl)-1H-imidazol-2-yl)benzyl)oxy)-9-(tetrahydro-2H-pyran-2-yl)-9H-purine;
2-(4-cyclopropyl-6-methoxypyrimidin-5-yl)-6-((4-(1-methyl-4-(trifluoromethyl)-1H-imidazol-2-yl)benzyl)oxy)-7H-purine;
2-(2-fluoro-6-methoxyphenyl)-6-((4-(1-methyl-4-(trifluoromethyl)-1H-imidazol-2-yl)benzyl)oxy)-9-(tetrahydro-2H-pyran-2-yl)-9H-purine;
2-(2-fluoro-6-methoxyphenyl)-6-((4-(1-methyl-4-(trifluoromethyl)-1H-imidazol-2-yl)benzyl)oxy)-7H-purine;
2-(2-fluoro-6-methoxyphenyl)-N-(4-(1-methyl-4-(trifluoromethyl)-1H-imidazol-2-yl)benzyl)-9-(tetrahydro-2H-pyran-2-yl)-9H-purin-6-amine;
2-(2-fluoro-6-methoxyphenyl)-N-(4-(1-methyl-4-(trifluoromethyl)-1H-imidazol-2-yl)benzyl)-7H-purin-6-amine;
2-(2-methoxypyridin-3-yl)-N-(4-(1-methyl-4-(trifluoromethyl)-1H-imidazol-2-yl)benzyl)-9-(tetrahydro-2H-pyran-2-yl)-9H-purin-6-amine;
2-(2-methoxypyridin-3-yl)-N-(4-(1-methyl-4-(trifluoromethyl)-1H-imidazol-2-yl)benzyl)-7H-purin-6-amine;
2-(4-methoxypyridin-3-yl)-N-(4-(1-methyl-4-(trifluoromethyl)-1H-imidazol-2-yl)benzyl)-9-(tetrahydro-2H-pyran-2-yl)-9H-purin-6-amine;
2-(4-methoxypyridin-3-yl)-N-(4-(1-methyl-4-(trifluoromethyl)-1H-imidazol-2-yl)benzyl)-7H-purin-6-amine;
N-(4-(1-methyl-4-(trifluoromethyl)-1H-imidazol-2-yl)benzyl)-2-morpholino-9-(tetrahydro-2H-pyran-2-yl)-9H-purin-6-amine;
N-(4-(1-methyl-4-(trifluoromethyl)-1H-imidazol-2-yl)benzyl)-2-morpholino-7H-purin-6-amine;
2-(4-cyclopropyl-6-methoxypyrimidin-5-yl)-9-methyl-N-(4-(1-methyl-4-(trifluoromethyl)-1H-imidazol-2-yl)benzyl)-9H-purin-6-amine;
2-(2-chloro-3-methylphenyl)-N-(4-(1-methyl-4-(trifluoromethyl)-1H-imidazol-2-yl)benzyl)-7H-purin-6-amine;
2-(3,5-dichlorophenyl)-N-(4-(1-methyl-4-(trifluoromethyl)-1H-imidazol-2-yl)benzyl)-7H-purin-6-amine;
2-(2,4-difluorophenyl)-N-(4-(1-methyl-4-(trifluoromethyl)-1H-imidazol-2-yl)benzyl)-7H-purin-6-amine;
2-(3,5-difluorophenyl)-N-(4-(1-methyl-4-(trifluoromethyl)-1H-imidazol-2-yl)benzyl)-7H-purin-6-amine;
2-(2,4-dichlorophenyl)-N-(4-(1-methyl-4-(trifluoromethyl)-1H-imidazol-2-yl)benzyl)-7H-purin-6-amine;
2-(2,6-dimethoxypyridin-3-yl)-N-(4-(1-methyl-4-(trifluoromethyl)-1H-imidazol-2-yl)benzyl)-7H-purin-6-amine;
2-(2-fluoro-5-(trifluoromethyl)phenyl)-N-(4-(1-methyl-4-(trifluoromethyl)-1H-imidazol-2-yl)benzyl)-7H-purin-6-amine;
N-(4-(1-methyl-4-(trifluoromethyl)-1H-imidazol-2-yl)benzyl)-2-(2-(trifluoromethyl)phenyl)-7H-purin-6-amine;
N-(4-(1-methyl-4-(trifluoromethyl)-1H-imidazol-2-yl)benzyl)-2-(3-(trifluoromethoxy)phenyl)-7H-purin-6-amine;
N-(4-(1-methyl-4-(trifluoromethyl)-1H-imidazol-2-yl)benzyl)-2-(4-(trifluoromethoxy)phenyl)-7H-purin-6-amine;
2-(2-fluoro-3-methoxyphenyl)-N-(4-(1-methyl-4-(trifluoromethyl)-1H-imidazol-2-yl)benzyl)-7H-purin-6-amine;
2-fluoro-5-(6-((4-(1-methyl-4-(trifluoromethyl)-1H-imidazol-2-yl)benzyl)amino)-7H-purin-2-yl)benzonitrile;
2-(3,5-dimethylphenyl)-N-(4-(1-methyl-4-(trifluoromethyl)-1H-imidazol-2-yl)benzyl)-7H-purin-6-amine;
2-(3,4-dimethoxyphenyl)-N-(4-(1-methyl-4-(trifluoromethyl)-1H-imidazol-2-yl)benzyl)-7H-purin-6-amine;
2-(4-fluoro-3-methoxyphenyl)-N-(4-(1-methyl-4-(trifluoromethyl)-1H-imidazol-2-yl)benzyl)-7H-purin-6-amine;
2-(2,4-dimethoxyphenyl)-N-(4-(1-methyl-4-(trifluoromethyl)-1H-imidazol-2-yl)benzyl)-7H-purin-6-amine;
2-(3-fluoro-2-(trifluoromethyl)phenyl)-N-(4-(1-methyl-4-(trifluoromethyl)-1H-imidazol-2-yl)benzyl)-7H-purin-6-amine;
3-(6-((4-(1-methyl-4-(trifluoromethyl)-1H-imidazol-2-yl)benzyl)amino)-7H-purin-2-yl)benzamide;
N-(4-(1-methyl-4-(trifluoromethyl)-1H-imidazol-2-yl)benzyl)-2-(4-(methylthio)phenyl)-7H-purin-6-amine;
N-(4-(1-methyl-4-(trifluoromethyl)-1H-imidazol-2-yl)benzyl)-2-(4-(trifluoromethyl)phenyl)-7H-purin-6-amine;
N-(4-(1-methyl-4-(trifluoromethyl)-1H-imidazol-2-yl)benzyl)-2-(3-(trifluoromethyl)phenyl)-7H-purin-6-amine;
2-(4-cyclopropyl-6-methoxypyrimidin-5-yl)-N-(1-(4-(1-methyl-4-(trifluoromethyl)-1H-imidazol-2-yl)phenyl)ethyl)-7H-purin-6-amine;
2-(4-cyclopropyl-6-methoxypyrimidin-5-yl)-N-(4-(pyridin-3-yl)benzyl)-7H-purin-6-amine;
2-(4-cyclopropyl-6-methoxypyrimidin-5-yl)-N-(4-(5-fluoropyridin-2-yl)benzyl)-7H-purin-6-amine; and pharmaceutically acceptable salts thereof.

28. A pharmaceutical composition comprising a compound of claim 1 and a pharmaceutically acceptable carrier.

29. A method for treating cancer in a subject in need thereof, the method comprising administering to a subject in need thereof a therapeutically effective amount of a compound of claim 1.

30. The method of claim 29, further comprising the step of administering simultaneously or sequentially to a subject in need thereof at least one other anti-cancer agent, anti-inflammatory agent, immunosuppressive agent, steroid, non-steroidal anti-inflammatory agent, antihistamine, analgesic, or a mixture thereof.

31. A method of treating a USP mediated disorder in a subject in need thereof comprising administering to the subject in need thereof a therapeutically effective amount of a compound of claim 1.

32-33. (canceled)

34. A method of treating a solid cancer with a USP mutation wherein the method comprises of administering to a subject a compound of claim 1.

35. The method of claim 29, wherein cancer is selected from Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

36. The method of claim 29, wherein the cancer is lung cancer, non-small cell lung cancer (NSCLC), colon cancer, breast cancer, bladder cancer, triple negative breast cancer, osteosarcoma, ovarian cancer, skin cancer, prostate cancer, pancreatic cancer, sarcomas, Ewing's sarcoma, hematological cancer such as Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), and Multiple myeloma (“MM”).

37. The method of claim 36, wherein the cancer is (i) colon cancer, (ii) pancreatic cancer (iii) breast cancer (iv) triple negative breast cancer or (v) prostate cancer.
